(19) United States
(12) Patent Application Publication
(10) Pub. No.: US 2015/0376707 A1
(43) Pub. Date: Dec. 31, 2015

(54) METHODS OF DIAGNOSING AND TREATING INFLAMMATORY BOWEL DISEASE

(71) Applicant: Cedars-Sinai Medical Center, Los Angeles, CA (US)

(72) Inventors: Stephan R. Targan, Santa Monica, CA (US); Jerome I. Rotter, Los Angeles, CA (US); Kent D. Taylor, Ventura, CA (US); Marla C. Dubinsky, Los Angeles, CA (US); Dermot P. McGovern, Los Angeles, CA (US); Talin Haritunians, Los Angeles, CA (US); Xiuqing Guo, Santa Monica, CA (US); Rebecca Gonsky, Los Angeles, CA (US); Richard Deem, Azusa, CA (US)

(73) Assignee: CEDARS-SINAI MEDICAL CENTER, Los Angeles, CA (US)

(21) Appl. No.: 14/847,705

(22) Filed: Sep. 8, 2015

Related U.S. Application Data
(63) Continuation-in-part of application No. 12/122,490, filed on May 16, 2008.
Application No. 12/122,490 is a continuation-in-part of application No. 13/144,376, filed on Jul. 22, 2011.
Application No. 13/144,376 is a continuation-in-part of application No. 13/358,424, filed on Jan. 25, 2012.
Application No. 13/358,424 is a continuation-in-part of application No. 13/521,199, filed on Jul. 9, 2012.
Application No. 13/521,199 is a continuation-in-part of application No. 13/521,622, filed on Jul. 11, 2012.
Application No. 13/521,622 is a continuation-in-part of application No. 14/007,381, filed on Sep. 25, 2013.
Application No. 14/007,381 is a continuation-in-part of application No. 14/007,391, filed on Sep. 25, 2013.
Application No. 14/007,391 is a continuation-in-part of application No. 14/538,673, filed on Nov. 11, 2014.
Application No. 14/538,673 is a continuation-in-part of application No. 14/007,400, filed on Sep. 25, 2013.
(60) Provisional application No. 60/938,796, filed on May 18, 2007.
(60) Provisional application No. 60/939,568, filed on May 22, 2007.
(60) Provisional application No. 61/144,290, filed on Jan. 13, 2009.
(60) Provisional application No. 61/436,129, filed on Jan. 25, 2011.
(60) Provisional application No. 61/294,635, filed on Jan. 13, 2010.
(60) Provisional application No. 61/295,309, filed on Jan. 15, 2010.
(60) Provisional application No. 61/467,872, filed on Mar. 25, 2011.
(60) Provisional application No. 61/467,893, filed on Mar. 25, 2011.
(60) Provisional application No. 61/467,779, filed on Mar. 25, 2011.
(60) Provisional application No. 61/467,881, filed on Mar. 25, 2011.
(60) Provisional application No. 61/467,899, filed on Mar. 25, 2011.
(60) Provisional application No. 62/020,899, filed on Jul. 3, 2014.

(51) Int. Cl. C12Q 1/68

(52) U.S. Cl. CPC C12Q 1/6883; C12Q 2600/112; C12Q 2600/154; C12Q 2600/118

(57) ABSTRACT

The present invention also provides various methods, kits and compositions for diagnosing, prognosing, and treating various conditions including but not limited to inflammatory bowel diseases, such as ulcerative colitis and Crohn's disease. Also, the present invention provides various methods, kits and compositions for determining susceptibility to or a low probability of various conditions including but not limited to inflammatory bowel diseases, such as ulcerative colitis and Crohn's disease. These methods, kits and compositions may involve detecting risk/protective variants or haplotypes, serological markers, increased or decreased gene methylation, and increased or decreased cytokine secretion.

### STATEMENT REGARDING FEDERALLY-SPONSORED RESEARCH

This invention was made with government support under Grant Nos. DK043211, DK046763, DK0062248, HL069757, RR00425, RR033176, and TR000124 awarded by the National Institute of Health. The government has certain rights in the invention.

### CROSS-REFERENCE TO RELATED APPLICATIONS

The contents of all the related applications cross-referenced herein are herein incorporated by reference in their entirety as though fully set forth.

This application is a continuation-in-part of U.S. patent application Ser. No. 12/122,490, filed on May 16, 2008, currently pending, which claims priority under 35 U.S.C. §119(e) to U.S. provisional patent application No. 60/938,796, filed on May 18, 2007, and U.S. provisional patent application No. 60/939,568, filed on May 22, 2007.

This application is a continuation-in-part of U.S. patent application Ser. No. 13/144,376, filed on Jul. 22, 2011, currently pending, which is the National Phase of International Application PCT/US10/20921, filed on Jan. 13, 2010, which designated the U.S., was published under PCT Article 21(2) in English, and claims priority under 35 U.S.C. §119(e) to U.S. provisional patent application No. 61/144,290, filed on Jan. 13, 2009.

This application is a continuation-in-part of U.S. patent application Ser. No. 13/521,199, filed on Jul. 9, 2012, currently pending, which is the National Phase of International Application PCT/US11/21180, filed on Jan. 13, 2011, which designated the U.S., was published under PCT Article 21(2) in English, and claims priority under 35 U.S.C. §119(e) to U.S. Provisional Application No. 61/294,635, filed on Jan. 13, 2010.

This application is a continuation-in-part of U.S. patent application Ser. No. 13/521,622, filed on Jul. 11, 2012, currently pending, which is the National Phase of International Application PCT/US11/21382, filed on Jan. 14, 2011, which designated the U.S., was published under PCT Article 21(2) in English, and claims priority under 35 U.S.C. §119(e) to U.S. Provisional Application No. 61/295,309, filed on Jan. 15, 2010.

This application is a continuation-in-part of U.S. patent application Ser. No. 14/007,381, filed on Sep. 25, 2013, currently pending, which is the National Phase of International Application PCT/US12/30611, filed on Mar. 26, 2012, which designated the U.S., was published under PCT Article 21(2) in English, and claims priority under 35 U.S.C. §119(e) to U.S. Provisional Application No. 61/467,872, filed on Mar. 25, 2011, and U.S. Provisional Application No. 61/467,893, filed on Mar. 25, 2011.

This application is a continuation-in-part of U.S. patent application Ser. No. 14/007,391, filed on Sep. 25, 2013, currently pending, which is the National Phase of International Application PCT/US12/30614, filed on Mar. 26, 2012, which designated the U.S., was published under PCT Article 21(2) in English, and claims priority under 35 U.S.C. §119(e) to U.S. Provisional Application No. 61/467,779, filed on Mar. 25, 2011 and U.S. Provisional Application No. 61/467,881, filed on Mar. 25, 2011.

This application is a continuation-in-part of U.S. patent application Ser. No. 14/538,673, filed on Nov. 11, 2014, currently pending, which is a continuation-in-part of U.S. patent application Ser. No. 14/007,400, filed on Sep. 25, 2013, currently pending, which is the National Phase of International Application PCT/US12/30616, filed on Mar. 26, 2012, which designated the U.S., was published under PCT Article 21(2) in English, and claims priority under 35 U.S.C. §119(e) to U.S. Provisional Application No. 61/467,899, filed on Mar. 25, 2011. U.S. patent application Ser. No. 14/538,673, filed on Nov. 11, 2014, currently pending, also claim priority under 35 U.S.C. §119(e) to U.S. Provisional Application No. 62/020,899, filed on Jul. 3, 2014.

This application is a continuation-in-part of U.S. patent application Ser. No. 13/358,424, filed on Jan. 25, 2012, currently pending, which claims priority under 35 U.S.C. §119(e) to U.S. Provisional Application No. 61/436,129, filed on Jan. 25, 2011.

### FIELD OF INVENTION

The invention relates to the field of genetics and medicine. More specifically, the invention relates to methods, kits and compositions for diagnosing, prognosing, and treating various conditions including but not limited to inflammatory bowel diseases, such as ulcerative colitis and Crohn's disease.

### BACKGROUND

All publications, patents, patent application, and literature references cited herein are hereby incorporated by reference in their entirety to the same extent as if each individual publication, patent, patent application, or literature reference was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

Thiopurines have been found to be useful in the treatment of inflammatory bowel disease (IBD), and may be metabolized by methylation by thiopurine methyltransferase (or TPMT). However, variation in thiopurine methyltransferase (TPMT) activity does not fully account for differences in interindividual clinical response to thiopurines in inflammatory bowel disease (IBD). Other genetic and immune biomarkers may also predict therapeutic outcomes with thiopurines.

### SUMMARY OF THE INVENTION

Various embodiments include a method of diagnosing susceptibility to Inflammatory Bowel Disease (IBD) in an individual, comprising obtaining a sample from the individual, assaying the sample to determine the presence or absence of a risk haplotype at the Janus kinase 2 (JAK2) genetic locus and/or SMAD family member 3 (SMAD3) genetic locus, and diagnosing susceptibility to IBD in the individual based on the presence of a risk haplotype at the JAK2 genetic locus and/or SMAD3 genetic locus. In another embodiment, the IBD comprises Crohn's disease. In another embodiment, the risk haplotype at the JAK2 genetic locus comprises JAK2 Block 1 Haplotype 1, JAK2 Block 2 Haplotype 1, and/or JAK2 Block 3 Haplotype 3. In another embodiment, the risk haplotype at the JAK2 genetic locus comprises SEQ ID NO: 1, SEQ ID NO: 2, SEQ ID NO: 3, SEQ ID NO: 4, SEQ ID NO: 5, SEQ ID NO: 6 and/or SEQ ID NO: 7. In another embodiment, the risk haplotype at the SMAD3 genetic locus comprises SMAD3 Block 2 Haplotype 4, SMAD3 Block 5 Haplotype 1 and/or SMAD3 Block 6 Haplotype 1. In another embodiment, the risk haplotype at the SMAD3 genetic locus comprises SEQ ID NO: 8, SEQ ID NO: 9, SEQ ID NO: 10, SEQ ID NO: 11, SEQ ID NO: 12, SEQ ID NO: 13 and/or SEQ ID NO: 14. In another embodiment, the risk haplotype at the SMAD3 genetic locus comprises SEQ ID NO: 15 and/or SEQ ID NO: 16. In another embodiment, the risk haplotype at the SMAD3 genetic locus comprises SEQ ID NO: 17, SEQ ID NO: 18, SEQ ID NO: 19, SEQ ID NO: 20, SEQ ID NO: 21, SEQ ID NO: 22 and/or SEQ ID NO: 23.

Other embodiments include a method of determining a low probability of developing Crohn's disease in an individual, relative to a healthy subject, comprising obtaining a sample from the individual, assaying the sample to determine the presence or absence of a protective haplotype at the Janus kinase 2 (JAK2) genetic locus and/or SMAD family member 3 (SMAD3) genetic locus, and diagnosing a low probability of developing Crohn's disease in the individual, relative to a healthy subject, based upon the presence of the protective haplotype at the JAK2 and/or SMAD3 genetic locus. In another embodiment, the protective haplotype at the JAK2 genetic locus comprises JAK2 Block 1 Haplotype 3, JAK2 Block 2 Haplotype 2, and/or JAK2 Block 3 Haplotype 1. In another embodiment, the protective haplotype at the SMAD3 genetic locus comprises SMAD3 Block 4 Haplotype 1, SMAD3 Block 5 Haplotype 2, and/or SMAD3 Block 6 Haplotype 2.

Other embodiments include a method of diagnosing a Crohn's disease subtype in an individual, comprising determining the presence of one or more risk variants at the janus kinase 2 (JAK2) genetic locus and/or SMAD family member 3 (SMAD3) genetic locus, and diagnosing the Crohn's disease subtype in the individual based upon the presence of the one or more risk variants at the JAK2 and/or SMAD3 genetic locus. In another embodiment, the one or more risk haplotypes at the JAK2 genetic locus comprises SEQ ID NO: 1. In another embodiment, the one or more risk variants at the JAK2 genetic locus comprises JAK2 Block 1 Haplotype 1, JAK2 Block 2 Haplotype 1, and/or JAK2 Block 3 Haplotype 3. In another embodiment, the one or more risk variants at the SMAD3 genetic locus comprises SMAD3 Block 2 Haplotype 4, SMAD3 Block 5 Haplotype 1, and/or SMAD3 Block 6 Haplotype 1.

Other embodiments include a method of treating Crohn's disease in an individual, comprising determining the presence of a risk variant at the janus kinase 2 (JAK2) genetic locus and/or SMAD family member 3 (SMAD3) genetic locus, and treating the individual based upon the presence of the risk variant at the JAK2 genetic locus and/or SMAD3 genetic locus.

Various embodiments include a method of determining the prognosis of Crohn's disease in an individual, comprising determining the presence or absence of one or more risk variants at the janus kinase 2 (JAK2) genetic locus and/or SMAD family member 3 (SMAD3) genetic locus, and prognosing a complicated case of Crohn's disease if the individual demonstrates the presence of one or more risk variants at the JAK3 genetic locus and/or SMAD3 genetic locus. In another embodiment, the one or more risk variants at the JAK2 genetic locus comprises JAK2 Block 1 Haplotype 1, JAK2 Block 2 Haplotype 1, and/or JAK2 Block 3 Haplotype 3. In another embodiment, the one or more risk variants at the SMAD3 genetic locus comprises SMAD3 Block 2 Haplotype 4, SMAD3 Block 5 Haplotype 1, and/or SMAD3 Block 6 Haplotype 1.

Other embodiments include a method of treating Crohn's Disease in an individual, comprising determining the presence of a risk variant at the janus kinase 2 (JAK2) genetic locus in the individual, and treating the individual by inhibiting the JAK2 signaling pathway. In another embodiment, the risk variant at the JAK2 genetic locus comprises SEQ ID NO: 1.

Other features and advantages of the invention will become apparent from the following detailed description, taken in conjunction with the accompanying drawings, which illustrate, by way of example, various embodiments of the invention.

In one embodiment, the invention provides a method of diagnosing susceptibility to Crohn's disease in an individual, comprising: obtaining a sample from the individual, assaying the sample to determine the presence or absence of a risk variant at the ZNF365 genetic locus, and diagnosing susceptibility to Crohn's disease in the individual based on the presence of the risk variant at the ZNF365 genetic locus. The risk variant can be selected from the group consisting of rs10740085, rs12768538, rs7068361, rs7071642, rs7076156, rs729739, rs10995271, rs12766391, rs10761659, and rs224120. Assaying of the sample comprises genotyping for one or more single nucleotide polymorphisms. The sample can be whole blood, plasma, serum, saliva, cheek swab, urine, or stool.

In another embodiment, the invention provides a method of determining a low probability of developing Crohn's disease in an individual, relative to a healthy subject, comprising: obtaining a sample from the individual, assaying the sample to determine the presence or absence of a protective variant at the ZNF365 genetic locus, and diagnosing a low probability of developing Crohn's disease in the individual, relative to a healthy subject, based upon the presence of the protective variant at the ZNF365 genetic locus. The risk variant can be selected from the group consisting of rs10740085, rs12768538, rs7068361, rs7071642, rs7076156, rs729739, rs10995271, rs12766391, rs10761659, and rs224120. Assaying of the sample comprises genotyping for one or more single nucleotide polymorphisms. The sample can be whole blood, plasma, serum, saliva, cheek swab, urine, or stool.

In a related embodiment, the invention provides a method of prognosing Crohn's disease in an individual, comprising: obtaining a sample from the individual, assaying the sample for the presence or absence of one or more genetic risk variants, and prognosing an aggressive form of Crohn's disease based on the presence of one or more risk variants at the ZNF365 genetic locus. The risk variant can be selected from the group consisting of rs10740085, rs12768538, rs7068361, rs7071642, rs7076156, rs729739, rs10995271, rs12766391, rs10761659, and rs224120. Assaying of the sample comprises genotyping for one or more single nucleotide polymorphisms. The sample can be whole blood, plasma, serum, saliva, cheek swab, urine, or stool.

In a further embodiment, the invention provides method of treating an individual for Crohn's disease, comprising: prognosing an aggressive form of Crohn's disease in the individual based on the presence of one or more risk variants at the ZNF365 genetic locus, and treating the individual, wherein the one or more risk variants are selected from rs10740085, rs12768538, rs7068361, rs7071642, rs7076156, rs729739, rs10995271, rs12766391, rs10761659, and rs224120. Assaying the sample comprises genotyping for one or more single nucleotide polymorphisms. The sample can be whole blood, plasma, serum, saliva, cheek swab, urine, or stool.

In one embodiment, the invention provides a method of diagnosing susceptibility to Crohn's disease in an individual, comprising: obtaining a sample from the individual, assaying the sample to determine the presence or absence of a risk variant at the FUT2 genetic locus, and diagnosing susceptibility to Crohn's disease in the individual based on the presence of the risk variant at the FUT2 genetic locus. The risk variant can be selected from the group consisting of rs602662, rs676388, rs485186, and rs504963. Assaying of the sample comprises genotyping for one or more single nucleotide polymorphisms. The sample can be whole blood, plasma, serum, saliva, cheek swab, urine, or stool.

In another embodiment, the invention provides a method of determining a high probability of developing Crohn's disease in an individual, relative to a healthy subject, comprising: obtaining a sample from the individual, assaying the sample to determine the presence or absence of one or more risk variants at the FUT2 genetic locus, and diagnosing a high probability of developing Crohn's disease in the individual, relative to a healthy subject, based upon the presence of one or more risk variants at the FUT2 genetic locus. The risk variant can be selected from the group consisting of rs602662, rs676388, rs485186, and rs504963 Assaying of the sample comprises genotyping for one or more single nucleotide polymorphisms. The sample can be whole blood, plasma, serum, saliva, cheek swab, urine, or stool.

In a related embodiment, the invention provides a method of prognosing Crohn's disease in an individual, comprising: obtaining a sample from the individual, assaying the sample for the presence or absence of one or more genetic risk variants, and prognosing an aggressive form of Crohn's disease based on the presence of one or more risk variants at the FUT2 genetic locus. The risk variant can be selected from the group consisting of rs602662, rs676388, rs485186, and rs504963. Assaying of the sample comprises genotyping for one or more single nucleotide polymorphisms. The sample can be whole blood, plasma, serum, saliva, cheek swab, urine, or stool.

In a further embodiment, the invention provides method of treating an individual for Crohn's disease, comprising: prognosing an aggressive form of Crohn's disease in the individual based on the presence of one or more risk variants at the FUT2 genetic locus, and treating the individual, wherein the one or more risk variants are selected from rs602662, rs676388, rs485186, and rs504963. Assaying the sample comprises genotyping for one or more single nucleotide polymorphisms. The sample can be whole blood, plasma, serum, saliva, cheek swab, urine, or stool.

Various methods include a method of prognosing inflammatory bowel disease (IBD) in an individual, comprising obtaining a sample, assaying the sample to determine the presence or absence of one or more risk variants at Chromosome 4, assaying the sample to determine the presence or absence of serological marker ANCA, prognosing an aggressive form of inflammatory bowel disease in the individual based on the presence of one or more risk variants at Chromosome 4 and the presence of serological marker ANCA. In another embodiment, the aggressive form of inflammatory bowel disease is characterized by an aggressive form of ulcerative colitis. In another embodiment, the one or more risk variants at Chromosome 4 are at the genetic loci of AFP, AFM, RASSF6 and/or PGM2. In another embodiment, the one or more risk variants at Chromosome 4 comprise SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:33, SEQ ID NO:34, SEQ ID NO:35, SEQ ID NO:36, SEQ ID NO:37, SEQ ID NO:38, and/or SEQ ID NO:39. In another embodiment, the presence of serological marker ANCA comprises a high level of serological marker ANCA as compared to a healthy subject. In another embodiment, the absence of serological marker ANCA is indicative of inflammatory bowel disease with Crohn's like conditions.

Other embodiments include a method of diagnosing an ulcerative colitis subtype in an individual, comprising obtaining a sample, assaying the sample to determine the presence or absence of serological marker ANCA, and diagnosing the ulcerative colitis subtype in the individual, wherein the presence of serological marker ANCA is indicative of an aggressive subtype of ulcerative colitis, and wherein the absence of serological marker ANCA is indicative of an ulcerative colitis subtype with Crohn's disease characteristics. In another embodiment, further comprising the presence of one or more risk variants at Chromosome 4. In another embodiment, further comprising SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:33, SEQ ID NO:34, SEQ ID NO:35, SEQ ID NO:36, SEQ ID NO:37, SEQ ID NO:38, and/or SEQ ID NO:39. In another embodiment, the presence of serological marker ANCA comprises a high level of serological marker ANCA as compared to a healthy subject.

Other embodiments include a method of diagnosing a Crohn's disease subtype in an individual; comprising obtaining a sample, assaying the sample to determine the presence or absence of one or more genetic risk variants located at Chromosome 15, Chromosome 18, and/or AK097193 genetic locus, and assaying the sample to determine the presence or absence of serological markers I2, OmpC and/or Cbir1, diagnosing the Crohn's disease subtype based on the presence of one or more genetic risk variants and the presence of one or more serological markers. In another embodiment, the one or more genetic risk variants comprise SEQ ID NO:40, SEQ ID NO:41, SEQ ID NO:42, and/or SEQ ID NO:43. In another embodiment, SEQ ID NO:40 is associated with the presence of antibody I2. In another embodiment, SEQ ID NO:41 is associated with the presence of antibody OmpC. In another embodiment, SEQ ID NO:42 and/or SEQ ID NO:43 is associated with the presence of antibody Cbir1.

Various embodiments herein also include a method of diagnosing susceptibility to Crohn's disease in an individual, comprising obtaining a sample, assaying the sample to determine the presence or absence of one or more genetic risk variants located at the genetic loci of FHIT, ETV4, ME1, WDR64, A2BP1, CDH2, HSPBP1, PPP6R1, and/or BRSK1, diagnosing susceptibility to Crohn's disease in the individual based on the presence of one or more genetic risk variants. In another embodiment, the one or more genetic risk variants are associated with the presence of serological marker ANCA. In another embodiment, the one or more genetic risk variants include SEQ ID NO:21, SEQ ID NO:45, SEQ ID NO:46, SEQ ID NO:47, SEQ ID NO:48, SEQ ID NO:49, and/or SEQ ID NO:50.

Various embodiments include a method of diagnosing susceptibility to granuloma in an individual with Crohn's disease, comprising obtaining a sample from the individual, assaying the sample to determine the presence or absence of at least one risk genetic variant, assaying the sample to determine the presence or absence of at least one risk serological marker, and diagnosing susceptibility to granuloma in the individual if the at least one risk genetic variant is present, or if the at least one risk serological marker is present, or if the at least one risk genetic variant is present and the at least one risk serological marker is present. In another embodiment, the at least one risk genetic variant is at the genetic locus of TGFb3, FTO, NPAS2, MUC1, IL10, LRAP, LRRK2, TNFSF15, or cytochrome P-450 cluster, or a combination thereof. In another embodiment, the at least one risk serological marker is selected from the group consisting of anti-Cbir1, ANCA, ASCA, anti-OmpC, and anti-I2. In another embodiment, the ASCA is present in high titre. In another embodiment, the at least one risk genetic variant includes SEQ ID NO:51, SEQ ID NO:52, SEQ ID NO:53, SEQ ID NO:54, SEQ ID NO:55, and/or SEQ ID NO:56. In another embodiment, the at least one risk genetic variant includes SEQ ID NO:57, SEQ ID NO:58, SEQ ID NO:59, SEQ ID NO:60, SEQ ID NO:61, SEQ ID NO:62, and/or SEQ ID NO:63. In another embodiment, the Crohn's disease is associated with a small bowel disease phenotype, an aggressive complicating phenotype, an internal penetrating disease phenotype, a stricturing disease phenotype, or a fibrostenosing disease phenotype, or a combination thereof. In another embodiment, the first and/or second sample comprises a nucleic acid from the individual.

Other embodiments include a method of diagnosing granuloma in an individual with Crohn's disease, comprising obtaining a sample from the individual, and assaying the sample to determine the presence or absence of at least one risk genetic variant, assaying the sample to determine the presence or absence of at least one risk serological marker, and diagnosing granuloma in the individual if the at least one risk genetic variant is present, or if the at least one risk serological marker is present, or if the at least one risk genetic variant is present and the at least one risk serological marker is present. In another embodiment, the at least one risk genetic variant is at the genetic locus of TGFb3, FTO, NPAS2, MUC1, IL10, LRAP, LRRK2, TNFSF15, or cytochrome P-450 cluster, or a combination thereof. In another embodiment, the at least one risk serological marker is selected from the group consisting of anti-Cbir1, ANCA, ASCA, anti-OmpC, and anti-I2. In another embodiment, the ASCA is present in high titre. In another embodiment, the at least one risk genetic variant includes SEQ ID NO:51, SEQ ID NO:52, SEQ ID NO:53, SEQ ID NO:54, SEQ ID NO:55, and/or SEQ ID NO:56. In another embodiment, the at least one risk genetic variant includes SEQ ID NO:57, SEQ ID NO:58, SEQ ID NO:59, SEQ ID NO:60, SEQ ID NO:61, SEQ ID NO:62, and/or SEQ ID NO:63. In another embodiment, the Crohn's disease is associated with a small bowel disease phenotype, an aggressive complicating phenotype, an internal penetrating disease phenotype, a stricturing disease phenotype, or a fibrostenosing disease phenotype, or a combination thereof.

Various embodiments include a method of diagnosing susceptibility to low bone density (LBD) in an individual with an inflammatory bowel disease (IBD), comprising obtaining a sample from the individual, assaying the sample to determine the presence or absence of at least one risk genetic variant, assaying the sample to determine the presence or absence of at least one risk serological marker, and diagnosing susceptibility to LBD in the individual if the at least one risk genetic variant is present, or if the at least one risk serological marker is present, or if the at least one risk genetic variant is present and the at least one risk serological marker is present. In another embodiment, the LBD is osteoporosis or osteopenia. In another embodiment, the at least one risk genetic variant is at the genetic locus of HLA, laminin, plexin, or NLR family, or a combination thereof. In another embodiment, the at least one risk genetic variant is SEQ ID NO:64 and/or SEQ ID NO:65. In another embodiment, the at least one risk serological marker is selected from the group consisting of anti-Cbir1, ASCA, and anti-I2. In another embodiment, the IBD is a perianal disease.

Other embodiments include a method of treating low bone density (LBD) in an individual with an inflammatory bowel disease (IBD), comprising obtaining a sample from the individual, assaying the sample to determine the presence or absence of at least one risk genetic variant, assaying the sample to determine the presence or absence of at least one risk serological marker, and treating LBD in the individual if the at least one risk genetic variant is present, or if the at least one risk serological marker is present, or if the at least one risk genetic variant is present and the at least one risk serological marker is present. In another embodiment, the at least one risk genetic variant is SEQ ID NO:64 and/or SEQ ID NO:65. In another embodiment, the at least one risk serological marker is selected from the group consisting of anti-Cbir1, ASCA, and anti-I2.

Various embodiments include a method of diagnosing susceptibility to an inflammatory bowel disease (IBD) subtype in an individual, comprising obtaining a sample from the individual, assaying the sample to determine the presence or absence of at least one risk genetic variant at the genetic locus of IFNG, diagnosing susceptibility to the IBD subtype based on the presence of at least one risk genetic risk variant at the genetic locus of IFNG. In another embodiment, the IBD is ulcerative colitis. In another embodiment, the IBD is associated with early surgical intervention. In another embodiment, the IBD is associated with colitis, a small bowel disease phenotype, an aggressive complicating phenotype, an internal penetrating disease phenotype, a stricturing disease phenotype, a fibrostenosing disease phenotype, or a fistulating disease phenotype, or a combination thereof. In another embodiment, the IBD is associated with at least one risk serological marker selected from the group consisting of ANCA, ASCA, anti-Cbir1, anti-I2, and anti-OmpC. In another embodiment, the at least one risk genetic variant is a “T” allele of SEQ ID NO:66. In another embodiment, the at least one risk genetic variant is associated with a lower level of IFNG DNA methylation relative to a healthy subject. In another embodiment, the at least one risk genetic variant is associated with a higher level of anti-Cbir1 relative to a healthy subject. In another embodiment, the at least one risk genetic variant is a “C” allele of SEQ ID NO:66. In another embodiment, the at least one risk genetic variant is associated with a higher level of IFNG DNA methylation relative to a healthy subject.

Other embodiments include a method of diagnosing inflammatory bowel disease (IBD) in an individual, comprising obtaining a sample from an individual, assaying the sample to determine the presence or absence of at least one risk genetic variant at the genetic locus of IFNG, assaying the sample to determine an increase or decrease in IFNG DNA methylation relative to a healthy subject, and diagnosing IBD in the individual based on the presence of at least one risk genetic variant at the genetic locus of IFNG and an increase in IFNG DNA methylation relative to a healthy subject. In another embodiment, the IBD is Crohn's disease or ulcerative colitis. In another embodiment, the at least one risk genetic variant is a “T” allele of SEQ ID NO:66. In another embodiment, the method further comprises determining the presence of a high level of anti-Cbir1 relative to a healthy subject. In another embodiment, the IBD is associated with severe ulcerative colitis conditions. In another embodiment, the IBD is associated with colitis, a small bowel disease phenotype, an aggressive complicating phenotype, an internal penetrating disease phenotype, a stricturing disease phenotype, a fibrostenosing disease phenotype, or a fistulating disease phenotype, or a combination thereof. In another embodiment, the IBD is associated with at least one risk serological marker selected from the group consisting of ANCA, ASCA, anti-Cbir1, anti-I2, and anti-OmpC. In another embodiment, the sample comprises a nucleic acid from the individual. In another embodiment, the sample is a body fluid. In another embodiment, the body fluid is whole blood, plasma, saliva, mucus, or cheek swab. In another embodiment, the sample is a cell or tissue. In another embodiment, the cell, wherein the cell is a lymphoblastoid cell line obtained from the individual and transformed with an Epstein Barr virus. In another embodiment, the cell is a mucosal T cell, a lamina propria T cell, or a peripheral blood T cell.

Other embodiments include a method of treating an inflammatory bowel disease (IBD) in an individual, comprising obtaining a sample from an individual, assaying the sample to determine the presence of at least one risk genetic variant at the genetic locus of IFNG, assaying the sample to determine an aberrant level of IFNG DNA methylation, and treating the IBD in the individual. In another embodiment, the IBD is Crohn's disease or ulcerative colitis. In another embodiment, the IBD is associated with early surgical intervention. In another embodiment, the IBD is associated with colitis, a small bowel disease phenotype, an aggressive complicating phenotype, an internal penetrating disease phenotype, a stricturing disease phenotype, a fibrostenosing disease phenotype, or a fistulating disease phenotype, or a combination thereof. In another embodiment, the at least one risk genetic variant at the genetic locus of IFNG is SEQ ID NO:66.

Various embodiments include a method of treating an inflammatory bowel disease (IBD) in an individual, comprising, obtaining a sample from the individual, assaying the sample to detect the presence or absence of at least one risk genetic variant at the genetic locus of IFNG, and/or assaying the sample to detect an increase or decrease in IFNG DNA methylation relative to a healthy individual, and treating the IBD in the individual. In another embodiment, the IBD comprises Crohn's disease (CD) or ulcerative colitis (UC). In another embodiment, the IBD is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof. In another embodiment, the individual is a human. In another embodiment, the sample comprises a nucleic acid from the individual. In another embodiment, the sample comprises a body fluid, cheek swab, mucus, whole blood, blood, serum, plasma, urine, saliva, semen, lymph, fecal extract, or sputum, or a combination thereof. In another embodiment, the sample comprises a cell or tissue. In another embodiment, the cell is a lymphoblastoid cell line obtained from the individual and transformed with an Epstein Barr virus. In another embodiment, the cell is a mucosal T cell, a lamina propria T cell, or a peripheral blood T cell. In another embodiment, the at least one risk genetic variant is the “T” allele of SEQ ID NO:66 or SEQ ID NO:67. In another embodiment, IFNG DNA methylation is IFNG promoter methylation. In another embodiment, further comprising assaying the sample to detect an increase of INF-γ secretion relative to a healthy individual. In another embodiment, further comprising assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC. In another embodiment, treating the IBD comprises conducting colectomy on the individual, upon detecting the presence of at least one risk genetic variant at the genetic locus of IFNG and/or a decrease in IFNG DNA methylation relative to a healthy individual. In another embodiment, treating the IBD comprises administering a TNF signaling inhibitor to the individual, upon detecting the absence of at least one risk genetic variant at the genetic locus of IFNG and/or an increase in IFNG DNA methylation relative to a healthy individual. In another embodiment, the TNF signaling inhibitor comprises an anti-TNF antibody. In another embodiment, the TNF signaling inhibitor comprises infliximab, adalimumab, certolizumab, certolizumab pegol, golimumab, etanercept, or onercept, or a combination thereof. In another embodiment, the TNF signaling inhibitor is administered topically, intravascularly, intravenously, intraarterially, intramuscularly, subcutaneously, intraperitoneally, intranasally, or orally. IN another embodiment, the TNF signaling inhibitor is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In another embodiment, the TNF signaling inhibitor is administered about 1-3 times per day, 1-7 times per week, or 1-9 times per month. In another embodiment, the TNF signaling inhibitor is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

Other embodiments include a method of treating an inflammatory bowel disease (IBD) in an individual, comprising genotyping the individual for a risk genetic variant at the genetic locus of IFNG, and if the individual is positive for the risk genetic variant, conducting colectomy on the individual, and if the individual is negative for the risk genetic variant, administering a TNF signaling inhibitor to the individual. Other embodiments include a method of treating an inflammatory bowel disease (IBD) in an individual, comprising obtaining a sample from the individual, contacting the sample with an oligonucleotide probe specific to a risk genetic variant at the genetic locus of IFNG, forming an allele-specific hybridization complex between the oligonucleotide probe and the risk genetic variant, detecting the allele-specific hybridization complex, and if the allele-specific hybridization complex is detected, conducting colectomy on the individual, and if the allele-specific hybridization complex is not detected, administering a TNF signaling inhibitor to the individual. In another embodiment, the individual is a human. In another embodiment, the risk genetic variant is the “T” allele of SEQ ID NO:66 or SEQ ID NO:67. In another embodiment, the oligonucleotide probe is labeled with a fluorescent dye, and wherein detecting the allele-specific hybridization complex comprises detecting fluorescence signal from the oligonucleotide probe. In another embodiment, the oligonucleotide probe comprises a reporter dye and a quencher dye. In another embodiment, further comprising conducting PCR amplification after forming the allele-specific hybridization complex. In another embodiment, detecting the allele-specific hybridization complex comprises detecting the electrophoretic mobility of the allele-specific hybridization complex.

Various methods herein include a method of predicting responsiveness to thiopurine treatment in an individual, comprising obtaining a sample from the individual, assaying the sample to determine the presence or absence of one or more risk variants at the HLA-DRB1, CREM, TAGAP, PLCL1, GPX4, SBNO2, MEF2A and/or LYSMD4 genetic loci, predicting responsiveness to thiopurine treatment based on the presence of one or more risk variants at the HLA-DRB1, CREM, TAGAP, PLCL1, GPX4, SBNO2, MEF2A and/or LYSMD4 genetic loci. In another embodiment, the individual has been diagnosed with inflammatory bowel disease. In another embodiment, the individual is a child. In another embodiment, the risk variants include SEQ ID NO:83, SEQ ID NO:84, SEQ ID NO:85, SEQ ID NO:86, and/or SEQ ID NO:87. In another embodiment, the presence of five or more said risk variants presents a greater probability of responsiveness to thiopurine treatment than the presence of four, three, two, one or none of said risk variants; the presence of four said risk variants presents a greater probability of responsiveness to thiopurine treatment than the presence of three, two, one or none of said risk variants; the presence of three risk variants presents a greater probability of responsiveness to thiopurine treatment than the presence of two, one or none of said risk variants; the presence of two risk variants presents a greater probability of responsiveness to thiopurine treatment than the presence of one or none of said risk variants; and the presence of one risk variant presents a greater probability of responsiveness to thiopurine treatment than the presence of none of said risk variants. In another embodiment, the individual has been diagnosed with ulcerative colitis. In another embodiment, the sample further comprises a high expression relative to a normal subject of pANCA. In another embodiment, the individual has been diagnosed with acute lymphoblastic leukemia and/or an autoimmune disorder. In another embodiment, the individual is an organ transplant recipient.

Other embodiments include a method of treating a disease in an individual, comprising determining the presence of one or more risk variants in the individual at the HLA-DRB1, CREM, TAGAP, PLCL1, GPX4, SBNO2, MEF2A and/or LYSMD4 genetic loci, and administering a therapeutically effective dosage to the individual of a composition comprising thiopurine, or a pharmaceutical equivalent, analog, derivative, and/or salt thereof. In another embodiment, the risk variants include SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92 and/or SEQ ID NO:93. In another embodiment, the disease is inflammatory bowel disease. In another embodiment, the disease is acute lymphoblastic leukemia and/or an autoimmune disorder. In another embodiment, the sample further comprises a high expression relative to a normal subject of pANCA.

Other embodiments include a method of predicting responsiveness to thiopurine treatment in an individual, comprising obtaining a sample from the individual, assaying the sample to determine the presence or absence of one or more risk variants at the ARL4C, IL1R2, JAK2, 19q13, TAGAP, CARD9, SNAPC4, 8q24 and/or HLA-DRB1 genetic loci, predicting responsiveness to thiopurine treatment based on the presence of one or more risk variants at the ARL4C, IL1R2, JAK2, 19q13, TAGAP, CARD9, SNAPC4, 8q24 and/or HLA-DRB1 genetic loci. In another embodiment, the individual has been diagnosed with inflammatory bowel disease. In another embodiment, the individual is a child. In another embodiment, the risk variants include SEQ ID NO:83, SEQ ID NO:85, SEQ ID NO:88, SEQ ID NO:89, SEQ ID NO:90, SEQ ID NO:91, SEQ ID NO:92 and/or SEQ ID NO:93. In another embodiment, the individual is male.

The above-mentioned and other features and advantages of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with various embodiments of the invention, a haplotype map and structure of SMAD3, including SMAD3 Blocks 1-3 and corresponding SNPs.

FIG. 2 depicts, in accordance with various embodiments of the invention, the genomic structure of the four isoforms of ZNF365 (A-D). Exon 4, unique to ZNF365D, harboring the associated SNP rs7076156 is also marked.

FIG. 3 depicts, in accordance with various embodiments of the invention, linkage disequilibrium and haplotype structure across the ZNF365 SNPs (generated in HAPLOVIEW). Region encompassing ZNF365 isoform D is noted. Top hits reported are marked with an asterisk (8, 10, 11). Rs7076156 is also marked, with rs7071642 immediately adjacent.

FIG. 4 depicts, in accordance with various embodiments of the invention, gels demonstrating expression of 379-bp ZNF365D was detected in ileum obtained from a CD patient undergoing small bowel surgery. ZNF365D expression is also observed in the adult kidney.

FIG. 5 depicts, in accordance with various embodiments of the invention, a table of ZNF365 SNPs associated with Crohn's disease.

FIGS. 6A-6B depicts, in accordance with various embodiments of the invention, a table of genotyped SNPs in 10q21.2.

FIG. 7 depicts, in accordance with various embodiments of the invention, graphical representation of an association between FUT2 and CD. Circles—The GWAS population. Squares—The independent case-control replication cohort.

FIG. 8 depicts, in accordance with various embodiments of the invention, principal component plot for components 1 (C1—y axis) and 2 (C2—x axis) in CD and controls. The circled cases and controls are on the ‘Caucasian’ axis and were included in logistic regression analysis.

FIG. 9 depicts, in accordance with various embodiments of the invention, a table listing the replication of confirmed and “nominally associated” CD susceptibility loci from CD GWAS meta-analysis (Barrett, J. C. et al., Nat Genet 40, 955-62 (2008)). Chr.—chromosome

FIG. 10 depicts, in accordance with various embodiments of the invention, a table listing novel loci associated with CD (cut off p=<1.0×10⁻⁴). Chr.—chromosome.

FIG. 11 depicts, in accordance with various embodiments of the invention, a table summarizing the association between FUT2 and CD in GWAS, confirmatory cohort of 1174 cases and 357 controls and the p value for association by the CD GWAS meta-analysis from Barrett et al. *P value calculated using logistic regression. **Combined p value calculated for p value in original GWAS and one tailed p value in independent replication. Synon.—synonymous.

FIG. 12 depicts, in accordance with various embodiments of the invention, graphical representation of the linkage disequilibrium and haplotype structure across the 6 FUT2 SNPs. Figure and data generated in HAPLOVIEW. Figures represent the LD in percent between SNPs as represented by D′.

FIG. 13 depicts, in accordance with various embodiments of the invention, two Chr.4 loci associated with the phenotypic difference of UC patients, one for UC severity as typified by ANCA level, the other for the expression of antibodies more characteristic of CD. If a listed SNP allele has an OR>1, it means that this allele's presence is associated with the UC subtype; if OR<1, it means this allele's absence is associated with the UC subtype. As an example, rs7668327 is a G/C SNP and the table shows the G allele with OR<1, so that the absence of G (i.e., the presence of C) is associated with the ANCA UC subtype.

FIGS. 14A-14B depict, in accordance with various embodiments of the invention, −log(10)p-values of CD ANCA analysis. FIG. 14A: Definition of ANCA phenotype. FIG. 14B: QQ plot. The ANCA analysis compared the lowest and highest tertile with the center tertile removed.

FIGS. 15A-15D depict, in accordance with various embodiments of the invention, antibody distribution divided into tertiles and scores are then summed. FIG. 15A: ASCA scores; FIG. 15B: anti-CBir1 scores; FIG. 15C: anti-I2 scores; and FIG. 15D: anti-OmpC scores.

FIG. 16 depicts, in accordance with various embodiments of the invention, top hits of various listed SNPs and corresponding alleles as associated with granuloma from performed GWAS.

FIG. 17 depicts, in accordance with various embodiments of the invention, top hits of various listed SNPs and corresponding alleles as associated with granuloma from performed GWAS.

FIG. 18 depicts, in accordance with various embodiments of the invention, allele specific differential methylation associated with the +2167 but not +2209 CpG site.

FIG. 19 depicts, in accordance with various embodiments of the invention, IFNG SNP is functionally associated with enhanced promoter methylation and decreased protein expression.

FIG. 20 depicts, in accordance with various embodiments of the invention, IFNG SNP is associated with increased time to surgery and decreased Cbir responsiveness.

FIG. 21 depicts, in accordance with various embodiments of the invention, enhanced nucleoprotein binding to IFNG rs1861494 “T” allele compared to “C” allele.

FIG. 22 depicts, in accordance with various embodiments of the invention, enhanced nucleoprotein binding methylated CpG.

FIG. 23 depicts, in accordance with various embodiments of the invention, a chart summarizing the IFNG research findings.

FIG. 24 depicts, in accordance with various embodiments of the present invention, schematic of IFNG gene showing conserved regions between human and mouse.

FIGS. 25A-25C depict, in accordance with various embodiments of the present invention, relationship of rs1861494 to serological markers and time to surgery. FIG. 25A, rs1861494 SNP allele distribution in IBD patients as a function of sero-reactivity to ANCA; FIG. 25B, rs1861494 T allele association with ANCA EU in UC patients (n=17 for C, n=39 for T); FIG. 25C, Time to surgery curve of medically refractive UC patients based on rs1861494 allele genotype (n=13 for C, n=153 for T). Bars represent means±SEM.

FIG. 26 depicts, in accordance with various embodiments of the present invention, rs1861494 T allele, compared to C allele, genotype is associated with more severe disease behavior in CD patients. Frequency of non-stricturing/penetrating vs. complicated (stricturing/penetrating) disease phenotype was determined in 44 CD patients.

FIG. 27 depicts, in accordance with various embodiments of the present invention, secretion of IFN gamma from IBD T cells stimulated with anti-CD3 antibody for 24 h (n=28 for C, n=104 for T). Bars represent means±SEM.

FIGS. 28A-28C depict, in accordance with various embodiments of the present invention, allele/strand specific methylation of IBD patients heterozygous for rs1861494. FIG. 28A, rs1861494; FIG. 28B, CpG at +2052 and +2007; FIG. 28C, Correlation of methylation of rs1861494 with methylation index of IFNG promoter (CpG sites at −54, −186, and −295 bp) (n=36). Bars represent means±SEM.

FIGS. 29A-29B depicts, in accordance with various embodiments of the present invention, EMSA analysis of rs1861494 regulatory binding proteins. PBMC were stimulated up to 4 h with PMA/ionomycin and nuclear protein extracts were obtained. FIG. 29A: EMSAs were performed for binding activity to the C or T SNP; FIG. 29B: kinetics of nucleo-protein binding to the non-methylated (C SNP), asymmetrically methylated (one strand, C/MC SNP) or symmetrically methylated (both strands, MC-MC SNP) oligonucleotide. Representative of 4 experiments.

FIGS. 30A-30C depict, in accordance with various embodiments of the invention, various genetic loci as part of a correlation analysis of IFNG expression.

FIG. 31 depicts, in accordance with various embodiments herein, genes found to be associated with both DMR and eQTL.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N. Y. 2006); Smith, March's Advanced Organic Chemistry Reactions, Mechanisms and Structure 7^{th} ed., J. Wiley & Sons (New York, N. Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N. Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N. Y., 2013); Köhler and Milstein, Derivation of specific antibody-producing tissue culture and tumor lines by cell fusion, Eur. J. Immunol. 1976 Jul. 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described.

The term “inflammatory bowel disease” or “IBD” refers to gastrointestinal disorders including, but not limited to Crohn's disease (CD), ulcerative colitis (UC), and indeterminate colitis (IC). Inflammatory bowel diseases such as CD, UC, and IC are distinguished from all other disorders, syndromes, and abnormalities of the gastroenterological tract, including irritable bowel syndrome (IBS).

“SMAD3” as used herein refers to SMAD family member 3.

“JAK2” as used herein refers to Janus kinase 2 (a protein tyrosine kinase).

“SNP” as used herein is an abbreviation of single nucleotide polymorphism.

“Haplotype” as used herein refers to a set of single nucleotide polymorphisms (SNPs) on a gene or chromatid that are statistically associated.

“GWAS” as used herein means Genome-Wide Association Study.

“Risk” as used herein refers to an increase in susceptibility to IBD, including but not limited to CD and UC.

“Risk variant” as used herein refers to genetic variants, the presence of which correlates with an increase or decrease in susceptibility to Crohn's disease. In some embodiments, risk variants of Crohn's disease include, but are not limited to variants at the ZNF365 genetic locus, such as “haplotypes” and/or a set of single nucleotide polymorphisms (SNPs) on a gene or chromatid that are statistically associated. In some embodiments, more preferably, risk variants can include, but are not limited to rs10740085, rs12768538, rs7068361, rs7071642, rs7076156, rs729739, rs10995271, rs12766391, rs10761659, and rs224120. In some embodiments, risk variants of Crohn's disease include, but are not limited to variants at the FUT2 genetic locus, such as “haplotypes” and/or a set of single nucleotide polymorphisms (SNPs) on a gene or chromatid that are statistically associated. In some embodiments, more preferably, risk variants can include, but are not limited to rs602662, rs676388, rs485186, and rs504963.

“Protective” and “protection” as used herein refer to a decrease in susceptibility to IBD, including but not limited to CD and UC.

“CD” and “UC” as used herein refer to Crohn's disease and ulcerative colitis, respectively.

“F_A” as used herein means frequency in CD.

“F_U” as used herein means frequency in controls.

“P” as used herein means the P value for that association.

“PAR” as used herein refers to population attributable risk, including an estimation of the proportion of cases in the population attributable to the given risk factor.

As used herein, “treatment” or “treating” should be understood to include any indicia of success in the treatment, alleviation or amelioration of an injury, pathology or condition. This may include but not limited to parameters such as abatement, remission, diminishing of symptoms, slowing in the rate of degeneration or decline, making the final point of degeneration less debilitating; improving a patient's physical or mental well-being, or preventing the onset of disease, such as Crohn's disease.

“Treatment” or “treating,” as used herein refer to both therapeutic treatment and prophylactic or preventative measures, wherein the object is to prevent, slow down and/or lessen the disease even if the treatment is ultimately unsuccessful. Those in need of treatment include those already with Crohn's disease as well as those prone to have Crohn's disease or those in whom Crohn's disease is to be prevented. For example, in Crohn's disease treatment, a therapeutic agent may directly decrease the pathology of IBD, or render the cells of the gastroenterological tract more susceptible to treatment by other therapeutic agents.

As used herein, “diagnose” or “diagnosis” refers to determining the nature or the identity of a condition or disease. A diagnosis may be accompanied by a determination as to the severity of the disease.

As used herein, “prognostic” or “prognosis” refers to predicting the probable course and outcome of IBD or the likelihood of recovery from IBD. The prognosis can include the presence, the outcome, or the aggressiveness of the disease.

As used herein, the term “biological sample” or “sample” means any biological material obtained from an individual from which nucleic acid molecules can be prepared. Examples of a biological sample include, but are not limited to whole blood, plasma, serum, saliva, cheek swab, urine, stool, or other bodily fluid or tissue that contains nucleic acid.

As used herein, the term “Jewish” refers to those individuals with at least one out of four grandparents of Ashkenazi Jewish origin, and vice versa the term “non-Jewish” refers to those individuals with no grandparents of Ashkenazi Jewish origin (see NIDDK IBDGC Manual by IBD Genetics Consortium (IBDGC) of the National Institute of Diabetes and Digestive and Kidney Diseases (NIDDK)). Moreover, in the biomedical field of population genetics, whether an individual is “Jewish” or “non-Jewish” is not determined according to whether the individual practices Jewish religion or not.

As used herein, the term “Caucasian” adopts the common understanding in the biomedical field of population genetics. “Caucasian” is one well-recognized term referring to a major group of human races (see NIDDK IBDGC Manual by IBD Genetics Consortium (IBDGC) of the National Institute of Diabetes and Digestive and Kidney Diseases (NIDDK); and Risch et al., Categorization of humans in biomedical research: genes, race and disease, Genome Biol. 2002; 3(7): comment2007.1-comment2007.12. Published online 2002 Jul. 1).

The inventors performed a genome-wide association study (GWAS) testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to Crohn's disease (CD) and ulcerative colitis (UC). These SNPs and haplotypes are suitable for genetic testing to identify at risk individuals and those with increased risk for complications associated with serum expression of anti-Saccharomyces cerevisiae antibody, and antibodies to I2, OmpC, and Cbir. The detection of protective and risk SNPs and/or haplotypes may be used to identify at risk individuals, predict disease course, and suggest the right therapy for individual patients. Additionally, the inventors have found both protective and risk allelic variants for Crohn's disease (CD) and ulcerative colitis (UC).

Based on these findings, embodiments of the present invention provide for methods of diagnosing and/or predicting susceptibility for or protection against inflammatory bowel disease including but not limited to Crohn's disease and ulcerative colitis. Other embodiments provide for methods of prognosing inflammatory bowel disease including but not limited to Crohn's disease and ulcerative colitis. Other embodiments provide for methods of treating inflammatory bowel disease including but not limited to Crohn's disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

As disclosed herein, the inventors constructed haplotypes for both the SMAD3 and JAK2 genetic loci and tested for associations in Crohn's Disease subjects. As described in Tables 1-6 herein, various haplotypes and variants were found to have statistically significant associations with Crohn's Disease.

In one embodiment, the present invention provides a method of diagnosing susceptibility to Inflammatory Bowel Disease (IBD) in an individual by determining the presence or absence of a risk variant at the SMAD3 and/or JAK2 genetic locus, where the presence of the risk variant at the SMAD3 and/or JAK2 genetic locus is indicative of susceptibility to IBD in the individual.

In one embodiment, the present invention provides a method of diagnosing a Crohn's Disease (CD) subtype in an individual by determining the presence or absence of a risk variant at the SMAD3 and/or JAK2 genetic locus, where the presence of the risk variant at the SMAD3 and/or JAK2 genetic locus is indicative of the CD subtype in the individual.

In one embodiment, the present invention provides a method of treating CD in an individual by determining the presence of one or more risk variants at the SMAD3 and/or JAK2 genetic locus, and treating the individual.

As disclosed herein, in the interest of identifying causal variants of Crohn's disease at 10q21, the inventors finely mapped the 10q21 region. The inventors genotyped 86 SNPs across the region of reported association (Chr. 10, position 63,798,139 to 64,219,617) in 1,683 CD cases and 1,049 non-IBD controls. Single marker and conditional analyses were performed using logistic regression (PLINK). ZNF365 isoform D expression was assessed using RT-PCR. Peak association with CD was observed within ZNF365 at rs7076156 and rs7071642, two SNPs in complete linkage disequilibrium (LD) (FIG. 5). Conditioning on nonsynonymous SNP rs7076156 (Ala62Thr) nullified all other significant associations and the threonine allele protected against CD (p=1.05×10⁻⁷; OR 0.71; 23.6% in patients with CD and 30.1% in controls). Four isoforms of ZNF365 (A-D) have previously been identified and rs7076156 is located in an exon unique to ZNF365 isoform D. The inventors further detected expression of this isoform in a terminal ileum resection specimen from a patient with CD.

As further disclosed herein, the inventors demonstrate significant associations between CD and the ZNF365 locus. Conditional analyses show that a coding variant (rs7076156; Ala62Thr) confers protection against CD. Furthermore, mRNA for ZNF365 isoform D is expressed in small intestine. Taken together these data show that this variant explains the CD association observed at 10q21.

In one embodiment, the present invention provides a method of diagnosing a low probability of developing Crohn's Disease in an individual, relative to a healthy individual, by determining the presence or absence of one or more protective variants at the ZNF365 genetic locus, where the presence of the one or more protective variants at the ZNF365 genetic locus is indicative of a low probability of developing Crohn's Disease in an individual. In another embodiment, the one or more protective variants comprise rs10740085, rs12768538, rs7068361, rs7071642, rs7076156, rs729739, rs10995271, rs12766391, rs10761659, and/or rs224120.

In one embodiment, the present invention provides a method of diagnosing a risk of susceptibility to Crohn's Disease in an individual, relative to a healthy individual, by determining the presence or absence of one or more risk variants at the ZNF365 genetic locus, where the presence of the one or more risk variants at the ZNF365 genetic locus is indicative of susceptibility to Crohn's Disease in the individual. In another embodiment, the one or more risk variants comprise the SNP rs10740085, rs12768538, rs7068361, rs7071642, rs7076156, rs729739, rs10995271, rs12766391, rs10761659, and/or rs224120.

In one embodiment, the present invention provides a method of treating Crohn's Disease by determining the presence of a risk variant at the ZNF365 genetic locus and treating the individual. In another embodiment, the present invention provides a method of treating Crohn's Disease in an individual by determining the aberrant expression of ZNF365 and treating the individual. In another embodiment, the risk variant comprises the SNP rs10740085, rs12768538, rs7068361, rs7071642, rs7076156, rs729739, rs10995271, rs12766391, rs10761659, and/or rs224120.

In another embodiment, the present invention provides a method of prognosing Crohn's Disease by determining the presence or absence of one or more risk variants at the ZNF365 genetic locus and prognosing a complicated form of Crohn's Disease based on the presence of the one or more risk variants at the ZNF365 genetic locus.

Crohn's disease (CD), one of the major forms inflammatory bowel diseases (IBD), is a chronic, debilitating disease characterized by recurrent gastrointestinal inflammation, postulated to occur as a result of an abnormal immune reaction to commensal flora in genetically susceptible individuals. The role of commensal flora in potentiating chronic gastrointestinal mucosal inflammation is substantiated by data from established rodent models of IBD such as the Il110^{-/-} mouse and the Hla-B27 transgenic rat that are disease free when kept in germ free environments but develop inflammation when raised under pathogen free conditions (Kim, S. C. et al., Gastroenterology 128, 891-906 (2005); Rath, H. C. et al., J Clin Invest 98, 945-53 (1996)) Furthermore, in both of these models, the bacterial load and the nature of the commensal flora can influence either the site or degree of gastrointestinal inflammation (Kim, S. C. et al., Gastroenterology 128, 891-906 (2005); Rath, H. C. et al., Infect Immun 67, 2969-74 (1999); Rath, H. C. et al., Gastroenterology 116, 310-9 (1999)). In human disease, antibiotic and probiotic therapy can be effective in modifying some of the manifestations of IBD (Gionchetti, P. et al., Gastroenterology 119, 305-9 (2000); Rutgeerts, P. et al., Gastroenterology 108, 1617-21 (1995)).

Through utilizing genome-wide association studies (GWAS), in addition to candidate gene approaches, considerable success has been achieved in identifying genetic loci that increase susceptibility to CD in populations of Northern European origin (Duerr, R. H. et al., Science 314, 1461-3 (2006); Hampe, J. et al., Nat Genet 39, 207-11 (2007); Rioux, J. D. et al., Nat Genet 39, 596-604 (2007); Yamazaki, K. et al., Hum Mol Genet 14, 3499-506 (2005)); Hugot, J. P. et al., Nature 411, 599-603 (2001); Ogura, Y. et al., Nature 411, 603-6 (2001)). To date more than thirty loci are definitively known to be associated with CD, although these loci only account for a minority of the genetic variance to CD in the Caucasian population (Barrett, J. C. et al., Nat Genet 40, 955-62 (2008)). A number of the CD susceptibility genes encode important components of the innate immune system genes such as NOD2 (Hugot, J. P. et al., Nature 411, 599-603 (2001); Ogura, Y. et al., Nature 411, 603-6 (2001)), the Toll like receptors (De Jager, P. L. et al., Genes Immun 8, 387-97 (2007); Saruta, M. et al., Inflamm Bowel Dis 15, 321-7 (2009)) and the autophagy genes ATG16L1 and IRGM, emphasizing the importance of the microbial-host interaction in the development of CD. Furthermore, antibodies to bacterial antigens have been identified that define certain sub-groups of CD patients, reinforcing the essential role that bacteria play in driving CD (Mow, W. S. et al., Gastroenterology 126, 414-24 (2004)).

As disclosed herein, a CD genome-wide association study (GWAS) was performed by the inventors, identifying a number of novel associations with CD. Considering the importance of the host-microbial interaction, the novel association with Fucosyltransferase 2 (FUT2), also termed secretor factor (Se), was of particular interest. FUT2 is a physiological trait that regulates the expression of the H antigen, a precursor of the blood group A and B antigens, on the gastrointestinal mucosa. Approximately 20% of Caucasians are non-secretors who do not express ABO antigens in saliva as they are homozygous for FUT2 null alleles (Kelly, R. J. et al., J Biol Chem 270, 4640-9 (1995)). Genetic variation in FUT2 has been implicated in susceptibility to Helicobacter pylori infection (Ikehara, Y. et al., Cancer Epidemiol Biomarkers Prev 10, 971-7 (2001)), Noroviruses (Norwalk virus) (Marionneau, S. et al., J Infect Dis 192, 1071-7 (2005); Thorven, M. et al., J Virol 79, 15351-5 (2005); Carlsson, B. et al., PLoS One 4, e5593 (2009)), and progression of HIV (Kindberg, E. et al., AIDS 20, 685-9 (2006)). FUT2 alleles have also been associated with circulating serum vitamin B12 levels (Hazra, A. et al., Nat Genet 40, 1160-2 (2008)). Furthermore non-secretion of ABO blood group antigens into body fluids has been shown to be associated with the development of oral candidiasis (Thom, S. M. et al., FEMS Microbiol Immunol 1, 401-5 (1989); Aly, F. Z. et al., Epidemiol Infect 106, 355-63 (1991)), rheumatic fever (Haverkorn, M. J. & Goslings, W. R. Am J Hum Genet 21, 360-75 (1969)), recurrent urinary tract infection (Kinane, D. F. et al., Br Med J (Clin Res Ed) 285, 7-9 (1982)), cholera (Chaudhuri, A. & DasAdhikary, C. R. Trans R Soc Trop Med Hyg 72, 664-5 (1978)) and infection with meningococcus (Blackwell, C. C. et al., Lancet 2, 284-5 (1986)), pneumococcus (Blackwell, C. C. et al., Lancet 2, 284-5 (1986)), and haemophilus influenzae (Blackwell, C. C. et al., Lancet 2, 687 (1986)). The data presented herein indicate an association between the non-secretor status associated FUT2 genotype and CD.

In one embodiment, the present invention provides a method of diagnosing susceptibility to Crohn's Disease in an individual, relative to a healthy individual, by determining the presence or absence of a risk variant at the FUT2 genetic locus, where the presence of the risk variant at the FUT2 genetic locus is indicative of susceptibility to Crohn's Disease in the individual. In another embodiment, the risk variant comprises the SNP rs602662, rs676388, rs485186, or rs504963. In one embodiment, the risk variant can be at loci including, but are not limited to ASHL, ARPC1A, RHOU, RBP1 and 2, TACR3, MMD2, NPSR1, ACER2, AP3D1, or SPG20.

In one embodiment, the present invention provides a method of treating Crohn's Disease by determining the presence of a risk variant at the FUT2 genetic locus and treating the individual. The risk variant comprises the SNP rs602662, rs676388, rs485186, and rs504963. In one embodiment, the one or more risk variants can be at loci including, but are not limited to ASHL, ARPC1A, RHOU, RBP1 and 2, TACR3, MMD2, NPSR1, ACER2, AP3D1, or SPG20.

In another embodiment, the present invention provides a method of prognosing Crohn's Disease by determining the presence or absence of one or more risk variants at the FUT2 genetic locus and prognosing a complicated form of Crohn's Disease based on the presence of the one or more risk variants at the FUT2 genetic locus. The risk variant comprises the SNP rs602662, rs676388, rs485186, and rs504963. In one embodiment, the one or more risk variants can be at loci including, but are not limited to ASHL, ARPC1A, RHOU, RBP1 and 2, TACR3, MMD2, NPSR1, ACER2, AP3D1, or SPG20.

In one embodiment, the present invention provides a method of diagnosing a high probability of developing Crohn's Disease in an individual, relative to a healthy individual, by determining the presence or absence of one or more risk variants at the FUT2 genetic locus, where the presence of the one or more risk variants at the FUT2 genetic locus is indicative of a low probability of developing Crohn's Disease in an individual. The risk variant comprises the SNP rs602662, rs676388, rs485186, and rs504963. In one embodiment, the one or more risk variants can be at loci including, but are not limited to ASHL, ARPC1A, RHOU, RBP1 and 2, TACR3, MMD2, NPSR1, ACER2, AP3D1, or SPG20.

In another embodiment, an individual with Crohn's disease having one or more genetic risk variants at CD associated loci specifically involved in the host-microbial interaction, exemplified by, but not limited to, SPG20 and FUT2, is treated by antibiotic and or probiotic based treatment therapies. In yet another embodiment, the antibiotic and probiotic treatments are administered as a preventative measure to individuals who have been identified as having a higher than normal risk of developing CD, based upon the presence of one or more genetic variants at CD associated loci specifically involved in the host-microbial interaction, exemplified by, but not limited to, SPG20 and FUT2.

In another embodiment, the present invention provides a method of prognosing Crohn's Disease by determining the presence or absence of one or more risk variants of genetic loci at SPG20 and FUT2, and prognosing pathogenesis, mediated in whole or in part by host-microbial interaction, based on the presence of the one or more risk variants at one or more of SPG20 and FUT2 genetic loci.

As disclosed herein, it has been found that response to ANCA has been associated with more aggressive disease behavior in ulcerative colitis (UC) patients, whereas sero-reactivity to ASCA, anti-CBir1, anti-I2, and anti-OmpC have been particularly associated with subtypes of Crohn's disease. There is also a hereditary component to expression of these antibodies. The inventors assessed the genetic contribution to IBD associated serological profiles in UC cases, with 1327 UC cases genotyped with the Illumina CNV370 or OmniExpress beadchips, and were sero-typed for ANCA, ASCA, anti-CBir1, anti-I2, and anti-OmpC by ELISA. The inventors performed regression analyses, adjusted for population stratification using principal components as covariates, testing for an association of UC with antibody response. A Z-score for ASCA, anti-CBir1, anti-I2, and anti-OmpC together was generated by adding the four Z-scores for each individual antibody for each subject. The Z-scores were calculated from within the UC cohort only. Association of UC was assessed with this combined score and with ANCA status alone.

As further disclosed herein, the results demonstrate two genome-wide significant associations with UC and (1) ANCA at chr.4 (rs1919469 plogistic=4.82×10⁻⁸, OR=1.90; rs10001225 plogistic=1.97×10⁻⁷, OR=1.77). An additional three SNPs within this region are also found to be associated with nominal significance (p<10-5); and (2) at a second region on chr.4˜37 Mb away, with the combined ASCA, I2, CBir1 and Ompc Z-score (rs2995965 plinear=1.35×10⁻⁹, β=0.82; rs1863284 plinear=1.71×10⁻⁷, β=0.85; rs2911920 plinear=6.29×10⁻⁶, β=0.61). RELL1, a homologue of RELT the TNF receptor that induces epithelial cell apoptosis is located at this locus. In general, these and additional observations disclosed herein support two chromosome 4 loci associated with the phenotypic differences of UC patients, one for UC severity as typified by ANCA level, the other for the expression of antibodies more characteristic of CD.

In one embodiment, the present invention provides a method of diagnosing susceptibility to inflammatory bowel disease (IBD) in an individual by obtaining a sample from the individual, assaying the sample to determine the presence or absence of one or more risk variants on Chromosome 4, and/or risk serological markers, where the presence of one or more risk variants at Chromosome 4 and/or risk serological markers is indicative of susceptibility to IBD in the individual. In another embodiment, the IBD is ulcerative colitis. In another embodiment, the one or more risk variants on Chromosome 4 include SEQ ID NOs: 24-39. In another embodiment, the risk serological markers include ANCA, ASCA, I2, Cbir1, and/or OmpC.

In one embodiment, the present invention provides a method of diagnosing an inflammatory bowel disease (IBD) subtype in an individual, by obtaining a sample from the individual, assaying the sample to determine the presence of one or more risk variants on Chromosome 4 and one or more risk serological markers, and diagnosing the IBD subtype in the individual based on the presence of one or more risk variants on Chromosome 4 and one or more risk serological markers. In another embodiment, the presence of SNP rs1919469 and/or rs10001225 on Chromosome 4 and ANCA is indicative of ulcerative colitis. In another embodiment, the ulcerative colitis is an aggressive form of the disease. In another embodiment, the presence of SNPs rs2995965, rs1863284, and/or rs2911920 on Chromosome 4 and risk serological markers ASCA, I2, Cbir1, and/or OmpC is indicative of Crohn's disease.

In one embodiment, the present invention provides a method of prognosing inflammatory bowel disease (IBD) in an individual, by obtaining a sample from the individual, assaying the sample to determine the presence of one or more risk variants and/or risk serological markers, and prognosing a severe form of IBD based on the presence of one or more risk variants and/or risk serological markers. In another embodiment, the present invention provides a method of prognosing a severe form of ulcerative colitis, by determining the presence or absence of one or more risk variants at Chromosome 4 and ANCA expression, where the presence of one or more risk variants at Chromosome 4 and a high level of ANCA expression relative to a normal subject are indicative of the severe form of ulcerative colitis.

In one embodiment, the present invention provides a method of treating IBD in an individual by determining the presence of one or more risk variants at Chromosome 4 and one or more risk variants, and treating the individual.

As disclosed herein, the inventors conducted a genome-wide association study (GWAS) on 1544 CD subjects serotyped for CD-associated antibodies (ASCA, anti-CBir1, anti-I2, and anti-OMPC). Serum antibody expression was measured by ELISA and levels were log transformed prior to analyses. Single nucleotide polymorphism (SNP) data were generated using Illumina technology (˜550K SNPs with MAF>0.05) at Cedars-Sinai Medical Center. Adjustment for population stratification was carried out using two principal components as covariates in the analyses (Eigensoft). The significance of association was tested using logistic regression for antibody positive or negative and linear regression for antibody level after transformation. To overcome multiple testing issues significance was defined to be p<2e-07.

As further disclosed herein, at the pre-defined level of significance, the inventors observed two significant associations: 1) expression of anti-I2 was significantly associated with three SNPs spanning 90 kb of chr. 15 that included the 3 region of human EST BF729345, among other ESTs (rs246336, OR for G allele and anti-I2 positivity, 1.8; p (logistic regression)=8.6e-08); and 2) Expression of anti-OMPC was significantly associated with rs6566234 on chr. 18 (beta coefficient for G allele was −0.28, p (linear regression)=1.4e-07), potentially in LD with CDH19. In addition, 3) anti-Cbir1 positivity was associated with gene AK097193 on chr. 1 (rs1022265 G allele OR for anti-CBir positivity 0.68 p (logistic regression)=7.6 e-07); and 4) ASCA positivity was associated with two SNPs on chr. 3 (rs291528 & rs291523, OR 1.9, p (logistic regression)=5e-07).

In one embodiment, the present invention provides a method of diagnosing susceptibility to a Crohn's disease subtype in an individual, by obtaining a sample from the individual, assaying the sample to determine the presence or absence of risk genetic variants and/or one or more risk serological markers, where the presence of one or more risk genetic variants and/or one or more risk serological markers are indicative of susceptibility to the Crohn's disease subtype. In another embodiment, the Crohn's disease subtype is associated with an aggressive form of the disease. In another embodiment, the one or more risk genetic variants are located on Chromosome 15 and the one or more risk serological markers include I2. In another embodiment, the one or more risk genetic variants are located on Chromosome 18 and the one or more risk serological markers include OmpC. In another embodiment, the one or more genetic variants are located on the AK097193 genetic locus and the one or more risk serological markers include Cbir. In another embodiment, the one or more genetic variants are located on Chromosome 3 and the one or more risk serological markers include ASCA.

In one embodiment, the present invention provides a method of prognosing an aggressive form of Crohn's disease in an individual by obtaining a sample from the individual, assaying the sample to determine the presence of one or more risk genetic variants and/or risk serological markers, and prognosing a severe form of Crohn's disease based on the presence of one or more risk genetic variants and/or risk serological markers.

In one embodiment, the present invention provides a method of treating Crohn's disease in an individual by obtaining a sample from the individual, assaying the sample to determine the presence of one or more risk genetic variants and/or risk serological markers, and treating the individual.

As readily understood by one of skill in the art, any number of sequences may also be used to obtain the various SNPs or genetic variants referenced herein, and the variants are not limited to the specific sequences or accession numbers provided herein. Examples of SNPs rs13148469, rs2050719, rs7760387, rs9399527, rs9784771, rs282792 are provided herein as SEQ ID NO:51, SEQ ID NO:52, SEQ ID NO:53, SEQ ID NO:54, SEQ ID NO:55, and SEQ ID NO:56, respectively. Similarly, examples of SNPs rs10440086, rs1352851, rs13148469, rs282792, rs443394, rs8091293, and rs10514090 are provided herein as SEQ ID NO:57, SEQ ID NO:58, SEQ ID NO:59, SEQ ID NO:60, SEQ ID NO:61, SEQ ID NO:62, and SEQ ID NO:63, respectively.

In accordance with low bone density studies referenced herein, examples of SNPs rs11576349 and rs4954555 are provided herein as SEQ ID NO:64 and SEQ ID NO:65, respectively.

As disclosed herein, the inventors identified clinical, serologic and genetic factors associated with granuloma formation in Crohn's disease (CD). 371 patients with CD who underwent disease-related surgical resection by a single surgeon were included in the study. Surgical samples were examined specifically for the presence or not of granulomas. Patients' demographic and clinical characteristics were collected by chart review, and samples drawn for IBD related serology (ASCA, anti-I2, anti-OmpC, CBir1 and ANCA) and genetic analyses. Genome-wide analyses were performed using Illumina technology. Standard statistical tests for association were used and genetic association was assessed both at the genome-wide level and against known IBD and Leprosy susceptibility loci.

As further disclosed herein, 34.7% of CD surgical samples were found to contain granulomas. Granulomas were not associated with CD disease behavior. High ASCA titer was associated with the presence of granulomas (p=0.02). Patients with granulomas were younger at time of surgery (29.9 vs. 37.6 years, p=5×10⁻⁷) and far less likely to have ever smoked (12 vs. 32%, p=7×10⁻⁵). 14 Single Nucleotide Polymorphisms (SNPs) were associated with granulomas at a level of nominal association at a genome-wide level (p<0.00005). These include a SNP adjacent to TGFb3, which has been implicated in the pathogenesis of stricturing Crohn's disease, and FTO, which is regulated by oral intake and is associated with raised body mass index. The strongest association was with NPAS2 (p=1×10⁻⁶), a core circadian gene that has been shown to modulate transcription of CX3CL1, a chemokine involved in CD pathogenesis. Amongst known IBD-associated loci, 7 were associated with granuloma formation (p<0.05), including: MUC1 (KL-6), also associated with granuloma-forming hypersensitivity pneumonitis; IL10, with known immunoregulatory function in the gut; and LRAP, associated with antigen presentation and LRRK2 a leucine-rich repeat kinase gene. One TNFSF15 SNP showed a trend towards association with the presence of granulomas (P=0.066), of particular interest given a recent report that TNFSF15 is associated with Leprosy, another granulomatous condition. Of the known Leprosy loci (in addition to LRRK2 and TNFSF15), the inventors identified association with granulomatous CD and SNPs across the cytochrome P-450 cluster. Thus, the inventors have demonstrated putative genetic and demographic associations with the presence of granulomas in CD including a number of genes associated with Leprosy suggesting unique pathways in the pathogenesis of this subset of CD.

In one embodiment, the present invention provides a method of diagnosing susceptibility to a subgroup of Crohn's disease in an individual, by obtaining a sample from the individual, and assaying the sample to determine the presence or absence of one or more genetic risk variants and/or risk serological markers, wherein the presence of one or more genetic risk variants and/or risk serological markers is indicative of susceptibility to the subgroup of Crohn's disease. In another embodiment, the subgroup of Crohn's disease is characterized by granuloma manifestations. In another embodiment, the one or more genetic risk variants are at the genetic loci of TGFb3, FTO, NPAS2, MUC1, IL10, LRAP, LRRK2, TNFSF15, and/or cytochrome p450 cluster. In another embodiment, the one or more risk serological markers include a high expression level of ASCA relative to a healthy subject.

In one embodiment, the present invention provides a method of treating Crohn's disease in an individual by determining the presence of one or more genetic risk variants associated with granulomas, and treating the individual.

As disclosed herein, the inventors identified 333 IBD subjects with bone density studies who had previously had genome wide association studies and IBD related serologies performed. Data on age, gender, ethnicity, disease distribution, surgeries, and smoking history were obtained from chart reviews. Osteoporosis, osteopenia, and normal bone mineral density (NBD) were defined by the WHO criteria based on DEXA scans. Standard tests for association between clinical characteristics, genetic markers and serologies were used. IBD related serology (ASCA, OmpC, I2, CBir-1, and ANCA) were obtained by ELISA and summarized into quartiles. Genetic data were generated using Illumina technology.

As further disclosed herein, of the 333 IBD study subjects, the inventors identified 252 cases of low bone density (LBD) and 81 cases of NBD. Disease location was not associated with LBD overall; however, perianal disease was associated with osteoporosis (P=0.021). Small bowel disease requiring surgery was associated with LBD (P=0.022), osteopenia (P=0.041) and osteoporosis (P=0.05). Smoking was not associated with bone density. Mean and median Anti-I2 titers were associated with LBD (P=0.023) and osteoporosis (P=0.006). On quartile analysis, anti-CBir1 titers were associated with LBD (P=0.036) and osteoporosis (P=0.0006); further, ASCA was associated with osteoporosis (P=0.03). 38 genetic loci achieved nominal level of genome wide significance (P<5×10⁻⁵) including multiple single nucleotide polymorphisms (SNPs) at the HLA (P=1.37×10⁻⁷) as well as genes involved in cell adhesion (laminin, P=4.41×10⁻⁵) and innate immunity (plexin, P=9.02×10⁻⁷; NLR family, P=7.39×10⁻⁶). Stepwise linear regression was performed and all but 2 SNPs (rs11576349 and rs4954555) fell out of the model. These two SNPs were independently associated with LBD (2.41×10⁻⁵ and 1.07×10⁻⁵) and together this 2 SNP model was highly associated with LBD (p-value linear regression 1.8×10⁻⁹) and explained 12.6 of the variance. Perianal disease is associated with osteoporosis; further, small bowel disease requiring surgery increases the risk for LBD. Anti-I2, anti-CBir1, and ASCA are associated with increased risk for LBD and/or osteoporosis. Genes including HLA, laminin and plexin are associated with LBD. Thus, patients with these risk factors may benefit from more aggressive screening and treatment for osteoporosis.

In one embodiment, the present invention provides a method of diagnosing susceptibility to a condition characterized by low bone density in an individual by obtaining a sample from the individual, assaying the sample to determine the presence or absence of one or more risk factors and/or risk serological markers, where the presence of one or more genetic risk factors and/or risk serological markers is indicative of susceptibility to a condition characterized by low bone density in the individual. In another embodiment, the individual is diagnosed with inflammatory bowel disease (IBD). In another embodiment, the one or more risk factors include genetic risk variants at the genetic loci of HLA, laminin, and/or plexin. In another embodiment, the presence of perianal disease is associated with an increased risk of osteoporosis. In another embodiment, the presence of small bowel disease requiring surgery is associated with an increased risk of susceptibility to LBD, osteopenia, and/or osteoporosis. In another embodiment, the one or more risk serological markers include I2, Cbir1, and/or ASCA.

In one embodiment, the present invention provides a method of treating a condition characterized by low bone density in an individual, by determining the presence of one or more risk factors and/or serological markers, and treating the individual.

As used herein, the term “IFNG” refers to the gene encoding IFN-gamma. Similarly, “IFNG production,” or “IFNG secretion” refers to the product expressed from the IFNG genetic locus.

An example of SNP rs1861494 is provided herein as SEQ ID NO:66 and SEQ ID NO:67.

“TNF” as used herein is an abbreviation of tumor necrosis factor.

As used herein, the term “TNF signaling inhibitor” (also interchangeably called as TNF blocker or inhibitor, anti-TNF reagent, agent, drug or therapeutic,) refers to any reagent that suppress responses to TNF and/or inhibits the TNF signaling, including inhibition of any molecular signaling step from the TNF ligand through its receptor to various downstream target molecules. A TNF signaling inhibitor can be a small molecule; a nucleic acid such as siRNA, shRNA, and miRNA; a nucleic acid analogue such as PNA, pc-PNA, and LNA; an aptamer; a ribosome; a peptide; a protein; an avimer; an antibody, or variants and fragments thereof. Examples of the TNF signaling inhibitor include but are not limited to CDP571, CDP860, CDP870, infliximab, adalimumab, certolizumab, certolizumab pegol, golimumab, etanercept, onercept, MAP kinase inhibitors, xanthine derivatives (e.g. pentoxifylline), bupropion, 5-HT2A agonist hallucinogens including (R)-DOI, TCB-2, LSD and LA-SS-Az, curcumin, and catechins.

As disclosed herein, the inventors determined what was the methylation status for IFNG rs1861494 SNP alleles and whether a functional relationship exists between allele specific methylation and gene expression. 154 IBD patients were genotyped for the IFNG rs1861494. DNA strand specific methylation levels for SNP +2109 and adjacent +2167 and +2209 CpG sites were determined by pyrosequencing. Allele and methylation-specific nucleo-protein binding was determined by EMSA. Levels of IFNG secretion and immune response to CBir were measured by ELISA.

As further disclosed herein, the wt rs1861494 T allele is un-methylated whereas the C allele displays 55% methylation. In adjacent CpG sites allele-specific DNA methylation was noted at the +2167, but not +2209, with decreased methylation of the C vs. T SNP allele DNA strands (p<0.001). The rs1861494 IFNG polymorphism is functionally associated with decreased IFNG production and levels of immune response to CBir. Allele-specific and methylation-sensitive alteration in DNA trans-factor binding patterns to the SNP was noted. Nucleo-protein binding to the unmethylated C SNP was lower than that seen for T SNP. However, methylation of the C allele strand markedly enhanced binding and the appearance of an additional nucleo-protein complex. These results link the same cis-regulatory IFNG variant with modulation of DNA strand methylation and transcription factor binding supporting a functional role for rs1861494 gene variant in regulating IFNG expression.

In one embodiment, the present invention provides a method of diagnosing susceptibility to inflammatory bowel disease (IBD) in an individual by obtaining a sample from the individual, assaying the sample to determine the presence or absence of one or more risk genetic variants and/or an increase in IFNG DNA methylation relative to a normal subject, and diagnosing susceptibility to inflammatory bowel disease based on the presence of one or more risk genetic variants and/or an increase in IFNG DNA methylation relative to a normal subject. In another embodiment, the IBD is ulcerative colitis. In another embodiment, the one ore more risk genetic variants include SNP rs1861494 with a “C” allele. In another embodiment, the presence of one or more risk genetic variants and/or increase in IFNG DNA methylation relative to a normal subject is associated with a decrease in levels of IFNG expressed relative to levels found in a healthy person.

In one embodiment, the present invention provides a method of diagnosing susceptibility to inflammatory bowel disease (IBD) in an individual by obtaining a sample from the individual, assaying the sample to determine the presence or absence of one or more risk genetic variants and/or a decrease in IFNG DNA methylation relative to a normal subject, and diagnosing susceptibility to inflammatory bowel disease based on the presence of one or more risk genetic variants and/or an decrease in IFNG DNA methylation relative to a normal subject. In another embodiment, the IBD is ulcerative colitis. In another embodiment, the one or more risk genetic variants include SNP rs1861494 with a “T” allele. In another embodiment, the presence of one or more risk genetic variants and/or decrease in IFNG DNA methylation relative to a normal subject is associated with a increase in levels of IFNG protein relative to levels found in a healthy person.

In one embodiment, the present invention provides a method of treating IBD in an individual by determining the presence of aberrant DNA methylation patters at the IFNG genetic locus, relative to a healthy subject, and treating the individual.

As described herein, the inventors explored the association of rs1861494 T/C SNP with severity of disease in IBD and found a significant association of the T allele to severity in both UC and CD. Furthermore, the rs1861494 T allele functionally correlated with increased IFN-gamma expression. In this context, the rs1861494 T/C polymorphism introduces a new CpG dinucleotide sequence that serves as an epigenetic target for DNA methylation resulting in altered transcription factor binding to this region that might have a functional consequence on transcription of IFN-gamma expression.

Mucosal expression of IFN-γ plays a pivotal role in IBD pathogenesis and IBD-risk regions flank IFNG. The conserved IFNG rs1861494 T/C, introduces a new CpG methylation site, and is associated with disease severity and lack of therapeutic response in other infectious and immune mediated disorders, and is in linkage-disequilibrium with a UC disease severity region. It seems likely that CpG-altering SNPs modify methylation and gene expression. This study evaluated the association between rs1861494 and clinical, serologic and methylation patterns in IBD patients.

Peripheral T cells of UC and CD patients were genotyped for rs1861494 and analyzed for allele-specific and IFNG promoter methylation. Serum ANCA and IFN-γ secretion were measured by ELISA and nucleo-protein complex formation by EMSA.

IFNG rs1861494 T allele carriage in IBD patients was associated with enhanced secretion of IFN-. T allele carriage was associated in UC with high levels of ANCA and faster progression to colectomy. In CD, it was associated with complicated disease involving a stricturing/penetrating phenotype. Likewise, IFNG rs1861494 displayed genotype specific modulation of DNA methylation and transcription factor complex formation.

This study reports the first association of IFNG rs1861494 T allele with enhanced IFN-secretion and known IBD clinical parameters indicative of more aggressive disease, as well as serological markers associated with treatment resistance to anti-TNF therapy in IBD patients. These data is useful prognostically as predictors of early response to anti-TNF therapy to identify IBD patients for improved personalized therapeutics.

In various embodiments, the present invention provides a method of administering a TNF signaling inhibitor to an individual. In one embodiment, the individual has already been diagnosed with an inflammatory bowel disease (IBD). In some embodiments, the method may consist of or may consist essentially of or may comprise: (a) genotyping the individual for a risk genetic variant at the genetic locus of IFNG; and (b) if the individual is negative for the risk genetic variant, administering the TNF signaling inhibitor to the individual, and if the individual is positive for the risk genetic variant, not administering the TNF signaling inhibitor to the individual. In other embodiments, the method may consist of or may consist essentially of or may comprise: (a) obtaining a sample from the individual; (b) contacting the sample with an oligonucleotide probe specific to a risk genetic variant at the genetic locus of IFNG; (c) forming an allele-specific hybridization complex between the oligonucleotide probe and the risk genetic variant; (d) detecting the allele-specific hybridization complex; and (e) if the allele-specific hybridization complex is not detected, administering the TNF signaling inhibitor to the individual, and if the allele-specific hybridization complex is detected, not administering the TNF signaling inhibitor to the individual. In various embodiments, the method further comprises conducting PCR amplification after forming the allele-specific hybridization complex.

In various embodiments, the present invention provides a method of treating, preventing, reducing the likelihood of having, reducing the severity of and/or slowing the progression of an inflammatory bowel disease (IBD) in an individual. In some embodiments, the method may consist of or may consist essentially of or may comprise: (a) genotyping the individual for a risk genetic variant at the genetic locus of IFNG; and (b) if the individual is positive for the risk genetic variant, conducting colectomy on the individual, and if the individual is negative for the risk genetic variant, administering a TNF signaling inhibitor to the individual. In other embodiments, the method may consist of or may consist essentially of or may comprise: (a) obtaining a sample from the individual; (b) contacting the sample with an oligonucleotide probe specific to a risk genetic variant at the genetic locus of IFNG; (c) forming an allele-specific hybridization complex between the oligonucleotide probe and the risk genetic variant; (d) detecting the allele-specific hybridization complex; and (e) if the allele-specific hybridization complex is detected, conducting colectomy on the individual, and if the allele-specific hybridization complex is not detected, administering a TNF signaling inhibitor to the individual. In various embodiments, the method further comprises conducting PCR amplification after forming the allele-specific hybridization complex.

In various embodiments, detecting the allele-specific hybridization complex comprises detecting the electrophoretic mobility of the allele-specific hybridization complex. In another embodiment, the oligonucleotide probe is labeled with a fluorescent dye. In some embodiments, the oligonucleotide probe comprises a reporter dye and a quencher dye. In still another embodiment, detecting the allele-specific hybridization complex comprises detecting fluorescence signal from the oligonucleotide probe.

In various embodiments, the present invention also provides a system for administering a TNF signaling inhibitor to an individual and/or for treating, preventing, reducing the likelihood of having, reducing the severity of and/or slowing the progression of an inflammatory bowel disease (IBD) in an individual. The system may consist of or may consist essentially of or may comprise: (a) an oligonucleotide probe specific to a risk genetic variant at the genetic locus of IFNG; and (b) a TNF signaling inhibitor. In one embodiment, the system further comprises a module configured for conducting PCR amplification. In another embodiment, the oligonucleotide probe is labeled with a fluorescent dye. In some embodiments, the oligonucleotide probe comprises a reporter dye and a quencher dye. In still another embodiment, the system further comprises a module configured to detecting fluorescence signal from the oligonucleotide probe.

In various embodiments, the present invention provides a method of treating, preventing, reducing the likelihood of having, reducing the severity of and/or slowing the progression of an inflammatory bowel disease (IBD) in an individual. The method may consist of or may consist essentially of or may comprise: (a) obtaining a sample from the individual; (b) assaying the sample to detect the presence or absence of at least one risk genetic variant at the genetic locus of IFNG, and/or assaying the sample to detect an increase or decrease in IFNG DNA methylation relative to a healthy individual; and (c) treating the IBD in the individual. In some embodiments, the method may consist of or may consist essentially of or may comprise: (a) obtaining a sample from the individual; (b) assaying the sample to detect the presence or absence of at least one risk genetic variant at the genetic locus of IFNG; and (c) treating the IBD in the individual. In other embodiments, the method may consist of or may consist essentially of or may comprise: (a) obtaining a sample from the individual; (b) assaying the sample to detect an increase or decrease in IFNG DNA methylation relative to a healthy individual; and (c) treating the IBD in the individual. In various embodiments, the method may consist of or may consist essentially of or may comprise: (a) obtaining a sample from the individual; (b) assaying the sample to detect the presence or absence of at least one risk genetic variant at the genetic locus of IFNG, and assaying the sample to detect an increase or decrease in IFNG DNA methylation relative to a healthy individual; and (c) treating the IBD in the individual. In some embodiments, treating the IBD comprises conducting colectomy on the individual, upon detecting the presence of at least one risk genetic variant at the genetic locus of IFNG and/or a decrease in IFNG DNA methylation relative to a healthy individual. In other embodiments, treating the IBD comprises administering a TNF signaling inhibitor to the individual, upon detecting the absence of at least one risk genetic variant at the genetic locus of IFNG and/or an increase in IFNG DNA methylation relative to a healthy individual.

In various embodiments, the IBD comprises Crohn's disease (CD) or ulcerative colitis (UC). In some embodiments, the IBD is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof.

In various embodiments, the individual is a human. In some embodiments, the individual is a mammalian subject including but not limited to human, monkey, ape, dog, cat, cow, horse, goat, pig, rabbit, mouse and rat.

In various embodiments, the sample comprises a nucleic acid from the individual. In some embodiments, the sample comprises a body fluid, cheek swab, mucus, whole blood, blood, serum, plasma, urine, saliva, semen, lymph, fecal extract, or sputum, or a combination thereof. In other embodiments, the sample comprises a cell or tissue. In accordance with the present invention, the cell can be a lymphoblastoid cell line obtained from the individual and transformed with an Epstein Barr virus. Still in accordance with the present invention, the cell can be a mucosal T cell, a lamina propria T cell, or a peripheral blood T cell.

In various embodiments, the risk genetic variant is the “T” allele SNP rs1861494 (for non-limiting examples, SEQ ID NO:66 and SEQ ID NO:67 herein). In various embodiments, IFNG DNA methylation is IFNG promoter methylation.

In some embodiments, the treatment methods described herein further comprise assaying the sample to detect an increase of INF-γ secretion relative to a healthy individual. In further embodiments, treating the IBD comprises conducting colectomy on the individual, upon detecting an increase of INF-γ secretion relative to a healthy individual, or administering a TNF signaling inhibitor to the individual, upon detecting no increase of INF-γ secretion relative to a healthy individual.

In other embodiments, the treatment methods described herein further comprise assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC. In further embodiments, treating the IBD comprises conducting colectomy on the individual, upon detecting an increase of the at least one risk serological marker relative to a healthy individual, or administering a TNF signaling inhibitor to the individual, upon detecting no increase of the at least one risk serological marker relative to a healthy individual. In one embodiment, the at least one risk serological marker is ANCA.

In some embodiments, the TNF signaling inhibitor comprises an anti-TNF antibody. In various embodiments, the TNF signaling inhibitor comprises infliximab, adalimumab, certolizumab, certolizumab pegol, golimumab, etanercept, or onercept, or a combination thereof.

Typical dosages of an effective amount of the TNF signaling inhibitor can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the TNF signaling inhibitor may be administered once a day (SID/QD), twice a day (BID), three times a day (TID), four times a day (QID), or more, so as to administer an effective amount of the TNF signaling inhibitor to the individual, where the effective amount is any one or more of the doses described herein.

In various embodiments, the TNF signaling inhibitor is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the TNF signaling inhibitor is administered once, twice, three or more times. In some embodiments, the TNF signaling inhibitor is administered about 1-3 times per day, 1-7 times per week, or 1-9 times per month. Still in some embodiments, the TNF signaling inhibitor is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the individual. In certain embodiments, the TNF signaling inhibitor is administered to a human.

In accordance with the invention, the TNF signaling inhibitor may be administered using the appropriate modes of administration, for instance, the modes of administration recommended by the manufacturer. In accordance with the invention, various routes may be utilized to administer the TNF signaling inhibitor of the claimed methods, including but not limited to aerosol, nasal, oral, transmucosal, transdermal, parenteral, enteral, topical, local, implantable pump, continuous infusion, capsules and/or injections. In various embodiments, the TNF signaling inhibitor is administered topically, intravascularly, intravenously, intraarterially, intramuscularly, subcutaneously, intraperitoneally, intranasally, or orally.

In various embodiments, the present invention provides a method of diagnosing an inflammatory bowel disease (IBD) subtype in an individual. The method may consist of or may consist essentially of or may comprise: (a) obtaining a sample from an individual; (b) assaying the sample to detect the presence or absence of at least one risk genetic variant at the genetic locus of IFNG, and/or assaying the sample to detect an increase or decrease in IFNG DNA methylation relative to a healthy individual; and (c) diagnosing the IBD subtype in the individual based on the presence of at least one risk genetic variant at the genetic locus of IFNG and/or an decrease in IFNG DNA methylation relative to a healthy individual.

In various embodiments, the present invention provides a method of predicting susceptibility to an inflammatory bowel disease (IBD) subtype in an individual. The method may consist of or may consist essentially of or may comprise: (a) obtaining a sample from the individual; (b) assaying the sample to detect the presence or absence of at least one risk genetic variant at the genetic locus of IFNG, and/or assaying the sample to detect an increase or decrease in IFNG DNA methylation relative to a healthy individual; and (c) predicting susceptibility to the IBD subtype in the individual based on the presence of at least one risk genetic variant at the genetic locus of IFNG and/or an decrease in IFNG DNA methylation relative to a healthy individual.

In various embodiments, the present invention also provides a system for diagnosing or predicting susceptibility to an inflammatory bowel disease (IBD) subtype in an individual. The system may consist of or may consist essentially of or may comprise an oligonucleotide probe specific to a risk genetic variant at the genetic locus of IFNG. In various embodiments, the risk genetic variant is the “T” allele SNP rs1861494. In one embodiment, the system further comprises a module configured for conducting PCR amplification. In another embodiment, the oligonucleotide probe is labeled with a fluorescent dye. In some embodiments, the oligonucleotide probe comprises a reporter dye and a quencher dye. In still another embodiment, the system further comprises a module configured to detecting fluorescence signal from the oligonucleotide probe.

In various embodiments, the IBD subtype comprises Crohn's disease (CD), ulcerative colitis (UC), or medically refractory UC (MR-UC). In one embodiment, the IBD subtype is associated with early surgical intervention or faster progression to colectomy. In another embodiment, the IBD subtype is associated with poor response, no response, and/or resistance to anti-TNF therapy. In some embodiments, the IBD subtype is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof.

In various embodiments, the IBD subtype is associated with at least one risk serological marker selected from the group consisting of ANCA, ASCA, anti-Cbir1, anti-I2, and anti-OmpC.

In various embodiments, the individual is a human. In some embodiments, the individual is a mammalian subject including but not limited to human, monkey, ape, dog, cat, cow, horse, goat, pig, rabbit, mouse and rat.

In various embodiments, the sample comprises a nucleic acid from the individual. In some embodiments, the sample comprises a body fluid, cheek swab, mucus, whole blood, blood, serum, plasma, urine, saliva, semen, lymph, fecal extract, or sputum, or a combination thereof. In other embodiments, the sample comprises a cell or tissue. In accordance with the present invention, the cell can be a lymphoblastoid cell line obtained from the individual and transformed with an Epstein Barr virus. Still in accordance with the present invention, the cell can be a mucosal T cell, a lamina propria T cell, or a peripheral blood T cell.

In various embodiments, the risk genetic variant is the “T” allele SNP rs1861494. In various embodiments, IFNG DNA methylation is IFNG promoter methylation. In one embodiment, the at least one risk genetic variant is associated with a decrease of IFNG DNA methylation relative to a healthy individual.

In one embodiment, the at least one risk genetic variant is associated with an increase of INF-γ secretion relative to a healthy individual. In another embodiment, the methods described herein further comprise assaying the sample to detect an increase of INF-γ secretion relative to a healthy individual. In still another embodiment, the methods described herein further comprise predicting susceptibility to and/or diagnosing the IBD subtype in the individual based on an increase of INF-γ secretion relative to a healthy individual.

In one embodiment, the at least one risk genetic variant is associated with an increase of ANCA relative to a healthy individual. In another embodiment, the methods described herein further comprise assaying the sample to detect an increase of ANCA relative to a healthy individual. In still another embodiment, the methods described herein further comprise predicting susceptibility to and/or diagnosing the IBD subtype in the individual based on an increase of ANCA relative to a healthy individual.

In one embodiment, the at least one risk genetic variant is associated with an increase of at least one risk serological marker relative to a healthy individual, wherein the at least one risk serological marker is selected from the group consisting of ANCA, ASCA, anti-Cbir1, anti-I2, and anti-OmpC. In another embodiment, the methods described herein further comprise assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC. In still another embodiment, the methods described herein further comprise predicting susceptibility to and/or diagnosing the IBD subtype in the individual based on an increase of at least one risk serological marker relative to a healthy individual.

“TPMT” as used herein is an abbreviation for thiopurine methyltransferase.

“HLA-DRB1” as used herein is an abbreviation for HLA class II histocompatibility antigen, DRB1-9 beta chain.

“CREM” as used herein is an abbreviation for cAMP-responsive element modulator.

“TAGAP” as used herein is an abbreviation for T-cell activation RhoGTPase activating protein.

“PLCL1” as used herein is an abbreviation for phospholipase C-like 1.

“GPX4” as used herein is an abbreviation for glutathione peroxidase 4.

“SBNO2” as used herein is an abbreviation for strawberry notch homolog 2.

“MEF2A” as used herein is an abbreviation for myocyte enhancer factor 2A.

“LYSMD4” as used herein is an abbreviation for LysM, putative peptidoglycan-binding, domain containing 4.

Various single nucleotide polymorphisms (SNPs) are used herein. Examples of SNPs rs2516049, rs3936503, rs212388, rs10196612, rs2024092, rs4663142, rs2310173, rs10758669, rs736289, rs4077515, and rs6651252, are provided herein as SEQ ID NO:83, SEQ ID NO:84, SEQ ID NO:85, SEQ ID NO:86, SEQ ID NO:87, SEQ ID NO:88, SEQ ID NO:89, SEQ ID NO: 90, SEQ ID NO:91, SEQ ID NO:92, and SEQ ID NO:93, respectively.

“Thiopurine drugs” as used herein, are purine antimetabolites widely used in the treatment of acute lymphoblastic leukemia, autoimmune disorders, and organ transplant recipients. Similarly, “thiopurine treatment” as used herein means treating a condition or disease in an individual through the administration of thiopurine, or a pharmaceutical equivalent, analog, derivative, and/or salt thereof, to the individual being treated. In some embodiments, a “risk variant” is a variant, or marker that is indicative of a likelihood of successful thiopurine-induced corticosteroid-free remission in the individual being treated with thiopurine.

As disclosed herein, the inventors tested associations of known IBD susceptibility loci and pharmacogenetic genome-wide association study (GWAS)-identified loci, as well as clinical and immune phenotypes, with thiopurine-induced corticosteroid-free remission in IBD, and developed a predictive model of remission. Corticosteroid-free remission at 26 weeks after thiopurine initiation was defined using the Harvey Bradshaw Index (HBI) for Crohn's disease (CD) and partial Mayo score for ulcerative colitis (UC). Serum was assayed for ASCA IgA and IgG, anti-OmpC, anti-CBir1, I2, and pANCA using ELISA. Clinical phenotypes included age, gender, IBD subtype (CD versus UC), disease duration at thiopurine initiation, and age at diagnosis. Genotyping was performed using Illumina technology. Univariate analyses tested associations of phenotype and genotype with remission. Stepwise logistic regression was performed to build predictive models.

As further disclosed herein, corticosteroid-free remission occurred in 56 of 122 subjects (45.9%) at week 26. Female gender (OR=0.37; 95% CI: 0.18-0.77; P=0.011) and pANCA (OR=0.23; 95% CI: 0.06-0.87; P=0.049) were negatively associated with corticosteroid-free remission at 26 weeks. Five known IBD susceptibility loci were associated with corticosteroid-free remission (P<0.05) (Table 10 herein). A single nucleotide polymorphism (SNP) at 15q31 tagging MEF2A (macrophage differentiation) and LYSMD4 (peptidoglycan binding) met the criteria for nominal association at the genome wide level for remission (OR=9.5; P=3E-05). The most predictive model of remission included the previously identified HLA-DRB1 locus (rs2516049), 7 novel “pharmacogenetic” GWAS loci, pANCA, disease duration, and a diagnosis of UC with an R-squared of 0.884, area under the curve [AUC] of 0.985, sensitivity of 0.929, specificity of 0.919, accuracy of 0.826, and positive likelihood ratio of 11.45. The probability of remission increased 7.3-fold when the number of predictors increased from 0-4 to 5-7 (95% CI: 2.43-21.66; P=0.0004). The combination of genotype with clinical and immune phenotypes is most predictive of corticosteroid-free remission after thiopurine initiation. Defining predictors of therapeutic efficacy to thiopurines allows identification of patients who will benefit most from this class of therapy, contributing to a more individualized approach to therapy.

In one embodiment, the present invention provides a method of predicting a therapeutic efficacy of thiopurines in an individual, by determining the presence or absence of one or more risk variants at the HLA-DRB1, CREM, TAGAP, PLCL1, GPX4 and/or SBNO2 genetic loci, wherein the presence of one or more risk variants at the HLA-DRB1, CREM, TAGAP, PLCL1, GPX4 and/or SBNO2 genetic loci is indicative of an effective therapeutic efficacy of thiopurines in the individual. In another embodiment, the risk variants are located at the genetic loci of ARL4C, IL1R2, JAK2, 19q13, CARD9, SNAPC4, and/or 8q24. In another embodiment, the individual has inflammatory bowel disease (IBD). In another embodiment, the effective therapeutic efficacy of thiopurines is a thiopurine-induced corticosteroid-free remission in IBD. In another embodiment, the one or more risk variants are associated with one or more antibody markers. In another embodiment, the one or more risk variants are listed in Table 10 herein. In another embodiment, the individual is a child.

In another embodiment, the present invention provides a method of treating IBD in an individual, comprising determining the presence of one or more genetic risk variants at the genetic loci of ARL4C, IL1R2, JAK2, 19q13, CARD9, SNAPC4, 8q24, HLA-DRB1, CREM, TAGAP, PLCL1, GPX4 and/or SBNO2, and then treating the individual by administering a composition comprising thiopurine, or a pharmaceutical equivalent, analog, derivative, and/or salt thereof.

The present invention is also directed to a kit to predict therapeutic remission to thiopurines. The kit is an assemblage of materials or components, including at least one of the inventive compositions. The exact nature of the components configured in the inventive kit depends on its intended purpose. For example, some embodiments are configured for the purpose of treating inflammatory bowel disease. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to predict therapeutic remission to thiopurines, or to treat IBD, or to perform organ transplantation. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a method of determining an individual as having susceptibility to a condition, comprising: obtaining a sample from the individual; assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine; detecting the risk variants or risk haplotypes in the sample, and/or detecting the serological markers in the sample, and/or detecting increased or decreased methylation at the gene in the sample, and/or detecting increased or decreased secretion of the cytokine; and determining the individual as having susceptibility to the condition. In various embodiments, the more risk variants or risk haplotypes are detected in the sample, the more susceptibility to the condition the individual has.

In various embodiments, the present invention provides a method of determining an individual as having a low probability of developing a condition, comprising: obtaining a sample from the individual; assaying the sample to detect one or more protective variants or protective haplotypes, and/or assaying the sample to detect one or more serological markers, and/or assaying the sample to detect increased or decreased methylation at a gene, and/or assaying the sample to detect increased or decreased secretion of a cytokine; detecting the protective variants or protective haplotypes in the sample, and/or detecting the serological markers in the sample, and/or detecting increased or decreased methylation at the gene in the sample, and/or detecting increased or decreased secretion of the cytokine; and determining the individual as having a low probability of developing the condition. In various embodiments, the more protective variants or protective haplotypes are detected in the sample, the lower probability of developing the condition the individual has.

In various embodiments, the present invention provides a method of determining an individual as having a condition, comprising: obtaining a sample from the individual; assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine; detecting the risk variants or risk haplotypes in the sample, and/or detecting the serological markers in the sample, and/or detecting increased or decreased methylation at the gene in the sample, and/or detecting increased or decreased secretion of the cytokine; and determining the individual as having the condition.

In various embodiments, the present invention provides a method of selecting a treatment for an individual with a condition, comprising: obtaining a sample from the individual; assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine; detecting the risk variants or risk haplotypes in the sample, and/or detecting the serological markers in the sample, and/or detecting increased or decreased methylation at the gene in the sample, and/or detecting increased or decreased secretion of the cytokine; and selecting a treatment for the individual.

In various embodiments, the present invention provides a method of predicting an individual as having responsiveness to a treatment of a condition, comprising: obtaining a sample from the individual; assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine; detecting the risk variants or risk haplotypes in the sample, and/or detecting the serological markers in the sample, and/or detecting increased or decreased methylation at the gene in the sample, and/or detecting increased or decreased secretion of the cytokine; and predicting the individual as having responsiveness to the treatment of the condition. In various embodiments, the more risk variants or risk haplotypes are detected in the sample, the more responsiveness to the treatment of the condition the individual has.

In various embodiments, the present invention provides a method of treating a condition in an individual, comprising: obtaining a sample from the individual; assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine; detecting the risk variants or risk haplotypes in the sample, and/or detecting the serological markers in the sample, and/or detecting increased or decreased methylation at the gene in the sample, and/or detecting increased or decreased secretion of the cytokine; and treating the condition in the individual with a treatment.

In various embodiments, assaying the sample to detect the risk variants or risk haplotypes comprises: contacting the sample with one or more allele-specific oligonucleotide probes targeting the risk variants or risk haplotypes; generating double-stranded hybridization complex through allele-specific binding between the risk variants or risk haplotypes and said allele-specific oligonucleotide probes; and detecting the double-stranded hybridization complex newly generated through allele-specific binding between the risk variants or risk haplotypes and said allele-specific oligonucleotide probes. In some embodiments, the method further comprises conducting PCR amplification of the double-stranded hybridization complex.

In accordance with the present invention, said allele-specific oligonucleotide probes may comprise about 10-15, 15-20, 20-25, 25-30, 30-35, 35-40, 40-45, or 45-50 nucleotides; they are either identical or complementary to a sequence segment encompassing the polymorphic position of a SNP as disclosed herein; and they are specific to one or the other allele at the polymorphic position. For a non-limiting example, rs10758669 has either A or C allele at its polymorphic position (e.g., “m” at nucleotide 301 of SEQ ID NO:1). Hence, an allele-specific oligonucleotide probe for the A allele at rs10758669 may comprise, for a non-limiting example, 21 nucleotides; and these 21 nucleotides are either identical or complementary to the sequence segment 281-301, 282-302, 283-303, 284-304, 285-305, 286-306, 287-307, 288-308, 289-309, 290-311, 291-311, 292-312, 293-313, 294-314, 295-315, 296-316, 297-317, 298-318, 299-319, 300-320, or 301-321 of SEQ ID NO:1 where nucleotide 301 is set as the A allele. Vice versa, an allele-specific oligonucleotide probe for the C allele at rs10758669 may comprise, for a non-limiting example, 21 nucleotides; and these 21 nucleotides are either identical or complementary to the sequence segment 281-301, 282-302, 283-303, 284-304, 285-305, 286-306, 287-307, 288-308, 289-309, 290-311, 291-311, 292-312, 293-313, 294-314, 295-315, 296-316, 297-317, 298-318, 299-319, 300-320, or 301-321 of SEQ ID NO:1 where nucleotide 301 is set as the C allele.

In some embodiments, said allele-specific oligonucleotide probes are labeled with one or more fluorescent dyes, and wherein detecting the double-stranded hybridization complex comprises detecting fluorescence signals from the fluorescent dyes. In some embodiments, said allele-specific oligonucleotide probes are labeled with a reporter dye and a quencher dye. In some embodiments, detecting the double-stranded hybridization complex comprises detecting the electrophoretic mobility of the double-stranded hybridization complex.

In various embodiments, the individual is Jewish, non-Jewish, Caucasian, non-Caucasian, Jewish Caucasian, non-Jewish Caucasian, male, female, a child, or an adult, or a combination thereof.

In various embodiments, the condition is inflammatory bowel disease (IBD), subtype of IBD, Crohn's disease (CD), subtype of CD, ulcerative colitis (UC), subtype of UC, aggressive form of IBD, an aggressive of CD, aggressive form of UC, complicated form of IBD, complicated form of CD, complicated form of UC, granuloma, low bone density (LBD), osteoporosis, osteopenia, acute lymphoblastic leukemia, autoimmune disorder, or organ transplantation.

In various embodiments, the condition is associated with early surgical intervention, severe ulcerative colitis, colitis, a small bowel disease phenotype, an aggressive complicating phenotype, an internal penetrating disease phenotype, a stricturing disease phenotype, a fibrostenosing disease phenotype, a fistulating disease phenotype, granuloma, low bone density (LBD), osteoporosis, osteopenia, or perianal disease, or a combination thereof.

In various embodiments, the sample comprises body fluid, cheek swab, mucus, whole blood, blood, serum, plasma, urine, saliva, semen, lymph, fecal extract, or sputum, or a combination thereof. In various embodiments, the sample comprises a tissue, a cell, a T cell, a mucosal T cell, a lamina propria T cell, a peripheral blood T cell, or a lymphoblastoid cell line obtained from the individual and transformed with an Epstein Barr virus, or a combination thereof.

In various embodiments, the risk variants or risk haplotypes are located at one or more genetic loci of interferon gamma (IFNG), Janus kinase 2 (JAK2), SMAD family member 3 (SMAD3), zinc finger protein 365 (ZNF365), fucosyltransferase 2 (FUT2), alpha-fetoprotein (AFP), afamin (AFM), Ras association (RalGDS/AF-6) domain family member 6 (RASSF6), phosphoglucomutase 2 (PGM2), AK097193, fragile histidine triad gene (FHIT), ETS variant 4 (ETV4), malic enzyme 1 (ME1), WD repeat domain 64 (WDR64), ataxin 2 binding protein 1 (A2BP1), cadherin 2 (CDH2), heat shock 70 kDa binding protein, co-chaperone 1 (HSPBP1), protein phophatase 6 regulatory subunit 1 (PPP6R1), BR serine/threonine kinase (BRSK1), Chromosome 4, Chromosome 15, Chromosome 18, transforming growth factor, beta 3 (TGFb3), fat mass and obesity associated (FTO), neuronal PAS domain protein 2 (NPAS2), mucin 1, cell surface associated (MUC1), interleukin 10 (IL10), endoplasmic reticulum aminopeptidase 2 (LRAP), leucine-rich repeat kinase 2 (LRRK2), tumor necrosis factor (ligand) superfamily, member 15 (TNFSF15), cytochrome P-450 cluster, major histocompatibility complex (HLA), laminin, plexin, NLR family, ADP-ribosylation factor-like 4C (ARL4C), interleukin 1 receptor, type II (IL1R2), 19q13, caspase recruitment domain family, member 9 (CARD9), small nuclear RNA activating complex, polypeptide 4, 190 kDa (SNAPC4), 8q24, major histocompatibility complex, class II, DR beta 1 (HLA-DRB1), CAMP responsive element modulator (CREM), T-cell activation RhoGTPase activating protein (TAGAP), phospholipase C-Like 1 (PLCL1), glutathione peroxidase 4 (GPX4), strawberry notch homolog 2 (Drosophila) (SBNO2), myocyte enhancer factor 2A (MEF2A), and LysM, putative peptidoglycan-binding, domain containing 4 (LYSMD4).

In various embodiments, the risk variants or risk haplotypes comprise one or more of SEQ ID NOs:1-67 and 83-93. In various embodiments, the risk variants or risk haplotypes comprise one or more of: C or A allele at rs10758669, T or A allele at rs3808850, C or A allele at rs1887429, A or G allele at rs2274471, G or A allele at rs7849191, G or A allele at rs3780374, A or C allele at rs10815160, T or A allele at rs9972423, G or A allele at rs2118611, C or G allele at rs11071933, G or A allele at rs1438386, G or A allele at rs718663, A or G allele at rs7163381, A or G allele at rs920293, A or G allele at rs745103, A or T allele at rs12439792, A or G allele at rs17293443, G or A allele at rs893473, C or A allele at rs2289263, G or C allele at rs2033785, G or A allele at rs11637659, G or A allele at rs10152307, G or A allele at rs4776900, A or G allele at rs7179840, A or G allele at rs11071939, G or A allele at rs16950687, A or G allele at rs10740085, G or C allele at rs12768538, G or A allele at rs7068361, G or A allele at rs7071642, A or G allele at rs7076156, A or G allele at rs729739, G or C allele at rs10995271, A or G allele at rs12766391, A or G allele at rs10761659, A or G allele at rs224120, G or A allele at rs492602, A or G allele at rs601338, A or G allele at rs602662, G or A allele at rs485186, A or G allele at rs504963, C or T allele at rs676388, G or C allele at rs7668327, G or A allele at rs10001225, G or A allele at rs4694164, G or A allele at rs2071098, G or A allele at rs1358592, C or A allele at rs4574378, G or A allele at rs1919469, G or A allele at rs12507775, A or C allele at rs1271392, G or A allele at rs2995965, G or A allele at rs1863284, A or G allele at rs2911869, A or G allele at rs2911920, A or C allele at rs1030349, C or T allele at rs246336, C or T allele at rs6566234, C or T allele at rs291528, C or T allele at rs291523, C or A allele at rs1973780, G or A allele at rs1728171, G or A allele at rs9449593, A or G allele at rs6690359, G or A allele at rs1019257, G or A allele at rs766613, A or G allele at rs10403164, T or C allele at rs13148469, G or A allele at rs2050719, C or A allele at rs7760387, A or C allele at rs9399527, C or T allele at rs9784771, C or T allele at rs282792, A or G allele at rs10440086, T or C allele at rs1352851, T or C allele at rs13148469, C or T allele at rs282792, C or T allele at rs443394, A or G allele at rs8091293, T or G allele at rs10514090, A or G allele at rs11576349, C or T allele at rs4954555, T or C allele at rs1861494, A or G allele at rs2516049, T or C allele at rs3936503, T or C allele at rs212388, T or C allele at rs10196612, T or C allele at rs2024092, T or C allele at rs4663142, T or G allele at rs2310173, T or G allele at rs10758669, T or C allele at rs736289, T or C allele at rs4077515, and T or C allele at rs6651252.

In various embodiments, the risk haplotypes comprise one or more of: JAK2 Block 1 Haplotype 1, JAK2 Block 2 Haplotype 1, JAK2 Block 3 Haplotype 3, SMAD3 Block 2 Haplotype 4, SMAD3 Block 5 Haplotype 1, and SMAD3 Block 6 Haplotype 1.

In various embodiments, the protective variants or protective haplotypes are located at one or more genetic loci of interferon gamma (IFNG), Janus kinase 2 (JAK2), SMAD family member 3 (SMAD3), zinc finger protein 365 (ZNF365), fucosyltransferase 2 (FUT2), alpha-fetoprotein (AFP), afamin (AFM), Ras association (RalGDS/AF-6) domain family member 6 (RASSF6), phosphoglucomutase 2 (PGM2), AK097193, fragile histidine triad gene (FHIT), ETS variant 4 (ETV4), malic enzyme 1 (ME1), WD repeat domain 64 (WDR64), ataxin 2 binding protein 1 (A2BP1), cadherin 2 (CDH2), heat shock 70 kDa binding protein, co-chaperone 1 (HSPBP1), protein phophatase 6 regulatory subunit 1 (PPP6R1), BR serine/threonine kinase (BRSK1), Chromosome 4, Chromosome 15, Chromosome 18, transforming growth factor, beta 3 (TGFb3), fat mass and obesity associated (FTO), neuronal PAS domain protein 2 (NPAS2), mucin 1, cell surface associated (MUC1), interleukin 10 (IL10), endoplasmic reticulum aminopeptidase 2 (LRAP), leucine-rich repeat kinase 2 (LRRK2), tumor necrosis factor (ligand) superfamily, member 15 (TNFSF15), cytochrome P-450 cluster, major histocompatibility complex (HLA), laminin, plexin, NLR family, ADP-ribosylation factor-like 4C (ARL4C), interleukin 1 receptor, type II (IL1R2), 19q13, caspase recruitment domain family, member 9 (CARD9), small nuclear RNA activating complex, polypeptide 4, 190 kDa (SNAPC4), 8q24, major histocompatibility complex, class II, DR beta 1 (HLA-DRB1), CAMP responsive element modulator (CREM), T-cell activation RhoGTPase activating protein (TAGAP), phospholipase C-Like 1 (PLCL1), glutathione peroxidase 4 (GPX4), strawberry notch homolog 2 (Drosophila) (SBNO2), myocyte enhancer factor 2A (MEF2A), and LysM, putative peptidoglycan-binding, domain containing 4 (LYSMD4).

In various embodiments, the protective variants or protective haplotypes comprise one or more of SEQ ID NOs:1-67 and 83-93. In various embodiments, the protective variants or protective haplotypes comprise one or more of: C or A allele at rs10758669, T or A allele at rs3808850, C or A allele at rs1887429, A or G allele at rs2274471, G or A allele at rs7849191, G or A allele at rs3780374, A or C allele at rs10815160, T or A allele at rs9972423, G or A allele at rs2118611, C or G allele at rs11071933, G or A allele at rs1438386, G or A allele at rs718663, A or G allele at rs7163381, A or G allele at rs920293, A or G allele at rs745103, A or T allele at rs12439792, A or G allele at rs17293443, G or A allele at rs893473, C or A allele at rs2289263, G or C allele at rs2033785, G or A allele at rs11637659, G or A allele at rs10152307, G or A allele at rs4776900, A or G allele at rs7179840, A or G allele at rs11071939, G or A allele at rs16950687, A or G allele at rs10740085, G or C allele at rs12768538, G or A allele at rs7068361, G or A allele at rs7071642, A or G allele at rs7076156, A or G allele at rs729739, G or C allele at rs10995271, A or G allele at rs12766391, A or G allele at rs10761659, A or G allele at rs224120, G or A allele at rs492602, A or G allele at rs601338, A or G allele at rs602662, G or A allele at rs485186, A or G allele at rs504963, C or T allele at rs676388, G or C allele at rs7668327, G or A allele at rs10001225, G or A allele at rs4694164, G or A allele at rs2071098, G or A allele at rs1358592, C or A allele at rs4574378, G or A allele at rs1919469, G or A allele at rs12507775, A or C allele at rs1271392, G or A allele at rs2995965, G or A allele at rs1863284, A or G allele at rs2911869, A or G allele at rs2911920, A or C allele at rs1030349, C or T allele at rs246336, C or T allele at rs6566234, C or T allele at rs291528, C or T allele at rs291523, C or A allele at rs1973780, G or A allele at rs1728171, G or A allele at rs9449593, A or G allele at rs6690359, G or A allele at rs1019257, G or A allele at rs766613, A or G allele at rs10403164, T or C allele at rs13148469, G or A allele at rs2050719, C or A allele at rs7760387, A or C allele at rs9399527, C or T allele at rs9784771, C or T allele at rs282792, A or G allele at rs10440086, T or C allele at rs1352851, T or C allele at rs13148469, C or T allele at rs282792, C or T allele at rs443394, A or G allele at rs8091293, T or G allele at rs10514090, A or G allele at rs11576349, C or T allele at rs4954555, T or C allele at rs1861494, A or G allele at rs2516049, T or C allele at rs3936503, T or C allele at rs212388, T or C allele at rs10196612, T or C allele at rs2024092, T or C allele at rs4663142, T or G allele at rs2310173, T or G allele at rs10758669, T or C allele at rs736289, T or C allele at rs4077515, and T or C allele at rs6651252.

In various embodiments, the protective haplotypes comprise one or more of: JAK2 Block 1 Haplotype 3, JAK2 Block 2 Haplotype 2, JAK2 Block 3 Haplotype 1, SMAD3 Block 4 Haplotype 1, SMAD3 Block 5 Haplotype 2, and SMAD3 Block 6 Haplotype 2.

In various embodiments, the serological marker comprises one or more of ANCA, pANCA, ASCA, anti-Cbir1, anti-I2, and anti-OmpC. In some embodiments, the serological marker in the individual is detected at a higher level relative to a healthy subject. In other embodiments, the serological marker in the individual is detected at a lower level relative to a healthy subject.

In various embodiments, the gene at which increased or decreased methylation is detected is interferon gamma (IFNG), Janus kinase 2 (JAK2), SMAD family member 3 (SMAD3), zinc finger protein 365 (ZNF365), fucosyltransferase 2 (FUT2), alpha-fetoprotein (AFP), afamin (AFM), Ras association (RalGDS/AF-6) domain family member 6 (RASSF6), phosphoglucomutase 2 (PGM2), AK097193, fragile histidine triad gene (FHIT), ETS variant 4 (ETV4), malic enzyme 1 (ME1), WD repeat domain 64 (WDR64), ataxin 2 binding protein 1 (A2BP1), cadherin 2 (CDH2), heat shock 70 kDa binding protein, co-chaperone 1 (HSPBP1), protein phophatase 6 regulatory subunit 1 (PPP6R1), BR serine/threonine kinase (BRSK1), Chromosome 4, Chromosome 15, Chromosome 18, transforming growth factor, beta 3 (TGFb3), fat mass and obesity associated (FTO), neuronal PAS domain protein 2 (NPAS2), mucin 1, cell surface associated (MUC1), interleukin 10 (IL10), endoplasmic reticulum aminopeptidase 2 (LRAP), leucine-rich repeat kinase 2 (LRRK2), tumor necrosis factor (ligand) superfamily, member 15 (TNFSF15), cytochrome P-450 cluster, major histocompatibility complex (HLA), laminin, plexin, NLR family, ADP-ribosylation factor-like 4C (ARL4C), interleukin 1 receptor, type II (IL1R2), 19q13, caspase recruitment domain family, member 9 (CARD9), small nuclear RNA activating complex, polypeptide 4, 190 kDa (SNAPC4), 8q24, major histocompatibility complex, class II, DR beta 1 (HLA-DRB1), CAMP responsive element modulator (CREM), T-cell activation RhoGTPase activating protein (TAGAP), phospholipase C-Like 1 (PLCL1), glutathione peroxidase 4 (GPX4), strawberry notch homolog 2 (Drosophila) (SBNO2), myocyte enhancer factor 2A (MEF2A), and LysM, putative peptidoglycan-binding, domain containing 4 (LYSMD4). In some embodiments, the increased or decreased methylation is detected in the promoter of the gene. In various embodiments, the increased or decreased methylation in the individual is relative to a healthy subject. In some embodiments, methylation is detected to be increased. In some embodiments, methylation is detected to be decreased.

In various embodiments, the cytokine is IFNγ. In various embodiments, the increased or decreased secretion of the cytokine in the individual is relative to a healthy subject. In some embodiments, cytokine secretion is detected to be increased. In some embodiments, cytokine secretion is detected to be decreased.

In various embodiments, the treatment specifically benefits those who have the risk variants or risk haplotypes, and/or the serological markers, and/or increased or decreased methylation at the gene, and/or increased or decreased secretion of the cytokine. In various embodiments, the treatment comprises one or more of: (a) conducting colectomy on the individual; (b) administering a TNF signaling inhibitor to the individual; and (c) administering a thiopurine, or a pharmaceutical equivalent, analog, derivative, and/or salt thereof, to the individual.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al. (“3’-minor groove binder-DNA probes increase sequence specificity at PCR extension temperature, “Nucleic Acids Research 28:655-661 (2000)) and Jarcho et al. in Dracopoli et al. (Current Protocols in Human Genetics pages 2.7.1-2.7.5, John Wiley & Sons, New York). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Kutyavin, et al., Nucleic Acids Research 28:655-661 (2000); Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Innis, et al., (Ed.), PCR Protocols, San Diego: Academic Press, Inc. (1990); Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well-known assays described above or another art-recognized genetic assay.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Methods of Using Smad3 and Jak2 Genetic Variants to Diagnose and Predict Inflammatory Bowel Disease

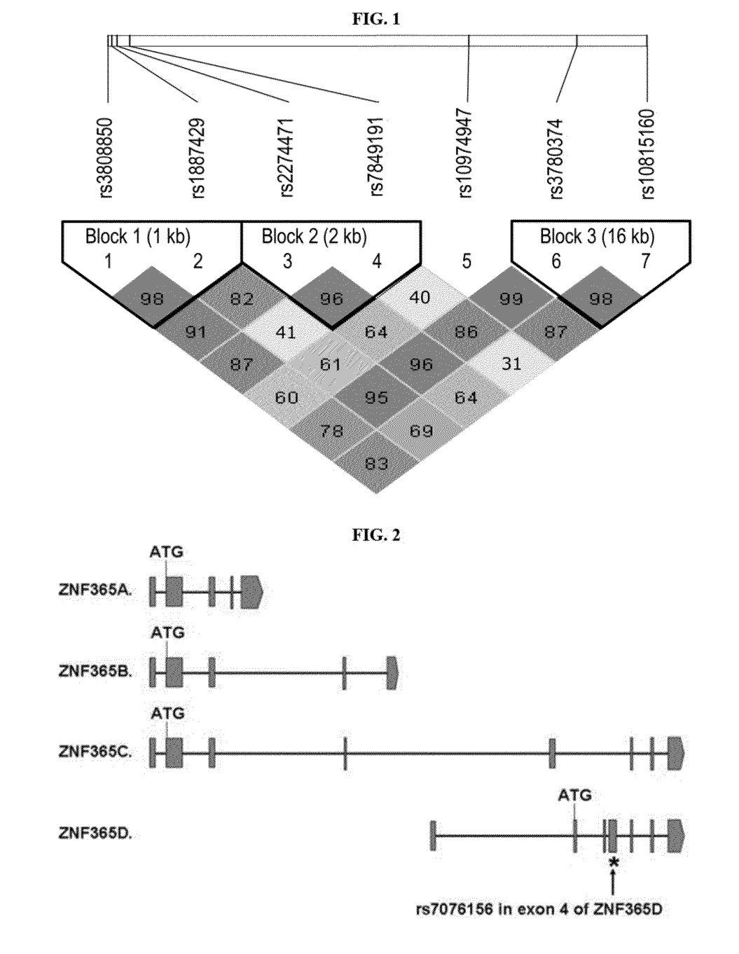

This non-limiting example relates to methods of diagnosing inflammatory bowel disease by determining the presence or absence of genetic variants at SMAD3 and/or JAK2 loci. In various embodiments, this non-limiting example provides a method of diagnosing a Crohn's disease subtype in an individual by determining the presence or absence of a risk variant at the SMAD3 and/or JAK2 loci. Exemplar data are shown in FIG. 1.

Table 1 describes various JAK2 haplotypes with statistically significant associations. The “B” corresponds with the Block number, and the “H” corresponds with the Haplotype number.

TABLE 1
Significant JAK2 haplotypes and variants

| JAK2 | | Case | Control | P | OR | PAR |
|---|---|---|---|---|---|---|
| best SNP from | [SEQ ID NO: 1] | 0.374 | 0.348 | 3.46 × 10⁻⁹ | 1.1 | 3.40% |
| GWAS (meta-analysis) | rs10758669 (allele C) | | | | | |
| haplotypes of JAK2 from | B1H1carrier | 0.683 | 0.640 | 1.8 × 10⁻² | 1.2 | 11.30% |
| Cedars | | | | | | |
| | B1H3carrier | 0.466 | 0.509 | 2.7 × 10⁻² | 0.8 | −10.20% |
| | B2H1carrier | 0.630 | 0.591 | 4 × 10⁻² | 1.2 | 10.60% |
| | B2H2homozygotes | 0.115 | 0.150 | 6.2 × 10⁻³ | 0.7 | −4.50% |
| | B3Hlcarrier | 0.664 | 0.710 | 1.1 × 10⁻² | 0.8 | −14.20% |
| | B3H3carrier | 0.496 | 0.451 | 2 × 10⁻² | 1.2 | 8.30% |

The JAK2 haplotypes referenced in Table 1 above and herein are defined in Table 2 below, where JAK2 Blocks 1-3 and haplotypes 1-3 are defined by listed SNPs and the corresponding allele. The “B” corresponds with the Block number, and “H” corresponds with the Haplotype number.

| | | Haplotype 1 | Haplotype 2 | Haplotype 3 |
|---|---|---|---|---|
| Block | SNPs | Alleles | Alleles | Alleles |
| 1 | Rs3808850 | T | T | A |
| | [SEQ ID NO: 2] | | | |
| | Rs1887429 | C | A | C |
| | [SEQ ID NO: 3] | | | |
| 2 | Rs2274471 | A | A | G |
| | [SEQ ID NO: 4] | | | |
| | Rs7849191 | G | A | G |
| | [SEQ ID NO: 5] | | | |
| 3 | Rs3780374 | G | G | A |
| | [SEQ ID NO: 6] | | | |
| | Rs10815160 | A | C | A |
| | [SEQ ID NO: 7] | | | |

| | |
|---|---|
| B1H1:TC | 0.42 |
| B1H2:TA | 0.30 |
| B1H3:AC | 0.28 |
| B2H1:AG | 0.38 |
| B2H2:AA | 0.36 |
| B2H3:GG | 0.26 |
| B3H1:GA | 0.44 |
| B3H2:GC | 0.28 |
| B3H3:AA | 0.28 |

Table 3 describes various SMAD3 haplotypes with statistically significant associations. The “B” corresponds with the Block number, and the “H” corresponds with the Haplotype number.

TABLE 3
Significant SMAD3 haplotypes and variants

| SMAD3 | | Case | Control | P | OR | PAR |
|---|---|---|---|---|---|---|
| best SNP | not identified | | | | | |
| from GWAS | | | | | | |
| (meta- | | | | | | |
| analysis) | | | | | | |
| haplotypes | B2H4carrier | 0.129 | 0.100 | 2.3 × 10⁻² | 1.3 | 3% |
| of SMAD3 | | | | | | |
| from Cedars | | | | | | |
| | B4H1carrier | 0.753 | 0.793 | 1.5 × 10⁻² | 0.8 | −15.90% |
| | B5H1carrier | 0.736 | 0.691 | 1.1 × 10⁻² | 1.2 | 12.10% |
| | B5H2carrier | 0.476 | 0.531 | 4.3 × 10⁻³ | 0.8 | −10.60% |
| | B6H1carrier | 0.504 | 0.452 | 8.2 × 10⁻³ | 1.2 | 8.30% |
| | B6H2homo | 0.051 | 0.082 | 1.2 × 10⁻³ | 0.6 | −3.20% |

### Table 4(a)-4(d) Haplotype Information of SMAD3

TABLE 4(a)
describes haplotype information on Block
2 of SMAD3, specifically for SMAD3
Block 2 Haplotype 4.

| SNPs | Alleles |
|---|---|
| Rs9972423 [SEQ ID NO: 8] | T |
| Rs2118611 [SEQ ID NO: 9] | G |
| Rs11071933 [SEQ ID NO: 10] | C |
| Rs1438386 [SEQ ID NO: 11] | G |
| Rs718663 [SEQ ID NO: 12] | G |
| Rs7163381 [SEQ ID NO: 13] | A |
| Rs920293 [SEQ ID NO: 14] | A |
| B2H1:AAGAAGA | 0.37 |
| B2H2:TAGAAGA | 0.22 |
| B2H3:TGCGAAA | 0.11 |
| B2H4:TGCGGAA | 0.06 |
| B2H5:TACGAAG | 0.06 |
| B2H6:TAGGAGA | 0.06 |

The “B” corresponds with the Block number,
and “H” corresponds with the Haplotype number.

TABLE 4(b)
describes haplotype information on Block 4
of SMAD3, specifically SMAD3 Block 4
Haplotype 1. The “B” corresponds with the
Block number, and “H” corresponds
with the Haplotype number.

| SNPs | Alleles |
|---|---|
| Rs745103 | A |
| Rs12439792 | A |
| Rs17293443 | A |
| B4H1:AAA | 0.52 |
| B4H2:GAG | 0.21 |
| B4H3:GAA | 0.15 |
| B4H4:GTA | 0.12 |

TABLE 4(c)
describes haplotype information on Block 5 of SMAD3,
specifically SMAD3 Block 5 Haplotype 1 and 2.
The “B” corresponds with the Block number,
and “H” corresponds with the Haplotype number.

| SNPs | Haplotype 1 Alleles | Haplotype 2 Alleles |
|---|---|---|
| Rs893473 | G | G |
| [SEQ ID NO: 15] | | |
| Rs2289263 | C | A |
| [SEQ ID NO: 16] | | |
| B5H1:GC | 0.48 | |
| B5H2:GA | 0.29 | |
| B5H3:AA | 0.23 | |

TABLE 4(d)
describes haplotype information on Block 6 of
SMAD3, specifically SMAD3 Block 6
Haplotype 1 and 2.

| | Haplo- | Haplo- |
|---|---|---|
| | type 1 | type 2 |
| SNPs | Alleles | Alleles |
| Rs2033785 [SEQ ID NO: 17] | G | G |
| Rs11637659 [SEQ ID NO: 18] | G | G |
| Rs10152307 [SEQ ID NO: 19] | G | A |
| Rs4776900 [SEQ ID NO: 20] | G | G |
| Rs7179840 [SEQ ID NO: 21] | A | G |
| Rs11071939 [SEQ ID NO: 22] | A | A |
| Rs16950687 [SEQ ID NO: 23] | G | A |
| B6H1:GGGGAAG | 0.29 | |
| B6H2:GGAGGAA | 0.24 | |
| B6H3:CGGGAAA | 0.24 | |
| B6H4:GAGAGAA | 0.11 | |
| B6H5:GAGGAGA | 0.07 | |

The “B” corresponds with the Block number,
and “H” corresponds with the Haplotype number.

TABLE 5
Additional associations with JAK2 Haplotypes

| Locus | Hap | F_A | F_U | CHISQ | DF | P |
|---|---|---|---|---|---|---|
| JAK2 | CTC | 0.3959 | 0.364 | 5.885 | 1 | 0.01527 |

SNPs that define the alternative JAK2 haplotype: rs10758669|rs3808850|rs1887429
rs10758669: C is the associated allele, other allele is A
rs3808850: T is the associated allele, other allele is T
rs1887429: C is the associated allele, other allele is A

### Table 5 describes information on additional JAK2 haplotype association.

TABLE 6
Additional associations with SMAD3 Haplotypes

| Locus | Hap | F_A | F_U | CHISQ | DF | P |
|---|---|---|---|---|---|---|
| SMAD3 | GC | 0.2391 | 0.2061 | 8.563 | 1 | 0.003432 |

SNPS that define the alternative SMAD3 haplotype: rs2118611|rs11071933
rs2118611: G is the associated allele, other allele is A
rs11071933: C is the associated allele, other allele is G

### Table 6 describes information on an additional SMAD3 haplotype association.

In various embodiments, the present invention provides a method of diagnosing susceptibility to Inflammatory Bowel Disease (IBD) in an individual, comprising: obtaining a sample from the individual; assaying the sample to determine the presence or absence of a risk haplotype at the Janus kinase 2 (JAK2) genetic locus and/or SMAD family member 3 (SMAD3) genetic locus; and diagnosing susceptibility to IBD in the individual based on the presence of the risk haplotype at the JAK2 genetic locus and/or SMAD3 genetic locus.

In various embodiments, IBD comprises Crohn's disease.

In various embodiments, the risk haplotype at the JAK2 genetic locus comprises JAK2 Block 1 Haplotype 1, JAK2 Block 2 Haplotype 1, and/or JAK2 Block 3 Haplotype 3.

In various embodiments, the risk haplotype at the JAK2 genetic locus comprises SEQ ID NO:1, SEQ ID NO:2, SEQ ID NO:3, SEQ ID NO:4, SEQ ID NO:5, SEQ ID NO:6 and/or SEQ ID NO:7.

In various embodiments, the risk haplotype at the SMAD3 genetic locus comprises SMAD3 Block 2 Haplotype 4, SMAD3 Block 5 Haplotype 1 and/or SMAD3 Block 6 Haplotype 1.

In various embodiments, the risk haplotype at the SMAD3 genetic locus comprises SEQ ID NO:8, SEQ ID NO:9, SEQ ID NO:10, SEQ ID NO:11, SEQ ID NO:12, SEQ ID NO:13 and/or SEQ ID NO:14.

In various embodiments, the risk haplotype at the SMAD3 genetic locus comprises SEQ ID NO:15 and/or SEQ ID NO:16.

In various embodiments, the risk haplotype at the SMAD3 genetic locus comprises SEQ ID NO:17, SEQ ID NO:18, SEQ ID NO:19, SEQ ID NO:20, SEQ ID NO:21, SEQ ID NO:22 and/or SEQ ID NO:23.

In various embodiments, the present invention provides a method of determining a low probability of developing Crohn's disease in an individual, relative to a healthy subject, comprising: obtaining a sample from the individual; assaying the sample to determine the presence or absence of a protective haplotype at the Janus kinase 2 (JAK2) genetic locus and/or SMAD family member 3 (SMAD3) genetic locus; and diagnosing a low probability of developing Crohn's disease in the individual, relative to a healthy subject, based upon the presence of the protective haplotype at the JAK2 and/or SMAD3 genetic locus.

In various embodiments, the protective haplotype at the JAK2 genetic locus comprises JAK2 Block 1 Haplotype 3, JAK2 Block 2 Haplotype 2, and/or JAK2 Block 3 Haplotype 1.

In various embodiments, the protective haplotype at the SMAD3 genetic locus comprises SMAD3 Block 4 Haplotype 1, SMAD3 Block 5 Haplotype 2, and/or SMAD3 Block 6 Haplotype 2.

In various embodiments, the present invention provides a method of diagnosing a Crohn's disease subtype in an individual, comprising: determining the presence of one or more risk variants at the Janus kinase 2 (JAK2) genetic locus and/or SMAD family member 3 (SMAD3) genetic locus; and diagnosing the Crohn's disease subtype in the individual based upon the presence of the one or more risk variants at the JAK2 and/or SMAD3 genetic locus.

In various embodiments, the one or more risk haplotypes at the JAK2 genetic locus comprise SEQ ID NO:1.

In various embodiments, the one or more risk variants at the JAK2 genetic locus comprise JAK2 Block 1 Haplotype 1, JAK2 Block 2 Haplotype 1, and/or JAK2 Block 3 Haplotype 3.

In various embodiments, the one or more risk variants at the SMAD3 genetic locus comprise SMAD3 Block 2 Haplotype 4, SMAD3 Block 5 Haplotype 1, and/or SMAD3 Block 6 Haplotype 1.

In various embodiments, the present invention provides a method of treating Crohn's disease in an individual, comprising: determining the presence of a risk variant at the janus kinase 2 (JAK2) genetic locus and/or SMAD family member 3 (SMAD3) genetic locus; and treating the individual based upon the presence of the risk variant at the JAK2 genetic locus and/or SMAD3 genetic locus.

In various embodiments, the present invention provides a method of determining the prognosis of Crohn's disease in an individual, comprising: determining the presence or absence of one or more risk variants at the Janus kinase 2 (JAK2) genetic locus and/or SMAD family member 3 (SMAD3) genetic locus; and prognosing a complicated case of Crohn's disease if the individual demonstrates the presence of one or more risk variants at the JAK3 genetic locus and/or SMAD3 genetic locus.

In various embodiments, the one or more risk variants at the JAK2 genetic locus comprise JAK2 Block 1 Haplotype 1, JAK2 Block 2 Haplotype 1, and/or JAK2 Block 3 Haplotype 3.

In various embodiments, the one or more risk variants at the SMAD3 genetic locus comprise SMAD3 Block 2 Haplotype 4, SMAD3 Block 5 Haplotype 1, and/or SMAD3 Block 6 Haplotype 1.

In various embodiments, the present invention provides a method of treating Crohn's Disease in an individual, comprising: determining the presence of a risk variant at the Janus kinase 2 (JAK2) genetic locus in the individual; and treating the individual by inhibiting the JAK2 signaling pathway.

In various embodiments, the risk variant at the JAK2 genetic locus comprises SEQ ID NO:1.

### Example 2

### Methods of Using Znf365 Genetic Variants to Diagnose Crohn's Disease

This non-limiting example relates to prognosing, diagnosing and treating of Crohn's disease. In various embodiments, this non-limiting example also provides prognosis, diagnosis, and treatment that are based upon the presence of one or more genetic risk factors at the ZNF365 genetic locus. Exemplar data are shown in FIGS. 2-6.

### Example 2-1

In the interest of identifying causal variants of Crohn's disease at 10q21, the inventors fine mapped the 10q21 region. The inventors genotyped 86 SNPs across the region of reported association (Chr. 10, position 63,798,139 to 64,219,617) in 1,683 CD cases and 1,049 non-IBD controls. Single marker and conditional analyses were performed using logistic regression (PLINK). ZNF365 isoform D expression was assessed using RT-PCR. Peak association with CD was observed within ZNF365 at rs7076156 and rs7071642, two SNPs in complete linkage disequilibrium (LD) (FIG. 5). Conditioning on nonsynonymous SNP rs7076156 (Ala62Thr) nullified all other significant associations and the threonine allele protected against CD (p=1.05×10⁻⁷; OR 0.71; 23.6% in patients with CD and 30.1% in controls). Four isoforms of ZNF365 (A-D) have previously been identified and rs7076156 is located in an exon unique to ZNF365 isoform D. The inventors further detected expression of this isoform in a terminal ileum resection specimen from a patient with CD.

As further disclosed herein, the inventors demonstrate significant associations between CD and the ZNF365 locus. Conditional analyses show that a coding variant (rs7076156; Ala62Thr) confers protection against CD. Furthermore, mRNA for ZNF365 isoform D is expressed in small intestine. Taken together these data show that this variant explains the CD association observed at 10q21.

### Example 2-2

A total of 1,683 predominantly Caucasian CD cases and 1,049 non-IBD controls were included in this analysis. CD subjects were recruited at Cedars-Sinai Medical Center Inflammatory Bowel Disease (CSMC IBD) Center and Wolfson Medical Center, Holon, Israel after diagnosis using standard clinical, endoscopic, and histological features (Mow, et al., Gastroenterology 2004; 126:414-24). Controls, also of Caucasian descent, were recruited through the CSMC IBD Center (IBD patients' unrelated acquaintances and spouses of cases with no personal or family history of IBD or autoimmune disease); as part of the Pharmacogenetics and Risk of Cardiovascular disease (PARC) Study, a multicenter pharmacogenetic study of statin response (Simon, et al., Am J Cardiol 2006; 97:843-50; Krauss, et al., Circulation 2008; 117:1537-44.); or from the National Laboratory for the Genetics of Israeli Populations at Tel-Aviv University; Tel-Aviv, Israel). All cases and controls provided informed consent prior to study participation and following approval of participating centers' institutional review boards.

### Example 2-3

The inventors applied a haplotype-tagging approach to the region previously associated with CD (chromosome 10, position 63,798,139 to 64,219,617) (Rioux et al., Nat Genet 2007; 39:596-604; Consortium WTCC. Genome-wide association study of 14,000 cases of seven common diseases and 3,000 shared controls. Nature 2007; 447:661-78; Barrett, et al., Nat Genet 2008; 40:955-62) using Tagger as implemented in Haploview (De Bakker P I. Tagger broad-mit 2004; Barrett, et al., Bioinformatics 2005; 21:263-5) and data from the International HapMap project, release 2. The inventors aimed to select SNPs compatible with the Illumina Infinium technology that tagged haplotypes with a frequency greater than 5% in the Caucasian population (Consortium I H. The International HapMap Project. Nature 2003; 426:789-796; Frazer, et al., Nature 2007; 449:851-61). Non-synonymous SNPs with a minor allele frequency in the Caucasian population >3% were also added to the initial genotyping panel of 86 SNPs. Genotyping for this study was performed as part of a project including a total of 7109 SNPs.

### Example 2-4

All genotyping was performed at the Medical Genetics Institute at Cedars-Sinai Medical Center using custom iSelect Infinium technology, following the manufacturer's protocol (Illumina, San Diego, Calif.) (Gunderson, et al., Pharmacogenomics 2006; 7:641-8; Gunderson, K L, et al., Methods Enzymol 2006; 410:359-76). Samples with genotyping success rates <98% or with gender discrepancies were excluded from analyses. The average genotyping rate of samples retained in the analysis was 99.9%. Twenty samples performed in duplicate yielded 100% concordance. SNPs were excluded if the test of Hardy-Weinberg equilibrium across the entire sample was p≦10⁻³; if the genotyping failure rate was >10%; if the minor allele frequency was <3%; or if the SNP had been selected for genotyping but was not found in the new dbSNP build at the time of analysis (dbSNP 129). These quality control steps left 78 SNPs in 10q21 for the analyses reported herein.

### Example 2-5

Single marker analysis for association with case/control status was performed using logistic regression (as implemented in PLINK v1.06) (Purcell, et al., Am J Hum Genet 2007; 81:559-75). Conditional logistic regression analysis was used to include allele load for the SNP being conditioned upon in the regression equation, and was performed using the condition function (PLINK).

### Example 2-6

Since ZNF365D has been reported to be expressed in kidney, commercially available total RNA extracted from human adult whole kidney tissue (Agilent Stratagene, La Jolla, Calif.) was used as a positive control for ZNF365D expression. Intestinal tissue was also collected from a Caucasian, non-smoking CD subject undergoing small bowel surgery at CSMC IBD Center for stricturing disease. There is a personal history of rheumatoid arthritis and a strong family history of autoimmune disease in this particular subject, and at the time of surgery the patient was being treated with anti-TNF medication (Humira). Tissue was stabilized for storage in RNA later (Ambion, Austin, Tex.) and stored at room temperature until total RNA was extracted using the RiboPure Kit, following manufacturer's instructions (Ambion, Austin, Tex.). Because ZNF365D had been previously reported to have a short poly-A tail (GenBank NM_{—}199452.2), cDNA was synthesized from the total RNA template using random nonamers and the AffinityScript Multiple Temperature cDNA Synthesis kit (Agilent Stratagene, La Jolla, Calif.). The presence of the ZNF365D isoform was detected in a standard PCR reaction using the FailSafe PCR premix selection kit (Epicentre, Madison, Wis.). A single amplicon band at the expected size (379 bp) was seen with the FailSafe premix buffer H and ZNF365 isoform D specific PCR primers (Forward—5′ ATG TCT GCG CTG GGT CAG ATA 3′ and Reverse—5′ CTC CTG CAT AGG GAG GTG 3′ in exons 2 and 4, respectively; Invitrogen, Carlsbad, Calif.). PCR was preformed according to the following conditions: 10 min at 95°; followed by 40 cycles of: 30 sec at 95°, 1 min at 55°, 30 sec at 72°; and a final extension for 10 min at 72°.

### Example 2-7

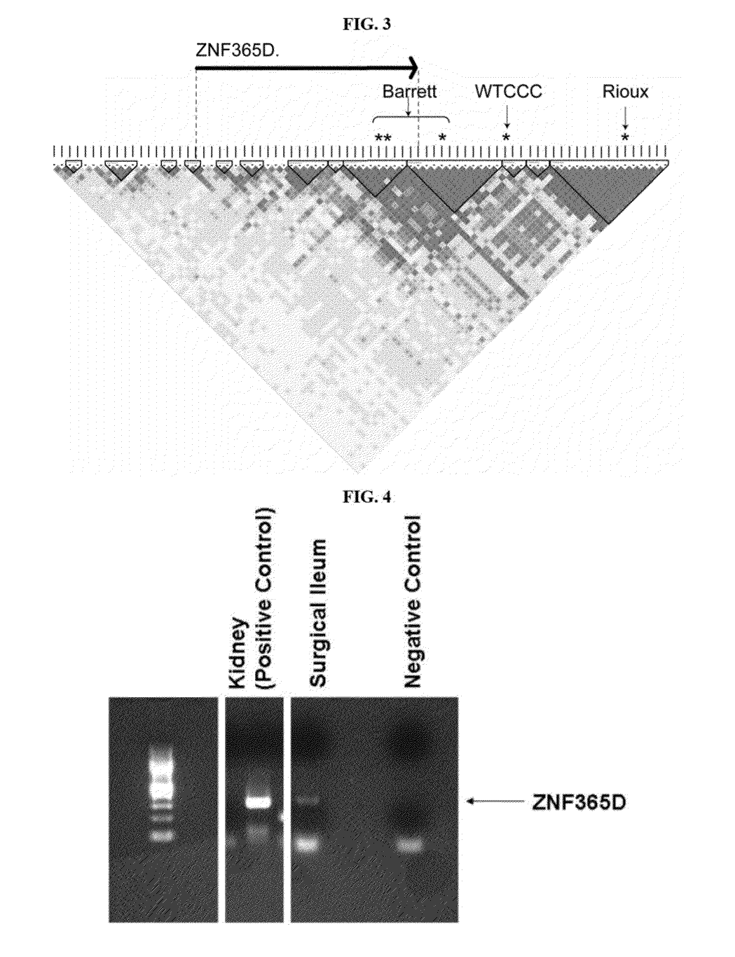

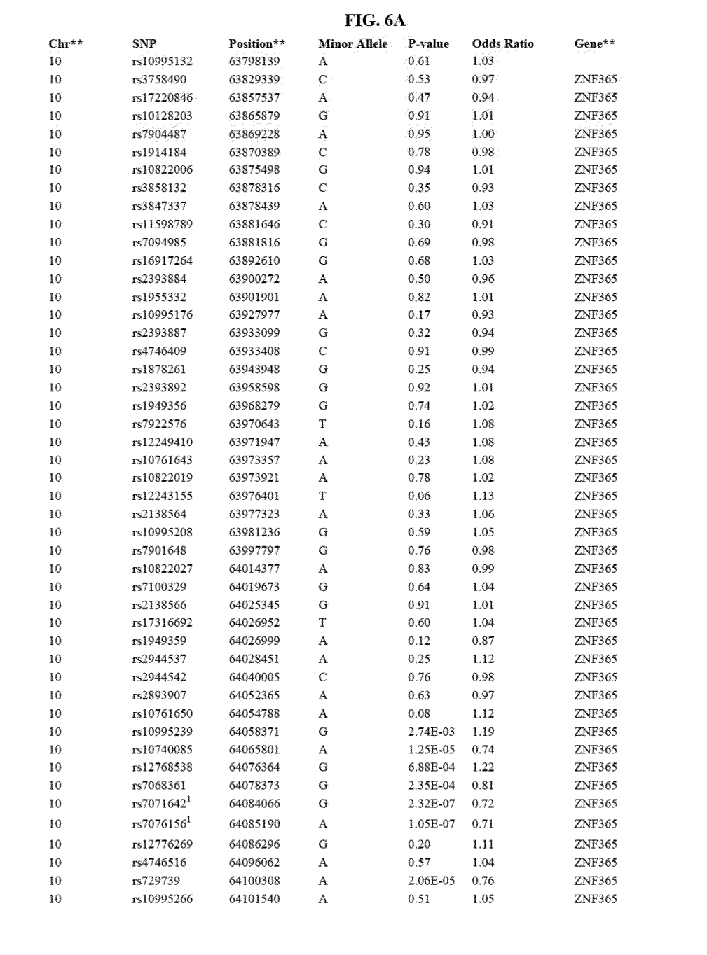

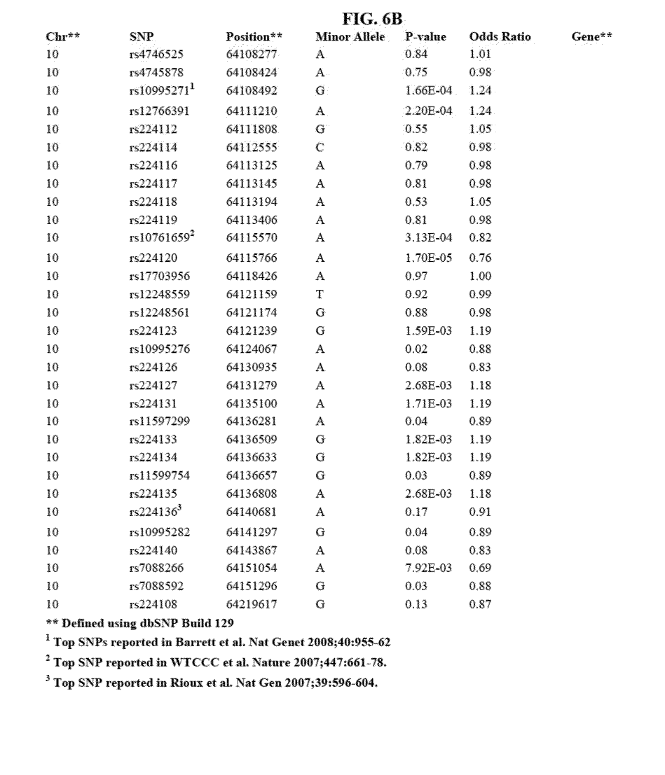

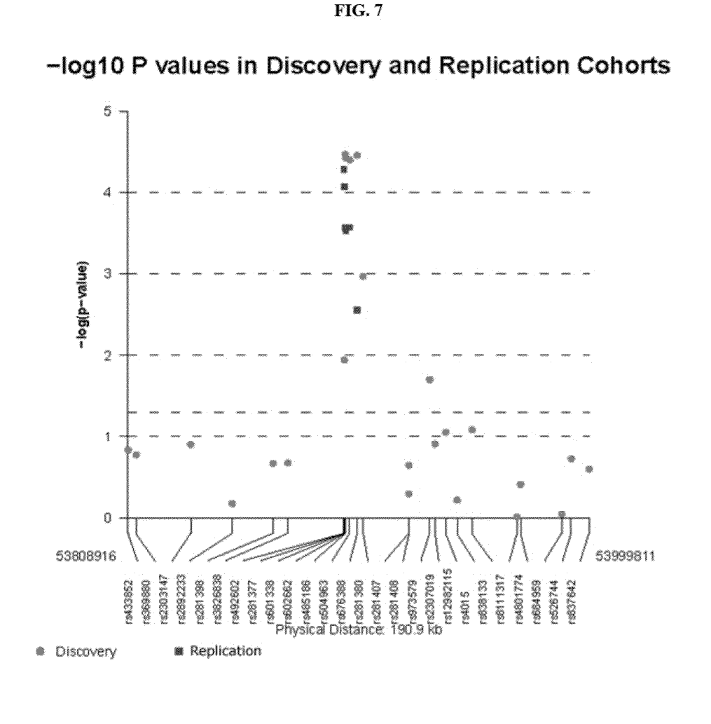

The inventors aimed to use a haplotype tagging approach to capture the major haplotypic variation in linkage disequilibrium with the 10q21 SNPs previous reported to be associated with CD (Rioux et al., Nat Genet 2007; 39:596-604; Consortium WTCC. Genome-wide association study of 14,000 cases of seven common diseases and 3,000 shared controls. Nature 2007; 447:661-78; Barrett, et al., Nat Genet 2008; 40:955-62). Seventy-eight SNPs from this region were included in the final analysis of 1,683 CD cases and 1,049 non-IBD controls (FIGS. 6A-B). Ten SNPs exhibited highly significant associations (p<0.001) with the peak association observed at two SNPs, rs7076156 (OR=0.71; p=1.05×10⁻⁷) and rs7071642 (OR=0.72; p=2.32×10⁻⁷). These 2 SNPs were in complete linkage disequilibrium with each other (LD; r²=1.0) (FIG. 5 and FIG. 3). The inventors also confirmed association of CD with the previously reported SNPs rs10761659 (p=3.13×10⁻⁴) and rs10995271 (p=1.66×10⁻⁴) (Consortium WTCC. Genome-wide association study of 14,000 cases of seven common diseases and 3,000 shared controls. Nature 2007; 447:661-78; Barrett, et al., Nat Genet 2008; 40:955-62).

In order to determine whether the multiple associations were due to the high LD in this region and to identify the SNP or SNPs with the largest contribution to CD susceptibility, the inventors examined the effect of conditioning the CD association on each SNP in turn (FIG. 5). Conditioning on the most significantly associated SNP rs7076156 reduced all other CD associations to non-significance and regressed all odds ratios (OR) to 1 (with OR=1.2 for rs729739; FIG. 5). The regression of OR to 1, along with the change in P-values to become non-significant, demonstrate that the association observed between multiple SNPs in ZNF365 and CD is due to the LD between the associated markers within this region and what is potentially the causal variant, rs7076156 (FIG. 5). Analyses of the association between the haplotypes formed by the genotyped SNPs did not provide any further insight into the association between CD and this region beyond that of the association between CD and rs7076156.

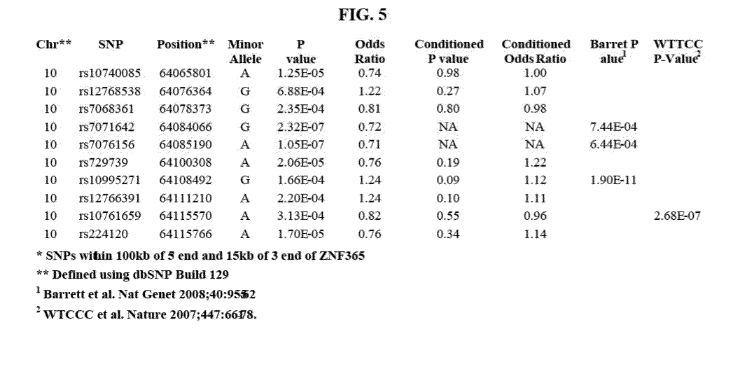

Four isoforms of ZNF365 (A-D) have been reported (FIG. 2) (Gianfrancesco, et al., Am J Hum Genet 2003; 72:1479-91). Rs7076156 is a nonsynonymous SNP (G>A; Ala62Thr) in exon 4 unique to ZNF365 isoform D. The minor allele (threonine allele) of Ala62Thr protected against CD (OR 0.71; FIG. 5) and had an allelic frequency of 23.6% in patients with CD and 30.1% in controls. In order to further elucidate a potential role for this functional variant in CD, the inventors focused attention on isoform D of ZNF365. RT-PCR was performed to evaluate the expression of ZNF365D in whole human kidney, a positive control tissue, and in human small intestine. The inventors confirmed previously reported expression of ZNF365D in the kidney (Gianfrancesco, et al., Am J Hum Genet 2003; 72:1479-91) and detected expression of ZNF365D in cDNA from ileum obtained from a CD patient undergoing small bowel surgery (FIG. 4).

### Example 2-8

The inventors have characterized the association between CD and SNPs in the 10q21 region and have identified an association between a nonsynonymous Ala62Thr SNP located in the ZNF365D isoform (rs7076156, p=1.05×10⁻⁷; OR 0.71). Conditional analyses further demonstrated that this SNP accounts for the associations of other SNPs in the immediate region, including those in previous reports and confirmed in this study (FIG. 5; rs10761659, p=3.13×10⁻⁴; rs10995271, p=1.66×10⁻⁴) (Consortium WTCC. Genome-wide association study of 14,000 cases of seven common diseases and 3,000 shared controls. Nature 2007; 447:661-78; Barrett, et al., Nat Genet 2008; 40:955-62; Franke, et al., Nat Genet 2008; 40:713-5). Thus, even though the LD between the ZNF365D Ala62Thr variant and the SNPs in some previous reports was low (r² between Ala62Thr rs7076156 and rs10995271 is 0.19 and rs10761659 is 0.37), this conditional analysis shows that the ZNF365D Ala62Thr variant accounts for the association observed in these reports. Since expression of the ZNF365D isoform has thus far not been reported in intestine, the inventors tested for and subsequently observed the expression of this isoform in human intestine from a CD patient undergoing surgery for strictures (FIG. 4). When taken together, these observations support expression of the ZNF365D isoform with the Threonine allele in human intestine is associated with CD.

In summary, the inventors provide evidence from both a genetic and expression perspective that ZNF365 is a convincing candidate gene for CD susceptibility, having demonstrated an association with a coding variant rs7076156 that confers strong protection against CD. Conditional analysis indicated the causal variant in the region is likely to be this nonsynonymous SNP that is located in an exon unique to one of four isoforms of this gene. Finally, the inventors have demonstrated expression in the ileum of a CD subject. When taken together, these observations point to this SNP as a causal variant for CD within the 10q21 region.

In various embodiments, the present invention provides a method of diagnosing susceptibility to Crohn's disease in an individual, comprising: obtaining a sample from the individual; assaying the sample to determine the presence or absence of a risk variant at the ZNF365 genetic locus; and diagnosing susceptibility to Crohn's disease in the individual based on the presence of the risk variant at the ZNF365 genetic locus.

In various embodiments, the risk variant is selected from the group consisting of rs10740085, rs12768538, rs7068361, rs7071642, rs7076156, rs729739, rs10995271, rs12766391, rs10761659, and rs224120.

In various embodiments, the risk variant is rs7076156.

In various embodiments, the risk variant is rs7071642.

In various embodiments, assaying the sample comprises genotyping for one or more single nucleotide polymorphisms.

In various embodiments, the sample is whole blood, plasma, serum, saliva, cheek swab, urine, or stool.

In various embodiments, the present invention provides a method of determining a low probability of developing Crohn's disease in an individual, relative to a healthy subject, comprising: obtaining a sample from the individual; assaying the sample to determine the presence or absence of a protective variant at the ZNF365 genetic locus; and diagnosing a low probability of developing Crohn's disease in the individual, relative to a healthy subject, based upon the presence of the protective variant at the ZNF365 genetic locus.

In various embodiments, the protective variant is selected from the group consisting of rs10740085, rs12768538, rs7068361, rs7071642, rs7076156, rs729739, rs10995271, rs12766391, rs10761659, and rs224120.

In various embodiments, the protective variant is rs7076156.

In various embodiments, the protective variant is rs7071642.

In various embodiments, assaying the sample comprises genotyping for one or more single nucleotide polymorphisms.

In various embodiments, the sample is whole blood, plasma, serum, saliva, cheek swab, urine, or stool.

In various embodiments, the present invention provides a method of prognosing Crohn's disease in an individual, comprising: obtaining a sample from the individual; assaying the sample for the presence or absence of one or more genetic risk variants; and prognosing an aggressive form of Crohn's disease based on the presence of one or more risk variants at the ZNF365 genetic locus.

In various embodiments, the risk variant is selected from the group consisting of rs10740085, rs12768538, rs7068361, rs7071642, rs7076156, rs729739, rs10995271, rs12766391, rs10761659, and rs224120.

In various embodiments, assaying the sample comprises genotyping for one or more single nucleotide polymorphisms.

In various embodiments, the sample is whole blood, plasma, serum, saliva, cheek swab, urine, or stool.

In various embodiments, the present invention provides a method of treating an individual for Crohn's disease, comprising: prognosing an aggressive form of Crohn's disease in the individual based on the presence of one or more risk variants at the ZNF365 genetic locus; and treating the individual, wherein the one or more risk variants are selected from rs10740085, rs12768538, rs7068361, rs7071642, rs7076156, rs729739, rs10995271, rs12766391, rs10761659, and rs224120.

In various embodiments, assaying the sample comprises genotyping for one or more single nucleotide polymorphisms.

In various embodiments, the sample is whole blood, plasma, serum, saliva, cheek swab, urine, or stool.

### Example 3

### Methods of Using Fut2 Genetic Variants to Diagnose Crohn's Disease

This non-limiting example relates to prognosing, diagnosing and treating of Crohn's disease. In various embodiments, this non-limiting example also provides prognosis, diagnosis, and treatment that are based upon the presence of one or more genetic risk factors at the FUT2 genetic locus.

### Example 3-1

The discovery cohort used in the GWAS included 1096 Crohn's Disease subjects and 3980 healthy population controls. The replication cohort consisted of 1174 Caucasian CD cases and 357 Caucasian healthy controls; all independent of the cohort in the GWAS. Cases were recruited from the Cedars-Sinai IBD Center and Pediatric IBD department and were diagnosed with CD according to standard clinical, radiological, endoscopic and histological criteria. Controls for the GWAS were obtained from the Cardiovascular Health Study (CHS), a population-based longitudinal study of risk factors for cardiovascular disease and stroke in adults 65 years of age or older, recruited at four field centers (Fried, L. P. et al., Ann Epidemiol 1, 263-76 (1991)). 5201 predominantly Caucasian individuals were recruited in 1989-1990 from random samples of Medicare eligibility lists, followed by an additional 687 African-Americans recruited in 1992-1993 (total n=5888). Controls used in the replication study were recruited through the IBD center (unrelated acquaintances and spouses of cases with no personal or family history of IBD or autoimmune disease) or recruited as part of the PARC project, a pharmacogenetic study of statin response (Krauss, R. M. et al., Circulation 117, 1537-44 (2008); Simon, J. A. et al., Am J Cardiol 97, 843-50 (2006)). All cases and controls provided informed consent prior to study participation and following approval of participating centers' institutional review boards.

### Example 3-2

All genotyping was performed at the Medical Genetics Institute at Cedars-Sinai Medical Center using whole-genome genotyping Infinium technology, following the manufacturer's protocol (Illumina, San Diego, Calif.) (Gunderson, K. L. et al., Nat Genet 37, 549-54 (2005); Gunderson, K. L. et al., Methods Enzymol 410, 359-76 (2006)). Cases were genotyped with either the Illumina Human 610Quad platform or the Illumina Human 317Duo platform. Controls were genotyped with the Illumina 370Duo platform. Samples with genotyping rates >98% were retained in the analysis. In addition, case and control cohorts were both investigated using Identity-By-Descent (Pi hat scores >0.5 as detected in PLINK (Purcell, S. et al., Am J Hum Genet 81, 559-75 (2007))) in order to identify cryptic relatedness, and related individuals were excluded. Following these QC steps, 1096 CD cases and 3694 controls were included in the study. Single nucleotide polymorphisms (SNPs) were excluded based on the following criteria: test of Hardy-Weinberg Equilibrium p≦10⁻³; SNP failure rate >10%; MAF <5%; and SNPs not found in dbSNP Build 129. SNPs were also examined in order to exclude case/control disparity in missingness (PLINK (Purcell, S. et al., Am J Hum Genet 81, 559-75 (2007))). 304,825 SNPs that passed QC criteria, and were available in all datasets, were included in the logistic regression association analysis. The 6 SNPs tested in the replication cohort were genotyped using TaqMan™ assay according to the manufacturer's instructions (Applied Biosystems, Foster City, Calif.).

### Example 3-3

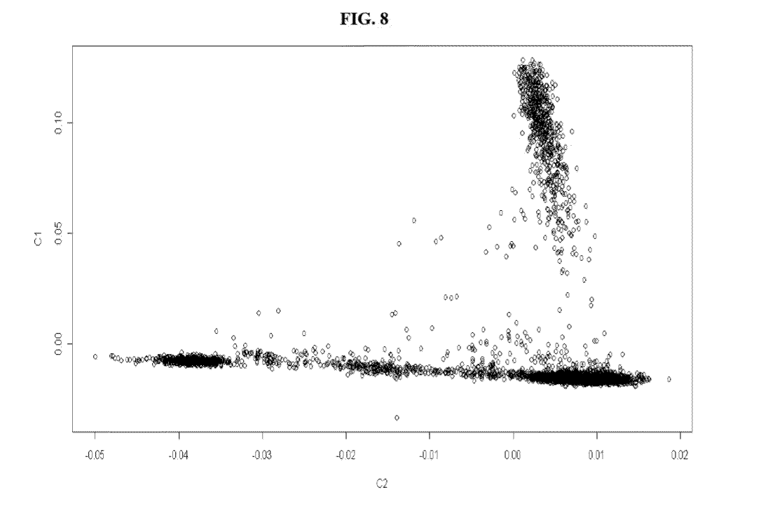

Population structure was detected using Multidimensional Scaling (MDS) (PLINK (Purcell, S. et al., Am J Hum Genet 81, 559-75 (2007))). In total, 10 principal components (PC) were calculated and plotted for graphical representation of population substructure within the cohort. Subjects with a PC1>0.025 represent African American subjects. To reduce false positive discovery due to population substructure, and the predominantly Caucasian make-up of the cases, these subjects were excluded from downstream analysis. This resulted in 896 CD and 3204 control subjects being carried forward for association testing with the CD phenotype using a logistic regression model in R (FIG. 8). All 10 principal components were carried into association testing as covariates. A logistic regression analysis correcting for population substructure was used to test for association between genotype and phenotype. Self-reported ethnicity data was used to confirm the identification of ethnicity based on cluster plots (FIG. 8). The association of the FUT2 SNPs with CD in the independent confirmation cohort was tested using logistic regression (as implemented in R).

### Example 3-4

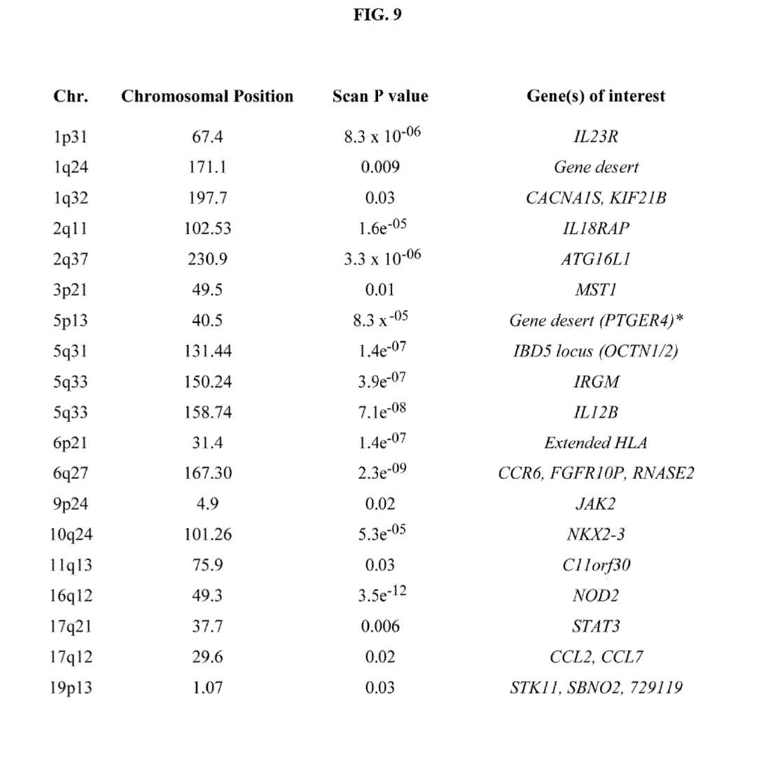

A CD GWAS meta-analysis previously identified or confirmed association with 30 loci and demonstrated nominal association with a further 10 loci (Barrett, J. C. et al., Nat Genet 40, 955-62 (2008)). The inventors confirmed association (uncorrected p value <0.05 and association with the previously identified risk allele) with 19 of these loci in the inventors' GWAS (FIG. 9) and these loci served as internal controls for the inventors' dataset. Three of these loci were from the nominally replicated list of SNPs (rs4807569, 19p13; rs991804, CCL2, CCL7; rs917997, IL18RAP) from the meta-analysis study, and the data presented in FIG. 9 therefore provide further evidence of their relevance in CD susceptibility. The IL18RAP association has previously been confirmed (Zhernakova, A. et al., Am J Hum Genet 82, 1202-10 (2008)). In this data set the inventors did not demonstrate association (p≦0.05) with CD and the other 21 loci identified in the GWAS meta-analysis including 10p11, 10q21, 12q12 (SLC2A13, LRRK2), 1p13 (PTPN22), 18p11 (PSMG2, PTPN2), 17q21 (ORMDL3), 13q14 (CCDC122), 9q32 (TNFSF15), 6p22 (CDKAL1), 6q21 (PRDM1), 8q24, 1q23 (ITLN1, CD244), 6p25 (LYRM4), 2p16 (PUS10), 6p25 (SLC22A23), 6q25, 2p23 (GCKR), 7p12, 21q21, 21q22 and 18q11.

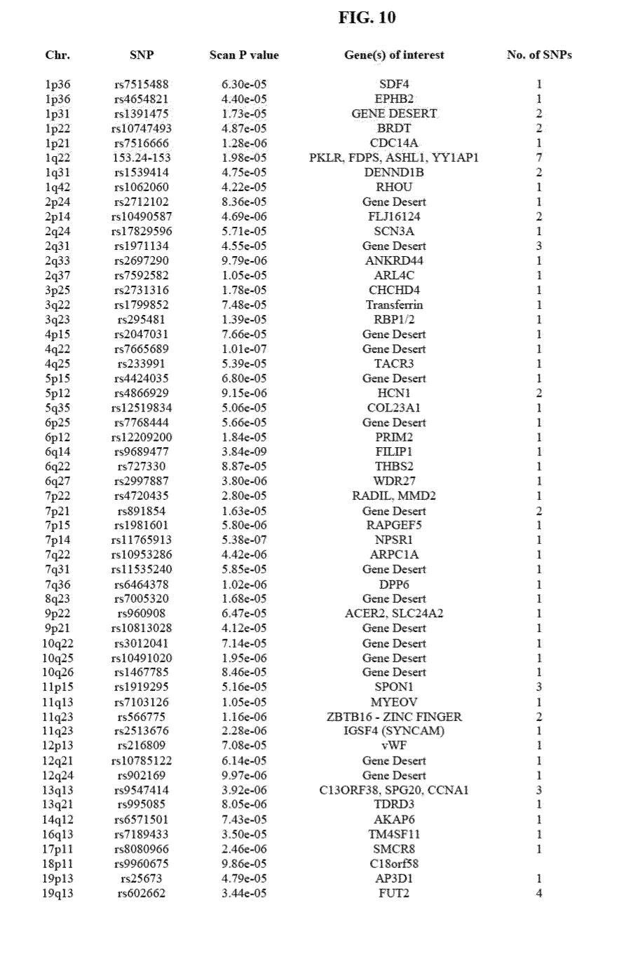

In addition, the inventors identified association between CD and a number of novel loci (FIG. 10). These include genes involved in tight junctions/epithelial integrity (ASHL, ARPC1A), Wnt and JNK1 signaling (RHOU), dendritic cell function (RBP1 and 2), Substance P signaling (TACR3), macrophage development (MMD2), asthma susceptibility (NPSR1) (Laitinen, T. et al., Science 304, 300-4 (2004)), integrin regulation (ACER2), and NK T cell biology (AP3D1). The inventors also identified two CD associated loci specifically involved in the host-microbial interaction namely SPG20 (endosomal trafficking) and FUT2.

### Example 3-5

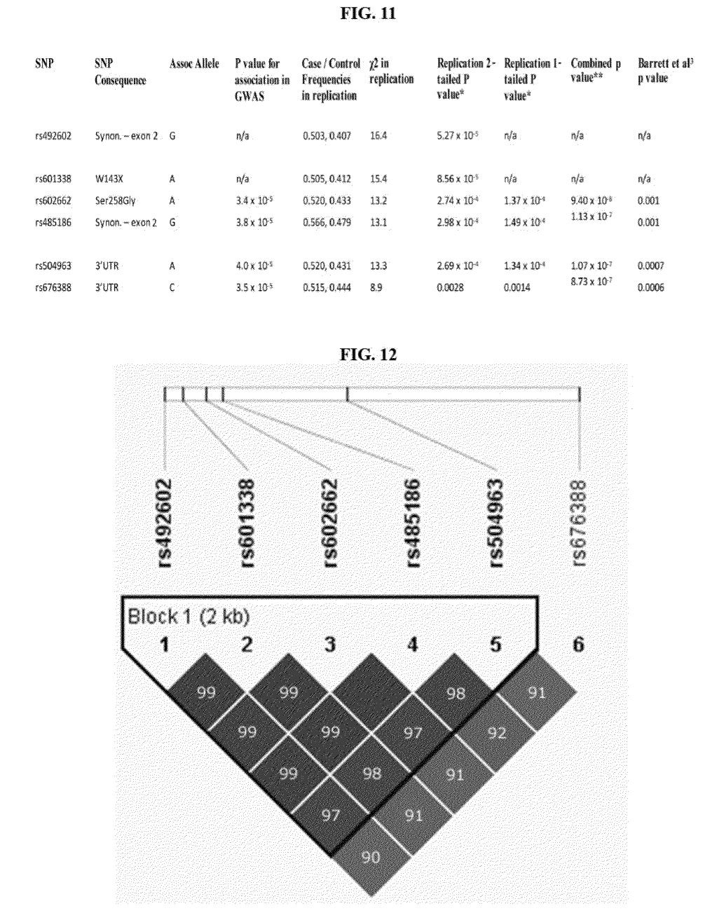

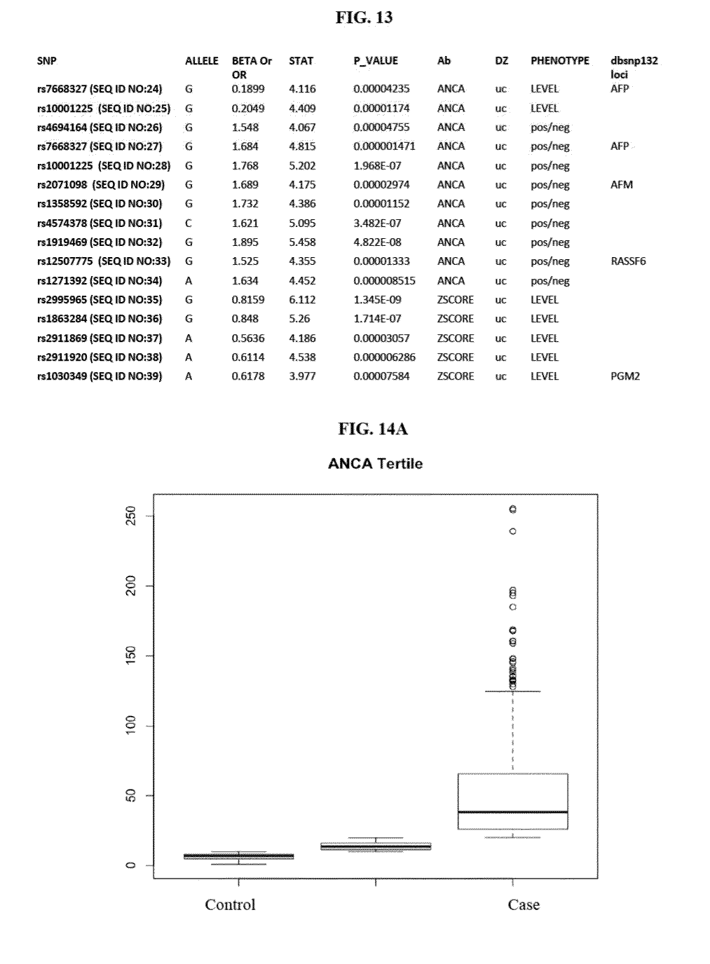

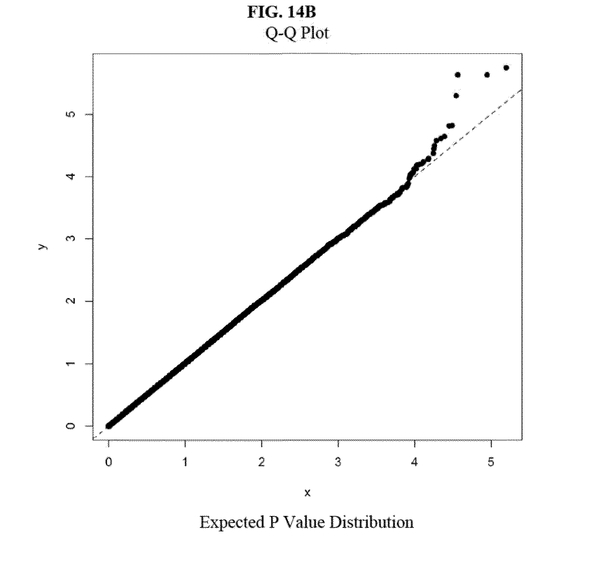

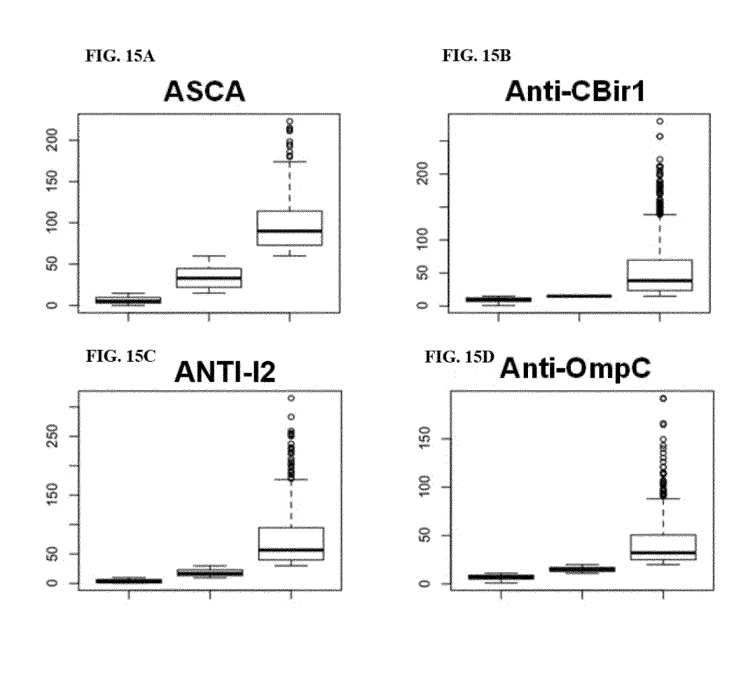

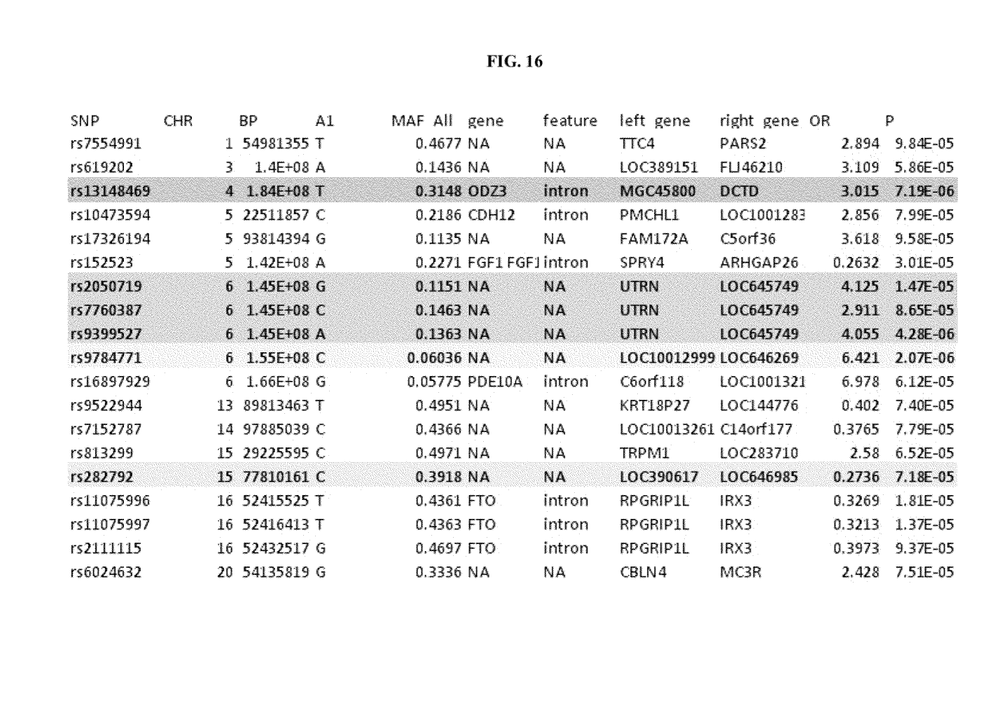

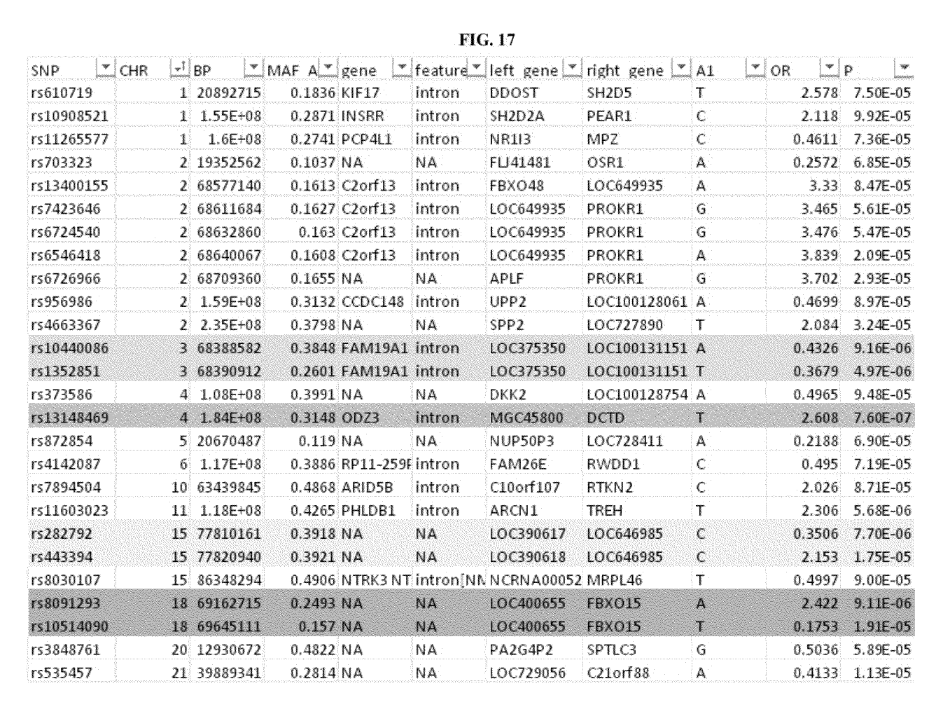

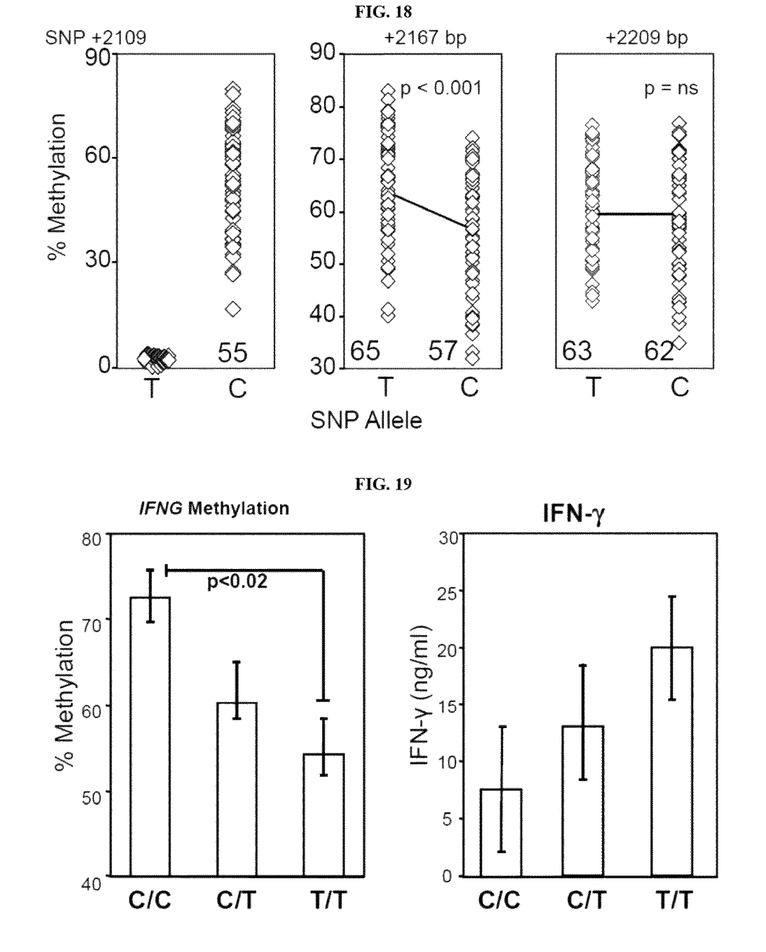

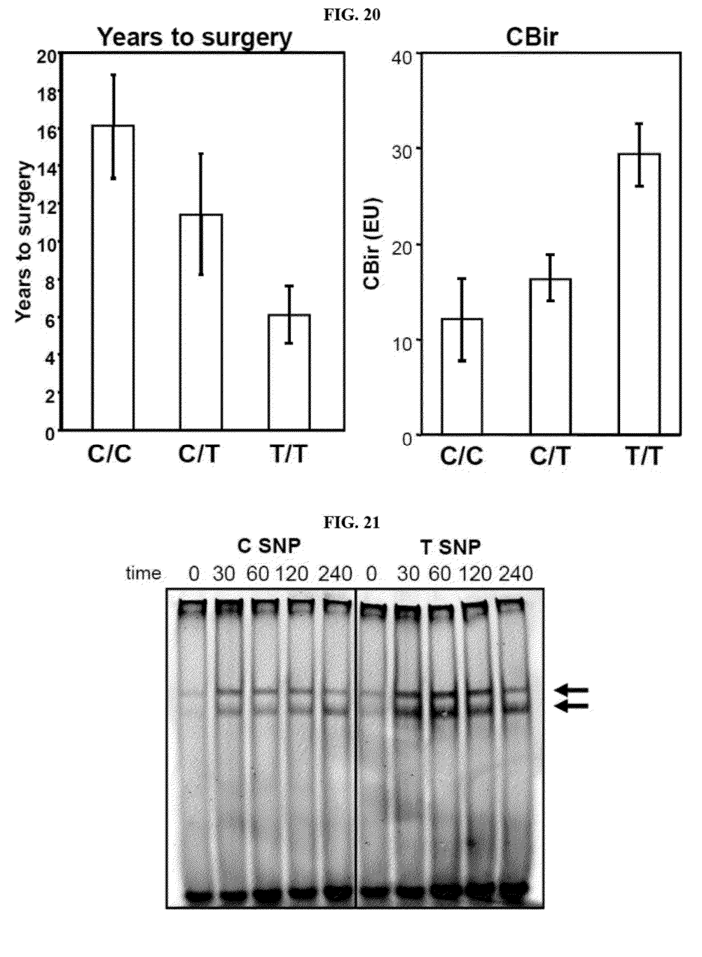

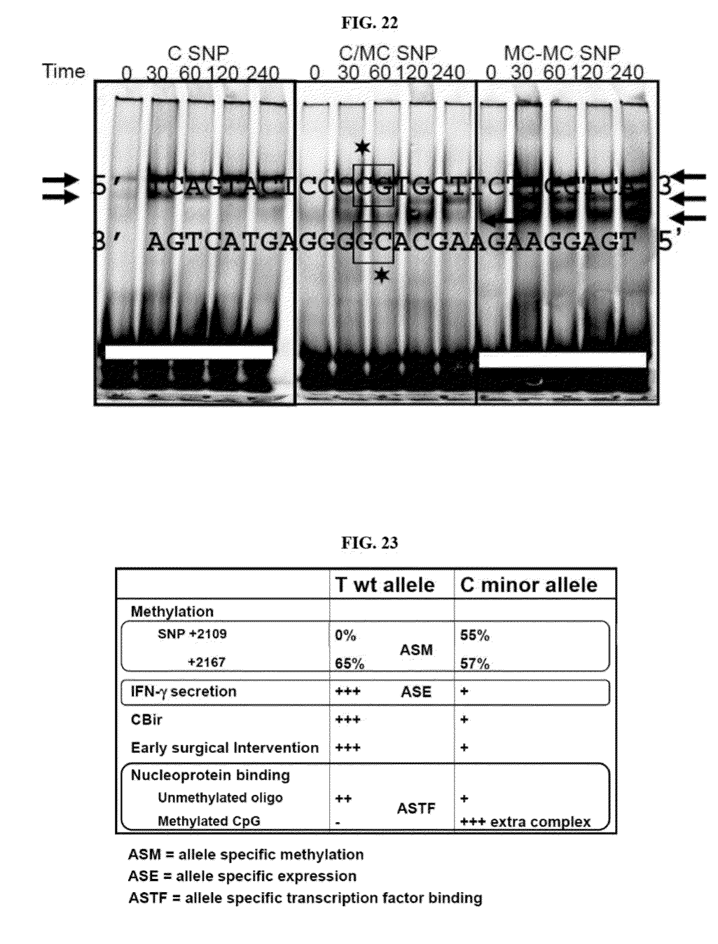

From the novel associations, the inventors first chose FUT2 as the leading gene for independent replication given the inventors' interest in the host-microbial interaction in CD pathogenesis and FUT2's known association with a number of infective processes. Furthermore FUT2 is located under a known peak of linkage for CD on chromosome 19 (van Heel, D. A. et al., Hum Mol Genet 13, 763-70 (2004)) and there were 4 SNPs with strong association to CD in the inventors' GWAS (FIGS. 11 and 12). In addition to these 4 SNPs (rs504963—3′UTR, rs676388—3′UTR, rs485186—synonymous exon 2 SNP and rs602662—Ser258Gly) identified in the GWAS, the inventors also genotyped rs492602 (synonymous exon 2) and rs601338 (W143X, the common null allele in Caucasians associated with the ABO non-secretory phenotype) in the independent confirmatory cohort. The inventors were able to replicate the initial association with the four SNPs from the discovery cohort, as well as demonstrate association with the additional two SNPs, including the allele for non-secretor status. Further evidence for the association between this locus and CD susceptibility is provided in the CD meta-analysis published by Barrett et al., (Barrett, J. C. et al., Nat Genet 40, 955-62 (2008)) in which all four of the originally identified SNPs are associated with CD (FIG. 11). The 6 SNPs included in the replication study are in strong linkage disequilibrium (FIG. 12).

### Example 3-6

In this study the inventors confirmed association with a number of known CD loci and provided further evidence for association to CD with two other loci previously only nominally associated with disease (19p13 and 1702). The region on 19p13 contains SBNO2 and GPX4 (glutathione peroxidase 4). Little is known about SBNO4, while GPX4 is known to protect cells against oxidative damage and may have a regulatory role in leukotriene biosynthesis (Villette, S. et al., Blood Cells Mol Dis 29, 174-8 (2002)). The 1702 locus is located in a cytokine gene cluster containing the CCL2, CCL8, CCL11 and CCL7 genes. These genes encode Cys-Cys cytokine genes which are involved in immunoregulatory and inflammatory processes and are therefore attractive candidate genes for CD susceptibility. This locus has previously been implicated in susceptibility to asthma (Batra, J. et al., J Med Genet 44, 397-403 (2007)) and Mycobacterium susceptibility (Thye, T. et al., Hum Mol Genet 18, 381-8 (2009)) as well as with HIV progression (Modi, W. S. et al., AIDS 17, 2357-65 (2003)).

Also disclosed herein, the inventors identified novel loci associated with CD, most notably FUT2. The inventors provided independent confirmation for association between FUT2 and CD in both the inventors' own cohort, and in the meta-analysis published by Barrett et al., (Barrett, J. C. et al., Nat Genet 40, 955-62 (2008)). This cumulative data provides strong evidence of the role of this locus in CD susceptibility. This gene is of particular interest, as it potentially extends knowledge regarding the scope of the host-microbial interaction in CD. Previous genetic associations with CD have highlighted the role of both the innate (Hugot, J. P. et al., Nature 411, 599-603 (2001); Ogura, Y. et al., Nature 411, 603-6 (2001); De Jager, P. L. et al., Genes Immun 8, 387-97 (2007); Saruta, M. et al., Inflamm Bowel Dis 15, 321-7 (2009).) and the adaptive immune systems' (Shen, C. et al., Inflamm Bowel Dis 14, 1641-51 (2008); Duchmann, R. et al., Eur J Immunol 26, 934-8 (1996)) interaction with the microbiome. The data presented herein extend this interaction to the mucus layer of the GI tract. FUT2 encodes the secretor type α (1,2) fucosyltransferase (also known as the Se enzyme) that is responsible for regulating the secretion of the ABO antigens in both the digestive mucosa and secretory glands. Approximately 20% of individuals are non-secretors who fail to express ABO antigens in both the GI tract and saliva (Kelly, R. J. et al., J Biol Chem 270, 4640-9 (1995)). The prevalence of the non-secretor status (Se−) is similar between populations (Pang, H. et al., Ann Hum Genet 65, 429-37 (2001)) although the point mutations that lead to Se− differ. The dominant non-secretor polymorphism in caucasians is the Trp143Ter (W143X) (Kelly, R. J. et al., J Biol Chem 270, 4640-9 (1995)) and it is this polymorphism that is implicated in CD in the replication cohort.

Pathogens utilize host cell surface molecules including oligosaccharides (synthesized by glycosyltransferases) for invasion. It is likely that the high prevalence of non-secretor phenotypes in the population occurs due to the absence of particular carbohydrate molecules in the mucosa, and this may have conferred some historical protection to infection as demonstrated with non-secretor status and protection from Helicobacter Pylori infection (Ikehara, Y. et al., Cancer Epidemiol Biomarkers Prev 10, 971-7 (2001)). Lactobacilli, a known commensal bacteria, bind to the precursor glycolipid GA1, implying a role of the GI mucosal glycolipid profile in the adherence of commensal and ‘beneficial’ bacteria, in addition to pathogenic organisms (Yamamoto, K. et al., Biochem Biophys Res Commun 228, 148-52 (1996)). Furthermore Lactobacilli can also displace pathogens such as Clostridium from mucus (Lee, Y. J. et al., Int J Antimicrob Agents 21, 340-6 (2003)) and inhibit the Shigella-host interaction (Moorthy, G. et al., Dig Liver Dis (2009)). Commensal bacteria likely induce glycolipid expression, as the fucosylglycolipid FGA1 is found in the small bowel of conventionally bred mice but not in germ-free mice (Iwamori, M. & Domino, S. E. Biochem J 380, 75-81 (2004)). Furthermore FGA1 expression is induced by administration of microbes (Lin, B. et al., Arch Biochem Biophys 388, 207-15 (2001)), and FUT2 transcripts in the ileum were induced in germ free mice 48 hours after administration of feces from conventionally bred mice (Lin, P. H. et al., Am Surg 66, 627-30 (2000)). Fut2-null mice do not express the fucosylglycolipid FGA1 in the cecum and colon, whereas normal mice do (Iwamori, M. & Domino, S. E. Biochem J 380, 75-81 (2004)). In the mammalian gut, blocking the CRK and JNK pathways inhibits the ability of bacterial colonization to induce fucosyltransferase activity and FUT2 mRNA expression, both of which are hallmarks of the adult mammalian colon (Meng, D. et al., Am J Physiol Gastrointest Liver Physiol 293, G780-7 (2007)). Commensal bacteria and probiotics may exert their protective effects through preventing adherence or even displacing pathogenic bacteria, thus emphasizing the potential role of FUT2 and non-secretor status on gastrointestinal bacterial profile (Collado, M. C. et al., Lett Appl Microbiol 45, 454-60 (2007)). It is likely that Se-individuals may thus have a disrupted immunogenic/homeostatic equilibrium that makes them more susceptible to the development of chronic mucosal inflammation, and changes in the microflora of IBD patients have been well-documented (Swidsinski, A. et al., Inflamm Bowel Dis 14, 147-61 (2008)). There are some data to support this concept, as Fut2 null mice display increased susceptibility to experimental yeast vaginitis and cervical mucins containing Fut2 are partly protected from induced vaginal candidiasis (Hurd, E. A. & Domino, S. E., Infect Immun 72, 4279-81 (2004)).

Although FUT2 is a strong candidate gene for CD susceptibility, given its tissue expression and its influence on the GI bacterial profile, the associations identified in FUT2 may reflect association with other genetic variants at this locus that are in linkage disequilibrium with these SNPs. The inventors therefore explored the LD pattern at this locus using the latest version of HapMap (Frazer, K. A. et al., Nature 449, 851-61 (2007)) and identified that LD (defined as D′>0.80) extends into neighboring genes, including interesting candidate genes that are also potentially involved in the host-bacterial interaction such as FUT1 (alpha-1-2-fucosyltransferase 1—FUT, genetic variation in pigs is associated with alterations in E. Coli adherence (Meijerink, E. et al., Immunogenetics 52, 129-36 (2000))) and RASIP1 (RAS interacting protein 1—a RAS effector localized to the Golgi membranes) as well as DBP (D-site of albumin promoter-binding protein) and FGF21 (fibroblast growth factor 21—involved in insulin sensitivity, adipocyte function and growth hormone signalling (Berglund, E. D. et al., Endocrinology (2009); Inagaki, T. et al., Cell Metab 8, 77-83 (2008))). The inventors believe that FUT2 is an attractive candidate gene at this locus, and have demonstrated association with a variant with a known consequence on gene expression.

In addition, the inventors have identified some novel loci for further investigation, including genes involved in tight junctions, Substance P signaling, macrophage development, dendritic cell function and NK T cell function.

The data disclosed herein provide strong evidence that non-secretor status increases CD susceptibility. The non-secretor variants from other ethnic groups have been well documented, and studies of these variants within the relevant IBD populations will help elucidate the exact role of FUT2 in CD susceptibility. Studies on the effect of FUT2 on clinical and serological phenotype, and particular its role on the microbiome of non-secretor individuals, may help investigators understand further the variation seen in commensal bacteria in individuals with CD, and also further determine those CD patients who might most benefit from probiotic or antibiotic based therapies for prevention and treatment of CD.

In various embodiments, the present invention provides a method of diagnosing susceptibility to Crohn's disease in an individual, comprising: obtaining a sample from the individual; assaying the sample to determine the presence or absence of a risk variant at the FUT2 genetic locus; and diagnosing susceptibility to Crohn's disease in the individual based on the presence of the risk variant at the FUT2 genetic locus.

In various embodiments, the risk variant is selected from the group consisting of rs602662, rs676388, rs485186, and rs504963.

In various embodiments, assaying the sample comprises genotyping for one or more single nucleotide polymorphisms.

In various embodiments, the sample is whole blood, plasma, serum, saliva, cheek swab, urine, or stool.

In various embodiments, the present invention provides a method of prognosing Crohn's disease in an individual, comprising: obtaining a sample from the individual; assaying the sample for the presence or absence of one or more genetic risk variants; and prognosing an aggressive form of Crohn's disease based on the presence of one or more risk variants at the FUT2 genetic locus.

In various embodiments, the risk variant is selected from the group consisting of rs602662, rs676388, rs485186, and rs504963.

In various embodiments, assaying the sample comprises genotyping for one or more single nucleotide polymorphisms.

In various embodiments, the sample is whole blood, plasma, serum, saliva, cheek swab, urine, or stool.

In various embodiments, the present invention provides a method of treating an individual for Crohn's disease, comprising: prognosing an aggressive form of Crohn's disease in the individual based on the presence of one or more risk variants at the FUT2 genetic locus; and treating the individual, wherein the one or more risk variants are selected from rs602662, rs676388, rs485186, and rs504963.

In various embodiments, assaying the sample comprises genotyping for one or more single nucleotide polymorphisms.

In various embodiments, the sample is whole blood, plasma, serum, saliva, cheek swab, urine, or stool.

In various embodiments, the present invention provides a method of determining a high probability of developing Crohn's disease in an individual, relative to a healthy subject, comprising: obtaining a sample from the individual; assaying the sample to determine the presence or absence of one or more risk variants at the FUT2 genetic locus; and diagnosing a high probability of developing Crohn's disease in the individual, relative to a healthy subject, based upon the presence of the one or more risk variants at the FUT2 genetic locus.

In various embodiments, the one or more risk variants are selected from the group consisting of rs602662, rs676388, rs485186, and rs504963.

In various embodiments, assaying the sample comprises genotyping for one or more single nucleotide polymorphisms.

In various embodiments, the sample is whole blood, plasma, serum, saliva, cheek swab, urine, or stool.

### Example 4

### Methods of Diagnosing Ulcerative Colitis and Crohn's Disease

This non-limiting example relates to methods of prognosing inflammatory bowel disease (IBD) in an individual by determining the presence of at least one risk genetic variant and/or at least one risk serological marker. In one embodiment, the presence of risk serological marker ANCA is indicative of an aggressive form of ulcerative colitis. In another embodiment, this non-limiting example relates to methods of diagnosing a Crohn's disease subtype in an individual, where the presence of risk variants and serological markers I2, OmpC and/or Cbir1 are indicative of the Crohn's disease subtype.

### Example 4-1

Ulcerative colitis (UC), a subtype of Inflammatory Bowel Disease (IBD), is a chronic inflammatory condition of the gastrointestinal tract with a complex genetic and environmental component. In UC particularly, the environmental factors and the role of bacteria in diseases pathogenesis remains unknown. Response to ANCA has been associated with more aggressive disease behavior in UC patients, whereas sero-reactivity to ASCA, anti-CBir1, anti-I2, and anti-OmpC have been particularly associated with subtypes of Crohn's disease. Furthermore, there is a hereditary component to expression of these antibodies.

The inventors assessed the genetic contribution to IBD associated serological profiles in UC cases. 1327 UC cases were genotyped with the Illumina CNV370 or OmniExpress beadchips, and were sero-typed for ANCA, ASCA, anti-CBir1, anti-I2, and anti-OmpC by ELISA. Regression analyses was performed, adjusted for population stratification using principal components as covariates, testing for an association of UC with antibody response. A Z-score for ASCA, anti-CBir1, anti-I2, and anti-OmpC together was generated by adding the four Z-scores for each individual antibody for each subject. The Z-scores were calculated from within the UC cohort only. Association of UC was assessed with this combined score and with ANCA status alone.

The results demonstrate two genome-wide significant associations with UC and (1) ANCA at chr.4 (rs1919469 p_{logistic}=4.82×10⁻⁸, OR=1.90; rs10001225 p_{logistic}=1.97×10⁻⁷, OR=1.77). An additional three SNPs within this region are also found to be associated with nominal significance (p<10⁻⁵); and (2) at a second region on chr.4˜37 Mb away, with the combined ASCA, I2, CBir1 and Ompc Z-score (rs2995965 pₗᵢₙₑₐᵣ=1.35×10⁻⁹, β=0.82; rs1863284 p^{linear}=1.71×10⁻⁷, β=0.85; rs2911920 pₗᵢₙₑₐᵣ=6.29×10⁻⁶, (3=0.61). RELL1, a homologue of RELT the TNF receptor that induces epithelial cell apoptosis is located at this locus. These observations support that these two loci contribute to the phenotypic difference of UC patients, one for UC severity as typified by ANCA level, the other for the expression of antibodies more characteristic of CD.

### Example 4-2

It has been reported that CD patients can be characterized by the association of disease phenotypes with the expression of antibodies to microbial antigens. For example CD associated serologies such as ASCA, I2, CBir1 and OMPc are associated with a more aggressive course of disease and an increased chance of surgery. It has also been previously demonstrated the heritable nature of these IBD associated antibodies.

The inventors conducted a genome-wide association study (GWAS) on 1544 CD subjects serotyped for CD-associated antibodies (ASCA, anti-CBir1, anti-I2, and anti-OMPC). Serum antibody expression was measured by ELISA and levels were log transformed prior to analyses. Single nucleotide polymorphism (SNP) data were generated using Illumina technology (˜550K SNPs with MAF>0.05) at Cedars-Sinai Medical Center. Adjustment for population stratification was carried out using two principal components as covariates in the analyses (Eigensoft). The significance of association was tested using logistic regression for antibody positive or negative and linear regression for antibody level after transformation. To overcome multiple testing issues significance was defined to be p<2e-07.

At the pre-defined level of significance, the inventors observed two significant associations: 1) expression of anti-I2 was significantly associated with 3 SNPs spanning 90 kb of chr. 15 that included the 3 region of human EST BF729345, among other ESTs (rs246336, OR for G allele and anti-I2 positivity, 1.8; p (logistic regression)=8.6e-08); and 2) Expression of anti-OMPC was significantly associated with rs6566234 on chr. 18 (beta coefficient for G allele was −0.28, p (linear regression)=1.4e-07), potentially in LD with CDH19. In addition, 3) anti-Cbir1 positivity was associated with gene AK097193 on chr. 1 (rs1022265 G allele OR for anti-CBir positivity 0.68 p (logistic regression)=7.6 e-07); and 4) ASCA positivity was associated with two SNPs on chr. 3 (rs291528 & rs291523, OR 1.9, p (logistic regression)=5e-07).

Examples of rs246336, rs6566234, rs291528, and rs291523 are provided herein as SEQ ID NO:40, SEQ ID NO:41, SEQ ID NO:42, and SEQ ID NO:43, herein.

These results show that GWAS of serum expression to microbial antibodies may lead to discovery of novel loci affecting CD course and thus targets for therapies for aggressive CD.

### Example 4-3

Crohn's Disease (CD), a subtype of Inflammatory Bowel Disease (IBD), is a chronic inflammatory condition of the gastrointestinal tract with a complex genetic and environmental component. It has been reported that combinations of genetic and serological markers, including antibodies to anti-Saccharomyces cerevisiae (ASCA), E. Coli outer membrane porinC (OmpC), Pseudomonas fluorescens protein (I2), and anti-flagellin (CBir1), are associated with complications of Crohn's disease. Severe CD is associated with the expression of more than one antibody as well as higher levels of antibody expression.

The inventors identified genes contributing to CD severity by conducting a genome-wide association study (GWAS) of antibody expression in serotyped CD subjects. 1537 CD cases with complete serum antibody profile were genotyped with the Illumina 610quad or OmniExpress beadchips at Cedars-Sinai Medical Center Medical Genetics Institute. Serum antibody for ANCA, ASCA, anti-OmpC, anti-I2, and anti-CBir1 expression was measured by ELISA and log-transformed prior to analyses. 303,147 SNPs with HWE >0.001, MAF>0.02, and GENO >0.02 (genotyping rate=0.9993) were included in analyses. Association for autosomal chromosomes of CD was assessed with Antibody Score using linear regression and with ANCA status using logistic regression.

An excess of significance in the tail of the distribution suggests that true positive results are present (below).

TABLE 7
SNPs associated with ANCA positive/negative

| CHR | SNP | Allele | OR | P | GENE(S) IN LD |
|---|---|---|---|---|---|
| 3 | rs1973780 | A | 1.63 | 3.6 × 10⁻⁶ | FHIT |
| | (SEQ ID NO: 44) | | | | Fragile histidine |
| | | | | | triad gene |
| 17 | rs1728171 | A | 0.45 | 3.2 × 10⁻⁶ | ETV4 |
| | (SEQ ID NO: 45) | | | | ETS variant 4 |
| 6 | rs9449593 | A | 2.1 | 7.7 × 10⁻⁶ | ME1 |
| | (SEQ ID NO: 46) | | | | Malic enzyme 1 |
| 1 | rs6690359 | A | 0.65 | 9.3 × 10⁻⁶ | WDR64 |
| | (SEQ ID NO: 47) | | | | WD repeat domain 64 |

TABLE 8
SNPs associated with Antibody Score in Crohn's Disease

| CHR | SNP | Allele | OR | P | GENE(S) IN LD |
|---|---|---|---|---|---|
| 16 | rs1019257 | A | −0.60 | 2.2 × 10⁻⁶ | A2BP1 (aka |
| | (SEQ ID NO: 48) | | | | RBFOX1) |
| | | | | | Ataxin 2 binding |
| | | | | | protein 1Trans- |
| | | | | | golg inetwork; |
| | | | | | associated with |
| | | | | | osteoarthritis |
| | | | | | and aortic plaque |
| 18 | rs766613 | A | 0.37 | 3.6 × 10⁻⁶ | CDH2 |
| | (SEQ ID NO: 49) | | | | Cadherin 2 |
| 19 | rs10403164 | A | −0.37 | 9.8 × 10⁻⁶ | HSPBP1, PPP6R1, |
| | (SEQ ID NO: 50) | | | | BRSK1 Heat |
| | | | | | shock 70 kDa |
| | | | | | binding protein, |
| | | | | | co-chaperone |
| | | | | | 1; Protein |
| | | | | | phophatase |
| | | | | | 6 regulatory |
| | | | | | subunit 1; |
| | | | | | BR serine/ |
| | | | | | threonine kinase |

These results show genes for antibody expression in CD subjects. These genes are novel with respect to current GWAS results for CD. Because antibody expression is further associated with disease severity, characterization of these genetic associations may add to the list of genetic determinants of CD as well as to the characterization of immune processes that affect CD phenotype.

In various embodiments, the present invention provides a method of prognosing inflammatory bowel disease (IBD) in an individual, comprising: obtaining a sample from the individual; assaying the sample to determine the presence or absence of one or more risk variants at Chromosome 4; assaying the sample to determine the presence or absence of serological marker ANCA; and prognosing an aggressive form of inflammatory bowel disease in the individual based on the presence of one or more risk variants at Chromosome 4 and the presence of serological marker ANCA.

In various embodiments, the aggressive form of inflammatory bowel disease is characterized by an aggressive form of ulcerative colitis.

In various embodiments, the one or more risk variants at Chromosome 4 are at the genetic loci of AFP, AFM, RASSF6 and/or PGM2.

In various embodiments, the one or more risk variants at Chromosome 4 comprise SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:33, SEQ ID NO:34, SEQ ID NO:35, SEQ ID NO:36, SEQ ID NO:37, SEQ ID NO:38, and/or SEQ ID NO:39.

In various embodiments, the presence of serological marker ANCA comprises a high level of serological marker ANCA as compared to a healthy subject.

In various embodiments, the absence of serological marker ANCA is indicative of inflammatory bowel disease with Crohn's like conditions.

In various embodiments, the present invention provides a method of diagnosing an ulcerative colitis subtype in an individual, comprising: obtaining a sample from the individual; assaying the sample to determine the presence or absence of serological marker ANCA; and diagnosing the ulcerative colitis subtype in the individual, wherein the presence of serological marker ANCA is indicative of an aggressive subtype of ulcerative colitis, and wherein the absence of serological marker ANCA is indicative of an ulcerative colitis subtype with Crohn's disease characteristics.

In various embodiments, the method further comprises assaying the sample to determine the presence of one or more risk variants at Chromosome 4.

In various embodiments, the one or more risk variants at Chromosome 4 comprise SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:33, SEQ ID NO:34, SEQ ID NO:35, SEQ ID NO:36, SEQ ID NO:37, SEQ ID NO:38, and/or SEQ ID NO:39.

In various embodiments, the presence of serological marker ANCA comprises a high level of serological marker ANCA as compared to a healthy subject.

In various embodiments, the present invention provides a method of diagnosing a Crohn's disease subtype in an individual; comprising: obtaining a sample from the individual; assaying the sample to determine the presence or absence of one or more genetic risk variants located at Chromosome 15, Chromosome 18, and/or AK097193 genetic locus; and assaying the sample to determine the presence or absence of serological markers I2, OmpC and/or Cbir1; and diagnosing the Crohn's disease subtype based on the presence of one or more genetic risk variants and the presence of one or more serological markers.

In various embodiments, the one or more genetic risk variants comprise SEQ ID NO:40, SEQ ID NO:41, SEQ ID NO:42, and/or SEQ ID NO:43.

In various embodiments, SEQ ID NO:40 is associated with the presence of antibody 12.

In various embodiments, SEQ ID NO:41 is associated with the presence of antibody OmpC.

In various embodiments, SEQ ID NO:42 and/or SEQ ID NO:43 is associated with the presence of antibody Cbir1.

In various embodiments, the present invention provides a method of diagnosing susceptibility to Crohn's disease in an individual, comprising: obtaining a sample from the individual; assaying the sample to determine the presence or absence of one or more genetic risk variants located at the genetic loci of FHIT, ETV4, ME1, WDR64, A2BP1, CDH2, HSPBP1, PPP6R1, and/or BRSK1; and diagnosing susceptibility to Crohn's disease in the individual based on the presence of one or more genetic risk variants.

In various embodiments, the one or more genetic risk variants are associated with the presence of serological marker ANCA.

In various embodiments, the one or more genetic risk variants comprise SEQ ID NO:21, SEQ ID NO:45, SEQ ID NO:46, SEQ ID NO:47, SEQ ID NO:48, SEQ ID NO:49, and/or SEQ ID NO:50.

### Example 5

### Methods of Diagnosing and Treating Intestinal Granulomas and Low Bone Density in Inflammatory Bowel Disease

This non-limiting example relates to methods of diagnosing inflammatory bowel disease (IBD) in an individual by determining the presence of at least one risk genetic variant and/or at least one risk serological marker. In one embodiment, the presence of at least one risk genetic variant is indicative of granuloma. In another embodiment, the presence of at least one risk genetic variant is indicative of low bone density (LBD).

### Example 5-1

### Granuloma

The inventors identified clinical, serologic and genetic factors associated with granuloma formation in Crohn's disease (CD). 371 patients with CD who underwent disease-related surgical resection by a single surgeon were included in the study. Surgical samples were examined specifically for the presence or not of granulomas. Patients' demographic and clinical characteristics were collected by chart review, and samples drawn for IBD related serology (ASCA, anti-I2, anti-OmpC, CBir1 and ANCA) and genetic analyses. Genome-wide analyses were performed using Illumina technology. Standard statistical tests for association were used and genetic association was assessed both at the genome-wide level and against known IBD and Leprosy susceptibility loci.

34.7% of CD surgical samples were found to contain granulomas. Granulomas were not associated with CD disease behavior. High ASCA titre was associated with the presence of granulomas (p=0.02). Patients with granulomas were younger at time of surgery (29.9 vs. 37.6 years, p=5×10⁻⁷) and far less likely to have ever smoked (12 vs. 32%, p=7×10⁻⁵). 14 Single Nucleotide Polymorphisms (SNPs) were associated with granulomas at a level of nominal association at a genome-wide level (p<0.00005). These include a SNP adjacent to TGFb3, which has been implicated in the pathogenesis of stricturing Crohn's disease, and FTO, which is regulated by oral intake and is associated with raised body mass index. The strongest association was with NPAS2 (p=1×10⁻⁶), a core circadian gene that has been shown to modulate transcription of CX3CL1, a chemokine involved in CD pathogenesis. Amongst known IBD-associated loci, 7 were associated with granuloma formation (p<0.05), including: MUC1 (KL-6), also associated with granuloma-forming hypersensitivity pneumonitis; IL10, with known immunoregulatory function in the gut; and LRAP, associated with antigen presentation and LRRK2 a leucine-rich repeat kinase gene. One TNFSF15 SNP showed a trend towards association with the presence of granulomas (P=0.066), of particular interest given a recent report that TNFSF15 is associated with Leprosy, another granulomatous condition. Of the known Leprosy loci (in addition to LRRK2 and TNFSF15), the inventors identified association with granulomatous CD and SNPs across the cytochrome P-450 cluster. Thus, the inventors have demonstrated putative genetic and demographic associations with the presence of granulomas in CD including a number of genes associated with Leprosy suggesting unique pathways in the pathogenesis of this subset of CD.

### Example 5-2

### Low Bone Density

The inventors identified 333 IBD subjects with bone density studies who had previously had genome wide association studies and IBD related serologies performed. Data on age, gender, ethnicity, disease distribution, surgeries, and smoking history were obtained from chart reviews. Osteoporosis, osteopenia, and normal bone mineral density (NBD) were defined by the WHO criteria based on DEXA scans. Standard tests for association between clinical characteristics, genetic markers and serologies were used. IBD related serology (ASCA, OmpC, 12, CBir-1, and ANCA) were obtained by ELISA and summarized into quartiles. Genetic data were generated using Illumina technology.

Of the 333 IBD study subjects, the inventors identified 252 cases of LBD and 81 cases of NBD. Disease location was not associated with LBD overall; however, perianal disease was associated with osteoporosis (P=0.021). Small bowel disease requiring surgery was associated with LBD (P=0.022), osteopenia (P=0.041) and osteoporosis (P=0.05). Smoking was not associated with bone density. Mean and median Anti-I2 titers were associated with LBD (P=0.023) and osteoporosis (P=0.006). On quartile analysis, anti-CBir-1 titers were associated with LBD (P=0.036) and osteoporosis (P=0.0006); further, ASCA was associated with osteoporosis (P=0.03). 38 genetic loci achieved nominal level of genome wide significance (P<5×10⁻⁵) including multiple single nucleotide polymorphisms (SNPs) at the HLA (P=1.37×10⁻⁷) as well as genes involved in cell adhesion (laminin, P=4.41×10⁻⁵) and innate immunity (plexin, P=9.02×10⁻⁷; NLR family, P=7.39×10⁻⁶). Stepwise linear regression was performed and all but 2 SNPs (rs11576349 and rs4954555) fell out of the model. These two SNPs were independently associated with LBD (2.41×10⁻⁵ and 1.07×10⁻⁵) and together this 2 SNP model was highly associated with LBD (p-value linear regression 1.8×10⁻⁹) and explained 12.6 of the variance. Perianal disease is associated with osteoporosis; further, small bowel disease requiring surgery increases the risk for LBD. Anti-I2, anti-CBir-1, and ASCA are associated with increased risk for LBD and/or osteoporosis. Genes including HLA, laminin and plexin are associated with LBD. Thus, patients with these risk factors may benefit from more aggressive screening and treatment for osteoporosis.

In various embodiments, the present invention provides a method of diagnosing susceptibility to granuloma in an individual with Crohn's disease, comprising: (a) obtaining a sample from the individual; (b) assaying the sample to determine the presence or absence of at least one risk genetic variant; (c) assaying the sample to determine the presence or absence of at least one risk serological marker; and (d) diagnosing susceptibility to granuloma in the individual if the at least one risk genetic variant is present, or if the at least one risk serological marker is present, or if the at least one risk genetic variant is present and the at least one risk serological marker is present.

In various embodiments, the at least one risk genetic variant is at the genetic locus of TGFb3, FTO, NPAS2, MUC1, IL10, LRAP, LRRK2, TNFSF15, or cytochrome P-450 cluster, or a combination thereof.

In various embodiments, the at least one risk serological marker is selected from the group consisting of anti-Cbir1, ANCA, ASCA, anti-OmpC, and anti-I2.

In various embodiments, the ASCA is present in high titer.

In various embodiments, the at least one risk genetic variant comprises SEQ ID NO:51, SEQ ID NO:52, SEQ ID NO:53, SEQ ID NO:54, SEQ ID NO:55, and/or SEQ ID NO:56.

In various embodiments, the at least one risk genetic variant comprises SEQ ID NO:57, SEQ ID NO:58, SEQ ID NO:59, SEQ ID NO:60, SEQ ID NO:61, SEQ ID NO:62, and/or SEQ ID NO:63.

In various embodiments, the Crohn's disease is associated with a small bowel disease phenotype, an aggressive complicating phenotype, an internal penetrating disease phenotype, a stricturing disease phenotype, a fibrostenosing disease phenotype, or a combination thereof.

In various embodiments, the sample comprises a nucleic acid from the individual.

In various embodiments, the present invention provides a method of diagnosing granuloma in an individual with Crohn's disease, comprising: (a) obtaining a sample from the individual; (b) assaying the sample to determine the presence or absence of at least one risk genetic variant; (c) assaying the sample to determine the presence or absence of at least one risk serological marker; and (d) diagnosing granuloma in the individual if the at least one risk genetic variant is present, or if the at least one risk serological marker is present, or if the at least one risk genetic variant is present and the at least one risk serological marker is present.

In various embodiments, the at least one risk genetic variant is at the genetic locus of TGFb3, FTO, NPAS2, MUC1, IL10, LRAP, LRRK2, TNFSF15, cytochrome P-450 cluster, or a combination thereof.

In various embodiments, the at least one risk serological marker is selected from the group consisting of anti-Cbir1, ANCA, ASCA, anti-OmpC, and anti-I2.

In various embodiments, the ASCA is present in high titer.

In various embodiments, the at least one risk genetic variant comprises SEQ ID NO:51, SEQ ID NO:52, SEQ ID NO:53, SEQ ID NO:54, SEQ ID NO:55, and/or SEQ ID NO:56.

In various embodiments, the at least one risk genetic variant comprises SEQ ID NO:57, SEQ ID NO:58, SEQ ID NO:59, SEQ ID NO:60, SEQ ID NO:61, SEQ ID NO:62, and/or SEQ ID NO:63.

In various embodiments, the Crohn's disease is associated with a small bowel disease phenotype, an aggressive complicating phenotype, an internal penetrating disease phenotype, a stricturing disease phenotype, a fibrostenosing disease phenotype, or a combination thereof.

In various embodiments, the present invention provides a method of diagnosing susceptibility to low bone density (LBD) in an individual with inflammatory bowel disease (IBD), comprising: (a) obtaining a sample from the individual; (b) assaying the sample to determine the presence or absence of at least one risk genetic variant; (c) assaying the sample to determine the presence or absence of at least one risk serological marker; and (d) diagnosing susceptibility to LBD in the individual if the at least one risk genetic variant is present, or if the at least one risk serological marker is present, or if the at least one risk genetic variant is present and the at least one risk serological marker is present.

In various embodiments, the LBD is associated with osteoporosis and/or osteopenia.

In various embodiments, the at least one risk genetic variant is at the genetic locus of HLA, laminin, plexin, NLR family, or a combination thereof

In various embodiments, the at least one risk genetic variant is SEQ. ID. NO.: 64 and/or SEQ. ID. NO.: 65.

In various embodiments, the at least one risk serological marker is selected from the group consisting of anti-Cbir1, ASCA, and anti-I2.

In various embodiments, the IBD is associated with perianal disease.

In various embodiments, the present invention provides a method of treating low bone density (LBD) in an individual with inflammatory bowel disease (IBD), comprising: (a) obtaining a sample from the individual; (b) assaying the sample to determine the presence or absence of at least one risk genetic variant; (c) assaying the sample to determine the presence or absence of at least one risk serological marker; and (d) treating LBD in the individual if the at least one risk genetic variant is present, or if the at least one risk serological marker is present, or if the at least one risk genetic variant is present and the at least one risk serological marker is present.

In various embodiments, the at least one risk genetic variant is SEQ. ID. NO.: 64 and/or SEQ. ID. NO.: 65.

In various embodiments, the at least one risk serological marker is selected from the group consisting of anti-Cbir1, ASCA, and anti-I2.

### Example 6

### Role of IFNG Methylation in Inflammatory Bowel Disease

This non-limiting example relates to method of diagnosing susceptibility to inflammatory bowel disease (IBD) in an individual by obtaining a sample from the individual, assaying the sample to determine the presence or absence of one or more risk genetic variants and/or an increase in IFNG DNA methylation. In one embodiment, this non-limiting example provides a method of diagnosing susceptibility to inflammatory bowel disease (IBD) in an individual by obtaining a sample from the individual, assaying the sample to determine the presence or absence of one or more risk genetic variants and/or an increase in IFNG DNA methylation relative to a normal subject, and diagnosing susceptibility to inflammatory bowel disease based on the presence of one or more risk genetic variants and/or an increase in IFNG DNA methylation relative to a normal subject. In another embodiment, the IBD is ulcerative colitis.

### Example 6-1

### Methylation

Epigenetic remodeling of chromatin via DNA methylation affects transcriptional activation. It has been demonstrated a distinct IFNG DNA methylation pattern in mucosal T cells from IBD patients and in peripheral T cells of a subset of UC patients. Decreased IFNG methylation was associated with increased IFNG production and seroreactivity to microbial antigens. GWA Studies identified UC-risk/severity regions linked to single nucleotide polymorphisms (SNP) flanking IFNG. One of the challenges of GWAS is to define the functional consequences of these genetic variations. Many disease-associated SNPs target CpG sites, which are relatively rare within the genome and serve as sites for DNA methylation. Recently, allele specific methylation was reported to preferentially occur at CpG sites adjacent to SNPs that alter CpG sites. The CpG (C/T) SNP rs1861494 (+2109) is located in a conserved regulatory region of the third intron of IFNG, within the same LD block implicated with UC and disease severity. Two adjacent CpG sites are found at +2167 and +2209 bp. Though typically both alleles contribute towards gene expression, monoallelic expression of IFNG protein has been reported. Moreover, it seems likely that variants that alter CpG sites not only alter methylation but may lead to unequal allelic expression.

The inventors determined what was the methylation status for IFNG rs1861494 SNP alleles and whether a functional relationship exists between allele specific methylation and gene expression. 154 IBD patients were genotyped for the IFNG rs1861494. DNA strand specific methylation levels for SNP +2109 and adjacent +2167 and +2209 CpG sites were determined by pyrosequencing. Allele and methylation-specific nucleo-protein binding was determined by EMSA. Levels of IFNG secretion and immune response to CBir were measured by ELISA.

The wt rs1861494 T allele is un-methylated whereas the C allele displays 55% methylation. In adjacent CpG sites allele-specific DNA methylation was noted at the +2167, but not +2209, with decreased methylation of the C vs. T SNP allele DNA strands (p<0.001). The rs1861494 IFNG polymorphism is functionally associated with decreased IFNG production and levels of immune response to CBir. Allele-specific and methylation-sensitive alteration in DNA trans-factor binding patterns to the SNP was noted. Nucleo-protein binding to the unmethylated C SNP was lower than that seen for T SNP. However, methylation of the C allele strand markedly enhanced binding and the appearance of an additional nucleo-protein complex. These results link the same cis-regulatory IFNG variant with modulation of DNA strand methylation and transcription factor binding supporting a functional role for rs1861494 gene variant in regulating IFNG expression.

### Example 6-2

### Methylation Clusters and CD Subgroups

The inventors identified distinct genome-wide methylation and RNA expression patterns in IBD. Matched CD3+LPL and PBL were isolated from 12 CD, 11 UC or 8 normal (NL) donors; DNA and RNA was extracted; differentially methylated regions (DMRs) were identified using Illumina 450k Infinium Bead Array; mRNA expression measured using Illumina expression array; data analysis using BRB Array Tools; Data was filtered 80% variance, less than 50% missing, and 2-fold differences across all samples, yielding 13,079 CpG sites. As a result, the inventors found genome-wide differentially methylated regions in mucosal vs. peripheral T cells, and in IBD compared to normal patients. The CD patients display a greater percentage of DMRs mapped within IBD GWAS vs. non-GWAS loci, and distinct methylation and mRNA expression profiles in patients requiring early surgical intervention. Combined epigenetic and expression profiling may stratify CD patients into distinct subgroups exhibiting molecular patterns that identify patients with different natural history of disease.

### Example 6-3

Epigenetic remodeling of chromatin via DNA methylation affects transcriptional activation. It has been demonstrated a distinct IFNG DNA methylation pattern in mucosal T cells from IBD patients and in peripheral T cells of a subset of UC patients. Decreased IFNG methylation was associated with increased IFNG production and seroreactivity to microbial antigens. GWA Studies identified UC-risk/severity regions linked to single nucleotide polymorphisms (SNP) flanking IFNG. One of the challenges of GWAS is to define the functional consequences of these genetic variations. Many disease-associated SNPs target CpG sites, which are relatively rare within the genome and serve as sites for DNA methylation. Recently, allele specific methylation was reported to preferentially occur at CpG sites adjacent to SNPs that alter CpG sites. The CpG (C/T) SNP rs1861494 (+2109) is located in a conserved regulatory region of the third intron of IFNG, within the same LD block implicated with UC and disease severity. Two adjacent CpG sites are found at +2167 and +2209 bp. Though typically both alleles contribute towards gene expression, monoallelic expression of IFNG protein has been reported. Moreover, it seems likely that variants that alter CpG sites not only alter methylation but may lead to unequal allelic expression.

The inventors determined what was the methylation status for IFNG rs1861494 SNP alleles and whether a functional relationship exists between allele specific methylation and gene expression. 154 IBD patients were genotyped for the IFNG rs1861494. DNA strand specific methylation levels for SNP +2109 and adjacent +2167 and +2209 CpG sites were determined by pyrosequencing. Allele and methylation-specific nucleo-protein binding was determined by EMSA. Levels of IFNG secretion and immune response to CBir were measured by ELISA.

The wt rs1861494 T allele is un-methylated whereas the C allele displays 55% methylation. In adjacent CpG sites allele-specific DNA methylation was noted at the +2167, but not +2209, with decreased methylation of the C vs. T SNP allele DNA strands (p<0.001). The rs1861494 IFNG polymorphism is functionally associated with decreased IFNG production and levels of immune response to CBir. Allele-specific and methylation-sensitive alteration in DNA trans-factor binding patterns to the SNP was noted. Nucleo-protein binding to the unmethylated C SNP was lower than that seen for T SNP. However, methylation of the C allele strand markedly enhanced binding and the appearance of an additional nucleo-protein complex. These results link the same cis-regulatory IFNG variant with modulation of DNA strand methylation and transcription factor binding supporting a functional role for rs1861494 gene variant in regulating IFNG expression.

### Example 6-4

### Materials

### Isolation of T Cells

Peripheral blood mononuclear cells (PBMC) were isolated from healthy volunteers or IBD patients by separation on Ficoll-Hypaque gradients. CD3⁺ T cells (PB T) were isolated using CD3-immunomagnetic beads (Miltenyi Biotech, Auburn, Calif.) and were at least 95% pure.

### Study Subjects

Patients with IBD were recruited through the Inflammatory Bowel Disease Center at Cedars-Sinai Medical Center. Diagnoses of Crohn's disease and ulcerative colitis were confirmed using standard clinical, radiological, endoscopic and pathological criteria. All subjects were Caucasian non-Hispanic with the average age of 41 for CD (range 15-78) and 46 for UC (range 11-77) and were genotyped for rs1861494. All genotyping was performed at the Medical Genetics Institute at Cedars-Sinai Medical Center using Infinium technology (Illumina, San Diego, Calif.). Control subjects were healthy individuals, free of medication and with no known personal or family history of autoimmune disease or IBD.

### IFN-γ Assay

IBD T cells were stimulated with anti-CD3 antibody for 24 hours. IFN-γ was measured by an amplified ELISA. Greiner Bio-One (Longwood, Fla.) ELISA plates were coated overnight with 100 μl of 5 μg/ml monoclonal anti-IFN-γ (BD Biosciences, Woburn, Mass.). Samples and standards were added for 24 h followed by addition of 100 μl of 2.5 μg/ml polyclonal biotinylated rabbit anti-IFN-γ (BD Biosciences) for 2 h. This was followed by addition of 100 μl of 1/1000 diluted alkaline phosphatase-conjugated steptavidin (Jackson ImmunoResearch Laboratories, West Grove, Pa.) for 2 h. Substrate, 0.2 mM NADP (Sigma-Aldrich, St. Louis, Mo.) was added for 30 min followed by addition of amplifier (3% 2-propanol, 1 mM iodonitrotetrazolium violet, 75 μg/ml alcohol dehydrogenase, and 50 μg/ml diaphorase; Sigma-Aldrich) for 30 min. Plates were read at 490 nm using an E max plate reader (Molecular Devices, Sunnyvale, Calif.).

### Microbial Antibody Responses

All blood samples were taken at the time of consent and enrolment. Sera were analyzed for expression of ASCA, anti-OmpC, anti-I2 anti-CBir1 antibodies in a blinded fashion by ELISA as previously described (see Targan et al., Antibodies to CBir1 flagellin define a unique response that is associated independently with complicated Crohn's disease, Gastroenterology 2005 128:2020-2028; Mow et al., Association of antibody responses to microbial antigens and complications of small bowel Crohn's disease, Gastroenterology 2004, 126:414-424; and Targan et al., High-titer antineutrophil cytoplasmic antibodies in type-1 autoimmune hepatitis, Gastroenterology 1995, 108:1159-1166, which are incorporated by reference herein in their entirety as though fully set forth). Antibody levels were determined and results expressed as ELISA units (EU/ml) relative to a Cedars-Sinai Laboratory standard that was derived from a pool of patient sera with well-characterized disease found to have reactivity to this antigen.

### Pyrosequencing

DNA was extracted from T cells using a QIAmp DNA isolation kit (Qiagen Inc., Valencia, Calif.). All samples were analyzed in a blinded fashion using the EpigenDx custom pyrosequencing service (EpigenDx, Inc., Hopkinton, Mass.). Briefly, bisulfite treatment of 2 μg of DNA was carried out using the EZ DNA methylation kit (Zymo Research, Orange, Calif.) according to manufacturer's instructions. Hot-start PCR was carried out with HotStart Taq (Qiagen Inc.) using 100 ng of bisulfite treated DNA. PCR and pyrosequencing primers are shown in Table 9.

TABLE 9
PCR and pyrosequencing primers and conditions

| | | | SEQ ID | PCR | | | |
|---|---|---|---|---|---|---|---|
| Assay CpG | Primers | | NO: | Conditions | | | |
| -295, -186 | Fwd PCR | GTGAATGAAGAGTT | 68 | 94° C. | 95° C. 15 s | × 45 | 72° C. |
| | | AATATTTTATTAGG | | 15 min | 52° C. 30 s | cycles | 5 min |
| | Rev PCR | Biotin- | 69 | | 72° C. 15 s | | |
| | | TTCCTTTAAACTCCT | | | | | |
| | | TAAATCCTT | | | | | |
| -295 | Seq | GTGAATGAAGAGTT | 70 | | | | |
| | | AATATTTTATTAGG | | | | | |
| -186 | Seq | GGTGGGTATAATGG | 71 | | | | |
| | | GTTTG | | | | | |
| -54 | Fwd PCR | GGATTTAAGGAGTTT | 72 | 94° C. | 95° C. 15 s | × 45 | 72° C. |
| | | AAAGGAAA | | 15 min | 52° C. 30 s | cycles | 5 min |
| | Rev PCR | Biotin- | 73 | | 72° C. 15 s | | |
| | | AAAACAATATACTAC | | | | | |
| | | ACCTCCTCT | | | | | |
| | Seq | TTAAAAAATTTGTGA | 74 | | | | |
| | | A | | | | | |
| Allele- | Fwd PCR | TTTGGGTGATTTTGT | 75 | 95° C. | 95° C. 30 s | × 45 | 72° C. |
| specific | | AAAGTTAGTAAGAGA | | 15 min | 46° C. 30 s | cycles | 10 min |
| | Rev PCR | CCATTAAAACAAACA | 76 | | 72° C. 30 s | | |
| | | ACCTCTCAT | | | | | |
| | Seq | AAACTACTTCTCAAT | 77 | | | | |
| | | ACTCC | | | | | |
| T-specific | | ACTACTTCTCAATAG | 78 | | | | |
| | | TCCCT | | | | | |
| C-specific | | ACTACTTCTCAATAG | 79 | | | | |
| | | TCCCC | | | | | |

Direct quantification of the ratio of unmethylated to methylated cytosines was determined for each site using Pyro Q-CpG software. The IFNG non-CpG cytosine at site −181 bp served as an internal control and revealed that bisulfite conversion of DNA was greater than 95%. Likewise, only slight variability was detected in DNA samples treated with bisulfite on different days. The naïve NK92 cell line demonstrated complete conversion following bisulfite treatment and served as a demethylation control.

### EMSA

PBMC from healthy volunteers were stimulated up to 4 h with PMA/ionomycin and nuclear protein extracts were obtained. Nuclear protein extract (3-6 μg) was incubated at 25° C. with 0.25 mg/ml poly (dI-dC), in 20% glycerol, 5 mM MgCl₂, 2.5 mM EDTA, 2.5 mM DTT, 250 mM NaCl, 50 mM Tris pH 7.5 for 10 min. Oligonucleotides 5′-IRD700-labeled (Integrated DNA Technology, Coraville, Iowa) were then added (250 fmol) and the binding reactions incubated for an additional 30 min. The DNA-protein complexes were separated from unbound probe on a pre-run native 6% polyacrylamide gel in low ionic strength buffer (22.3 mM Tris pH 7.4, 22.3 mM Borate, 0.5 mM EDTA pH 8.0) and analyzed with Odyssey infrared imaging system (Li-Cor Biosciences). The rs1861494 oligonucleotides used were (polymorphic nucleotides are bolded, only upper strand is shown):

(SEQ ID NO: 80)
5′-TCAGTACTCCCTGTGCTTCTTCCTCA-3′,
(SEQ ID NO: 81)
5′-TCAGTACTCCCCGTGCTTCTTCCTCA-3′.

For methylated oligonucleotides, the following sequence was used with either methylation of one or both strands:

(SEQ ID NO: 82)
5′-TCAGTACTCCCC(Me)GTGCTTCTTCCTCA-3′.

### Statistical Analysis

Tests for statistical significance was performed using JMP Statistical Software (SAS Institute GmbH, Heidelberg, Germany) as follows: Test for significance between of rs1861494 SNP and ANCA, IFN-γ secretion and methylation levels was calculated by parametric Student's T test; test of association and trend using Fisher's exact test, cox proportional hazards model and Kaplan-Meier Survival Curves and Log-Rank Test.

### Ethical Considerations

All studies involving human subjects were approved by the Institutional Review Board at Cedars-Sinai Medical Center.

### Example 6-5

### IFNG Polymorphisms and Methylation Associated with IBD Disease Severity Rs1861494 T Allele Carriage was Increased Among IBD Patients with More Aggressive Disease

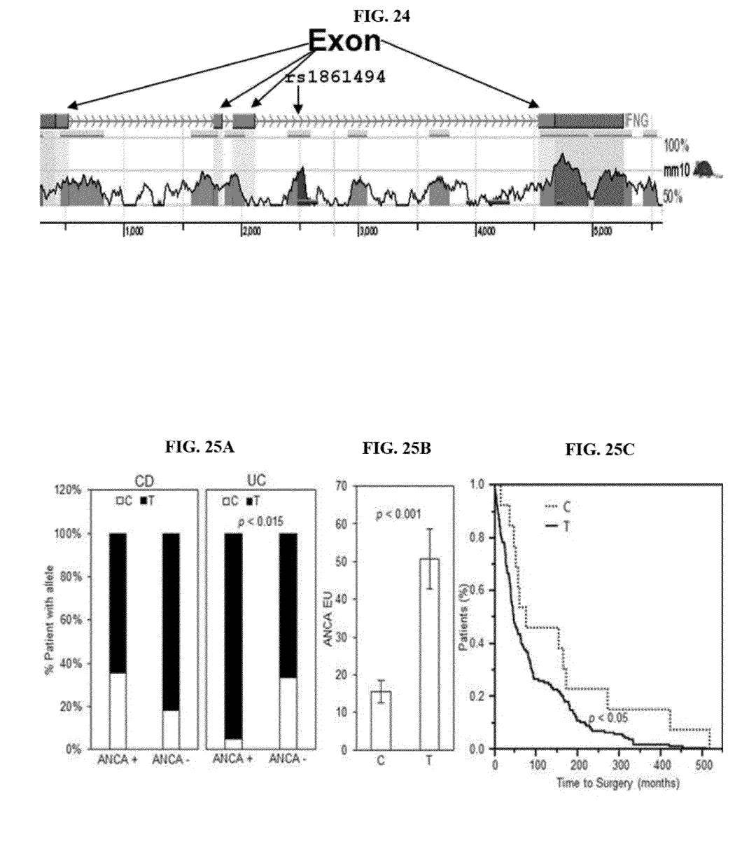

The IFNG +2109 SNP rs1861494 is located within a conserved regulatory region of the third intron of IFNG (FIG. 24). Disease-specific response to microbial and auto-antigens is associated with IBD. Sero-positivity to ANCA is detected in the majority of UC patients, but only a small percentage of CD patients, and is associated with a more aggressive disease phenotype in UC. In order to investigate whether rs1861494 was associated with disease severity in IBD, the IBD patient population was first stratified by disease phenotype and the SNP allele distribution was assessed in individuals with sero-reactivity to ANCA. It was found, in UC populations, the proportion of T (TT and TC) versus C allele (CC) carriers was significantly higher in the ANCA positive compared to ANCA negative patients (p<0.015) (FIG. 25A), and 95% of ANCA positive were T allele carriers (FIG. 25A). It was also found that the rs1861494 T allele carriers were associated with a significant increase in ANCA levels (p<0.001) (FIG. 25B) and an increase in the need for earlier surgical intervention for disease management (p<0.05) (FIG. 25C) in UC patients.

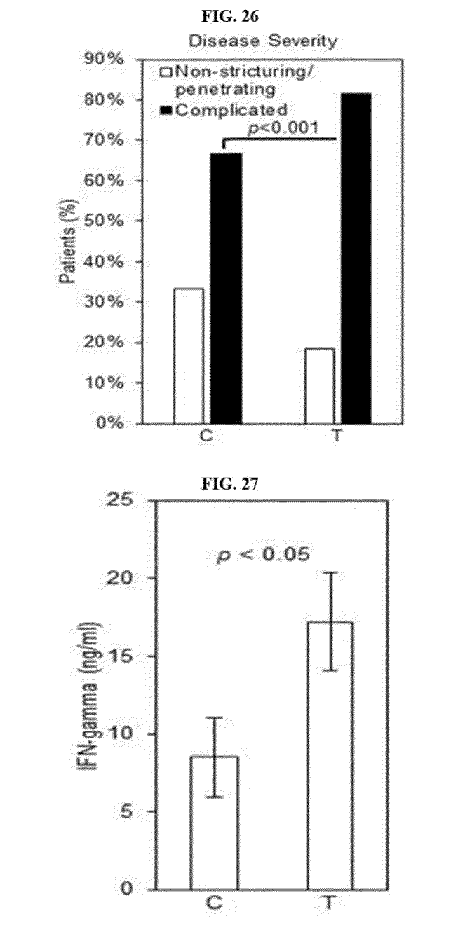

In CD populations, no significant association between rs186194 and ANCA was detected. However, a significant increase of complicated disease behavior, structuring/penetrating phenotype, was found in CD patients carrying the T allele (FIG. 26). No significant association between rs186194 and other CD serological markers (ASCA, OmpC, CBir, and I2) was detected. Thus, although the clinical manifestations differed, T allele carriers were associated with a more severe disease course for both UC and CD.

Rs1861494 T Allele Carriage was Associated with Increased IFN-γ Protein Expression

Mucosal expression of IFN-γ is key not only to the development and maintenance of inflammation but, additionally, the absolute levels of IFN-γ influence the severity of disease. Therefore, it was hypothesized that IBD T allele carriers would display a greater level of IFN-γ. Indeed, IFN-γ secretion was enhanced in IBD patients carrying the T allele compared to patients homozygous for the C allele following stimulation of peripheral T cells (FIG. 27).

### Methylation of Rs1861494 was Allele-Specific

The rs1861494 C to T SNP (+2109 bp) introduces a new potential CpG methylation site. An additional two CpG methylation sites reside 52 and 102 bp upstream at +2052 and +2007 bp.

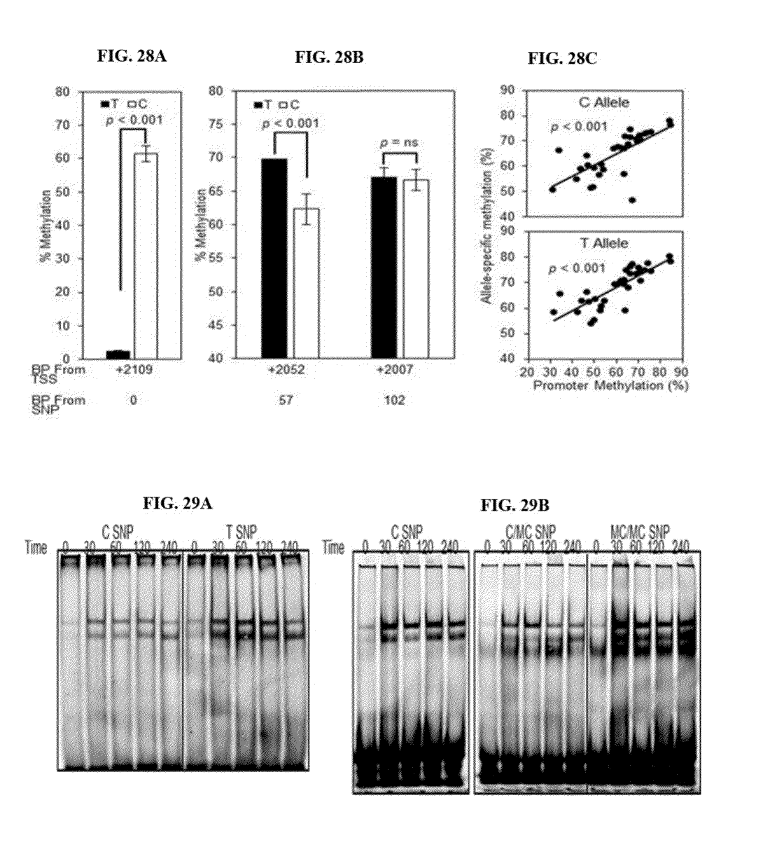

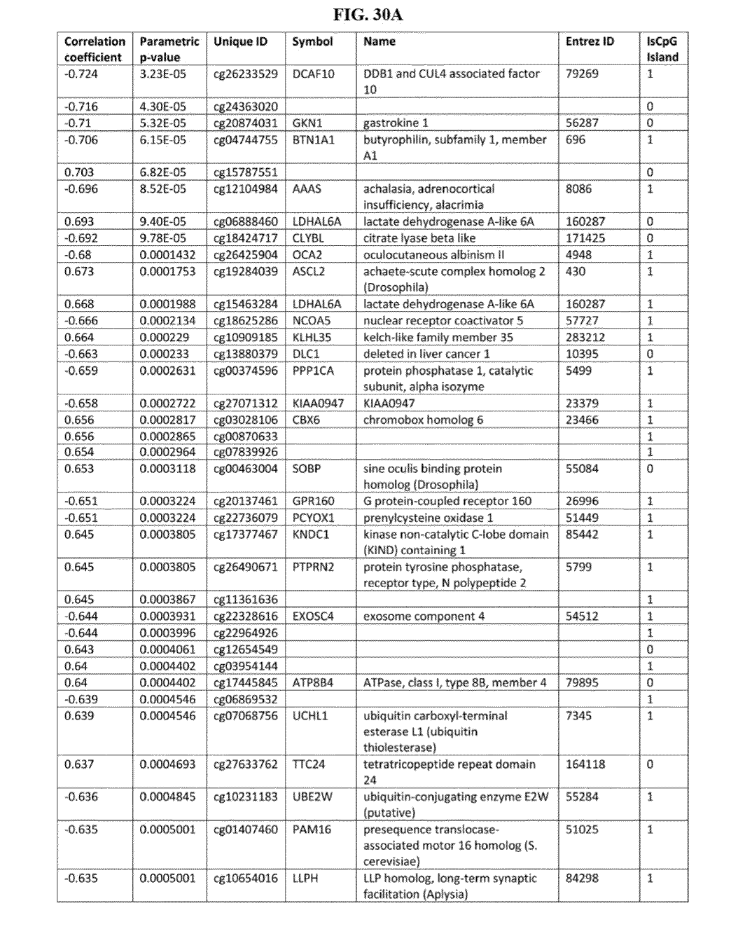

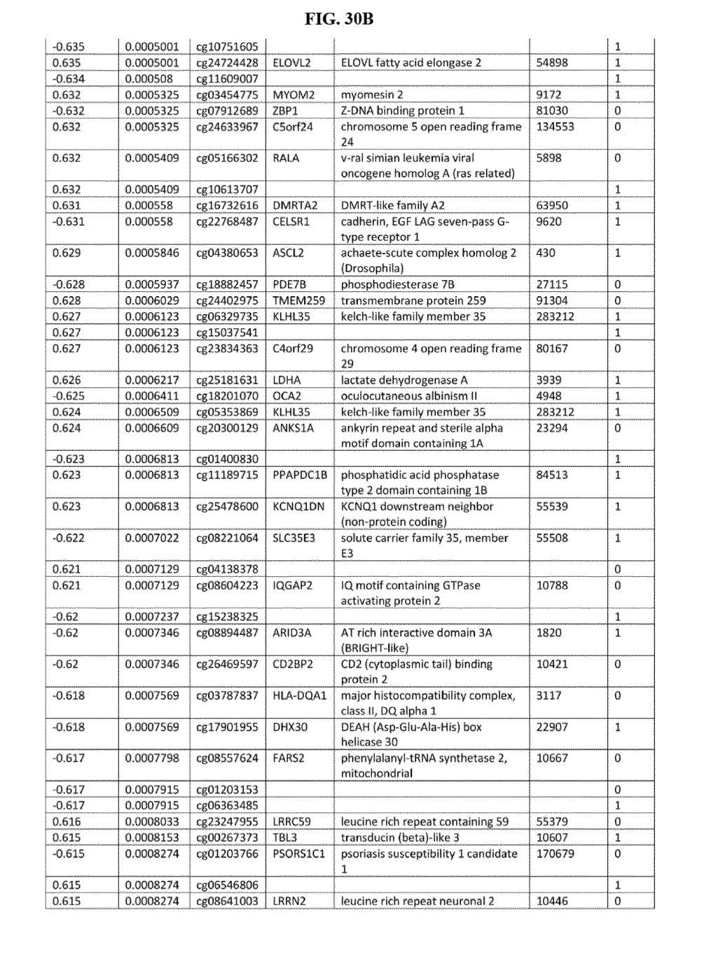

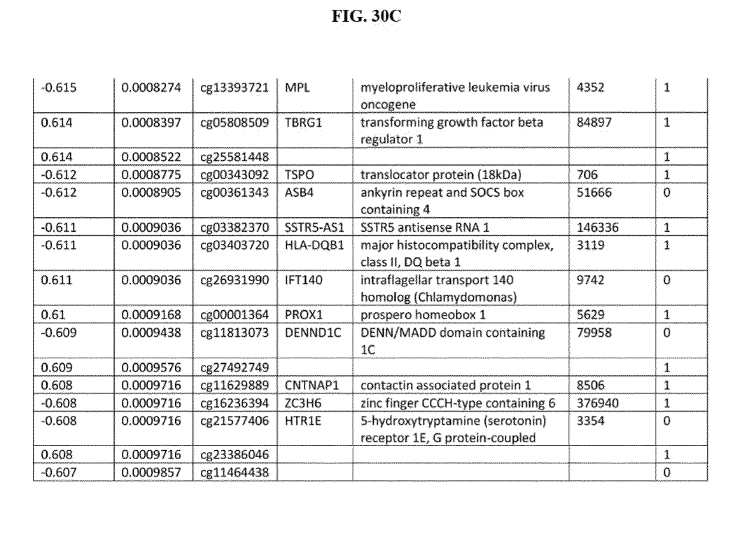

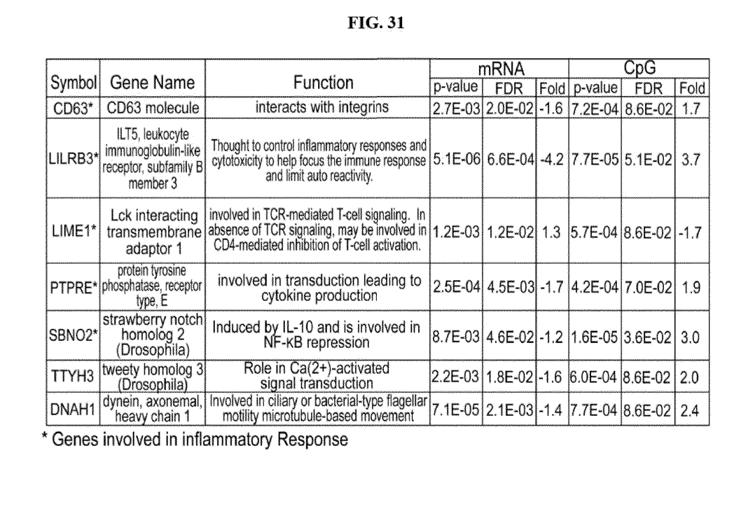

DNA methylation is considered an epigenetic marker for expression competency, with reduced methylation usually correlated with enhanced gene expression. It is generally presumed that for most expressed genes each of the two alleles contributes equally. However, introduction of a new potential IFNG CpG methylation SNP might lead to allelic methylation differences, ultimately affecting IFN-γ expression levels. It has been suggested that unequal allele methylation occurs preferentially in sites adjacent to CpG SNPs. In order to ascertain the methylation status of rs1861494, allele/strand specific pyrosequencing methylation analysis was carried out in IBD patients heterozygous for rs1861494. Strand specific methylation levels for each of the two adjacent upstream CpG sites, as well as the rs1861494, were determined. The minor rs1861494 C allele did function as a methylation site (average methylation 60%, FIG. 28A). As expected, no significant methylation was seen for the non CpG common T allele. Furthermore, a significant decrease in the methylation of the +2052 bp CpG site was detected when comparing the minor C to the common T allele strands (p<0.001) (FIG. 28B). No difference was seen for the +2007 bp CpG site (FIG. 28B). A significant inverse correlation between the de novo IFNG expression and the corresponding overall methylation index (MI) of the promoter IFNG region (−54, −186, and −295 bp) previously demonstrated. Therefore, the relationship between the overall MI of the three sites adjacent to rs1861494 and the MI of the IFNG promoter region was examined. There was a highly significant correlation (p<0.001) between the MI of these two regions for both the T and C alleles (FIG. 28C), suggesting that the methylation status of the rs1861494 region paralleled that of a promoter region known to contribute to regulation of gene expression.

### Allele-Specific Nucleo-Protein Binding was Detected to Methylated Rs1861494 DNA-Binding Sites

To investigate how the C to T SNP influences transcription factor binding to the SNP rs1861494 region, nuclear extracts were prepared from resting or activated PB T cells. Nucleo-protein binding assays were carried out using dsDNA oligonucleotides corresponding to the common T or minor C SNP allele sequence. Activation-dependent binding of nucleo-protein complex to the T or C SNP oligonucleotide was detected (FIGS. 29A-29B). Although similar complexes were seen for the common T and minor C oligonucleotides, binding to the T SNP was enhanced compared to the C SNP allele (FIG. 29A).

To investigate how methylation of the rs1861494 CpG influenced transcription factor complex formation, binding assays were set up using a non-methylated, asymmetrically methylated (one strand) C SNP or symmetrically methylated (both strands) C SNP oligonucleotide. Asymmetrical methylation of one C oligonucleotide strand displayed a pattern similar to that seen for the unmethylated oligonucleotide (FIG. 29B, C SNP vs. C/MC SNP). In contrast, symmetric methylation of both C oligonucleotide strands markedly enhanced binding and the appearance of an additional protein complex (FIG. 29B, C/MC SNP vs. MC/MC SNP). These results suggested that both the specific allele sequence and methylation levels could modulate binding of transcription factors to the rs1861494 region and likely regulate IFN-γ expression.

### Example 6-6

Mucosal expression of IFN-γ and other pro-inflammatory cytokines is critical not only to the development and maintenance of inflammation but, additionally, the absolute amount of IFN-γ appears to modulate the severity of Crohn's disease. This study examined the association between the IFNG rs1861494 T/C polymorphism in IBD. IFNG rs1861494 T allele carriage in IBD patients was associated with enhanced secretion of IFN-γ. In addition, IFNG rs1861494 T allele carriage was associated with clinical/serological parameters indicative a more severe disease course in both UC and CD patients. In UC, T allele carriage was associated with seropositivity and higher levels of the IBD associated autoantibody ANCA. This may be the first study to report a genetic association, outside the HLA region, with ANCA levels. Among patients with medically refractory UC, T allele carriage was associated with a severe disease course that progressed more rapidly toward colectomy. In the CD patient population, T allele carriage was associated with a complicated disease behavior, characterized by stricturing/penetrating phenotype.

These findings are consistent with previously reported studies demonstrating an involvement of IFNG rs1861494 in severity and treatment resistance in other infectious and immune disorders. In tuberculosis, rs1861494 T allele carriage has been associated with susceptibility as well as a more severe microscopy-positive and bacterial positive form of the disease. Furthermore a recent study demonstrated the presence of persistent, elevated levels of IFN-γ in T allele carriers following anti-tuberculosis treatment supporting an association with therapeutic resistance. These data suggests a possible mechanistic role in which elevated IFN-γ expression in T allele carriers may lead to a worse prognosis for the resolution of active disease or accelerated progression to complicated and severe disease. Likewise, in hepatic schistosomiasis, T allele carriage has been associated with poorer control of disease and higher risk of developing severe and extended hepatic fibrosis. In chronic myeloid leukemia, T allele carriage has been associated with poorer response to Imatinib therapy and slower progression of complete cytogenic response.

One of the major challenges in treating IBD is the inter-individual variability of response to therapeutic intervention. The association of rs1861494 T allele carriage with disease severity may help identify patients at risk for aggressive disease before complications occur and thereby reduce the need for surgery. In CD, half of all patients will develop stricturing or penetrating complications within the first 20 years of disease. Most of these patients will go on to require surgery with up to a 61% of CD patient requiring surgery at 10 years. In UC, the expression of ANCA autoantibodies is associated with a complicated disease course and the need for surgical intervention. In fact, a subset of UC patients expressing high levels of ANCA are more likely to develop chronic pouchitis after ileal pouch-anal anastomosis. Moreover, studies have suggested that ANCA reactivity may be associated with poor response to anti-TNF therapy in UC patients. ANCA seronegative UC patients have been shown to be more likely to initially achieve clinical response to anti-TNF therapy than ANCA seropositive patients. Likewise, in children ANCA reactivity has been independently associated with primary nonresponse to anti-TNF therapy. Similar findings have been shown in the CD population in which patients who displayed seropositivity to ANCA were more likely to fail anti-TNF therapy. The initial lack of response to anti-TNF therapy is of clinical importance in CD and even more so in UC. It is likely that lack of primary response to anti-TNF therapy at least in part may reflect that TNF is not the primary mediator of the inflammatory process in this group of patients. Since patients who are seropositive to ANCA are more likely to fail anti-TNF therapy, the present findings of rs1861494 association with disease severity, elevated ANCA and IFN-γ protein levels, suggest that IFN-γ might be what is driving active inflammation in this subset.

IFNG is located on chromosome 12 and is highly conserved through evolution. T cell production of IFN-γ is determined primarily at the transcriptional level through cis and trans factor binding regions. DNA methylation of CpG dinucleotides provides an additional level of regulation and has been associated with transcriptional silencing. The rs1861494 T/C polymorphism is located in the third IFNG intronic region which has been previously reported to possess a T-bet binding site and enhancer activity. Functionally, T allele carriage was correlated with enhanced IFN-γ secretion. The T to C substitution introduces a new CpG dinucleotide sequence that was associated with increased DNA methylation and decreased IFN-γ expression. Electromobility shift assays indicated more intense binding of nuclear protein complex to the T compared to C allele following T cell activation. Moreover, methylation of the CpG sequence within the context of oligonucleotides complementary to the C allele, demonstrated altered nuclear complex formation and the presence of an additional complex. More intense binding of nuclear protein complex to the T and C alleles has been previously reported in nuclear extracts from the Jurkat cell line and additional complex was detected binding the C allele in PHA-blasted T cells. This report is the first to demonstrate methylation dependent binding of nucleo-protein in the context of the rs1861494 C allele and suggests that nucleo-protein binding to this region may be regulated on two levels: one via allele specific affinity for DNA-binding proteins and a second regulated by DNA-methylation-mediated nucleo-protein binding.

Distinct epigenetic IFNG DNA methylation patterns in patients with IBD compared to normal controls has been previously reported. Decreased IFNG methylation is associated with patients requiring surgery and, in UC, is functionally correlated with enhanced IFN-γ secretion and a higher sero-reactivity to what are commonly thought of as “CD-associated” antigens. These data, together with GWAS discovery of UC risk regions on chromosome 12 upstream and downstream of IFNG, suggest that IFNG may play a more central role in the pathogenesis of UC than was originally proposed. One of the fundamental challenges of GWAS is to define the functional consequences of these genetic variations. Surprisingly, many SNPs associated with disease are in fact CpG sites, which are statistically underrepresented in the human genome. Although CpG sites are underrepresented in most non-coding DNA, they are preserved in promoter and other regulatory genomic regions. Thus, in addition to differences detected in DNA methylation patterns in disease, CpG SNPs can introduce sites for allele-specific DNA methylation, the functional consequences of which may affect gene expression via epigenetic mechanisms. Recent studies have suggested that allele specific methylation occurs preferentially in sites adjacent to CpG SNPs. IFNG rs1861494 sits within a region adjacent to two upstream CpG sites. C allele specific methylation at the polymorphic +2109 bp site and differential DNA methylation of the adjacent +2052 CpG site were observed, but no allele specific differences were detected for the +2007 bp site. More importantly, a significant correlation was observed between the average methylation index across all three sites for both the C and T alleles and the IFNG promoter methylation index. Methylation of the IFNG promoter region has been extensively studied and is believed to play a major role in overall regulation of IFNG expression. While it has been generally reported that enhanced IFN-γ expression corresponds with a decrease in IFNG methylation for some promoter region such as the −22 CNS, it has been demonstrated that the reverse appears to be the case. These finding suggests that epigenetic modulation via methylation of IFNG expression encompasses cooperative interaction across multiple regions.

The data presented here link a genotype-dependent association of the conserved IFNG rs1861494 SNP with allele specific DNA methylation, transcription factor complex formation and IFN-γ protein expression associated with an altered and more aggressive course of disease progression in IBD. Moreover, the data demonstrated an association of rs1861494 with seropositivity and higher levels of ANCA autoantibodies, a biomarker for poorer therapeutic response to anti-TNF therapy. These findings suggest that IFNG rs1861494 may provide new insight into stratifying IBD patients based on whether their mechanism of disease is more dependent of IFN-γ expression and less dependent on TNF. This would be of great prognostic value not only in selecting patients likely to respond to anti-TNF therapy but in potentially identifying patients likely to fail primary anti-TNF therapy and initiate evaluation of other treatment options.

### Example 6-7

### Methylation and IBD Subgroups

As disclosed herein, the inventors have shown that IBD patients display distinct IFNG methylation, correlated with enhanced IFN-γ secretion and seroreactivity to microbial antigens. As further disclosed herein, the inventors integrated genome wide expression/methylation quantitative trait loci (eQTL/mQTL) and GWAS in order to identify molecular signatures associated with clinical response. They profiled for differentially methylated regions (DMRs) (Illumina-450k InfiniumArray) and eQTL (Illuminaexpression array) in CD3+peripheral T cells from 11 CD and 10 UC patients genotyped for the 163 IBD-risk loci. All patients were matched for ethnicity and age and had undergone surgery.

In accordance with various embodiments herein, no genome-wide DMRs were seen between CD and UC patient populations. However, when samples were stratified based on IBD-risk vs. non-risk SNP genotype, 10/163 loci displayed DMRs, of which 2 also showed eQTL: rs6074022 and rs941823. The rs941823 lies within a long non-coding RNA sequence, whereas rs6074022, resides within the CD40 promoter, −7 kb upstream of the transcriptional start site, and was, therefore, chosen for further study. The rs6074022 T (non-risk) to C (risk) SNP introduces a new CpG methylation site, potentially resulting in altered expression. This was confirmed by real-time PCR using IBD patient mRNA isolated from EBV transformed cell lines. The rs6074022 risk SNP functionally correlated with a significant decrease in CD40 expression (2.3% vs. 1.8% of housekeeping gene EF1α, p<0.05). In addition to altered CD40 gene expression, distinct DMRs (147 CpGsites p<0.001, false discovery rate <0.05) and eQTL (460 transcripts p<0.01, false discovery rate <0.05) were associated with rs6074022. DMRs were enriched in gene bodies or intergenic sites. More than half of DMRs (55%) were defined as regulatory sites. Moreover, DMRs differed between UC and CD patients when stratified for carriage of either the risk and non-risk rs6074022 alleles. CD patients harboring the risk allele were likely to require earlier surgical intervention (p<0.02) for disease management. The rs6074022 functionally impacts not only on CD40 expression but additional target molecules detected by mQTL/eQTLupstream and downstream of the CD40 signaling pathway, i.e. antigen presentation, cytokines and transcriptional regulators. Thus, the inventors have shown a mechanistic role for the rs6074022 SNP in defining altered disease susceptibility and natural history in IBD.

The CD40 IBD risk SNP rs6074022 Displays:
Attenuated CD40 expression
Earlier progression to surgery

rs6074022 SNP Variation is Associated with:
DMRs between risk vs. non-risk and CD vs. UC
Differential mRNA expression

CD40 is a key mediator in immune and inflammatory processes. Data suggests a functional association of the CD40 IBD risk rs6074022 with altered epigenetic and expression profiles which may aid in stratifying IBD patients to predict altered pathobiology and course of disease.

In various embodiments, the present invention provides a method of diagnosing susceptibility to an inflammatory bowel disease (IBD) subtype in an individual, comprising: (a) obtaining a sample from the individual; (b) assaying the sample to determine the presence or absence of at least one risk genetic variant at the genetic locus of IFNG; and (c) diagnosing susceptibility to the IBD subtype based on the presence of at least one risk genetic risk variant at the genetic locus of IFNG.

In various embodiments, the assaying the sample comprises using an oligonucleotide probe specific to a risk genetic variant at the genetic locus of IFNG.

In various embodiments, the oligonucleotide probe is labeled with a fluorescent dye.

In various embodiments, the IBD comprises ulcerative colitis.

In various embodiments, the IBD comprises Crohn's disease.

In various embodiments, the IBD is associated with early surgical intervention.

In various embodiments, the IBD is associated with colitis, a small bowel disease phenotype, an aggressive complicating phenotype, an internal penetrating disease phenotype, a stricturing disease phenotype, a fibrostenosing disease phenotype, a fistulating disease phenotype, or a combination thereof.

In various embodiments, the IBD is associated with at least one risk serological marker selected from the group consisting of ANCA, ASCA, anti-Cbir1, anti-I2, and anti-OmpC.

In various embodiments, the at least one risk genetic variant is a “T” allele of SEQ ID NO:66.

In various embodiments, the at least one risk genetic variant is associated with a lower level of IFNG DNA methylation relative to a healthy subject.

In various embodiments, the at least one risk genetic variant is associated with a higher level of anti-Cbir1 relative to a healthy subject.

In various embodiments, the at least one risk genetic variant is a “C” allele of SEQ ID NO:66.

In various embodiments, the at least one risk genetic variant is associated with a higher level of IFNG DNA methylation relative to a healthy subject.

In various embodiments, the present invention provides a method of diagnosing inflammatory bowel disease (IBD) in an individual, comprising: (a) obtaining a sample from an individual; (b) assaying the sample to determine the presence or absence of at least one risk genetic variant at the genetic locus of IFNG; (c) assaying the sample to determine an increase or decrease in IFNG DNA methylation relative to a healthy subject; and (d) diagnosing IBD in the individual based on the presence of at least one risk genetic variant at the genetic locus of IFNG and an increase in IFNG DNA methylation relative to a healthy subject.

In various embodiments, the IBD comprises Crohn's disease or ulcerative colitis.

In various embodiments, the at least one risk genetic variant is a “T” allele of SEQ ID NO:66.

In various embodiments, the method further comprises assaying the sample to identify a high level of anti-Cbir1 relative to a healthy subject.

In various embodiments, the IBD is associated with severe ulcerative colitis conditions.

In various embodiments, the IBD is associated with colitis, a small bowel disease phenotype, an aggressive complicating phenotype, an internal penetrating disease phenotype, a stricturing disease phenotype, a fibrostenosing disease phenotype, a fistulating disease phenotype, or a combination thereof.

In various embodiments, the IBD is associated with at least one risk serological marker selected from the group consisting of ANCA, ASCA, anti-Cbir1, anti-I2, and anti-OmpC.

In various embodiments, the sample comprises a nucleic acid from the individual.

In various embodiments, the sample is a body fluid.

In various embodiments, the body fluid is whole blood, plasma, saliva, mucus, or cheek swab.

In various embodiments, the sample is a cell or tissue.

In various embodiments, the cell is a lymphoblastoid cell line obtained from the individual and transformed with an Epstein Barr virus.

In various embodiments, the cell is a mucosal T cell, a lamina propria T cell, or a peripheral blood T cell.

In various embodiments, the present invention provides a method of treating inflammatory bowel disease (IBD) in an individual, comprising: (a) obtaining a sample from an individual; (b) assaying the sample to determine the presence of at least one risk genetic variant at the genetic locus of IFNG; (c) assaying the sample to determine an aberrant level of IFNG DNA methylation; and (d) treating the IBD in the individual.

In various embodiments, the IBD comprises Crohn's disease or ulcerative colitis.

In various embodiments, the IBD is associated with early surgical intervention.

In various embodiments, the IBD is associated with colitis, a small bowel disease phenotype, an aggressive complicating phenotype, an internal penetrating disease phenotype, a stricturing disease phenotype, a fibrostenosing disease phenotype, a fistulating disease phenotype, or a combination thereof.

In various embodiments, the at least one risk genetic variant at the genetic locus of IFNG comprises SEQ ID NO:66.

In various embodiments, the present invention provides a method of treating an inflammatory bowel disease (IBD) in an individual, comprising: (a) obtaining a sample from the individual; (b) assaying the sample to detect the presence or absence of at least one risk genetic variant at the genetic locus of IFNG, and/or assaying the sample to detect an increase or decrease in IFNG DNA methylation relative to a healthy individual; and (c) treating the IBD in the individual.

In various embodiments, the IBD comprises Crohn's disease (CD) or ulcerative colitis (UC).

In various embodiments, the IBD is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof.

In various embodiments, the individual is a human.

In various embodiments, the sample comprises a nucleic acid from the individual.

In various embodiments, the sample comprises a body fluid, cheek swab, mucus, whole blood, blood, serum, plasma, urine, saliva, semen, lymph, fecal extract, or sputum, or a combination thereof.

In various embodiments, the sample comprises a cell or tissue.

In various embodiments, the cell is a lymphoblastoid cell line obtained from the individual and transformed with an Epstein Barr virus.

In various embodiments, the cell is a mucosal T cell, a lamina propria T cell, or a peripheral blood T cell.

In various embodiments, the at least one risk genetic variant is the “T” allele of SEQ ID NO:66 or SEQ ID NO:67.

In various embodiments, IFNG DNA methylation is IFNG promoter methylation.

In various embodiments, the method further comprises assaying the sample to detect an increase of INF-γ secretion relative to a healthy individual.

In various embodiments, the method further comprises assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC.

In various embodiments, treating the IBD comprises conducting colectomy on the individual, upon detecting the presence of at least one risk genetic variant at the genetic locus of IFNG and/or a decrease in IFNG DNA methylation relative to a healthy individual.

In various embodiments, treating the IBD comprises administering a TNF signaling inhibitor to the individual, upon detecting the absence of at least one risk genetic variant at the genetic locus of IFNG and/or an increase in IFNG DNA methylation relative to a healthy individual.

In various embodiments, the TNF signaling inhibitor comprises an anti-TNF antibody.

In various embodiments, the TNF signaling inhibitor comprises infliximab, adalimumab, certolizumab, certolizumab pegol, golimumab, etanercept, or onercept, or a combination thereof.

In various embodiments, the TNF signaling inhibitor is administered topically, intravascularly, intravenously, intraarterially, intramuscularly, subcutaneously, intraperitoneally, intranasally, or orally.

In various embodiments, the TNF signaling inhibitor is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof.

In various embodiments, the TNF signaling inhibitor is administered about 1-3 times per day, 1-7 times per week, or 1-9 times per month.

In various embodiments, the TNF signaling inhibitor is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

In various embodiments, the present invention provides a method of treating an inflammatory bowel disease (IBD) in an individual, comprising: (a) genotyping the individual for a risk genetic variant at the genetic locus of IFNG; and (b) if the individual is positive for the risk genetic variant, conducting colectomy on the individual, and if the individual is negative for the risk genetic variant, administering a TNF signaling inhibitor to the individual.

In various embodiments, the present invention provides a method of treating an inflammatory bowel disease (IBD) in an individual, comprising: (a) obtaining a sample from the individual; (b) contacting the sample with an oligonucleotide probe specific to a risk genetic variant at the genetic locus of IFNG; (c) forming an allele-specific hybridization complex between the oligonucleotide probe and the risk genetic variant; (d) detecting the allele-specific hybridization complex; and (e) if the allele-specific hybridization complex is detected, conducting colectomy on the individual, and if the allele-specific hybridization complex is not detected, administering a TNF signaling inhibitor to the individual.

In various embodiments, the individual is a human.

In various embodiments, the risk genetic variant is the “T” allele of SEQ ID NO:66 or SEQ ID NO:67.

In various embodiments, the oligonucleotide probe is labeled with a fluorescent dye, and wherein detecting the allele-specific hybridization complex comprises detecting fluorescence signal from the oligonucleotide probe.

In various embodiments, the oligonucleotide probe comprises a reporter dye and a quencher dye.

In various embodiments, the method further comprises conducting PCR amplification after forming the allele-specific hybridization complex.

In various embodiments, detecting the allele-specific hybridization complex comprises detecting the electrophoretic mobility of the allele-specific hybridization complex.

### Example 7

### Methods of Predicting Thiopurine Response

This non-limiting example relates to methods of predicting therapeutic efficacy of thiopurines in an individual by determining the presence of one or more risk variants. In one embodiment, the effective therapeutic efficacy of thiopurines is determined by the presence of risk variants at the genetic loci of HLA-DRB1, CREM, TAGAP, PLCL1, GPX4, SBNO2, MEF2A and/or LYSMD4. In another embodiment, the risk variants are located at the genetic loci of ARL4C, IL1R2, JAK2, 19q13, CARD9, SNAPC4, and/or 8q24. In another embodiment, the individual is has been diagnosed with inflammatory bowel disease.

### Example 7-1

### Therapeutic Remission to Thiopurines in IBD

As disclosed herein, the inventors tested associations of known IBD susceptibility loci and novel “pharmacogenetic” genome-wide association study (GWAS)-identified loci, as well as clinical and immune phenotypes, with thiopurine-induced corticosteroid-free remission in IBD, and developed a predictive model of remission. Corticosteroid-free remission at 26 weeks after thiopurine initiation was defined using the Harvey Bradshaw Index (HBI) for Crohn's disease (CD) and partial Mayo score for ulcerative colitis (UC). Serum was assayed for ASCA IgA and IgG, anti-OmpC, anti-CBir1, I2, and pANCA using ELISA. Clinical phenotypes included age, gender, IBD subtype (CD versus UC), disease duration at thiopurine initiation, and age at diagnosis. Genotyping was performed using Illumina technology. Univariate analyses tested associations of phenotype and genotype with remission. Stepwise logistic regression was performed to build predictive models.

As further disclosed herein, corticosteroid-free remission occurred in 56 of 122 subjects (45.9%) at week 26. Female gender (OR=0.37; 95% CI: 0.18-0.77; P=0.011) and pANCA (OR=0.23; 95% CI: 0.06-0.87; P=0.049) were negatively associated with corticosteroid-free remission at 26 weeks. Five known IBD susceptibility loci were associated with corticosteroid-free remission (P<0.05) (Table 10 herein). A single nucleotide polymorphism (SNP) at 15q31) tagging MEF2A (macrophage differentiation) and LYSMD4 (peptidoglycan binding) met the criteria for nominal association at the genome wide level for remission (OR=9.5; P=3E-05). The most predictive model of remission included the previously identified HLA-DRB1 locus (rs2516049; SEQ ID NO:83), 7 pharmacogenetic GWAS loci, pANCA, disease duration, and a diagnosis of UC with an R-squared of 0.884, area under the curve [AUC] of 0.985, sensitivity of 0.929, specificity of 0.919, accuracy of 0.826, and positive likelihood ratio of 11.45. The probability of remission increased 7.3-fold when the number of predictors increased from 0-4 to 5-7 (95% CI: 2.43-21.66; P=0.0004). The combination of genotype with clinical and immune phenotypes is most predictive of corticosteroid-free remission after thiopurine initiation. Defining predictors of therapeutic efficacy to thiopurines allows identification of patients who will benefit most from this class of therapy, contributing to a more individualized approach to therapy.

### Example 7-2

TABLE 10
SNPs associated with corticosteroid-free
remission with thiopurines at week 26

| SNP | Chromosome | P Value | OR | Gene of Interest |
|---|---|---|---|---|
| (SEQ ID NO: 83) | 6 | 0.0033 | 3.35 | HLA-DRB1 |
| rs2516049 | | | | |
| (SEQ ID NO: 84) | 10 | 0.019 | 0.40 | CREM |
| rs3936503 | | | | |
| (SEQ ID NO: 85) | 6 | 0.042 | 2.04 | TAGAP |
| rs212388 | | | | |
| (SEQ ID NO: 86) | 2 | 0.043 | 0.50 | PLCL1 |
| rs10196612 | | | | |
| (SEQ ID NO: 87) | 19 | 0.044 | 0.44 | GPX4, SBNO2 |
| rs2024092 | | | | |

### Example 7-3

### Genotyping

Genotyping was performed at the Medical Genetics Institute at Cedars-Sinai Medical Center: using Illumina Human610 and OmniExpress chips for CD samples, and using Illumina HumanCNV370 and OmniExpress chips for UC samples. 191,264 SNPs were common among platforms, passed quality control, and were included in the analyses. Principal components analysis using Eigenstrat was conducted to examine population stratification.

### Example 7-4

### Univariate Associations

### 1 Genetic Loci

Known IBD susceptibility SNPs (GWA significance) from:
adult CD meta GWAS (71 loci)
adult UC meta GWAS (47 loci)
pediatric IBD GWAS (2 loci)

Pharmacogenetic based GWAS SNPs
Single SNP associations

### 2. Demographic and Clinical Phenotype Data

Student's t-test for continuous variables

Fisher exact test for categorical variables

### Example 7-5

### Predictive Models

1. Multiple Logistic Regression models of remission:
Model I: Clinical Only—Including Age, Gender, IBD subtype: UC vs. CD, Disease duration, TPMT activity, Starting dose AZA ≧2.5 mg/kg/day or equivalent
Model II: Genetics only—Including known IBD Susceptibility SNPs (“Top Hits” p<0.05) & SNPs from Pharmacogenetic GWAS (p<1×10⁻⁵)
Model III: Genetics+Clinical (Final model)
   2. Clinical Utility Measures: sensitivity, specificity, AUC, positive likelihood ratio

### Example 7-6

TABLE 11
Clinical phenotypic associations with therapeutic
outcomes to thiopurines (week 26)

| | | No | OR | P |
|---|---|---|---|---|
| Clinical Variable | Remission | remission | (95% CI) | value |
| IBD Subtype-UC vs. CD | 9:54 | 17:47 | 0.46 | 0.14 |
| | | | (0.19-1.13) | |
| Gender: Males N (%) | 56% | 38% | 2.17 | 0.046 |
| | | | (1.03-4.23) | |
| Mean disease duration | 8.86 | 13.63 | — | 0.12 |
| at thiopurine initiation | | | | |
| (months) | | | | |
| Mean age at diagnosis | 11.37 | 11.22 | — | 0.84 |
| (years) | | | | |
| TPMT activity | 31:8 | 22:7 | 0.81 | 0.73 |
| (Normal: Intermediate) | | | (0.26-2.57) | |
| Starting dose AZA | 40:22 | 19:37 | 3.54 | 0.002 |
| ≧2.5 mg/kg/d vs. | | | (1.66-7.57) | |
| <2.5 mg/kg/d | | | | |

### Example 7-7

TABLE 12
Known IBD susceptibility loci associated
with remission at week 26 (p < 0.05)

| Gene(s) of | | CD or | | | |
|---|---|---|---|---|---|
| Interest | Chromosome | UC | SNP | OR | P value |
| TAGAP | 6 | CD | rs212388 | 2.48 | 0.003 |
| 19q13 | 19 | CD, UC | rs736289 | 0.45 | 0.008 |
| CARD9, | 9 | CD | rs4077515 | 0.53 | 0.026 |
| SNAPC4 | | | | | |
| HLA-DRB1 | 6 | CD, UC | rs2516049 | 1.90 | 0.036 |
| JAK2 | 9 | CD, UC | rs10758669 | 1.78 | 0.043 |
| IL1R2 | 2 | UC | rs2310173 | 2.00 | 0.046 |
| 8q24 | 8 | CD | rs6651252 | 0.41 | 0.047 |

### Example 7-8

TABLE 13
Model 1 results, Clinical phenotype only

| | OR | | Pseudo | |
|---|---|---|---|---|
| Variable | (95% CI) | P value | R² | AUC |
| IBD Subtype: UC vs. CD | 0.76 | 0.74 | 0.23 | 0.74 |
| | (0.15-3.82) | | | |
| Mean age at diagnosis (years) | 0.93 | 0.43 | | |
| | (0.77-1.12) | | | |
| Gender: Male vs. Female | 4.09 | 0.03 | | |
| | (1.16-14.4) | | | |
| Mean disease duration at | 0.99 | 0.45 | | |
| thiopurine initiation (mos.) | (0.95-1.02) | | | |
| TPMT activity | 0.51 | 0.41 | | |
| (Normal:Intermediate) | (0.10-2.53) | | | |
| Starting dose AZA | 2.56 | 0.16 | | |
| ≧2.5 mg/kg/d vs. <2.5 mg/kg/d | (0.71-9.22) | | | |

### Example 7-9

TABLE 14
Model II results, Genotype only (GWAS p < 1 × 10⁻⁵; Top hits p < 0.05)

| Gene(s) of | | OR | | Pseudo | |
|---|---|---|---|---|---|
| Interest | Variable | (95% CI) | P value | R² | AUC |
| AR4LC | rs4663142 | 0.15 | 0.00014 | 0.4 | 0.82 |
| | (GWAS) | (0.06-0.39) | | | |
| 19q13 | rs736289 | 7.09 | 0.017 | | |
| | (Top Hits) | (1.44-34.8) | | | |
| JAK2 | rs10758669 | 2.74 | 0.044 | | |
| | (Top Hits) | (1.04-7.24) | | | |
| IL1R2 | rs2310173 | 2.18 | 0.11 | | |
| | (Top hits) | (0.84-5.65) | | | |
| HLA-DRB1 | rs2516049 | 1.94 | 0.15 | | |
| | (Top Hits) | (0.79-4.74) | | | |
| CARD9, | rs4077515 | 2.40 | 0.18 | | |
| SNAPC4 | (Top Hits) | (0.67-8.60) | | | |
| TAGAP | rs212388 | 1.84 | 0.24 | | |
| | (Top Hits) | (0.67-5.06) | | | |
| 8q24 | rs6651252 | 1.00 | 0.99 | | |
| | (Top Hits) | (0-inf) | | | |

### Example 7-10

TABLE 15
Model III, Genotype + Clinical Phenotype

| Gene(s) of | | | | | Pseudo | |
|---|---|---|---|---|---|---|
| Interest | Variable | OR | (95% CI) | P value | R² | AUC |
| AR4LC | rs4663142 (GWAS) | 0.09 | (0.01-0.58) | 0.015 | 0.53 | 0.87 |
| 19q13 | rs736289 (Top Hits) | 1.52 | (0.08-29.7) | 0.78 | | |
| JAK2 | rs10758669 (Top Hits) | 4.64 | (0.69-31.2) | 0.12 | | |
| IL1R2 | rs2310173 (Top hits) | 3.50 | (0.57-21.5) | 0.18 | | |
| HLA-DRB1 | rs2516049 (Top Hits) | 5.15 | (0.75-35.2) | 0.10 | | |
| CARD9, | rs4077515 (Top Hits) | 1.58 | (0.14-17.2) | 0.71 | | |
| SNAPC4 | | | | | | |
| TAGAP | rs212388 (Top Hits) | 2.90 | (0.48-17.7) | 0.25 | | |
| 8q24 | rs6651252 (Top Hits) | 1.00 | (0-inf) | 0.99 | | |
| | IBD Subtype: UC vs. CD | 0.50 | (0.06-3.84) | 0.51 | | |
| | Gender: Male vs. Female | 3.06 | (0.54-17.4) | 0.21 | | |
| | Mean disease duration at | 0.98 | (0.94-1.01) | 0.25 | | |
| | thiopurine initiation (months) | | | | | |
| | Mean age at diagnosis (years) | 0.95 | (0.73-1.23) | 0.70 | | |
| | TPMT activity | 0.91 | (0.08-10.8) | 0.94 | | |
| | (Normal:Intermediate) | | | | | |
| | Starting dose AZA | 7.91 | (1.15-54.5) | 0.04 | | |
| | ≧2.5 mg/kg/d vs. <2.5 mg/kg/d | | | | | |

### Example 7-11

TABLE 16
Model III (genotype + clinical): Demonstration of diagnostic utility for remission to
thiopurines

| Gene(s) | | | | | | Positive |
|---|---|---|---|---|---|---|
| of | | Pseudo | | | | Likelihood |
| Interest | Variable | R² | AUC | Sensitivity | Specificity | Ratio |
| AR4LC | rs4663142 (GWAS) | 0.53 | 0.87 | 0.76 | 0.84 | 4.7 |
| 19q13 | rs736289 (Top Hits) | | | | | |
| JAK2 | rs10758669 (Top Hits) | | | | | |
| IL1R2 | rs2310173 (Top hits) | | | | | |
| HLA- | rs2516049 (Top Hits) | | | | | |
| DRB1 | | | | | | |
| CARD9, | rs4077515 (Top Hits) | | | | | |
| SNAPC4 | | | | | | |
| TAGAP | rs212388 (Top Hits) | | | | | |
| 8q24 | rs6651252 (Top Hits) | | | | | |
| | IBD Subtype: UC vs. CD | | | | | |
| | Gender: Male vs. Female | | | | | |
| | Mean disease duration at | | | | | |
| | thiopurine initiation (months) | | | | | |
| | Mean age at diagnosis (years) | | | | | |
| | TPMT activity | | | | | |
| | (Normal:Intermediate) | | | | | |
| | Starting dose AZA | | | | | |
| | ≧2.5 mg/kg/d vs. <2.5 mg/kg/d | | | | | |

### Example 7-12

### Overall

7 known IBD susceptibility loci (“top hits”) were significantly associated with thiopurine-induced corticosteroid-free remission at week 26, with p<0.05. Additionally, the ARL4C locus met nominal genome-wide significance for association with remission at week 26 (p=3.11E-06). The model combining clinical phenotype and genotype was the most predictive of corticosteroid-free remission (LR:4.7), and TPMT activity was not associated with corticosteroid-free remission.

### Example 7-13

TABLE 17
Description of Genetic Loci

| Gene(s) of | | | |
|---|---|---|---|
| Interest | SNP | Details | Role |
| ARL4C | rs4663142 | Member of ADP-ribosylation factor family of GTP- | Lymphoid/myeloid |
| 2q37/IBD | (GWAS) | binding proteins. May be involved in lymphoid/myeloid | differentiation & |
| | | differentiation and intracellular transport. Expressed in | intracellular |
| | | T cells, NK cells, and dendritic cells. | transport |
| IL1R2 | rs2310173 | Cytokine receptor belonging to interleukin 1 receptor family. | Pro-inflammatory |
| 2q12/UC | (Top Hit) | Acts as a decoy receptor to inhibit the activity of its ligands | |
| | | (IL1a, IL1b, IL1R1) | |
| JAK2 | rs10758669 | Signal Transducers and Activators of Transcription (STAT)- | Signal transduction |
| 9p24/IBD | (Top Hit) | Janus kinase (JAK) pathway controls signal transduction | (e.g., IFN-g) |
| | | between cell surface receptors & the nucleus | |
| 19q13 | rs736289 | Associated with CD, UC | Unknown |
| IBD | (Top Hit) | | |
| TAGAP | rs212388 | T cell activation Rho GTPase activating protein. Shared risk | T cell activation |
| 6q25/CD | (Top Hit) | locus in celiac disease | |
| CARD9, | rs4077515 | Caspase-associated recruitment domain family, member 9. | Cell apoptosis, |
| SNAPC4 | (Top Hit) | Small nuclear RNA activating complex, polypeptide 4, | RNA Pol |
| 9q34/CD | | required for RNA polymerase II/III snRNA | |
| 8q24 | rs6651252 | Associated with CD | Unknown |
| CD | (Top Hit) | | |
| HLA-DRB1 | rs2516049 | HLA class II histocompatibility antigen, encodes the most | Antigen |
| 6p21/IBD | (Top Hit) | prevalent beta subunit of HLA-DR. Presents peptides | presentation |
| | | derived from extracellular proteins | |

In various embodiments, the present invention provides a method of predicting responsiveness to thiopurine treatment in an individual, comprising: obtaining a sample from the individual; assaying the sample to determine the presence or absence of one or more risk variants at the HLA-DRB1, CREM, TAGAP, PLCL1, GPX4, SBNO2, MEF2A and/or LYSMD4 genetic loci; and predicting responsiveness to thiopurine treatment based on the presence of one or more risk variants at the HLA-DRB1, CREM, TAGAP, PLCL1, GPX4, SBNO2, MEF2A and/or LYSMD4 genetic loci.

In various embodiments, the individual has been diagnosed with inflammatory bowel disease.

In various embodiments, the individual is a child.

In various embodiments, the risk variants comprise SEQ ID NO:83, SEQ ID NO:84, SEQ ID NO:85, SEQ ID NO:86, and/or SEQ ID NO:87.

In various embodiments, the presence of five or more of said risk variants presents a greater probability of responsiveness to thiopurine treatment than the presence of four, three, two, one or none of said risk variants; the presence of four said risk variants presents a greater probability of responsiveness to thiopurine treatment than the presence of three, two, one or none of said risk variants; the presence of three risk variants presents a greater probability of responsiveness to thiopurine treatment than the presence of two, one or none of said risk variants; the presence of two risk variants presents a greater probability of responsiveness to thiopurine treatment than the presence of one or none of said risk variants; and the presence of one risk variant presents a greater probability of responsiveness to thiopurine treatment than the presence of none of said risk variants.

In various embodiments, the individual has been diagnosed with ulcerative colitis.

In various embodiments, the sample further comprises a high expression relative to a normal subject of pANCA.

In various embodiments, the individual has been diagnosed with acute lymphoblastic leukemia and/or an autoimmune disorder.

In various embodiments, the individual is an organ transplant recipient.

In various embodiments, the present invention provides a method of treating a disease in an individual, comprising: determining the presence of one or more risk variants in the individual at the genetic loci of ARL4C, IL1R2, JAK2, 19q13, CARD9, SNAPC4, 8q24, HLA-DRB1, CREM, TAGAP, PLCL1, GPX4, SBNO2, MEF2A and/or LYSMD4; and administering a therapeutically effective dosage to the individual of a composition comprising thiopurine, or a pharmaceutical equivalent, analog, derivative, and/or salt thereof.

In various embodiments, the risk variants comprise SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92 and/or SEQ ID NO:93.

In various embodiments, the disease is inflammatory bowel disease.

In various embodiments, the disease is acute lymphoblastic leukemia and/or an autoimmune disorder.

In various embodiments, the individual demonstrates a high expression relative to a normal subject of pANCA.

In various embodiments, the individual is male.

In various embodiments, the present invention provides a method of predicting responsiveness to thiopurine treatment in an individual, comprising: obtaining a sample from the individual; assaying the sample to determine the presence or absence of one or more risk variants at the ARL4C, IL1R2, JAK2, 19q13, TAGAP, CARD9, SNAPC4, 8q24 and/or HLA-DRB1 genetic loci; and predicting responsiveness to thiopurine treatment based on the presence of one or more risk variants at the ARL4C, IL1R2, JAK2, 19q13, TAGAP, CARD9, SNAPC4, 8q24 and/or HLA-DRB1 genetic loci.

In various embodiments, the individual has been diagnosed with inflammatory bowel disease.

In various embodiments, the individual is a child.

In various embodiments, the risk variants comprise SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92 and/or SEQ ID NO:93.

In various embodiments, the individual is male.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Description of the Invention. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Many modifications and variations of the invention as hereinbefore set forth can be made without departing from the spirit and scope thereof, and therefore only such limitations should be imposed as are indicated by the appended claims. Accordingly, the invention is not limited except as by the appended claims.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of determining an individual as having susceptibility to a condition, comprising:
obtaining a sample from the individual;
assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine;
detecting the risk variants or risk haplotypes in the sample, and/or detecting the serological markers in the sample, and/or detecting increased or decreased methylation at the gene in the sample, and/or detecting increased or decreased secretion of the cytokine; and
determining the individual as having susceptibility to the condition.

2. The method claim 1, wherein assaying the sample to detect the risk variants or risk haplotypes comprises:
contacting the sample with one or more allele-specific oligonucleotide probes targeting the risk variants or risk haplotypes;
generating double-stranded hybridization complex through allele-specific binding between the risk variants or risk haplotypes and said allele-specific oligonucleotide probes; and
detecting the double-stranded hybridization complex newly generated through allele-specific binding between the risk variants or risk haplotypes and said allele-specific oligonucleotide probes.

3. The method claim 1, wherein the individual is Jewish, non-Jewish, Caucasian, non-Caucasian, Jewish Caucasian, non-Jewish Caucasian, male, female, a child, or an adult, or a combination thereof.

4. The method claim 1, wherein the condition is inflammatory bowel disease (IBD), subtype of IBD, Crohn's disease (CD), subtype of CD, ulcerative colitis (UC), subtype of UC, aggressive form of IBD, an aggressive of CD, aggressive form of UC, complicated form of IBD, complicated form of CD, complicated form of UC, granuloma, low bone density (LBD), osteoporosis, osteopenia, acute lymphoblastic leukemia, autoimmune disorder, or organ transplantation.

5. The method claim 1, wherein the condition is associated with early surgical intervention, severe ulcerative colitis, colitis, a small bowel disease phenotype, an aggressive complicating phenotype, an internal penetrating disease phenotype, a stricturing disease phenotype, a fibrostenosing disease phenotype, a fistulating disease phenotype, granuloma, low bone density (LBD), osteoporosis, osteopenia, or perianal disease, or a combination thereof.

6. The method claim 1, wherein the more risk variants or risk haplotypes are detected in the sample, the more susceptibility to the condition the individual has.

7. The method claim 1, wherein the risk variants or risk haplotypes are located at one or more genetic loci of interferon gamma (IFNG), Janus kinase 2 (JAK2), SMAD family member 3 (SMAD3), zinc finger protein 365 (ZNF365), fucosyltransferase 2 (FUT2), alpha-fetoprotein (AFP), afamin (AFM), Ras association (RalGDS/AF-6) domain family member 6 (RASSF6), phosphoglucomutase 2 (PGM2), AK097193, fragile histidine triad gene (FHIT), ETS variant 4 (ETV4), malic enzyme 1 (ME1), WD repeat domain 64 (WDR64), ataxin 2 binding protein 1 (A2BP1), cadherin 2 (CDH2), heat shock 70 kDa binding protein, co-chaperone 1 (HSPBP1), protein phophatase 6 regulatory subunit 1 (PPP6R1), BR serine/threonine kinase (BRSK1), Chromosome 4, Chromosome 15, Chromosome 18, transforming growth factor, beta 3 (TGFb3), fat mass and obesity associated (FTO), neuronal PAS domain protein 2 (NPAS2), mucin 1, cell surface associated (MUC1), interleukin 10 (IL10), endoplasmic reticulum aminopeptidase 2 (LRAP), leucine-rich repeat kinase 2 (LRRK2), tumor necrosis factor (ligand) superfamily, member 15 (TNFSF15), cytochrome P-450 cluster, major histocompatibility complex (HLA), laminin, plexin, NLR family, ADP-ribosylation factor-like 4C (ARL4C), interleukin 1 receptor, type II (IL1R2), 19q13, caspase recruitment domain family, member 9 (CARD9), small nuclear RNA activating complex, polypeptide 4, 190 kDa (SNAPC4), 8q24, major histocompatibility complex, class II, DR beta 1 (HLA-DRB1), CAMP responsive element modulator (CREM), T-cell activation RhoGTPase activating protein (TAGAP), phospholipase C-Like 1 (PLCL1), glutathione peroxidase 4 (GPX4), strawberry notch homolog 2 (Drosophila) (SBNO2), myocyte enhancer factor 2A (MEF2A), and LysM, putative peptidoglycan-binding, domain containing 4 (LYSMD4).

8. The method claim 1, wherein the risk variants or risk haplotypes comprise one or more of SEQ ID NOs:1-67 and 83-93.

9. The method claim 1, wherein the risk variants or risk haplotypes comprise one or more of:
C or A allele at rs10758669,
T or A allele at rs3808850,
C or A allele at rs1887429,
A or G allele at rs2274471,
G or A allele at rs7849191,
G or A allele at rs3780374,
A or C allele at rs10815160,
T or A allele at rs9972423,
G or A allele at rs2118611,
C or G allele at rs11071933,
G or A allele at rs1438386,
G or A allele at rs718663,
A or G allele at rs7163381,
A or G allele at rs920293,
A or G allele at rs745103,
A or T allele at rs12439792,
A or G allele at rs17293443,
G or A allele at rs893473,
C or A allele at rs2289263,
G or C allele at rs2033785,
G or A allele at rs11637659,
G or A allele at rs10152307,
G or A allele at rs4776900,
A or G allele at rs7179840,
A or G allele at rs11071939,
G or A allele at rs16950687,
A or G allele at rs10740085,
G or C allele at rs12768538,
G or A allele at rs7068361,
G or A allele at rs7071642,
A or G allele at rs7076156,
A or G allele at rs729739,
G or C allele at rs10995271,
A or G allele at rs12766391,
A or G allele at rs10761659,
A or G allele at rs224120,
G or A allele at rs492602,
A or G allele at rs601338,
A or G allele at rs602662,
G or A allele at rs485186,
A or G allele at rs504963,
C or T allele at rs676388,
G or C allele at rs7668327,
G or A allele at rs10001225,
G or A allele at rs4694164,
G or A allele at rs2071098,
G or A allele at rs1358592,
C or A allele at rs4574378,
G or A allele at rs1919469,
G or A allele at rs12507775,
A or C allele at rs1271392,
G or A allele at rs2995965,
G or A allele at rs1863284,
A or G allele at rs2911869,
A or G allele at rs2911920,
A or C allele at rs1030349,
C or T allele at rs246336,
C or T allele at rs6566234,
C or T allele at rs291528,
C or T allele at rs291523,
C or A allele at rs1973780,
G or A allele at rs1728171,
G or A allele at rs9449593,
A or G allele at rs6690359,
G or A allele at rs1019257,
G or A allele at rs766613,
A or G allele at rs10403164,
T or C allele at rs13148469,
G or A allele at rs2050719,
C or A allele at rs7760387,
A or C allele at rs9399527,
C or T allele at rs9784771,
C or T allele at rs282792,
A or G allele at rs10440086,
T or C allele at rs1352851,
T or C allele at rs13148469,
C or T allele at rs282792,
C or T allele at rs443394,
A or G allele at rs8091293,
T or G allele at rs10514090,
A or G allele at rs11576349,
C or T allele at rs4954555,
T or C allele at rs1861494,
A or G allele at rs2516049,
T or C allele at rs3936503,
T or C allele at rs212388,
T or C allele at rs10196612,
T or C allele at rs2024092,
T or C allele at rs4663142,
T or G allele at rs2310173,
T or G allele at rs10758669,
T or C allele at rs736289,
T or C allele at rs4077515, and
T or C allele at rs6651252.

10. The method claim 1, wherein the risk haplotypes comprise one or more of: JAK2 Block 1 Haplotype 1, JAK2 Block 2 Haplotype 1, JAK2 Block 3 Haplotype 3, SMAD3 Block 2 Haplotype 4, SMAD3 Block 5 Haplotype 1, and SMAD3 Block 6 Haplotype 1.

11. The method claim 1, wherein the serological marker comprises one or more of ANCA, pANCA, ASCA, anti-Cbir1, anti-I2, and anti-OmpC.

12. The method claim 1, wherein the serological marker in the individual is detected at a higher or lower level relative to a healthy subject.

13. The method claim 1, wherein the gene is interferon gamma (IFNG), Janus kinase 2 (JAK2), SMAD family member 3 (SMAD3), zinc finger protein 365 (ZNF365), fucosyltransferase 2 (FUT2), alpha-fetoprotein (AFP), afamin (AFM), Ras association (RalGDS/AF-6) domain family member 6 (RASSF6), phosphoglucomutase 2 (PGM2), AK097193, fragile histidine triad gene (FHIT), ETS variant 4 (ETV4), malic enzyme 1 (ME1), WD repeat domain 64 (WDR64), ataxin 2 binding protein 1 (A2BP1), cadherin 2 (CDH2), heat shock 70 kDa binding protein, co-chaperone 1 (HSPBP1), protein phophatase 6 regulatory subunit 1 (PPP6R1), BR serine/threonine kinase (BRSK1), Chromosome 4, Chromosome 15, Chromosome 18, transforming growth factor, beta 3 (TGFb3), fat mass and obesity associated (FTO), neuronal PAS domain protein 2 (NPAS2), mucin 1, cell surface associated (MUC1), interleukin 10 (IL10), endoplasmic reticulum aminopeptidase 2 (LRAP), leucine-rich repeat kinase 2 (LRRK2), tumor necrosis factor (ligand) superfamily, member 15 (TNFSF15), cytochrome P-450 cluster, major histocompatibility complex (HLA), laminin, plexin, NLR family, ADP-ribosylation factor-like 4C (ARL4C), interleukin 1 receptor, type II (IL1R2), 19q13, caspase recruitment domain family, member 9 (CARD9), small nuclear RNA activating complex, polypeptide 4, 190 kDa (SNAPC4), 8q24, major histocompatibility complex, class II, DR beta 1 (HLA-DRB1), CAMP responsive element modulator (CREM), T-cell activation RhoGTPase activating protein (TAGAP), phospholipase C-Like 1 (PLCL1), glutathione peroxidase 4 (GPX4), strawberry notch homolog 2 (Drosophila) (SBNO2), myocyte enhancer factor 2A (MEF2A), and LysM, putative peptidoglycan-binding, domain containing 4 (LYSMD4).

14. The method claim 1, wherein the increased or decreased methylation is detected in the promoter of the gene.

15. The method claim 1, wherein the increased or decreased methylation in the individual is relative to a healthy subject.

16. The method claim 1, wherein the cytokine is IFNγ.

17. The method claim 1, wherein the increased or decreased secretion of the cytokine in the individual is relative to a healthy subject.

18. The method claim 1, wherein the sample comprises body fluid, cheek swab, mucus, whole blood, blood, serum, plasma, urine, saliva, semen, lymph, fecal extract, or sputum, or a combination thereof.

19. The method claim 1, wherein the sample comprises a tissue, a cell, a T cell, a mucosal T cell, a lamina propria T cell, a peripheral blood T cell, or a lymphoblastoid cell line obtained from the individual and transformed with an Epstein Barr virus, or a combination thereof.

20. A method of predicting an individual as having responsiveness to a treatment of a condition, comprising:
obtaining a sample from the individual;
assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine;
detecting the risk variants or risk haplotypes in the sample, and/or detecting the serological markers in the sample, and/or detecting increased or decreased methylation at the gene in the sample, and/or detecting increased or decreased secretion of the cytokine; and
predicting the individual as having responsiveness to the treatment of the condition.

21. The method claim 20, wherein the more risk variants or risk haplotypes are detected in the sample, the more responsiveness to the treatment of the condition the individual has.

22. A method of treating a condition in an individual, comprising:
obtaining a sample from the individual;
assaying the sample to detect one or more risk variants or risk haplotypes, and/or assaying the sample to detect one or more serological markers, and/or assaying the sample to detect increased or decreased methylation at a gene, and/or assaying the sample to detect increased or decreased secretion of a cytokine;
detecting the risk variants or risk haplotypes in the sample, and/or detecting the serological markers in the sample, and/or detecting increased or decreased methylation at the gene in the sample, and/or detecting increased or decreased secretion of the cytokine; and
treating the condition in the individual with a treatment.

23. The method of claim 20, wherein the treatment specifically benefits those who have the risk variants or risk haplotypes, and/or the serological markers, and/or increased or decreased methylation at the gene, and/or increased or decreased secretion of the cytokine.

24. The method of claim 20, wherein the treatment comprises one or more of:
(a) conducting colectomy on the individual;
(b) administering a TNF signaling inhibitor to the individual; and
(c) administering a thiopurine, or a pharmaceutical equivalent, analog, derivative, and/or salt thereof, to the individual.

25. A method of determining an individual as having a low probability of developing a condition, comprising:
obtaining a sample from the individual;
assaying the sample to detect one or more protective variants or protective haplotypes, and/or assaying the sample to detect one or more serological markers, and/or assaying the sample to detect increased or decreased methylation at a gene, and/or assaying the sample to detect increased or decreased secretion of a cytokine;
detecting the protective variants or protective haplotypes in the sample, and/or detecting the serological markers in the sample, and/or detecting increased or decreased methylation at the gene in the sample, and/or detecting increased or decreased secretion of the cytokine; and
determining the individual as having a low probability of developing the condition.

26. The method claim 25, wherein the more protective variants or protective haplotypes are detected in the sample, the lower probability of developing the condition the individual has.


SEQUENCE LISTING (93 sequences)

SEQ ID NO: 1 - DNA, 601 bp, Homo sapiens
tgaataggcaactatatgtctaacagttttaagttaaattttttaaaagtagcacatagttataatggtcattttacatattttgccaaataattatcctcacttgaaagaggttattaattcttggcttacaaagggaaaactgttttggttttcaagcaccaaagtttaatacatccagttatctcaagggaatgagggatctgttcactggcaatatcttttttgttttccttttctttttgtgattgaaagtaaggataagggaactgatgtagagacaaggacatgctgaagtacmgaggaggtattggaaaatttctttgccatagccaaccaaattttggaataaatggtactaaaatatctggatgatttgtagtaaaaaaaacattgcttgcttgtctgtctacttgattaataccccttgcctttgtcttttggcttataatcctacatttggaataaataagcctctttccatattttattcctttctgccttcaggaaagtacatcttccctgttttggaaagaaacaaactaactcctggacttctccccagcccttcgtttgagcctgtttccctttccaaggactt

SEQ ID NO: 2 - DNA, 601 bp, Homo sapiens
tgaattcaccctgaatcttttaaataatgaactagagctgtataagagggagggatttacaggaaaattgcattcctacggctactgcttcataggtaatatatgggcaaagttagtaagaatttctaattattaattgtgactcaatggcttagaatatcaaggccgcaattacaaacagtgactaccaaaagagttccttttttcctgcaaacaaaaactgctactgaggtaaaagctagaacacagacccacataatgaagaagagctaaagatgagcttaacgtcatgggtaaatgwaaagcatcacaactgtggaaatggaaatgtgatattttgtgaggggaagaaaatgcttgttttagaactctgaaacagtattactttcattataagtgatgtttctatattttgcagtgatatacttatgtaactaggcatggtatctcttactgtccacagcctgtttgcagggagaagccatttgaaattggtgaattgcagggagaagccatttggaagtggtgaatattgaggatatttttgtgggggagcaggttattttcttaatattagagatgtgggtatggaagacaaggt

SEQ ID NO: 3 - DNA, 501 bp, Homo sapiens
cagcccggcacctagtagacactcagtaaatatttgaatgaatgaatgaggccctttggcagccgggaagcccgtcacagccgttgtctccaccctctcccgggctacaccaggccacgccggtaaagttgtttctcctctaggagaaacattcgctaggccttgttgccaacccgcaggcggctgggcgcttcatcccaccctcacccctttccagccaaggtggctgatgggagtcaggctctcgaggkcgcattgccacgaaacagcgtgtgtgagcgcgttgtccccggcccccggcgccacttcccctcggcctagcagcctggactggggaaggacgggtctgctgtacccgggaggtggaaggaaaagccgaaagcggagaagtgtgcgggaggggagtctccgcgcggaggcagcccggcctcctccagtgcaggctgcgcgctggggagccagccagggtgagtcactcccggccgcttccctggagggcgg

SEQ ID NO: 4 - DNA, 801 bp, Homo sapiens
gtttttcgtttctctgcaaagtcacgttatgtgtaaagccaacaaaaaaatgtttcttaaaggatccaagacgaacaacaaaacaccccaaaattactctcccagatcaacattaaaagtttagcactgacattttcagaagagtaagcaacaatgagccctgcctctcttcacacgcttccaaccttccctacgcacgcactcccagctccagacaccttcgcctttactgcagcgataaaggacagcacacccaccgtcgcgggaggaggaggaagagcctctttttctgcagaagagaaacagttcacatcttgttcctgttgcctgcttctgaaacctaaaaacagaagcagcagttaactactaccacataaatccaggggaaacaaatgcttttyctaggctgtttaagtgtctagaaaattttaaaaaggaataaggaggcgggagcgagggaattataagacacaaatggaagcaaatatcaccagcaagactcgttctaggtgtcgtcttaaagaggaagatttttctggctcagaagtttgctgccactagcagggggcaggcgggaccaaaaggtaggaagggggtggagagacagctgctgccccccacccagaagccaggcagagataacacccaccggctacacagatcgggcgaccagagcgcgccctcgagcccgcaggggcggctcctacccggcgggtcaaggtctgcgaagccccgggacgggcacggctctgaacgacctccaggctgggccctcgggggccctccagtgtgggctcagct

SEQ ID NO: 5 - DNA, 713 bp, Homo sapiens
gttttgagttggttcatggtttcaatattagatacctacacccgtttctacaatgtcatgtctttgtaaagatgtattttcagaggttgctccgataaaaagcaactactacatgaaaatgaatgtagaacaggaaatgacagtgtggtattgtccaatttgattctaaggtttgataagttctacagtgctctacactaagttgtaaggacataactactaagtaagttgttcaaacctatttcttttggcctgggggtgtcgtgaaaaatttgctaagactcagagctccctgaatgggtttggaaacctctgccttagcttttagtttgcttttctgataaaattattgatataaagctagtaccagctgatcatggcttaatatatttcctctcctatgtaatatgtttataacttctgcccagttctttcattttaaaactaatcctattgctaacttgttgtactagtcacttcctggtttttcctgtactggtcttctgcttctayttctgttcatctccttaaactccgaatgccccaagtgaaagttaccattttctaccccttaattttcttctaatcatgtaggctttaaattaggcatcatctttaaactacttctttcatttacctcttattcccattcaaaatgtccatatttcctttctttctcatatctctgctttccctcctttactgtgtgaatc

SEQ ID NO: 6 - DNA, 501 bp, Homo sapiens
atgatccattattatcctgtcaataatcataactttattctccattattcagttaaaattattaaatttcatttatattatccctacaccaaaaataatcaaaacacaaaaacacaaggcttcctgagaattaaaatgaacgaaatctattcatctcttttgctacccctacaattctaggcctaccagcagtagtattaatcattctgtttcccactgtttccaacctctagtcatcaaattagtaaccrattattttctattcaacgatgattaatccaacctgcactaaaacagataataattacccataacattaaaggacaaacctgatcccttataataatatccctaattatcttcactgcctcaaccaacctccttgggcttctaccccattcatttacaccaactatccaattatcaataaacctgggtatagcaatccccctgtgagcaggcgcagtaattacagtcttctgctttaaaacgaaaaactcc

SEQ ID NO: 7 - DNA, 601 bp, Homo sapiens
tggcattaattgttctctaatttgggtaggtttgaaatttttcataataaatattaaaaatactttttgaggaaatgtcgtcatgataaacattagaactgcgttcatctaattacttttagggtatgagtgaaactaaaaagtaagactcatctaaaaataatcatctgttttggccatatccccagtgatacatgaatttaattgtttttattgttttcagaatgaaaaattaaagaggttaaataaaattagctggcaatcaaaaaatattagttatatgaagattcagagatttggktatacttaatgaatacaagctaataaatatttattaagactctactgtgctccaaatactatacagggaggtagggatatgaagataggcttagcatcagaaaaggactgtcccttagtagaaaggctgaactaaaaggaaaagtttgacgtgataggcaaattcaactgacaggtccactacccatagtcccttcaagagctcataattaggaaaagtcatatgtataatatggcactttcaataattaagtgcaataagtgtaagtgcaataattaaggtactgggaaaacataacag

SEQ ID NO: 8 - DNA, 601 bp, Homo sapiens
gggctggaaagtgggataaggactaaggacttctgtctctggattcagcatgctgattatggggaaaagcagacatcaattcctcatgtcttgtaagcatcactgtcctccaaagttggagcccagctcagacagctgcatagtattttatagagaaggtgtgttgttgaaatgttgattgtgcaaattgaattgtgttttaagactactgggaaaaccatttaaaggaatcctattgtaagtcattttgaaaaatggagaattacaccacctccccaaggattttctgttttggtaaatwtcatgtggcaagattacttgaagaagggttccagtgttttgctagcttataatggttactgcataaggaaggtttactcgccagaggtccgcacacattggagaactgggggaatatgtaatacttgtgcagaaatgttttgattcatgtactcagaagatgggatctggatgacacctccagcctattgattcccaagtaaggtgtgatgacaccaccaccagcgaggtgtgctatgaggaatttttgttgatgatgaaaagtgaatgctaactgattgcacaccacgtgccccatacc

SEQ ID NO: 9 - DNA, 610 bp, Homo sapiens
agtgcatggcaagcattccacacaaaaagcatttgcacacacaaagaacttacaatgaaaattatgtcttacaagttaacaagccatgagaatgttctaatcatcaagcattaaaaatacccaaatgcatattcatcccatgcrttcttcgacaagtatttgtgaagtgaccatgctttatcaggcaccgtgctaggtgctagactccagagacacgaagacgaataataactggtcttcgtcaacaagaactcaattgtgagacaaaagccaacttgaggaaaattaagcctatttcaaacatgccttgatctgtgtaacattcataatacataatatcttctttgatctccacagttttctctctacctccagatcagtgcaatctctggttttccacttttttttttctttccaaatgaaaatagagcacaactgtcattttcaatctactaagtcataaaccacaaggcaggctgcaaggaccaaatgctccgttttagaacgagcccagtgcagagaacagagtccacaccacaaggcagggagctgtggtccagatgagtgacatgcccttagtgacctgggtcctggcctccgacccagagac

SEQ ID NO: 10 - DNA, 601 bp, Homo sapiens
cacaaatacttgtcgaagaacgcatgggatgaatatgcatttgggtatttttaatgcttgatgattagaacattctcatggcttgttaacttgtaagacataattttcattgtaagttctttgtgtgtgcaaatgctttttgcgtggaatgcttgccatgcactgaaaaatgctttgtgatagcagttttgaaaaattgagtcattttgccctgattgaagctgggtgaccttgtgcaagtcactttaaccctctgggacttgtggccctcatctgtaaaatgggtagaaggtcagagtgsacctcgtggcctgagaggctagtaccccatgctgcatgtaagtcttacatcctcctctttggtgattggatactgaccctctccccaggggttcctgcacagagctggaattctctctggtgtctttgcattccagtgggtggaatgccttgtttctcagtagcaatgagaaatgttaatgtgaggttatgaaagcaagccaagagccatggaataattaaaacacacacgcacacaaagaaggaaaagcgagagggggaaacacacttaatggacaagggatgagtaaaacccaatgga

SEQ ID NO: 11 - DNA, 610 bp, Homo sapiens
tacagaagtccagagatgagagacatgctcttctgattactcctacccaccctttggtcttggctcctatgtcttttccttgggggaaccttcccagaccaccaccccttgctcaaatggaatcaggctccctggacttctcatgcatgactcctagcatttgcagtgttacccttgtgagtgcttcctctttgctcactactatatccagagcccagcacagcataagttgccagtaaaatgtgctgaatgaacaaatgagttaatgccactgaatctgtatcagccttgccatgactccaggagggtggttaaccagacattatcttccttgctttactgatgagggaatcacggcccagagggcatgtctctcacctgggcttggggcactgcgagaggacccaagygccccaaagggggccatgcctttttcttgttggattctttggcaaccagcacggctctgagcacacagcagatgtggattcaaagtagatggaaatagcaactaaatgacctcagagatggagccaaaacaacttttctgtttgcttggcatcacatttccatcaaagcagactgtggagggaggccattctgtttgcat

SEQ ID NO: 12 - DNA, 694 bp, Homo sapiens
tggatttttctagtaacttcaaggggtaaagactttgaattttttttttggagacagagtctcgctttgtcacccaagctggagtgcagtggtgcgatcacagctcactgcagccttgacctcccaggctcaagcaatcctcctgcctcagcccctcaggtagctgggattataggcacacgctaccacgcctggctattttttgtatattttgtggaggtggggtttcaccgtgttgcccaggctggtctcaaactcctgagctcaagcgatctgcccaccttggcctcccaaagtgctgggattaaaggcgtgagccactgtccctggccaagactctgggtttagtaagcctcttgcaactcactaggagtggatttctaggacctccccactttcttgcttgttgtttcttttgctagccgtaagtccctggagtacactgggtactttatgggaaaaagaggaacagtgtgggacagactttgccctgrgctgcttaacgtgatgtggggaacagaggtgataagagcctgtggcagctcaggcagccctgccatgacctggagttggggaggggtatcaaggataacaagtcttatacacatgccatgctgtttctttggatcaatagatggataatttttaaaaccccctgtatgtatcaaactcactttggagatgtaaggcatgg

SEQ ID NO: 13 - DNA, 701 bp, Homo sapiens
ttgaagagagagcttgctcttggggcagtttgtactgggcgtttagggttataaaaacctagatttgctgaaaacggctctcttttatcttctgacaagaacatttgtgaagaaaacaaaacagaccttcagccatttagtctatacaccgagagaatggtttctacagtatgcctgatttctctgggtctttctaacacrtccatgtatgtgcttgtcagcttctagtgcctccatttcttgatatgaggaaattttgcttaaaaaaaaatctctactgataggacctctgcatattcatgcatgtttttcctccagaactttgtgtcccaccactgtgctaggcagtttacaaatgccatctgatttcattcctgccactacagtctaaggctatattgtccgatacctatccaccaccctcatgtggctatttaaatgttgggtcattaaaattaaatacaattacaaatgctgtccctcagtcacaccagccgcatttcaagtgcacgggtaggctagtggccatcagatcagctcatatagagcagcacgttttcatcaccaccaaaaattctattgggtagcactggtctaaggtccggacatatgtccccattttgtagatgaggaagctgagtcctacagaggttaagttgaccacttagaagaatgaacagatgaaaccttttcattttaca

SEQ ID NO: 14 - DNA, 601 bp, Homo sapiens
aggaaaccaagagaaaccagccaccacccacagctgacagggtctggggggaacagcaaaagagctgacccgcacagtacccccaggctaactgaagagtccctgaagcctacagatcaatttctcttttctaagaaggaaaggggagtataaccttcagtttcctcatgtgtaaaatgaaaaggtttcatctgttcattcttctaagtggtcaacttaacctctgtaggactcagcttcctcatctacaaaatggggacatatgtccggaccttagaccagtgctacccaatagaatttytggtggtgatgaaaacgtgctgctctatatgagctgatctgatggccactagcctacccgtgcacttgaaatgcggctggtgtgactgagggacagcatttgtaattgtatttaattttaatgacccaacatttaaatagccacatgagggtggtggataggtatcggacaatatagccttagactgaagtggcaggaatgaaatcagatggcatttgtaaactgcctagcacagtggtgggacacaaagttctggaggaaaaacatgcatgaatatgcagaggtcctatcagtagagat

SEQ ID NO: 15 - DNA, 748 bp, Homo sapiens
aaatcgaagattaatggctcaatctaggaaacatttaagattctatgtgcaggaagaacagggctgggcaccccttggtatatgtaaatgtgtcctcagttttgctttataggatctctttccacaactagttttaccatgtctacttcagcctcattaaggagttaacactgaccagttgcttcctagaagcaagttttatggtaagcactctatatttaaatactttaatcctgtaggacagggcctcatcttccaaaagatgacaatgaagctcagagaggttaggagtattccacaaggtcacccagcaggcaggggagcagagctgaactctgaactctaaacattaaagcccagcttcttcccactgtgtcaagctgttgttctgcgtgccccagccgccttgcagggagacacggtggaaacaacaggagactggaaattgggacttgcattggaaagctggctcctctccttaccacctgggccacctcaggycaagtgcctcatctccgggggcccatttcctcattggtataaaggggccatgtgggaagtggttcctgtagagattgcacatgtaggcccttcataaaccggtaagcgccacactgacatacagtgtcacccataacctttcattcacgaaccagaaaatcctgcccaatgatgttgccgaacatccactcctgaattttaacgttattactggaacaagcttgtgcaaggtcaggacactgtaaga

SEQ ID NO: 16 - DNA, 501 bp, Homo sapiens
ttgtttggtaacaatggaggggaacgttaaacaagagttgacgacgggaagtgaatcactgaattctcaaacaggctccgtggaaggagcaaagaactgtgggaggggcccctggcagggcctggcggggtgcaggggaatctttggaggctgctctcttctcaccccgccaaaggacaccctttcaactctgcctggctgtaaccaggtagcagcttcatatgtcaggccccatctagctcccaaatcamccccgctacttccgagtttccctggtgttctctgctccttggattaagcaggagggagaaatggagaaagagccttttttctacaaacactgacccaactttgtccccatacaccttaaagtctgaccctgaagaggcagctacaggctctaacagaaggaaaaggctcattcttctctccccgctcaggcatcagctacttaaatggagatgtcctcaaaggaaaatataaaacacagagacaatgtct

SEQ ID NO: 17 - DNA, 636 bp, Homo sapiens
tcctttctgtctaagggtacagacctcctattgtacctaaaaaaaaaaaggttctggttcattggggcaattcacctaagacagcaaagaacccctatgggcaggaaaacaggaattccacacaagcaagaaaggaggacttcagagaagccaggcccacacttaacaatgtcasggtggcaacagccctcctcgattaagccggcctcatcaactaagtggcccttgtctgaaagctgcatccctaaaacagctacaatcaggaacaagcaaagggttccaggtgaggcgtcctcccgctgggtcctagatacttacaactctccccggtatcagcccacccagtcccagctgagattcagagcacctgcccacccaccacctcacccccacttcctagacaggcctaatttacaggcatagaaactcaaaatgctgaacaggcccaagtagtggaggtaaaatgttgcagccacagtatctgaatgtgaggctcctgcactcttggccaccagcatagtgcgctgaggtgcaggtggggacagagcccttggaggtccaattatcttccagctcaaacagtaacccactctgtggctctctgctaagaatgacctctctccatcctcccctcct

SEQ ID NO: 18 - DNA, 601 bp, Homo sapiens
tatacagggcacccattctttgttaccatttgggatggaaactgcttatataagcttgtaaagctttggttcagaaagcaagagaatgtgtatggaacgttagcaattgctgtcatgcaaatgctgcattttttgcatgtaaaggtacctctttacctccatggtccttaaactaactttttcaagaaacatggtcatttcccacccttcctttacttccagctccccattcatacccaacagtggctaactgaaaggtttgcaaatcctgaaggctttggggcttcttttaaaaagaagrgactgagttgtaaatgtcataagactgagatggttgtaaatgtcccaaccatactgtgtgacatttgctaggaattgttgtaaaatcactggagtgagcgcttgtcctgaggcactgcctctccgcgtgaatgtcactgagtcgcccggttgacccgttgcatgttatgcagcacttgtgacattgttttatctctctagccggtgactcataattaaggaagtcaaaactgtgcaattaacacaaaacctcctcatttgccctcttcacctctccccaacccccgcccccagtcaggaa

SEQ ID NO: 19 - DNA, 727 bp, Homo sapiens
ttagagtttttgaaaaatatttgatttttttaaattgacaaataattgtccatattcatgggatacatagtgatgttttgatacatattaatgtcattatggtgatcacatcaggttaattagcatctccatcctgtcaagcatttatcatttctttgggttggaagcattcaatatcctccttctatttgaaactatgtaatatatgaatgttactatgtatgttactatataagcattcaatattctcctatttgaaactatgtaatatatgattatacatagtaacaatcatatattacataataattgtaaactatgtaataataatattgtaatatatgattgctagatatttgaagctatataatatattattgttaagtataatcatcttacagtggtttaggacactagaactgatttttcctctttagctgtaattttgagacctttaacaaatttctccccatccctctgttccctttacacttttcagcytctagtatcttctattctgtttttacaaagttagagttttggggctgataaatgtttttacctaaatactcatttttgtttccaccaaatggaacatcatgttgtcgtagaatgaaggaatcctttctgtcctatgggttattttcttctccctcaaactgacaatgcaaattaatttgattccccaaagtagtttggaaacagagaagactctaagtgttggcta

SEQ ID NO: 20 - DNA, 601 bp, Homo sapiens
agccaatgggaggaaacctaatttcctgccacaggaaattgttctgctagggaagaatatatttaaggattaaaaccttctgtcagaggaacttgagggagttgctagagaaagacctttgggggtctctcatcaggaagccagtgggtgtgggagagatctcagtcataaatggggaggaggcccaacggcaggttcctgagactgtactcctaatgctagcagctggtcttgggctgggaggaagggctgggccagggagaagagtgggtcataaatagaccattttctgcaaccaagyatatttatggtaacatcagctcagaattaagacttatttattggatttacttaatgggcccagtgccctataaggcctgggagagtgtgttctggacacatcagctatattaaatccagcagctgaaactcccaattcactcttattactttggtaaggattgctacatattttgagtggggcagagggagaaagaaagaacaatgccagggctctgggcagcccagactccgaaccaagaggtgttgttgctgatgcgtaagaagtgaatgagatgtcttccctgtcgtgagtttggga

SEQ ID NO: 21 - DNA, 601 bp, Homo sapiens
cattgaacttgcaacctaactgctgagtgaggacacgtcccttagagacaacaaataataacacctggtctgcacagaagaagatagtagggaccaaagggtgggtctgagcctgcagatcttcagaggacagaaaggggtgaggcggaggcaagtctggacgcctccctggagggggtggggcttaaccctcaccttgaaggaccaggataagttagcctggcagaggctctaggagtacacatttcagacttgggagttgatccggtcctgcgtgagcaaaggcagttatgatccaagygggagtcagaggtggacaggggcgaggacaacagaaccaagagctggaacctctactccaagacctggagctcctacagccacggatgctagcatcatggtgtgcatgtgtgatgtctttgcaaaaggtgtctcagagccaagctgtgaaggccttttaacagaccaccttccttctgattcccagagaccccaccccctggctacctgagtgaagatggagaaaccagtgaccaccagatgaaccacagcatggacgcaggtcagtcatgcagggtcatgctcttattcttaactgatt

SEQ ID NO: 22 - DNA, 715 bp, Homo sapiens
gggcgggtagacgtgagtgaatgaaaattcccccaaactttatgcccaggagaacgagggtctgtggtcagctaagcatacgtgtgcagtttggtgcaaagccacggacagccatgcagactccagccccctgacatttatagacctgtttcagatatacttatttgagtgtttcatcttataattgtgtaatttttcttaaaatagcaacatayacggaatttgaaaataaagtcaaagtgcccataatcccaccaccctaacataactgtctatgtgtgattacagtcctgatgtttgtgcatcaggcatatatatctctgtgccttgccactgagatcatagtgcatatctggtgttttcattctcttttctccttatgttattttgtaaacacgtttccgtatttaggtgtcttgtttatcgttgtgtgttatctcatgatgttttggagtggagggtccattgtttccgcaacatccctccatggttagactgctgggtcagtttttccaagactcaattttctaacccagtagggtccttcagttctccagcttgggataggctttggcatggaacctgccaaagggcttcaaacaaatgaaatatccatttagtttttattgaggggagatttttatattctggtctggtagttttagaaaatgtaagttggattttgtttggaactttattgctgaggcttaatttg

SEQ ID NO: 23 - DNA, 501 bp, Homo sapiens
caaagggcttcaaacaaatgaaatatccatttagtttttattgaggggagatttttatattctggtctggtagttttagaaaatgtaagttggattttgtttggaactttattgctgaggcttaatttgtggctcaggaaaagaaatagttaatatttcttggcgaatcaaagtggattggactggtttttgtgcccatttgcttccagaccttgactgacagcgtcagatgaagaaactcatcatttggratattaggagatgcttgaaaccctttagaacaggctgacctgggcagtgtgggatgtgactttgggagatctgttttaacatttttgtaaaataatatcaccatgagtgccgggctttgcagagggaaggtcagattcctgatatgaaaggactgagttattaaaaacaaactgaaggctagaagcgaagttaacacttgagaaattaaaggccatgatggtgaacttgactccgggctttcagcttttg

SEQ ID NO: 24 - DNA, 601 bp, Homo sapiens
agaaaaggcaaaagtggggagaaagcactaaaacgggagacaagttaaaatttctttttaattgataggtcacgttctcactctatttgcctttaagggaagaaagcaatcaagttaatatgttttccttcattgtatagtatgtaactacggacactattagaggagggatttgtgtagcacttaggacattatacttgataatttccaagggtctttctagatttaaaagtctgattctaacgtagtaataaaaataaaggcccaattttctctttaatattgcctgaagatattactstattattgcattaaaattaaacattcacacattgtttgcactgctaaataaaattatgtaatttcttcttctttccttcctccttcccccatccctctctatttccctttccccttccttctttcctggccttttttccttctttctttgttcccttctccctccctcccctttcttcctttttctaaagctggctttgagatcctttattaaagaataaatctttaaaacttatactttattttccctgttgcaggaaaaaatcatgtcctacatatgttctcaacaagacactctgtc

SEQ ID NO: 25 - DNA, 874 bp, Homo sapiens
cagactcagtgcattaaatgccatctagatgttgacaacacatgtttatagctccagcctggatttcttccttgacttacaggctcttacgtcagcttcctccttaacatctccatgctgataatgtatagacaacacaaacttagcacattacaaattgaatgtgaatctactccttgtcccaaatttgtacctccatcttgtctatctgtctgtttttatgagtcagacattttggatttattgatgacccctctcttgcatatcacccacacattcaatctatgagcaaagcttgtcggctctacctttaaaaagtatccagaatttgactatttctcaatactttggtaccagtcacaatcatatgcctcactggggttattgcaatagattcctaattggtctttttacaagtgaccttgctccaccctcaaccctattctcaaaagtgaacattatgtcacttttccactcagaatcctctagtggcttcccacytctctcagagtaaaatgcaaagtctgtccaatgaccctacatgataatatctttctgaccttttttgttactcctctgatcaggtacagaggtctccctgctgctcttcaagttaccaatcaatgtcctgctcagggttttgccctcatgacccttctggcttagaagaccctcagctatgtgtgaggctcacttcatcaccaacttcaagcttctactcgtagttatctgctaaatgatgatgttttcctcaactaccttattttaattcagaaatgccattcccagtccccacaacacttatcaccatctgacaaacactagattttacttgtttattttctgtctacctcactagagtgtaagctccatg

SEQ ID NO: 26 - DNA, 1001 bp, Homo sapiens
ctattatgttctctgcctcaagtaccagctctgacaacacatgggcagactgggaactaatgtccccagaagcaatctcttcaactagtgagagatggagctggtgatagttcttaagtgtctccagtggtgtgaagtctcaggtgcccacagtagtgacccacctatcagcacactcttaattggctttcttctcttccttgcctcatcttcttatttccttactctgctttcagggattgccttccaaatagactctttctacccaattctggttttagagttggttttggaagaaaccaaaccaagacaatggtcaagcaagtttatttctccatgagagccttttgaatagaaacctgaatcttgtgagggagtaagcctcaggaatatttgaaggaggagaaactaacaaatccaaagggcttgaggtgggaacatacttggcaattttaaagaatagcaagaaggccatgtggctggagtggggcatgtgaaggyagagagttaggagatgccttcagagagttaggctggggctgatccttgtgggcgatgatagagatttaggattttattctcagtgtcatgtgaaaacattggatggttgaggtgggcattaatgtaatatgatttaagttttaaaatattctggaaattaagtgtagcatattgagcatacatatttatcttttgtgcaataaacatataaagacaaagagaactagaaaggaagtcacaacgcggaagaagtattcaacaacattttgaaatctagaaatatagaaaagacctaacagatttaacagactagagaaagttgaaacctaagagaagtgggggagagagaggctgttataggttgaattgtgtcccctaaaagatgatgtgttgtggtcctaacctctgctacctcagaacatgaccttatttagaacaagagtctttgcagatttagtcatgttaacatgaagtcattaggtgggccttaatccaatata

SEQ ID NO: 27 - DNA, 601 bp, Homo sapiens; identical to SEQ ID NO: 24

SEQ ID NO: 28 - DNA, 874 bp, Homo sapiens; identical to SEQ ID NO: 25

SEQ ID NO: 29 - DNA, 801 bp, Homo sapiens
ttcttccttttaccttgttttacaacttgctcttcttgtctctttctattttttcttttccttcccatcttaccctcccttcccttcccttcccttcccttgtctacattcattgcttcctgtttcttccctcacccgtcttctctctttcatttttattttttatagctttttatatgaagttgccagaaggaacccatttgtcttcgcccctacacttctaactgttgctgttcattttgaggaggtggccaaatcatgttgtgaagaacaaaacaaagtcaactgccttcaaacaagggtgggtatagcatttgttccatgaagaggataagaaatcactcaataccacagatccagaccctgacttatattatagcaaaaggcttgcttaccatatrtgatgttctcttgtcacattcagtgattttcaaaccattggtcaccaggtgctgatctaaatattctctttacttgagactcttgaatgacagccagtcattttcagggtctctcttccagctttgttttctttgtaaattggaaggaagccctggtgagtgaatgcccagggtgaatgatgacattttatcttttatctttgacaatttgtagtcttttgactacaaacaacatggtataatctcagtagggatggccaagctcacagcatgctgccccaccaagggagcacttatgcacctttttaccaagtgcaattcaatcatcagcaagactggtcctacctttgtgatgtgccgtaagtgcatatttcttttaatatgtgtgtttactgaaata

SEQ ID NO: 30 - DNA, 605 bp, Homo sapiens
tttgccaagagtgtttatggattacttaattataaagtttgcatgttgaaatcttaaagaaacaagcagattgacatgacttgtttaaaaatgcagtattcattaatgaattcaatccatcagcaatatttcttcattgtcatagatttcctcagttttatcagggccatagttgcttggaaaaaatattcaccagaaaaaggtttctttttaatagtaattacttggaaagaatataaagttcttcatttctctcaaatgatagaacactttccataaaggttctcacgaagtaatgaagactgctaaacccacatcttttcttgttttcttcagctrcattaaatgtcaatctcgttagagaagtttactagatgcgggtggaaaatcctatttacaatttacatattccaggtataaggataagataagtaagccacaatgactactacagtgtacatgagaaggcttatctttttcagaggtcattactgtaaatagaatcattgaaaattgcaagacattaatttgtggaatgaatatacagagagagctgatatattgaagcaacacacacaggaatattccactttagcctcacttcc

SEQ ID NO: 31 - DNA, 501 bp, Homo sapiens
tgattacaacagcctgagtcactaagtacattttatagatggtagagtattgagtttcaagaggttcaagtgacttgcctgatgtcacacagtcagtaaggacagggctaagtgtgaaattcaggccaactgtttgcaaaaacaataacaattttccctataacagccactgctcgttaggataaggtggactaggtctgactaattaagctgataaccagcgctcttgctaaacaactaagcctacaagkttattggtaccttcccactggaatcttatattcatggttcaagaactctgaatttgtctttctctcgagaatccatttaattcacttcaatttatattcatggagtattttactaaatactaatattttactaaataaaaagtatatgcttttattaaatcatggcttcttaatatgtatctattactattactattactaaatactaaatattttactaaataaaaagtatatgcttttattaaatcat

SEQ ID NO: 32 - DNA, 601 bp, Homo sapiens
gggttccttttccagtttcaagtcagatccacagagcaaatttcgcattttcatcaccaagaagagagctaaacagatatattagttatttgcctgtgggaacatacaccctttcttgtgggcagggagcctgggaaatcccctcgtatttcaaaaattcaattgtcaaaaaacaaaagccaattttgagtaatctttgagattttactataattctgtgtcaaagcagagtattgaggaaaagtttagtttgctatattagcctaagatattgagcttaaattaaatatccagaataatrgatccagaaaagctattcttattttagagagtttttggcgtattacttttttttttaaagaaatgttgtattcagttgctaatgcccaggataaaaattagatctatggttctcaaactccagggggcctcaaaatttcctggaaaacttactaaaacgcacagactcccccaccccgagtttcagatccagtaagtctgagatgaggcctgagaatctgcacttctaacaaattaccagtgatgccgatactctggttcagggttcacactttgagaaccacttgattaggttgactgt

SEQ ID NO: 33 - DNA, 601 bp, Homo sapiens
ccaagatgttagacatgctactgttattattatgactttaaattgtgaattgaattgggtggtattgttagtgaaacagggaaccttattttatattacattaatggaattgtaactcacttacttcatggttatagaagtgtccattaatagaggctctatttttctgtctttcttttctgtccatcatcagaggcttcatcctttttctcaccagagctgcttcactcatggttctatagagcactggggagtctggttcatcctttgcatgtggcttcagggtgttgctgtgataagrtaaatagtctgggaagaatagattataagctcatatcattcagctgcctgtgtcctagcatcctgagtggatttgtgtatgctttcattaattttaggaatattgctattttctggctgtgtaaaagcattatagtccactgcttagctgcatatttacttattaaatgttgcctgagactgactcagcaaattctgccagtatctcatataaaattcttatgtctcattcttatgtttcataagaaatgatcacaatgatcatgtttggggtttgggcattcctttaatcttaatttat

SEQ ID NO: 34 - DNA, 8812 bp, Homo sapiens
tataaactccttctgaacagtttcatccacatgcatgtttaagtcaccagataagtgctggttactcctaaatataaagctcaaggtcttacctttctctttgtttccaatcatggattcttaattgtttcagtttatggaaccctgtctgctcttcagaatttttgaggaccctaaaaaacttttgcttatgtggattatatttattaatgtttaccatattagaaattaaaactgagaaaagtttcaatatttacatattaatgtatttaaaataataataaacttgttacatgttaatcttacaacatattttgtgaaaaaaaaactgttttccaaaacaacaacagagaaaagtgtactaaaaaaagaccattgtcttatggtttgaaaatcttaatatctagcttaatagaaagtatctagattttcatatccattcctgcatgcaatctgtaatagtgagaaaaactggcctcatatacatatgtacttggaaagaggaatattttaattgttcctcctttttaagattttcagataattatggatattcttttttgatattacaccaaaactaaaaagtagtagttccttaagggttagttgcaatgtgaaacctgaaaccacatcagtgaacttttcatactctattataataaaatatgctggtctattttgaaatttgaatcgatcttttacccatgcaagattttgtgacatgcattggtcatttggaagacattggtttactgagttatgcagatgttccgaatgttgacagatttttcacatcatatgaaaaaataacatttgttaatatggcgactgatctcaccagacaagtgttaaagtataggtaaacctttccaaattctattttttaattgaaaactcaattttcacatgagcaaaacatgcaaaggaaacactgcatgtttcctttgaagtgacaggttcattttgttcatttttgagaaaatgtcagccaaagatacaaaccatagtttacctgtctgttgttcttccaaataaaaatggcattccataaaaagtcgccagctcaaatggcgattcaaacaattttcacaactgttattccttgcgataagcattatagactgatatgcagcataaatgactcatgaaatttctagaaatacaattttaaaagcataaacatatgaaatatgagaagataaaccagtcaaaagttgtacataacttgatattgaaaactatgaactattattgagagaaactaaagattattttaataaataaagagagaatctatgatcttaggtcagaagactaagtattatttagattttaattctccccaaactgacatataatattaactcaaactcaaaatatcagctagaggaaagaggtggagcaagaaggccaaatagatggctctaccaattgtttccccatgtgggaataccaagtttaacaactatctacacaaagggaacaccttcataggaaccaaaaatcgagtgagcactcacagcgcttggttttgaattcctatcactgaaagaggcactaaagagggtgggaaagacagtcttgaattcctgatgccacacctcctctattccccagcaacaaccacttctatgcacttgggagagggatggggtagcgattgtgagcctttccattgaactcagtgctgccctgtcacagtggaaagcagaacggggctgtactcagcttatgcctgcacgtggagcaaacattccgggcaggccctcgccagaggggaattgccatcccagcagtcagagcttgaattccagtaagtgacataaacacaagctggagtgctctggggccctaagtgaacttagtgggccgtccaggacccaaagactgcaattcctaggcaagttctaagtgctcttgttcctcagggaagaacaagcagccatacccatatagcatgttgtgggcttttgagactcagatgtgcgggcttgagataacaaccagcacattcccagctatgctggctatgagaagtgactgctaaagcttgaggaaagcagagggaaaagtaaaggaggggacttcgtcttggaccttaggtaccagctcagccatagtaggatagcgcaccaagtgggttcttaggggttcctgattacaggccttggtcttagactgaatttctggacctgtcctaagccagaggggagccgactgccctaaagggtgagtcccaggcctggcaacatttaatacaagttaactgaagagccccttccttgggccttaacggaatgtgagtagtagcctggcagtactccatgtgggtctgtggtggtggtggcaatggggtgaagctccttggcctgtgcgaagtggagggaagagtgggaaggaccgtggcatgtgatttgagtgccagctcagctgcagtagaataaaacatcaggtagatttctaagcatttgaatccagtccttagctcccagaaagcatctctgtacctctctggggtcttggggaactcagcaccctgaaaggaaggaaacgaggatggctggcttcatcacctgctgactctagcgtcctgggaccttgcacaaacataggcagtaactaggagaaagagagatatgtgacctttcagagaattcaaaatagccattttgaggaaactcaaagaaattcaagacaacacagagaagtcatacagaattctaccagataaatttaacaaagagattgaaataattgaaaataatcaaacaaattctggagttaaaaaatgcaactgatatactgaagaatgcaccagaatctctaaatagcacaatttatcaaggagaagaaaagattagtgacaggccatttgaaaatacagagaggagacaaaagaaaaaaaaatagaaaagaatgaaacatgcctacaagatctaggaaatagcttcaaaagggcaaatataagagttatgggcattaaggagaagatagaaaaagaaatagaagtagaaattttgtgaaaagggaaaataacagagaatttccaaaatatagagaaagataccaataagaaggttacacactaccaagtggatttaacccaaagatgactacttcaaggcatttaatcaaactcccaaaggccaaggataaagaaaagatcctaaaagaggatacacacacacacacacacacacacacacacacacacacacacaccatatacaatgccgcagacagcagacttttcagcagaaaccttacaggccaggcaagagtggcatgacatatttaaagtgctaaagaaaagaaattttactctagaatagtatatccagtgaaaatatccttaaaacatgaaggagaaataaagacattcccagacaaacacaagctgagggatttcatcaacaccagacctgtcctacaagaaatgctaaagagactactgcaatcagaaagaaaaagatgttaattaggaagaaaaacatcaccctaaggtataaaactcactggcaatagtaagtatagggaaaaacttagactatcataacactgtaactgtggcgtgtaaaccactcttaagtagaaagactaaaagatgaactaataaaaaataataactacaaaaactttttaagacagaaacagtacaaagatcactgaaacaataaaattttttaaatcaggtgatgaaattaaagtgtagagtttttcttagtttacttcttgcatctttgataaatcgtttacacaagtactataaagctgttatcagcttaaaataataggaaataagatagtatttgcaagcctcatggtaaccttgaatcaaaaaaacatatagcagatacacaaaaaataaaaagcaagaaattaaattataccactgggaaaattaccttcagtaaaaggaagacaggaaggaaagaaagaaagaagaagagaccgtaacaccatcagaaaatgaacaagaaaattataagagtaataccttacttatccgtaataacattgaatgtaaatagactaaactctccaatcaaaagacatagagtggatgaatggatagaaaagtacgacccaatgatttgttgtctgcaacaaacacaaactataaagatacaaatagactgaaaacaaaaggatggaaaaagatattccacatcaatgaaaaccaaaaaagaggagtagctatacatacatatatatgtataaatatatatgaaatatatatakagagagaaatccactttttttttgatttgggtcttctttctttttttcttagtctgtctaatgattcgttaattgtgtttatttattcaaaaaaacaactttttctgccattaattttttgtatttttttcatttaaaatttatttccactctgatctttattatttctcttctattagtttatatatatatatatattttatgtttatatatatatatatttgatatatatatatgtttcaagacaaaaactctaagagccaaaaatcactatataatgataaaggagtcaattcagcaacgggatataacaattgtagatacatgtgcacccaccactgtagcacccagatataggaagcaaatattaatagagctaatgagagagaccgatcacaataaaataataggtggatacttcaacgctctactttgagcactggaataatcttccagacagtaaatcaataaagaaacatctgacttaatctgcactataaaccaaatagacctaatagatatttacagagcatttcatccaatggctacagaatatacattcttttcctcaacacgtggattatactcaacaatagaccatatgttaagtcacaaaacaagtcttaaaacattaaaaaatcttgaagtgatattcatcatcttctctgacaacagtggaaacaaaccctagaaacaaaaacaaatgagcaattttgggaactgtacaaacacacagaaattaaatgatatgctcctgaataaccagtgggtcaatgaagagattaaggaggaaatttaaaaatgtcttgaaacaaatgacaatggaaacacaatgtaacaaaactaatgggatacagaaaaagtaatactaagagggaagtttatagctatgagtgcctacagcaaaaaacaagaaaaacttcaaataaataacctaacaatacatcttaaagaactacaaaaacaggagcaaatcaaaccccaaactagcagaacagaaataataaagatcagagtggaaataaattttaaatgaaaaaatacaaaaaattaatggaacaaaaagttgggtttttgaatacataaatgaaattaacgaatctttaaccatactaagaaaaaaagatggaagacccaaatgaataaaataagagattaaaaaaggagatattacaactgatgctgcagaaatttaaagaatcattagtggctattatgagcaactacatgccaataaattagaaaaatcttaaaaataaataaataaatttctaggtacataaaatctaacaagtttgaacaatgaagaaatccaaaacctgaacagaccaataacaaacaatgagatcaaagccataataaagtttcccagcaaagaaaagcccaaaactcaaaggcctccctgtttaattccaacatttaaagaaataataccaatcctactttaattactctgaaaagttgagcaggagggaatacttccaaactcattctatgaggccagtattactctgataccaaaaccaaacacacacacacacactcacacacacacacagagagagagagagagaaaactacaggccaatatctttgttgaatattgatgcaggatatcctcaacaaaatactagcaaactgaattcaacaatgcatttaaaagatcattcatgatggccaagtgggttttttccagagatacaatgatagtttaacatacacaaatcaatcgaaatgatacatcatgtcaacagaatgaaagacaaatactatatgaccatttcaaatgatactgggaaagcatttgataaaattcaacatccctcgtgataaaaccctcaaaaaactgaacatagaaggaacatatctcaattcaataaaagccatattaaagactcacagctagcatcatactaaatggggaaaagctgaaagccttttatctgagaactggaacataacaaggatgcccgcttttaccactgttattcgacatagtactgcaagtcccagatggagtatcagtgatgagaaagaaatgaagagcgtccaaattagaaagaaagaagtcaaattatcccttttttagatgttataatcttatatttgaagaaaactaaagactcaaccaaaaatgattagaactaaaaaacaaattcattaaagttccaggatacaatattaacatacaaaaattagtagcatttctatatgcttacagcacaatcaaaaaaaaagctacaaagtaatccaatttataatagctacaaataaaataaagtacctaggaattaaccaaataagtgaaagttatttacaatgaaactataaaacattgttgcaaaagattgaaatgacacaaaaaatagaaagtgttccatgttcacggattagaagaatcaatattgttaaaatgttcatgctacccaaagcagtctacagattcaatacaatcactttcaaaataccaacaacattctttacagaaatagaaaatacagctctaggacttatatggaagaatcacattatctgactgtaaattatactacagtggtgtagtaaccaaaatcgaatggtactggcataaaaacagacaaatagaccatagaataaagaatcccaaaataaactcagacatctacagtgaactcctttttgataagggttccaagaacatacattggggaaagaacagtctcttcattaaatggtgctgggaaaattggatacctatatgcagaagaaagaaactacactcctatctcttaccatatacacaaatcaaatcaaaatacagatgtaaactaagacctcaaactattaaactacttaagaaaacattggagaaactcttcaggacatgggtttgggcaaagatttcttgagtaataccctacaagcatggacaaccaaagcaaatatgaacaaatggaataacatctagttaaaatgcttctgcacagaaaaaagcaaatcaacaaagtgaagagaatgcacagaatgggagaaaatatttgtgaactacccatctgacaagcgattagtaaccaggatatatatggagctcaaacaactccctaggaaaaaagtcagataatctgttttaaaaatgggaaaaagatgtgaatagatatttgtcaaaagaagacatacaaatgtcaaacaagcttaagaaaatgtgctcaacatcgttgatcatcatataaatgcaaatcaaaaccacaacgagatatcatctaacctcagttaaaatgatttttatccaaaggataggcaataacaaatgctggcaaggatgtgaagaaaaggaactcctcttggtgggaacgtaaattagtacaaacacaatggagaacactgttggtgggaatgtaaattagtacagccactatgaagaaaagtttgcagattcctcaaaaaactaaaactagcactaccctatgacccagcaatcccacagctaggtatataaccaaaaggaagaaaatcagtgttagtgttatctgcatgcccatgtttattgcagcactatccacaatagccaagatttgaatgcaacctaagtgtccatcaacagatgcatggataagggaaatgtggtacatatacacaatggaatactattcagccataaaaaagaatgagatcctgtcatcctgtcattgcaataatatggatggaactggaggtcattatgttatttaaaataagtcagacagggaaagtcaaacttcacgtgttctcacttatttgtgaaaactaaaaattaaaacaattgaactcatggagatggagtagaagaagggatatcagagaataggaagggtagtcagtggggttggtgggaacagagtgatggttagtgggtacaaaataagtcagagggaatgaataagacccgatgtttgctagcacaacagggtgactatagtcaaaagttattttaaaataactaagagagtataattgtatttttttaacatagaggataaatgcttgagggaatggataccccatttgcccgatgtgattactgtgtattgcatgcctgtatcaaaatatctcatgtaaccccaaaatatacacaccctactatgtactcacaaaaaattaaaaattatatatatgttcatatatgacacatatatcagctaaatttttaagtaaaaaatggcaagctaatcctaaaatttatatggaaattcaaagaacctagaagagcaaaacaactttgcaaaataataataatattagataattaatacaacctgactgcaaggtatattataaaggaagcgtaatcaatgttatgtggtattgagataatgttaggcaaatgtatccatgaaacaggataattttctggaaaagatctacatacaaatgaacagattattttctacaaaagtagcaaaattcagttgagaaagtgtggtatagtaaattttattcacacaatctaaattaagcctccataaagtttatttaaacagaagaaataggtcatagtgcccccaaaatttgggaactactcaagattcctgggattgttggaccaacatgaaat

SEQ ID NO: 35 - DNA, 1127 bp, Homo sapiens
agaaagggtagaactgaatgaaggtctactcaggctagctttctcttttctggcagcatcatcaggcacattgaatttcacatttttttttattttttagttttttgagacagagccttgttctgtcacacaggctggagtgcagtggcatgatcttggctcactgcagcctctgcttcctgggttcaagaaattctcctgcctcagcctcttgagtagctgagattacagtcgcacgccatcatgcctggctaatttttgtatttttaatagagagggggtgtcgccatgttggtcaggctggacttgaactcttgacctcaggtaatacacctgccttggcctcccaaagtgctaggattacaggcatgagccaccgtgcccagctgaacttcacatatttttaaatgtaaaaaacatttttaaatggaagtcagtcatgattttcttcacaaacatttcatttcactgcaattctagcttttctccccattaagaatagagagtgtgttagtccattttgtgtttctataaaggaataccagagactgggtaatttgtttttaaaaaagaagtttatttgtctcatggctctgcaggctgtagaagcatggcatcagcatctgctttgcttcaggggaggcctcaggaagcttttactcatggaagaaggtgaagggaaagcaagtgtgtcacatgaagagagagggtgcgagagagagagagagagtggggagatgccacactcttttatacaacaagatctcagctgaacttaytaccactcattaccttgaggaaagcaccaagccattcatgaaggatccatccccatggcccagcacctcccactaggccccatcaccaacactggggaatcacatttcaacatgagatttggagtgaacaaatatcccagccatatcaaaagggttattttcaattgttattgtgaacgtgttctcttctgtcttgtcaaagctcattaacactgcacactcaatctgcagacaaaatggtggacaagattgttgaatgggtcagtgaatatccgtccaaggctgaggcacttctgtagggctcaacaagctgtggcaatcaacaacagtccaaccgagcaacacaca

SEQ ID NO: 36 - DNA, 831 bp, Homo sapiens
tttattatgaaacgtcactttttaaaagatataatgtttcacatattttaaatcgatttatttggagacaagtcttttgtcgtaggatcaagtatgcgtctaagaggaaaatagacggtaggtatatggaaagtgtgttttctgtggcttttctggttggcttacttgctctgtttgctctaaagactattaggaggaaartgataactaagaaaacagaaagtttgttcaaaatggtgtggaggaaaaaggaaggaaagaaaatttttatctgaagaatgagagcccctttaaattatcaggccccaaaaggcactgaatgaaacagcagtcacatcactcccccttgaactaaataattatctcttgaagccacttgccatgtgggctctagactaactgatgccaagtagccatcaaatgccatatgccagacactataacactatatcctgcagtcccacagtgcatggccaatcattaagcaatgttattctctgaaaaccagtgagaattcctgtcaaagaactttacatcagaccattccttgttccctttgcctttagaaacctgcttacaacaaaagctaacagagcactccccagtgcaactgggaagagcgtctcaacagctgtcttccctttggctcaagtaaattctttaaattatattttgtgcctcagcctcctccttttaggtcaacaaggacatcaactaatacctccaggtgtgtgcagctttagtattcacataaataagattttttaaagccttaatttttagcaaccgtcactaatcattaagcccattcacattttctaaatcgagtgt

SEQ ID NO: 37 - DNA, 601 bp, Homo sapiens
tatttatataaataatatacgcttatatatgtatatttgactaaaaatttaacttaatttggaacagcgttgctactgaaaatgaagttgatatagtgcttttatcccacaggtttgtttacatattaggggtttagcaaatagattttggtgttaaaggaaagggtgccctcttcaggcaatatgtctcatattgctaaagattttgactagaaaaatgtggacttaaaaaaatactaaaaactcatagaatcacaccacgaagaggcattttgtttggtgttctgtgtctattcttacycagtacaaagcattttatttttagaaaactccaggtaaggatttttaaatttccatttcataatgtgacttagtttagttttttttaaatctctatttcctttttatcttaatatctatttcttttcaacatatgagataccttaggctcctttcttggaagatttaaggtacaactacagaaatgcttgtcatgggatacctcttttggcctgtattaacagcagtgatgggaaagcattccagaattcgatttcttttcaaaaaggaaaaacctactaagatctaagatttaatgggg

SEQ ID NO: 38 - DNA, 3900 bp, Homo sapiens
aaacattgaatggttaattttatgtaacattaatggtatatctaatacacagatacaagagattgtaatgaacaagagaggtagaatttttaaaaaggctcagaaaagtaaattaacaaccccaaatcacagaatcaatctgatggataaacattaaatattagttttattaatggtaaaacctggattgaggctttgcatcaaaaatarcacattgtccacatacattgacttccacctcttctaaaaccacactaaaatggaaataaagggatatatttggaagacataaatccacaaggacaaagagaatgagacgaggtgggaaaccacagcaaattgtggagggtggaaagtggttggaagagtggggactgtcatggcagccttgagagaactgaatcccacactggctgtgggaaagccaagagcaactagttcaaaggtagactccctctaccacacacagattcaggaattgatagacccagatacttctgcaagtcaaggtgaaggtggcttagaccagaaagattgaacaaaaatctgtttaagaagcagttagatctctagactccttgtaccccatgttagtggtgattgcccgtttactacacacaccagcagaaggctggagatttattctctagcatttacattgaggatttcttgactgaggaaccctagatatggattaagttcaggacaccaaagtgaagataggaggaataaataaaaattgatatactatgaatattgataccaccccatgcttcttgttgatctattttcctggaatgctggtagaaccttatgagattgtaacctgcccataaagaaagaacaagaactattgatgttgacagttcccccagtgaaatgctcagccaagtcacctaacagtgaagcccatatagcacgcagaacttctgatcaactttttgtcccctacccttaaatacgagatgacagccgaaagtcaccacattttgagcaaaccctccaatatgaaagacaaaggccaaaataaagaaaccacaagaagaaaataatctatatgaaagagagatttatgcatggaaaagataatgtgaaaaaaacacaaaattaatatcttaagaaagatgaggaaaaattgtgcttttcttatagttaaatatgagattatatgtatataagaatattattatcttataattaaaaataagagaagatttagggaatagaaaatagttcatgaaaattaaaaacataatagtaaaaatgaaaatttccaaagcatgtttagaagaagaaaaagcgcaggaagtcttcctaaaagcagaacaaaaaaaattaaaaaataaagagaaaatttttttttgacaggatcttgctctgttgcccaggctggagtgcagtggcataaacacagctcactgcagcctcaacctcttgacctcaagcaatcctcccacttcagcctcctgagtagctgggagcacagccatttgccactaggctcagctaatttttaaaaattctttgtagagatgggggtcccaccatcttgcccaggctggtctcaaactcctgggctcaagcgatcctcctgccctagtctcccgaagtgctgggattacaggcatgagccaccacacccagcctagaaaagtgttttataaatgagaaatcaaaggagtaatttaggaattacagaaaaggagagcagagaaagtggagggaaagaatcatcaaagaaataattcaagaaaaattcccagatgtaaatttccaaattaaaagggccctctgagtacacagtgaaatacatctaagacccatattaagacacatgatagtgaaatttcagagcattcttgccaaatgagggagtgaacccaagaagatgacatgggatctgagaaacaggtgattcataagagaaaaatgaaaagtatctctagaaccatgttacaggaagacattgaaatgacctctgcgcagctgacctatcagtcaatcccaatggagcaggtcagaaagcaccaggagagacagcttcataaagttcagaatggtagaacacctaatggatttgaaagcactgagaggagattgactcaactgtaggaaaagttgggaataaattagcattaggtattagaaatgtaggccgtcttttaaaaaggcaattatataagttctagagaaataaattgtcaggaaaggaaaataagtaatatatgatttagctgccaacagtgtttccattttacatggccataaaatagaaacactgaatattgatctaaccaaacttacaacatagtgggagggtgagacattgggaggagtgtttgtgtgtctgtgtctagacgtattttttaaaatgtaaaatcaggcctggcactttgattcacacctgtaatcccagcactttgggaggccgaggtagggggaacgctttgagcccaggagtttgggaccagcctgggcaagacagggagaccccatctctacaaaattttttgtttgtttgtttttgagatggagtttcactcttgttgcccaggctggagcacaatggcatgatctcggcctattacaacctctgcctcctgggttcaagcgattctcctgcctcagcctccttagtagctgggattacaggcatgcaccaccacacccggctaattttgtatttttagtagagacgatgttggtcaggctggtcttgaactcctaacctcaggtgatctgcccacctcggcctcccaaagtgctgggattacaggtgtgagtccccacgccccgtcaaaaaaaatttttttttaattagctgggcatctggtccccgctacttgggaggctgaggtgggaggatcacttaaacctgggaggttgaggcttcagtaaggcgtgattgtgccacttcactccagcctaggtgatagagtgagaccctgtctcaaaaaataaaaataaaaagtaaaatcatctctacagtgggaaataagtgtttaaaagtaaaacaaacaaacaaacaaacaaacaccaaggcatatctatataggaatgttcttcagaactattgagggaaatacaaaagaattggttgaaaagaatgaaatgtagttgcattattggaaatggtgaagttgtagagctgccatttttcttaacaagccttttagaattatgtaactctttaaattatggacgtgtataactttcattcaaaaatataaaacaaaaaaatctgtattgaagaagaggctgtgaggtgaagtcaaatgggctgcacattaatttggcccacttggtctttattcaagagttgaagtctggggcaccccaaagtttagaggtacctgagaaggcacagccagaactgtaggagaaaaacagagagtgtggtgtcttggaagccaagtgggtcgagtgtttcaaggacaaattgtcagctttctcaaatgcttccaagaagaaagtaagaaagttcataaattggccaggcacagtggctcacgcctgtaatcccagcactttgggaggtccaggcgggcggatcacaaggtcaggagattgagatcatcctggccaacgtggtgaaaccctgtctctactaaaaatacaaaaattagctgggtgtggtggtgcatgcctgtagtctcagctactcaggaggctgaggcaggagaattgcttgaacctgggaggtggaggttgcgatgagctgagatcgggccactgcgcttcagcctggagataaagcgagactccatctcaaaaaaaaaacaaaaaacaagaaacaaaaaagaaagttcagaaattaccataggaatgaagaatacacaaatcccaggtattttcttgggatttccagccgacagaaaaaaagccattggcttcctagtt

SEQ ID NO: 39 - DNA, 888 bp, Homo sapiens
gggtgcgctgcagggggtgggagtgagcgaggaggaattgcggggctagggtgggttgctggaacctggcacgtgtgagaagggtttgccatcgtctctgatgcatccaggctacagccagccgtgagttcctgctgcgcgccaggcacatgtcagatgctgggataccaggaagtgcacccagataggggcctgaagactatgtagaaactcagagggtcaagaacagccagaaaaaaatcttgaaaaagaaaagcaaagtaggaagaccagtggaacataagatagagaccagaaacaaaaatacacgtgtatggacaatttatttcagaggcggggctgcagagcacaggaaggaactatttttggttaatggtgctcggtcaamgggatatccacgtagggaaaagaaaaaagggaacttggccccctacctcccatcacacacagctctgaactccagatcaattgtatctctaagtgcgaaaggtagtgtagcaggaggagccgcagacaaaactcctcagacaccggattaaagaaggaagaggtttttattcggccgggagcgtcggcagactcgcgtcttaagagccgagctccctgaaaaagaaattcctagccattttaagggcttacaacgctaaggggtctgtctacgtgaaagggtcatgatagatcaagtaagcgtgaggaacgtgactgggggctacatacatcagctaacagaacaagaagttttacagtgctttctcatacgatgtctggaatttacggataacgccagtagttttggtcaggggttaatattattattattattttaaccaccagggccaggtggtggcgccaaggtcgtctagctatttatcttacttctgttttttt

SEQ ID NO: 40 - DNA, 4552 bp, Homo sapiens
ggaacagtaaaatacccacgttaaaagctctttctctgtgcctccttagcaccctctgatccttcatagagcccagcaaaaatcaaccatactataagcatccttcacctgccttgcagctaagctgaaggacaatgaggacaatgatgccgtcctttctaccccatgtcccaagctcccagagcagtgcttggtaaaaaaaaaagggtacttactaaaatgttggctgaataaaaggaagactttgaaaaattaagttattttgtacccctatagtgtgaattctttttattgaaatttaatacacttacagaaaaaaatgtacatattataattgttcatagagctcagtgaattttcacaaactgaacaaatctgtaaaccacacccagattgagaaagggaacatgacaaacaccctgcaagtctcccttgtgccctgtgctggggggtgccaacaccaccacctcctagtctgatgatgtactaggagattcacagactcagcctgtagtcatactcatggctatgttttactacagcgaaaggctaggaagcaaaatcagcaaaggaaaaaagcgcatggacaaagtccagggaaaccaggtgtaagcctcccacagtcctctcccagcagagtcacataggacacaattcctccagcaatcagttgtgacaacccatgtgcaatgttgtatacaagggaagctcattacagactcagtgttcagggttctcattgggagctgctcacctcatcctctgcctaacacatatgaaaatctgactcccagaaggaaagcagatgttgggcataacccacattgtttgtacgaacagtttaggcacagtgagccactcttatcatttagggagtaatgagaaccctcctgaaatccaagctttcagatgccagccaagggccagccttgcaaacaggcctgctttctcatctcttttcacacagtccatctccttattgaggcttctttacagcaaaattattattagtaggaccacatgcaaaagggtgagaccaacctcaaatatggacctcagttcttcctggtaggggtcctggacttaaccagctaaaataaatcccattaaccataaagtctatcttaggaagccaggtggcttcctccttaattcctttttgttgttgttgttgagatggagtcttgctctgtcacccaggctggagtgcagtggcgtgatcttggctcactgcagcctccacctcctgggttcaagcgattctcctgcctcagcctcccgagtagctgggactacaggtgcacaccaccacacccagctaatttttttatttttgtagggactacaggtgcacaccaccacacccagctaatttttttatttttgtatttttacttttcaccatgtcggtcaggctggtctcaaactcctgacctcaggtgatctgcccacctcagcctcccaaagtgccgggattacaggcatgagccaccgtgcccagcccttaattgctgtctgaaccttttttttttttttcaacctgacctgaggcatcaattcaggcacagtataactctgaccagtaagcggaagctaacccaaatgctttccagggctgaggggacatcatgagagttgtggtcatgtacagaagcacagtcccaggggcacaggtggtacccctgtaaacaaagaatttgcaatattagggcacctcaagcaggacatccattaggctgtacaggtgaacagacctcctaccaggggaactcctgcccaagtgtttccagtcccatctgaaaagttcccagagtccttggcttccaagggactgcctgaaggcagtgagtcccaaacagttttgcttgttcaggatacaagttgatccatctcaygagcatggatgacatttgtgggagatcttcatggaaagcatagaagctagacccactctgttgtcagtctgaactcaaagcactcacagcacatggaaaggcagcactgggaattttccttcagcaagaggaggcttccagggatcaagcctccccaggtgccaggctttaattattttctccaatattacaaaaacagtcgtgccctgttcagcaatcataacattgcattttttcaaccatctcttactctcagctaggcctttcctctggaagggttgtatgtaagaggcacaaaagcatttttgtagataaactttttttttttttttttgagatatggtctggctctgttgcccaggctagagtgcggtggcacgattttggctcactgcaacctccgcctcctgggctcaagccatcctcctacctcagcctcttaagtagctggaaccaaaggcacatgccatcatgtccagctaattttttgcattttttgtagacacaggatttcaccatgttgccaggctggtcttgaactcctgaactcaagcaatctgcccacctcggcctcccaaagtgctgggatcatatgcatccatcactgcgcctggccaacatttttttttttaactatcatcctagagttcaagtagataaacgtttttatgcaacttaaccagaaagacattcagaaattggtaattatgtgtggagggttgaattatgtccccacaaaaattcatgttaaagccctaaccatttacgtgtccttaaaaatgtgagtatatttgaagatagggcctgtataggtaattaagttaaaatgggatgtctaggatgggcccttaatccaacctgagtggtatccttattaaaagagaagattaggacacagacacccagacagagggaccatacaaggacacagtaagaaggcagccatctgcaagccaagcagagaggcctcagaagaatctaaagctgtcgacaccttgtcctggactttcagcctccagaactgcaggaaaataagtttctattgtgtaagtcactcagtttgtggtactttgttatggtatccttagcaaactaataaaggatgaaaccctgaatttttaaaaatttcaaaatgtgtttacataatcttctaccccttttgtcctgattattcaatgagcagactagaaaagcctaatacctttctggggacagctacgttttaataatccacctaccttactaagctgtgtgtgctgggagaaaagtgagggaagtagagtcaggctgtcagtccactgttgcaagctgcaactaatcccaaaagctgaatgcaacaagtcaacagcagtgagagaagccccagagaaggtttgagtctggtctgtcaaaagcaggaggagaggtcatagtccttgcgatgtgcccttccccagcttggagcgtttggcaaatattagaagttaggaatgcaattagcctctgtattagagacaaagagcctatcagcagcttgatcttagatggtcccattagagaataagcccttttcagccaggttccaggacgttttcatagtttagcgctccacggagggggtccttaatctccaacggccccaacgtctgagtcctggtcattgagcctgtgcctgctttcagttcagcatagctcgtgttgaggctaaaacagctcaaggcagacagtagcaagtttcagcttagagtcagggccaggccattaggatctgagaatttgagattctcaacaagcagctgctttttttcagcagcagtgaaagcatgctgcatggccagacagctgtggtccaagtgacatgcaaagctgcctttttatcttgttggtcttagagtccaattgacccactcaaaaaaaatgcatattagactagacaatcatcagcctctaaggggagacatttgattttgacaagggttcattagcaatttaactgtattttcattttccaaacagattgcttctctttacatgttgtctccctctttcccgaggctccagtaagcacgaatcacagcaaaaataatcagttacagcctgttctggttccaatatccgaagaatttaaattccaacctatccactagtggcaaaaaactagaaagctgtatcccactaacacctttcatattttatttttgagtagctttatttgattagggtgatccctctagcatttatgaaattaacaagcataaaactttttttttaagagatgagtagctgggactacaggcgcacgccactgcgtccagctaaggtatgttttttaagcaatgaaatcaaccagtaaaaacaagttcaaatatttttcattcacacactcaaggcctgctgagcatccactgcatgtgttttacttgttccattagaaaggtttattaaaaaatggcggggcacagtggctcatgactgtaatcccagcacttagggaggccaagatcgcttgagcccaggagttcaagactagtctgggcaacatggtaaaaccctgtctctatttaaaaaataaacaaatacaagcaagtttttttttaaaaaatgatatattaaagaaaatcctcctaattctcttatgtcttttcatagctgtttccatgttctcatattcccatcct

SEQ ID NO: 41 - DNA, 601 bp, Homo sapiens
ttgttctaaaaatgtcttttcagtttttcaagttgacgaagcacttaagccccaggttcttaaaaattatttgaagtaaaacttaactttcatctattctgtctggagaatgaattaaggcaatatttcctttacgtcatgattgaaagtcctatttgcaaacttctgctattatgttttagtaaaaatattttatagaagagttttaggaactctgatttagttttcaaaggtaactcctagaaaagtacttttgaaaaattataatttttactctatatttcagaagcagtaaggagaygctatagattgatagctctttatctccactgacattcagtgatggagcttctttcttcatttctagctatgaagattaagcacctcataaagttgcagtataaatacctagaaattcatctgtgcctttttctgcatgatattatgttgacaagctggtatcaagcaacctaacacacttaaagattaacattaaaactgatataaggttttacatttaaatctttaatccatcttgagttaattttttgtatatggtgatacataggggtccagtttcagtcctctgcatatgatagcc

SEQ ID NO: 42 - DNA, 1416 bp, Homo sapiens
aatgcataaaaagtattagaactctccaaaagttcctacgccatttacacctccagtattgaaaatgatacaaagatgaaatgcatagtaagttgtaaaaaataatacaatttaaaatagtaaaaaataaaactaagaaacaaaaccaaaaataaaatttgacatatgaaaaagtatattattacaaggatagattgtgggcaattgcacagagataatccctaagaactggcagactttcacggtcattaactatattttgaagtctttcatcacaatgaatagttgcgtttttcttttagggcatgactctcttcagataatatgtttagattcatttccaatgtggcactgctctttatgaaattcttctatgattctatgtatactgatatgagcatttcctattaaagctttctaccttctgtgccatgcttctgtgttgtttagggtttacagaaatccatttcacatgccctcatatacagaacacaagtttggcagaaacaatgctggtgatcgaacagcaatgccattgtgaacattacatcttcttatcctactgtgcacatagtaatttttgaaacagtcagtaagctttactggcttcttcaggcaaattcagctttaattcattaaaacctcctgaaatgtcataagtaggaaggtatatgatgcatttttaaactgaagttttctttgttgctgtatgcagtggctaatccactcatttgacttttccaccaaatgcaaataaagacaataacaagataatagttttgcctaacatgacgtaactatcctgcatacaactgaaaatgcactaatcccttccccagactttggctttccggatttcaacattcgggattttaatcttttggggttgtgaatttcacaattttagacattaggaattttgatctttcaggatttaaacttttggaatttcatcattcaggattatggcatttgaaattgtgtcttccaggattatgatycaaaggcaaggcactgatgtaaattcctgtctagagctgaagggctgagaactaggagcatcaaaggcaggagaagataaatgtcgcaactcaagaagttaggcaaagttaattcaaacttcttccaccttcttgctctattcaggccttcaaagggttagatgatgcccactcaacatcggagagggccgtctgctttattagtccaccaattcaaatgtttatctcttctagaaacaccatcacagacacacccagatattttaacctggtatttggccccctggggcccagacaatttggcatataaaattaagtatcacagtgcggatagtatatttacttaaaaatgttgtgaatattagcttgttaattaaaaatctatggattttactttttaaaaatctggatttcc

SEQ ID NO: 43 - DNA, 1250 bp, Homo sapiens
agctttggtaatagatcccagattaatttttagcccaagcaacaggaaattcctcttaggccttagggccttaccctgcagaaatatttttgttggtgcctatgttagtcaggattgtccagagaaacagaatcaataggagatagatagatagatagatagatagatagatagatagatagatagatagatgatagatagatgtttgatagaaagacagataaaagagattactataaattattggctyctgcagttatggaggcagagaagtcccacattctgccacctgaaagctggagacccaggtaagctcttgatgtagttccaaggcctgagagcaagagagccaatggagagccaatcataatccagaaaggtacaagccctatcactagaatcctgaatgttgaaatcccaagagatcaaaatccccaaaatgtaattctagaaaaaataattttaaaattatctaaaatatttatttacatttttaaaggaggatttatctaagaaacataaaaacatgaatgaatacttcataggccactttatacgataaaataagcaataatatacatatgtttgtgagtataaacactcagatattctaacaacagtcacatgggtataacagttatgagcagatatactgtgttcataaagaggtcaaaaagggaaatgtataaacacatatcactgtggttggtaattgtgtgcacctagctttataactgaggtcatctgaaacaccatgacaaaccacctaagtcttttgatgggaccaatcaaaaccaccatttacaccatcccccaaagagctaagatcttgagaaattctatctttcacaatgtagatgtacagaaaggatatctctttatttattgaggaagtttcagcatttttatgtatatgcataatgtttaaatacagtccacattgtgataatgtacttcatggaatcaaatttgaaaatgcataaaaagtattagaactctccaaaagttcctacgccatttacacctccagtattgaaaatgatacaaagatgaaatgcatagtaagttgtaaaaaataatacaatttaaaatagtaaaaaataaaactaagaaacaaaaccaaaaataaaatttgacatatgaaaaagtatattattacaaggatagattgtgggcaattgcacagagataatccctaagaactggcagactttcacggtcattaactatattttgaagtctttcatcac

SEQ ID NO: 44 - DNA, 1029 bp, Homo sapiens
caacaaacagactgccttggagggtgggaggtaggaaacaggaacagcaggagactgagctccataaaatgtaattgtatttctactgctgcagacatagaaaattatgcctaaagaagccccagagtataaattggctaggaacattgtgttctttctaggcactgttttaagatggttagtgacaaataagaaggttcataagaaaggtggttgggtctgaaaacagatttggagactaattgatggtgggaatgttttgcctggaaaaaggaagacttgtagagggaagatagtaacagagtcactgtttccaaatatctgaagggctggcaggtggaaagatattaaatacattctatgtggctccagagggcagatctgtgagtatcggactacaaagtggcagatatcattagctctccataataacgggtcaaagtgaaaaaaaatgaacacttaaagagtgccttccaagggggtgagcttcccatccctggattcactcaagcacagagcacatggccacaattcttcaagaggatatctacatttggtcagtgcatgggccacttggatttgtggtgcctctctttacacatttgacccatttgatcattaccgccattttcaggttttacaagttctgtaacaaatagtggggggttttttgtgatcagtattgcaatgtttgggtgactgcctagtgaaacatattgttttcattgttaagttcatggaaataaaaggtaaaaccttttctttttaacttagaggaaaccacactgtatttatttttttctcattcccagcctacttcctgcttttcctgcagctcagcattgcatattgatggtccttgggctttgcttgcctcttcacatgtgttttatttttgcctgcaaagtatttttggtgatttgctttattgtgactgtaaagtattktttaatgagtttgaatgctgttaggcatattgctcattctctaggcctatacagttgccacgactgccttctacctaggctt

SEQ ID NO: 45 - DNA, 1650 bp, Homo sapiens
agttctcaagattattaagacttaggatgtgtcctcaaggagtttgtagtttgggcagggattgagatcttgccctactgtgtggccagagcataagatggcaggaaagggggctggggtccagacctaattcaaaggaaaggccggggttctggtatcctttcatcacttggcatttccaggtttcagaggaagaaaatyaagttaaaatcctgattccccatttctccttctggcatctctgattggcatcctttacaaggatggaatttcatgacttgtgtatttgtgtacttgtgaatcagtgaccctcttgttttggactttatgggagacttgcgtggggagagggaattgtgctcattcacctgggggggttggggtgatccccacttgtcaaaggtgtttgaaccagagcaacgccatcttgagtagcagctgggcaaaatgaggctgagacctgctgggttgcattcccaggaggttaggcattcttagtcacaggatgagacagaagatcagcacaagatacaggtcccaaagaccttgctgataaaagagaatgcagtaaagaagatggccaaaacccacccaaaccaagatggccacgaaagtgatctctggtcgtcctcactgttcattatatgttaattataatacattagcatgctaaaagacactcctaccatcaccatgacagtttacaaataccatggcaccttccggaagttaccctatatagtctaagaagggtagggactttcagttctgggaaatctctgcccttttcccaggaaactcatgagtaatccatccctttttagcatataataaagaaataactatagccgggtgtggtggctcatgcctgtaatcccagcactttgggaggctgaggcaggaggatcacttgaggtcaggagttcaagaccagcctggccaacatgatgaaaacctgtctctactaaaaatataaaaaattagcctgatgttgtggcgcacagctgtaatcccaactacttgggaggctgaggcaggagaattgctagaaaccaggaggcggaggttgcagtgagctgaattgcaccactgcactccagcttggacaacagagcgagactccgtctcaaaaaaaaaaaaaaaaaaccggaacaataagcatactcagtcaagcaactcatgccgccgctctgcctatgcagtatccattctttattcatttagtttcttttttttttttttgagacggagttttgcttttgttgctcaggttggagtgcaatggcgcgatcttggctcactgccacctccacctcctgggttccagcgattctcctgcctcagtctcctgagtagctgggattacaggcgtgagtcaccacgcccggctaattttgtatttttagtagagatggggtctctccatattgatcaggctggtctcgaactcctgacctaaggtgatccgcctcctcggcctcccaaagtgctggaattacaggagtgagccaccacgcccagcccgtttactttcttaataaacttgctttcactttactctataaactcgccccgaattctttcttctgcaaggtccaagaatcctctcttgggatctggattgggccccctttcaggtaacatactcat

SEQ ID NO: 46 - DNA, 1001 bp, Homo sapiens
taatcgtaagaaaagaaattcactcaagtttctcatccataaatggttacctgttcaaatctgaaacatgatatcaacctgatgaacatcaacctgatgattatcaaaaagctcatgattttataaatatatgcccaatcaccgtatcatcctgctgaggcttgggaggaggagggaaactgataggaacatgcagctcatgatatctcttgtgccaggaattatcaaagtcctttgtctctgacctaagagtttcatgtcttctgtcaatatctatgaaattgtggctaacttgttagcttgcaagtagagtaaaatctcagatctttcacagtttcatgacagtacaagattccccagtgggcctgctttgtgatgttctgagaatctttcctcaaggcccagacagagctcacttctcctattcttttaatgattttataaacaactaatttcctattttgatctaatttctgcttaaatactagggtggtttctgtytcctgtactgaaccctgaataataaacatgtccactataccatatcagactttcactttaaaccttcaaaatgccattcattattaagcactgttctcaacagggaaatatttaccacttaatcatcatttggaaacaattttgtttttgcttttttttaggtaacaatctagctctgtagcccaggatgcagtgcagtagtgcaatcgcagctcactgcaacctctgcctcctggattcaagcaattctcctgcctcagcctcttgagtcaggggggatcacaggcacacaccaccatgcccggctaatgtttgtatttttagtagagatgaagtttcaccatgttggccaagctggcctcgaactcctgggctcaagtgatctgcctgcctcagcctcccaaagtgctgggattaggggcatgagccaccacacccagccctggaaacaatatttttaattgtcccatcactgtcacagaacttgatctgatgtgtga

SEQ ID NO: 47 - DNA, 1262 bp, Homo sapiens
acactaaaactaaaaacatgttacgattgagacctggagtctcctacagataatttaaagagaaataagatatctttctagcaaaagttgagatcattcatgctcagagatgaagagacagaagagagaatctttcctatttcttttaaatgtaatgagtgtattaagatgatactcattcatgcatctcagtagcaccacttgggaaaattacttaatatctttccatttggtttcttcatctgtaaatgtaggaatgtttagcccagagggccattataaagatgaaaggaggtcattcatttggttatttaacaaatacttatttatttattgagtgtctattgtgttagatactgttctgagttctgagttctagggacaaagcattgaacaaggaggaccagggccctgcaatgagaaagttgacatgctaggggaagagacagacaaacaagcaaagtaacaagtacataagaaaacagtatgataagtatgrtaagaaataggaaacacagggctgggtgtaatggctcaaacctgtaatcccagcactttgggaggttgagccaggaggattgcttgaggccaggagttcaaaaccagcctaggcaaaatagcaagaccccatctctacaaaaacaaagaaaaagaagaagaaaagaaatagaaaacatagtggtgcagtgtgattgaatctcacctctgaggaggtgacataaaagctgaagcctgaatgatcaggggaaccagccccgtgaagatctaggaaaaggaaaagggcaatttaggcagaaagaagagcaaactgggctgggtgccgcagttcatgcctgtaatcccagcactttgggaggctgaggtgggtggattatttgaggttaggagttcgagatcagcctggccaacatggtggaaccctgtcactactaaaactacaaaaattagcctggcatggtggtaggcacctgtaaccccagctacttgggaggctgaggcaggagaatcgcttgaacctgggaggtggaggttgcagagagccgagattgcaccactgcactccagcctgggcaatagagcgagactcagtctcagaaaagaagaagaagaaaaaaaaagcaaactaggcacatacatgtttttggtttttttattgaagctatattattatttacagaaaagggaacagacaacatcccttcaaactctttcatataaattagctttcatcacattaagcagaaagcatataaatcgccatg

SEQ ID NO: 48 - DNA, 665 bp, Homo sapiens
gcaaattatgttttgatgcaataactcaaaacaatgtgtacctgtccacaaatttaacattacctttgggcccaaattgaaggacttgtggcagaatagctatccttagtgcccagacccctcatttgacacttatctacatacaaccatcatgagtttttcttcaccgtgcctgcatgtgttcatgtccttatgcctcattatcccaccttgatcataaactccttcaaggcaacggccatgcctgatcattcattttatgtccttctgtgttaacccaatgccttgtccatcatcagaacagggcaggggctggttgacagatgagtattcatgccaacaaaaacactcttccygtcgtacagtttaaagagcccatttgtctgttatgacatatcactcgctttggtgtttgttactggagtgcccaaccagtcagcgttgataggtcactggacacattatataatgtgctgaaattaactgaattaatattgtcaaaatggcttggcttttaaatgcaggcttctccttcaaagacacgcagcatggtctggactctgaaagacaccacttttgcataaaaaatacctgcttattatgactgttacagagaaccttattttagtcactgatcctgaaagtcactttaaaaaaaaaaaaaa

SEQ ID NO: 49 - DNA, 2115 bp, Homo sapiens
tatttatttataaagcacacaattgtttaaaaagatttattatgcatatggattatatataagtatataatacactataaagtagagaatggtgaagcaatatggaactatacatttgtaagattttcatgtattacatgaagtagaacaatgttaactgtaagtggactgtgaagagttaaggacgcatatgttaatttttagcacgattacttaaaaataatactaaaacagtatagctaaaaagtcaataggaaaattaaaatggaattctaaaaggtattcaagtttataaaaaaaggcaataaaaaagaaacacagaaacaacaaacagagaaaactaatataaaacaaataataaaatggtatactcacatycactcaagcaaagaattatgtcgcgtagtaattcattaaatactctaatcaaaggcagaggttgttagaatgaataaaaagaacaaaacccaaacatttgctatctataagtcatatgtttaaaatataaaaacagtgtggctgggaggcttcaagttgatccccatgatttgcgcctcttctagtcatgtccttgcataatcttttccccttgaatatcagcaggatccagggctttgttctaaccaatagattatggcaaaggtaaccagatggatgttattacatgtacatagttactgtacataagattgtaatgtccatcttgctaggaaactctctctccctccttggctttgaagacacatgctgccatgttatgagctgccatggaaggagtgccatcagcacaggatgtagggcagcctgtggctgatggccatcaagaaatggaggctcagagtcctacagcctgaaagaacccgaattctgccagcagtcacctaagcttggaatgagatccttccccagtggaacttctaatgagaacacggcccttgcaaacatcctgcttgcagacttgcagaggactcagtgaggtgtgtttggataacatagaaagtgtagatgatgcatgggttttattttaagaggctaaattttttatagtaatgttatacggtaaaagataactaatccagattttgatatctggaagtgggtgtattaatttgctagggctactgtaacaaaatatcttaaactagatggcttaaacaagagaaattcatcacctcacagttctgaaggctggaagtccaagaccaaggtactggcaatgctggtttcttctgagggttgtgagggagagctgatttcatgcctttcttctagcttctggtggtttactggaaatctttggcctttcttggcttatatatgtatcacctgatctctgccttcattttcttatggcattcttcctgtgtgcatgtctgcttctgtggataaatttcttctttttatgagagcatagtcatattgcattagagcccaccataaggaccttacattagcttaacctaatttgcaaagactctatttctaaataaagtcatacttacaggtattgggggttaggactccaatgtcttttcaggggaaatgattgaaacagtatcagtggttactaccgaaacaatacctaagagtgtggaagtgactttggaacaaggaagtgggaagactagaggattttgaggactgtgataaagaaagattaagttgccttgaactgacttagtagaaatctggaccttgaggaaactctcattgagggtgacagagaaggtgaggaacataggagtacttgtggaagagagacccatgttatgcagttgcagaaagcttagtgaaatttctcccttagttatgtttaaagaagaacacctaagtaataaaccttattatttaactaaggagatatccaagtaaagtgttgaaggtgctgcctgttccttttgctgtgtatagtaaaatgtgagaggaaagagataaattgaaagaagaaagattaagacaaaagcaaccaggacttgatgatttaaaaaatcttcagcctctccagatggcaaaagattctacaattaattaatgacttttgagcaaagtcagcaacaatcaggagaaatgctctttctttaactgtaaggagtacataagcttgaaattactggg

SEQ ID NO: 50 - DNA, 701 bp, Homo sapiens
gcttggcgctttgcagtcaggaatgaagcccacacccagccacaggcctgctcctgagacacagtgcagcctccatcaacggcacctggctcagacactcccgccagaatctctgccagccctgtccagaagccaggtgtggctgctcctgcactagccagaagggaacctgggcaggggcccctccaccgccaactcttggcccgacgaccggcgggctctgattggtagaggagctagaccaccagcgggttctgattggtgtacaaaacagattcccagagagagttctgatttgtggagctaatgtcacatgattaccctacagcaaaggatgctgggaagggaaatggcggctaccctagggaaaatttagatgtctaacctgggaaattcctcaaacctagaattcaggtttgccaggtggccgaaaaaaggacagatgtcccacacactgttgctcaggaaagacagggaagagtaaatgacacaatggctctrtagatgataaagagcaacacttttattattcttccagtggctccccaagtcccttaaacccgtgcccctcctcccgtcccccatgcaccctccaaaggagatgacaaaggcggcagtgaaggaggagaaggaggaagagaaacacctttattggaagagctgtgtggacaagagaacggggatgagagtgagagcatggg

SEQ ID NO: 51 - DNA, 701 bp, Homo sapiens
tctcctgtctccaatgtggtttttgttttgttttgttttgttttttgagacagtcttgctctgtcgcccaggctggagtgcagtggcgtaatctcagctcactgcaacctctgcctcctgggttcaagccattctcctgcctcagcctcctgagtagctgggattacaggtgcacaacaccacgcccagctaatttttgtatttttagtagagacggggtttcaccatgttggccaggatggtctcaatctcttgacctcgtgatctgcccgcctctgcctcccaaagtgctgggattacaggcatgagccaccgcgcccggccactgacgtgttgttaaaagagccgtgactgaagtcccaccacccagctttgcaagtgctataatctgacctcagttccaaatacctatttaaacgtcaggaacaaagggcttttttttattttttcaaaacatcagtctttaagctttaaaaatgtttccatggagagtaactcagygtcaagagatgtgcagaaaatccaaagaagggtaaggttgcccctggtatcaaggaaataagaaccagaagtcatcaggacagtgaagatatttagcaaagaaatgtaagtgggaaaacatgtaataaagttagttttgtgatttaataaataaataaaatgtttgcgtatgcatcgcttcccagaagcagccaggcatc

SEQ ID NO: 52 - DNA, 623 bp, Homo sapiens
ctggtctgcagttggaaggaaggcaatgctatcacagacagttcactcctaaaccacaagctactgcagccttctcaagcatgagccaagccccattaatctgattcaaaacctacagagattgcatatcaatttctcaatgtaatcccattggagtttttcatgctagacttgggattaggggcaattgccccctttacacacactctctcgtttcataggttgaactcaacaagatagctcactgcaaagaagtcctctccaaattctacttatcacgcccaataatagccactttttagatcgttaaaaagatctaaaaaaatcgtgtggcttgagagtcattcaatcagtgaactgacttacaattcattttagaaggtgtcatctagtgtctttggcctcaattgaaagacaggacaagtcccattttaatgtcctgttataggaataacctaatgaaatatttgtacatacacaggttgaggttagatggcaacaagagtgttgtaacataggagctgcaatatygacagcttgcttaatccaaatagaaagtgattctgctaatacaggcaccttcaatttagctcacccttagaaatgaattgacattattcttt

SEQ ID NO: 53 - DNA, 501 bp, Homo sapiens
ttctgtattcttagctaaaatcccatctcaaatcacatcttttgaaagaagacttctgtgagtatgattaggggcttgaaggtgccatctataactcctccctgtgaagattctggccaattaagtttaggtaccaaaaataaaaaaaaaaaacccacaaaaaaacaataaataggaatggaagcatcactttatttgtttgggaaagcacatgtaaccatccagagctctgataatggctggaatagcamgacgctggagtggagaattctatttcctagcccagatctagatcttgaaacttacattcttaggtgtgccatgtgtgagaaagagcaaaaagaagagaggaatgaagagagatggccttcaaagctgactgagcctgctgcttagaaggtgacctcaagccttcccacattagaaataatgaagataatttctttcatcctacactaatcattctcttcttcaaacaccattttaacttagaggtaacat

SEQ ID NO: 54 - DNA, 601 bp, Homo sapiens
aaagtacagggagaatttggagttccttccaaactccgggtggaagaaaaggtccttgtacaactctggggtttcagcagcagaaattaatgtcttctgtagtccaactcatgaactcaactcagttctggtaaaaatcagtccccaggcctcacaataaggtataggagagagactctgactgatccagcctgggcaagatggataccttgcctggattgtccactgatggaccaagacacagagtcacatagtatggtcttgaccagcagagggttgaacagagtagcaaggctagaamtggaaatgctgacatccttttgagccaggcagtcagtgaagagagggctgcagaaagaagttttacctgtagcccagtgttgctaataaaatgttgaaaaactatctgagggcagccagacctgttcccacaagtgatagctggttttcatttggggatcttcactttgtgacaagccggatttctatttccatcagataaaggctggcaataatgaacatcttcctgataactctcatcacggttttccaaggagctgaaaatggtaattaatgcaaaagctgtgtttattggttcgtg

SEQ ID NO: 55 - DNA, 1001 bp, Homo sapiens
ggagtctccctctgttgcccaagctggattgcaatggcatgatctcggctcactgcaacctccacctcctgggttccacccatcctcctgcctcagcccccaagtagctgggactacaggtgtgcgccaccgcacctggctaatttttgtatttttagtagcgattgggtttctccatgttggccaagctggtttcaaactcctgacttcagcctcccaaagtgctgggattacatatgtgagtcaccctgcccagcctgtcttggttttttgatcgttaaataatctccctctcctcttgctttctctgttttctcactctgatataattactgatggatcttctaatttgcttatcttgtttgctgctactcctgtctcagtgtcctcttctgctttggggaagatttttttcaacgtcacccttcatatttagttttacggttgtgccatctttgtttcaatatccaggaggctctttcttgttctctgaatattaayttttttatcaccctcattcttacatgcatcttgactctcaccttttctgaggatattaactaatagcaagtagtttgttttcgacattttcttctgtttccttcaagttactcttatttgggctctgcatttcatcttagaggcttggcctctgatgcttagtgatccctggctattcatatttagacataaaacactaaatgtttcttgtaagttctcagtgctggtgggcttcaaaatagggtgaccgtgagcgttaattttatgtgtcaacatgactggactacaggatacacagacatttgatcaaacattgttcttagtgtttttggatgagtttaatgtttaaatccataaactgagtaaagcaaattgccccctctggaatgtgatgggccttatccaatctcaggcctgaatagaaaaaaagaccgaccctcctgcaagtaagagagaacgcctcctgcctgagaaggggttcagaacagggacattgtttt

SEQ ID NO: 56 - DNA, 1948 bp, Homo sapiens
ttctcaaactctatgggggcagattttatgtactgaagtgtttcatgatctactgtgtgttggactgttaatttgttctttttctttttctaaaattaatttcccttctcccttccttccttccttctttccttccttccttccctccttcccttccttccttccttcccttccttcctttcctccttcattcctttcctttcctttcttccttccttcttcccttctttcctttcctttttctcttctcttctcttctcttctctctttgagatggagtctcgcattgttgcttaggctggtctaagctctggcctcaagcagtcctcctgccttggcctcccaaagtgctgggattacagacaggagccactgtacctggcttaacttgttttttcaatgagactttgggacaacatactgagtttgggggttgtgtgagtcagacagattagcactatgctgtaatcataaataacacctaaatctcacctgtttaaatcagtaaagagttattttttcttttgctgcaatttcatccccgctttggcaggggaatctttttcacgtctcatgcagatatggaggctgacagggacctcactctctgttactggtctccatggcaggaagaaggaagggaatgtgataaattgtatgtggaatttttgtttgtttcattttgctttgctattattcaactttcttgaggtataattgaagtatcataaactgcatatatttaaaatgtaccttttgatgcattttgagatatgcatgtacctttgatatcctcaccatgatgaagagaataaacattttcataacccccaaaaggttccttgtttcacatgcctctctccactcctgtcttcatgcaaccagggacctgctatctgtcaatgtaggttgatctgcatgttccggaattttttttttttttttaataaacggaagcacacagcacttactcttcttctactggcttctttcatgcagctcaatgatttgaggaatcactcatgttgtatgtgcctcaacagttcattctgttttattgctgagtagtattccattgtatggctataccacaatttgtttatctattcctgcacatggaaagttggtttgtttctggtttrtaatgatcacaaatcaagctgataagaacatttgggtacaagtctttgtgtagacttgtgcttttatttatcttggatgcatacttaggagtgaagatgtcccaaagtggttgtaccatttcacattcccttagcagtttataagagttccagttgctccacatcttcaccaacacatggtatgatcagttgaaaaacttttcccagcactttgggaggccgaggtgggtggatcacaaggtcaggagatcgagaccatcctggctaacacagtgaaaccctgtctctattaaaaatacaaaaaattagctgggcatggtggcaggtgcctatagtcccagctacttgggaggctgaggcaggagaatggcgtgaacccgggaggcggagcttgcagtgagctgagattgcgccactgcactccagcctggaagacagagcgaggctccatctcgaaacaacaaaaaacacaacaaaaaactgttcacccttcaaataggtggatagtggtatctcagtgtagttttaatttgcattttcctaatgattaattatattgagcatttttaatgtgtttattggctgtattgtggagtatccttgggtgctgtgctccggggtttgctctggtgtgtcaacttggtagaatgacttttatccaatttttttccagtgaggcttctagttagtacaatatctacacactgtagcactagttagcatccccgttcctactttgtttttatctgaaccagtttagcccttccatttctctcctgacctgtggctagtttatgggctcccta

SEQ ID NO: 57 - DNA, 1125 bp, Homo sapiens
cctaagagagaaaggtgtcattgaaagaattccgtctgtaaaattattttggggtttcttaaattcacatagtcttgtacatagtttaatgtacatggccttggcattgtgtaaaatgttatttggacactctggaagaagaggatagcaaatttgattctagggttataagactggagggttaattataatataaatcartcaggtaattgggagaagattggaggaaatttgatacatgtttatgtgtttatcaagtgattgcatctgcttttcactctctgctgccaccatctggccccaaaacctttgtatgagccacaatagtcacatccctgggacatctttgtgccccctccagtctcttttccacacggcagccaatatcatttcttatgattctctactggttcatgtcattcctacacttggcatcactcattagttggactttggataaacagaaagcatcaagctcttgcctcctttccagcttcatctcatgtcttccctcactcgtaacgctctgcttatgccagccttctctctggcccttgactatatggagttctttccagcctcagggtatttgcaacctgctgttctgctacacaggacatctgccattcacactttctatctctcactcctgctcatcccttagatctcagctaaacataactttcttggatggcactcctttaaagcccagccacccgaccccagaatttaaattggcctctccccatccttaatctaatttccaacagggcatactgaccttttcctttgtataacttattataatttgtggtgtcatgttttctatatgcttgtttaacctctgtcttccccaatagacagttaactttataaagccatggacggtgtccatcttttttttttttttaagtgctgtactctgcctcagacaagatgtggttggtgcttaattaatagttgttgaatcaatgattacatcaacaaacgaacacttagtatgtgacaagcactgggctaagtgcataatactcatgattttatttaatcctcacaataaccttttgagttaattatcattaatgtcattcacaaatgaggaaataatacttcttcatgcagtga

SEQ ID NO: 58 - DNA, 605 bp, Homo sapiens
ttgattatgtttttccaggttcactttcttccttgggatgtcacagaactgaactctatcagaaatatgctagggcatgttttattctcttctgcatcctctgtcagaaacagccaacaagtgccattttcaagtcgctacagatctcacctagacccttggaaatatcaggttctgctggattctgatcaagagtgcccacgcagtaacttagaatctgaccagcatgccaggactcacatctagctcttcccaaaggcacacgtgggcagggattctcaaatcatctggggatgtcgataaaccacagatgytgactcagtgagtgtggggtgaggactgacactcttaatttccaaaagctcccaggtgccactgtgggtctttgggactgcattttgaatggaaaggtgctagggtatttttaatgacatcctaacgaaattatgccattagggatttaactgtgtattccacagctttactttccttttcaatttaacctctttagttcaacaatgtaacacttgtggaactccacaaattaggatgcaaaatgaacaagaaaagggatttagttttaactgagatgtcccttcacaaga

SEQ ID NO: 59 - DNA, 701 bp, Homo sapiens; identical to SEQ ID NO: 51

SEQ ID NO: 60 - DNA, 1948 bp, Homo sapiens; identical to SEQ ID NO: 56

SEQ ID NO: 61 - DNA, 622 bp, Homo sapiens
aagattgtaaggcagagaaaagaatgctgagccatgagaatggtaagaggctgatcaatggtgttgtgggacatgctaagaggtcttcacttcgctatattggggataggaagcagcaatgtgctaagacctacctgtcagcaaatattactgccatggtcccaaggccttaggcatgatagagctcaataaatgctgggtgaatgaacaaataaatgaatgcataagccatgaaataattggataaaattctctaggtgcataaggagaatgaattaaagcagtcaaggctagaagtaagaagaccatttaggaggttattgcatataagaaatgctaaggtatgaactayagcagtgtctgtaggactagaaaggagggaacaaattcaggagcaattcagaaaacagaattgatagagtcacagtcctcacatggccaccagcttatcttcctgaaagaacattgtttattaggccatttggctgaacaaaaacgttcagcagtttcccacaccttgtagaatatacatactgtaaagtttgctacctacattcacggtccttcatcagcttctagctctacatctgccccagaataactattcatcaaatgctgaata

SEQ ID NO: 62 - DNA, 914 bp, Homo sapiens
ttgaaacaaagaaagtagagagaatattatagtgaactcccacaaatctagaaacaagctttaatcattgtgaacatgctttcaatcttttctaatcaccccacttgtttttattgtatttacttatttatatattttaattaatttttgctgcaggattttaaagcaaattccagatatcataccacctctccactgaaaccagtatgttgtcctaaatgaaaatcatattaaaattttacataacaatatggaattaacctaacaattttgaccttaatcacttagtattatttgaatagtatttaatatctgcttcatatctgaaagtttccaattgaatcaaacaaaatattcttcttataccatatttgtttaattcagcatttaaataagcacaagtattaattttggttgttatgtcttctacatctattttactctccattttttttcttctttaatcatcaaaaccagccctaaagaatgtccaactttccrcatttagtttattgcttcttccttatatgagttaacttgttcctctatttcttccagatgttgcaaaagtagttagacctagaaaattaattagctaacagttcaattacgtctgtacatgataaattttagaggccatgcctcacactctccatttcatcacattttgaggcacttatgcatctgtggtctcttttggtgatgctgatatcactgggtaacgtgcagtctgaacttttctcataacattccccatcaactcttcacctaatgcttacatcatccactgatccatcgaatgttgtctaaattcattattacattacagatcgcaatacagtatttttctaattttatcatcccttatggagtcttttccaaaaattagaaagttaacctaaaagaaaactcag

SEQ ID NO: 63 - DNA, 582 bp, Homo sapiens
ggcagaggaggtccttcgaagctctctgggagcacaagcctctgagcagggatctgggtgaggggagcaaggggctctgtgaagacccagggaccagattccaggcagaagccctcagagcagaagtgggattgaagggctccaagagcagaataaagaccagtgtagccagactggggagaggggaggccccatctcacatgggttgcttctgaatgtgatgtggggcagtggcctgctatgatgktcttttaaaaagcactagaactgttgaatgaaatcctgatgccaggagggccagggtggaattggggagcccactgaggctgtggcaggagtctgggaagagtctgagaggggtgatgaggcagggaggtggcagatttgggagaaacgtaggaggcagagatacaaggaccagcagaagggaacagaggagcaaggagagacctgcttacagctgagccatcggggagagctggtgccttctgctgaagtaggatgaacatgtcttggggggaaatcaagacttctgctttttaaagttaagtgatcaggggtgggtggcagagtctcgaccta

SEQ ID NO: 64 - DNA, 1001 bp, Homo sapiens
ctcctatgccctgatggggaactctctagacagacaaatgaatagctgaggcttctcatcacccatatacccacattctctctctctttttttttttttttttaaacagagtctccttctgttgcccaggctggagtacagcctgggctcactggatcctctgcctcctgggttcaagtgattctcctgcctcagtctctcaagtaggtgggactacaggtgcatgccaccaggcccgactaatttttgtatttttagtagagacggggtttcaccatgatggccaggttggcctcgaactcctgacctcaagtgatcctcccaccttagcctcccaaagtgctaggattacaggcgtgagccaccatgcccggctgcacactctcttacctttcccacaccttgaaatttccctcactgcccaccctaaatacagtaccatgagacgcaaatcttagattagatttcaaaggaagggcagtgtcacttagttccactaargcctacctgtatcctctgtggcctagactggaggtggatatggggagactgatttccaagatagacatagctctgttatcttcactccctgagtccaagcccagctgaatacctaagtggcttctcattctttttttttttttttttgagacggagtcttgccctgtcgcccaggctggagtgcagtgtcatgatcttggctcactgcaacctctgcctcccaggttcaagcaattctcctgcctcagcctcttgagtagctgggattacaggtatgtgccaccccacctggctaatttttgtatttttagtagagacagggtttcaccatgttggccgggatggtctcgatctcctgacctcgtgatccacctgtctcggcctcccaaagtgctgggattacagatgtgagccaccacgcccagccgtggcttctcattcttatccttagagattgctggcatccccggcctcttaccactcttcccatccttccatca

SEQ ID NO: 65 - DNA, 753 bp, Homo sapiens
atacatgagatttctcatatatatatatttatatgacatcatgagattatagatctaatatataattttattatataatgttatatattatatttattatatataataatatatatttataatatataatctcatgatgtcatgtatatctgataatcactatgtgatatatgtgctcacacatacaaacacatttttttatgttgttatatgtatttcatattataatatgtatgtattcacaaaatttacccaaaagtgtattttccctagttccatatctttataacttgggtgcattctgatgtactctagtctatacttttttgtatttcatttcaaaaaatgcttgttataactgaatgcattgattttatatgctataatgggtctacccatagactgaaaaatttggctctagttgaagtttctcaaaaatcagtgtgtattagaaatacttgggaagctttttacaaagctaggttcctgaggcccctcccyctaagattctgacttgttgcatcatagaaaaggcccaggaacctacattttttggctcaatatatggaaaataaaaacaacagtgctcagtagacggtgctaaggtccatatgagaagttcacacagtgtgttaaatccatgttcagagtgaagagtgtttgctgtcctgaacagagtctgcactcctccaactgcacctctcaatgggtgattatttcaaagatgtcaagaaagaaaagccaactcataaa

SEQ ID NO: 66 - DNA, 620 bp, Homo sapiens
tctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgagcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactcccygcttcttcctcacctacttcctcttcactggatttgtcaactcacctgtctttacgcaatagttacaatgccagcatttctctacattacatacttcagcgattctcttactggctttgcaaagtcacccaaacacgaatggaaata

SEQ ID NO: 67 - DNA, 1001 bp, Homo sapiens
agtctaaggagttctccagctgagatattctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgagcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactcccygcttcttcctcacctacttcctcttcactggatttgtcaactcacctgtctttacgcaatagttacaatgccagcatttctctacattacatacttcagcgattctcttactggctttgcaaagtcacccaaacacgaatggaaatagtaaggtagagtttcagccatgagttgagttcatagctttagcaactgttaaatagctaatgtctactttctggagaataaatgctttgcaagaccctcggcaatgaaaccaaagaaagaatttaaatagcctcaccgaataattagtcagcttttcgaagtcatctcgtttctttttgttgctattgaaaaacttgacattcatgtcttccttgatggtctccacactcttttggatgctctggtcatctttaaagtttttaaaaagtttgaagtaaaaggagacaatttggctctgcattatttttctgtcactctccttggaaggaaagagcacaaacagaggatgatgt

SEQ ID NO: 68 - DNA, 28 bp, Homo sapiens
gtgaatgaagagttaatattttattagg

SEQ ID NO: 69 - DNA, 24 bp, Homo sapiens
ttcctttaaactccttaaatcctt

SEQ ID NO: 70 - DNA, 28 bp, Homo sapiens
gtgaatgaagagttaatattttattagg

SEQ ID NO: 71 - DNA, 19 bp, Homo sapiens
ggtgggtataatgggtttg

SEQ ID NO: 72 - DNA, 23 bp, Homo sapiens
ggatttaaggagtttaaaggaaa

SEQ ID NO: 73 - DNA, 24 bp, Homo sapiens
aaaacaatatactacacctcctct

SEQ ID NO: 74 - DNA, 16 bp, Homo sapiens
ttaaaaaatttgtgaa

SEQ ID NO: 75 - DNA, 30 bp, Homo sapiens
tttgggtgattttgtaaagttagtaagaga

SEQ ID NO: 76 - DNA, 24 bp, Homo sapiens
ccattaaaacaaacaacctctcat

SEQ ID NO: 77 - DNA, 20 bp, Homo sapiens
aaactacttctcaatactcc

SEQ ID NO: 78 - DNA, 20 bp, Homo sapiens
actacttctcaatagtccct

SEQ ID NO: 79 - DNA, 20 bp, Homo sapiens
actacttctcaatagtcccc

SEQ ID NO: 80 - DNA, 26 bp, Homo sapiens
tcagtactccctgtgcttcttcctca

SEQ ID NO: 81 - DNA, 26 bp, Homo sapiens
tcagtactccccgtgcttcttcctca

SEQ ID NO: 82 - DNA, 26 bp, Homo sapiens
tcagtactccccgtgcttcttcctca

SEQ ID NO: 83 - DNA, 801 bp, Homo sapiens
cgccggatgtttgggtcttctggagctctctcctgctcttctggtatatccacaaacagtcttgtcaaaactggatcctgatacttcatacaattacttgcttcgggacctcttgagattcatttctcgtttcttcatttcctcaccaaatgctggagtccattgagtgagaaggggctagcctcagccatgtgtttactttggaggcctagtcaagagcttttctagaacatcctgtgggaatcagtggacttgccccaatattttctcattattttcacactattcactgttcccattcaatgcggctgctgagctattattttgctttctgtctaggcactggtaaccctggctggagcgccatcttggctgattctccatatcttattgtccttccrtgactcacatgtgctactgtccgttaaccatgctctttgtcacaaagcacaattctcagcttccccccagctcctactccttctccccaacacttgcacttacagaaatgtttccttcccatttcccttttctgtatctaccatcttttatctgccctaatatctacaaaaatatcctcagttctcttgaagtaaaaatggacacttttaaaattgttcatgtaagtatggatgtatatatctataaatttatgtatacatatttatgtattcatggatattggtgaataaatgtcagggtagccctactttttacacccgcttctctggtcacaatacagatgaactgtctgcatttgggtgggaggcagtgagagaagcatcgattgattggcatgat

SEQ ID NO: 84 - DNA, 601 bp, Homo sapiens
ggcaatctctatgttgggactaagaatttgatcctgttcatgctttgtctttctacttccaccttcagtgcctttttctgttcttttccggtgctctcttttctctcttttctcctctcccccatgcctcttgtggtatcatgctacctcatccttcctatcatgaccaaggcatttgatgaactcattcctgccagctcttctcagagtctgagtcagacaggaccacagggagaaaagactcttgaatcatgggacttacatttgggttaaagcacagaccaaatgctgtatgtaagarcctacctctgcaaggctgcccctgctcccactattgcacccatcctgccaggggccgactgggctgacaccatcattcccgtcactcccaccactcccctcccctcccagaattgcaatagtatgtcaaatctgggaccccgatcagggatctgggaagttgagaaagcatccagttggcaaactaagatatgggtttcaccctaagaaaagagttttaaatgctggctgtgcatgaaaattgcctgggaaattttagaagaatgtaagcgcctgtgccccatctgaggtcaatcaggtc

SEQ ID NO: 85 - DNA, 1057 bp, Homo sapiens
  misc_feature (383)..(383): n is a, c, g, or t
gaaaaaaaaataaggtcgagtaagcattaagaatcaagaattagtggatgatttctaaaatagtaaaagtagaatgtatgttattaccttctaaaaggaggcaaaagaaaattctgaaagcaggtaaaagaaaatttgaccaattccacgagtgaggaagagagataaaaaaagaagcaaagtttaaaaatgcagacaagaggcagaaaccaaactggttactctgacagagagaatgtaatgcatagaattgctaactacctggagaaatgaaaaggcaaaatggaaacagtgagggatcgtggatgtagcaaaggcgggaagcagccgtcgcacctcggctaggagaacaaagggaagaagttggaattcctaacagtttrggggaaagccttgcagagcaaaacccgtgagaatcaagtgatgctcagcccggttgggatcccagagatcagagcggggtccctgtgaggctgagtccctgacctctgaagaggggagggacgctgggcaacaggcactcacggccctaagagagtgtgatgaggccagttctgagaatcttggaaaaaactgaaaactgatctcaattcctgctactggatgggactgccactgccaacctgaagacacggacaggtgtctgacgggaaccgaatgaaagaaagcagaactaggacaaaccaggaaggagcaagtcccttccccctcctccagcctgcagcggcccaatcagcaggacctcacagggagccaggcagcaaggcaggcattgatttgcaacgtcccagcccagcatctcggggtggaggatggcaggtttgggactgagaaaccaaagctccatactcggcacagcctgccctgggctttgcttccacacactcccctctgtgagctttggaactcccaagacatccatgtttcaacttaacaagttgtggggcctctttcataagaaatggaggcattcccattctctcccttgaatgaggagatacaatgtcccagtcatcaatgctgccatctcctgaaaacaggccactgtccatctctg

SEQ ID NO: 86 - DNA, 601 bp, Homo sapiens
aactctctgatgctgagactaaaagtaattcatcttcctaaagactttctatctttgggagggtgacatttcctgtctggcttttctgatgattcttacaacttaattacttttggcatgtccagttgcttgctttatttactacattgtataggtcaaacacttgaacctcaggtttatgtttgttttatcaaacttagagaatataattattcagagagtaattttcagattagcacaaacatacattttctactagagagtatctattcttaaagctcacttgtgagacattatattyttcctgttgtgaggatgagatggcattctctttgggatctaaattgcttctgaggcaggcacttttgtgtattgttatctctataccccgaagaacaactggagtgcaggaaaagcagggttttagacaaatgaaagtgggatatgccgtctgggctgatggctacgcttggcatcaccaaatgggagacttaagagccttctgcagtcacggctgcagtggctgattattcttccagccatgtctcttccttccttcttctcttctcttctctcctcttctctcctcttctctcttctc

SEQ ID NO: 87 - DNA, 996 bp, Homo sapiens
acacttggacggcacgtagtcggcgtaggtctctgtgtgccccagctcctccgcctcctcctcctcagcctcgtcctcctcctctggctggctctgcacactctgctcctgcgtgcgtggcgggagctcagcggagactgcaggcctgagcggccgcgggcactgggctcccccaggggcaggggccaggctgaccatgggcagctgagtgacccaggggtgggagacggctctgtctgcccctgcagcaaggtgctcccccagggcctccatctcctcagctataaaatgggcactcccagcttctaggccagccaagcgctccccacaatggagatgcctgtgctgagccctctccttaggtcccatgagagggcaggggagggagctctcggctgggcccagctcacctggtagctgacgagaagcggggtgctgtgggagggcagaaagtcctcgaagcctgcaaacggcctgctgagctggaacagctggaagggragcaggatgtcagcccgggccagacgggacaggtcacagctggtgggccctcgcagcctggccaccagagggaggctccccactgccccgtcctcgcctccccatcctcgctccccagggccctgggtcttgctctcctgctgttctcagggtgaccctggtgcctcccaagcctccccagtgggcacctccctggggcctaaggctcgctcctcctggccagcacctggctgccttctcactgatggccagtgtgcctgcctggagtgcccgggaggaaggtgcccggctagttctgacagacaacacagcttggtcccgcgggagctgccaggaaggctgtggacaggggtctgacaggagagacccactgtgtgccagccccatgcagtgcaggaggccctcgggggcctgggagacaaggggttgggagctccctgtcgagggggaagaggagacagaggcagtgggggcgggcgggcctgggccttgtcc

SEQ ID NO: 88 - DNA, 601 bp, Homo sapiens
aaggactgcagtccaggaggagggaacacaccagtggatgtggcccaggggttgaaatcgggaagggtggttctccaagaagaatccaggatctgtcaccaaaagagcaactctagtgaggcaacagaaacaacatatgttggcataattcataatgatgatgtttccgttattgggaaccctggggagacaggattttgtgtttttggagtgattttcttgactctctttaatgcctgccccatagatagccagcttctcatgagactgtttcttgtttctctttcttctctgccacagrcagggccgaggctgagccccaacccaagatgagcctttggtttagtagcctgggacctggacatttggaaagagacagatcagactggaagcctctccatccggcttccacagattacctttgtgttacttccttttcccctagtcaagacttgctttaagttttaaggtggtaggaaggagatattttagtaaagtatccgatgtatctttttggtgctatgctaattattaccaagagcctcagtggttttgactgcagcctccacattttgcaccaccatgatggggtttagcaata

SEQ ID NO: 89 - DNA, 951 bp, Homo sapiens
ggaaaactgtaatctcaaactacttccagagaagtacaactatcattcctgtctactctccactactttgtcagagcttttaaacattttactagcttgcaattttactgcgtggaagacttgcaagtcatctgcaagggtgaggtttccctgtgtgctcactcattttaggtcatggagaagtattcatttgtcctgatggttggttttggtctccgggtttcctgtctagacccgctaaatgcctactggtctcatggctgcattttgaagtcatcaggttagacttggggcatgtttgaagtaggatctgaatccaggggaggcaatggcttcctgacaagggtagtttctcaaaacctgggattttcactcctgcaagttaggagtgggcatgatgagaatataaaatcatgaagcaactcaaatcagacttacttagtatactagaacaaggagggtatcccttgatgttggcagggacaaagaaagtgaatttkctgtttaaatatcattaaggaagtgtgtggaagtcagtcacgaaggggtggtgcagactaaaacaccaaaaggttcaaatgagctctgagtcaatcaatgcgtgattaataagtaaggaatcacttcagggaattaggggtgtttcttttattaaaaaaaattaaaaatagaactatcatacaatccagcaatcttactactgggtatatatccaaaggaaataaaatgagtatgcggaagagatatctgcacccccccaccccgtttattgccgcactattcataatagccaagatgtagaacaaacctaagtgtccaacagtgaatgaatgggtgaagaagatgtggtgcatatacacaatggaatactattcagctatataaaagaaggaaatgttgttctttgtgacaacatgaatgaatctggaggacgctatgctaaatgaaata

SEQ ID NO: 90 - DNA, 601 bp, Homo sapiens; identical to SEQ ID NO: 1
  misc_feature (301)..(301): n is a, c, g, or t

SEQ ID NO: 91 - DNA, 746 bp, Homo sapiens
tgagtgtccccatctgggtccttgctcagtgagagccagactttctgctataaccaggccctgaaygagagagaaccagagtcatgtgctgtgcaccaaatgcagtgggaagacacgagggatgcctcggtgaccttcttttcaaacaggagtctttcgctggtaacctgcctctgaccttccatcagccagatgttatcttcccaaggcaacagtgttccagaattctttctgaggtggccctggcagcttctgggtccttctattctgatttccagttttctttttccatttatttatttatttatttatttatttatttatttatttattaataagggtcttgctttgttgcccaggctggagtgcagtggcacgaacacagctcactgcagcctcaaactcctgggctccagtgatcctcccgccccagcctcctgtgtagctgggactacgggcatgcaccaccatgcctggctaactttttatagagatggggtctcactatgttgcccaagctggtctcaaactagcagactcagtgattctcctgcctcagctttctgagtagctgggaccacaagtgagcaccattatgcctggctaatttttatagagatagggtctcactatgttgcccaagctggcctagactagtgggacagtgactcactcactcggactgatcgctggcatgtgtgattagagtgtgcatgtatgcaaccctgcaggaaggccgccgtggt

SEQ ID NO: 92 - DNA, 805 bp, Homo sapiens
ctgctgtgctggtgggagggccactggccaacctgctctcacccagcttgggctggccatagtgccaggcagttgccctcagctcctctgcccattccagccatggaggtggctaaccttgcaggctccagggcgtgatgggagtgtccaggaggcagaggccagagggggtcatgctgtagggggagttcctctctcagaccccatctgtgcctcccccgctgcgtatctgggtgacatggatgccaacgggcaggagcaagggctgggaaaggctgcggaggggccctcggctggtctggtggggagcggggtcactgtgtctctgctgcctccctgggggatccagcgtggggatgggacatggagctgggcacctctcctggatgcccctgggctgatccccctcgatgccctgacgagagccctggcccagcgtctgagaaggagtgggagctgggcccgggtgggggcaggagccggcatcgctctcagcactgccccacactccccagcctgcctgctgaggccatgtcggactacgagaacgatgacgagtgctggarcgtcctggaaggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtcagtgttgccccgcggggccccagccccaaacccaccacagaatcagctcggaagtggctgcacgctttgccaccctgacccttaacc

SEQ ID NO: 93 - DNA, 801 bp, Homo sapiens
ttatatgcctcggtttcctcatctaataaaaaagcaaaaagtaattaggctgagttacctatgcttggtctgttgaagtagtttgaatttttctgaatcaggatgaatgcaaattgcctcattatttcactaattaataatgtctgcttggaaatgcaactatcagtgcacaataaatgataaagcttataattatagatgagagcaattgcttctttttttgttcttaccttcatttcagtttatcttcctgagctatagctacctctgtaagggttaggaaaacatttaaaatatctaaacagtgattcctttctcaagcgttcggaaattgatgaatcatcttactctgcagcattcaaagtaactgtgtgcttgacacataggaggaaaagtgggcyctttgaaatgtgagcagagcaccgatcgtgtcacctgcccggcgcgtgacctgttcactttccacctgaccctctgtgcccaatgtcacctggagcctgaaacctggaagggaaaagtccttgactggagtcagtggagagaaaatcatcacctgcttgggcacaaacatgcctggtgactgcgtcactgtctgttaagtgggatcagctaatagttgcacagcgggccttcaaagcatcacggcagaactgatgaagttcctgtaatgcattccctgtgtgaaggccttggactttagagctcaagtgcgccaggagcattttgagcttgccgacaggctctcccatggattctatttggttatacggcagtggaaaagaattgactttcattttcgtc